(19) United States
(12) Patent Application Publication
(10) Pub. No.: US 2025/0268877 A1
(43) Pub. Date: Aug. 28, 2025

(54) 2H-INDAZOLE DERIVATIVES AS IRAK4 INHIBITORS AND THEIR USE IN THE TREATMENT OF DISEASE

(71) Applicant: BIOGEN MA INC., Cambridge, MA (US)

(72) Inventors: Emily Anne Peterson, Belmont, MA (US); Magnus Pfaffenbach, Chelsea, MA (US); Fang Gao, Somerville, MA (US); Philippe Bolduc, Weymouth, MA (US); Zhili Xin, Lexington, MA (US); Ryan Evans, Somerville, MA (US)

(21) Appl. No.: 18/269,086

(22) PCT Filed: Dec. 21, 2021
(86) PCT No.: PCT/US2021/064651

§ 371 Date: Jun. 22, 2023

Related U.S. Application Data
(60) Provisional application No. 63/128,967, filed on Dec. 22, 2020.

(51) Int. Cl. A61K 31/4439; A61K 31/444; A61K 31/501; C07D 405/14; C07D 471/04

(52) U.S. Cl. CPC A61K 31/4439; A61K 31/444; A61K 31/501; C07D 405/14; C07D 471/04

(57) ABSTRACT

This disclosure relates to 2H-indazole derivatives of formula (I), or pharmaceutically acceptable salts thereof, in which all of the variables are as defined in the specification, capable of modulating the activity of IRAK4. The disclosure further provides methods to their preparation, to their medical use, in particular to their use in the treatment and management of diseases or disorders including inflammatory disease, autoimmune disease, cancer, cardiovascular disease, a disease of the central nervous system, disease of the skin, an ophthalmic disease and condition, and a bone disease.

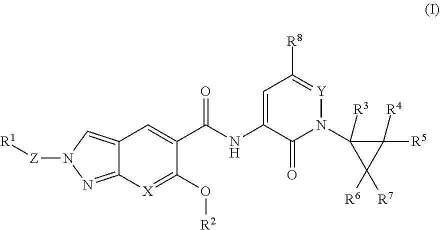

### RELATED APPLICATIONS

This application is a 35 U.S.C. § 371 national stage filing of International Application No. PCT/US2020/057921, filed on Oct. 29, 2020, which in turn claims priority to U.S. Provisional Application No. 62/927,809, filed on Oct. 30, 2019. The entire contents of each of the foregoing applications are expressly incorporated herein by reference.

### FIELD OF THE INVENTION

The present disclosure relates to 2H-indazole derivatives and pharmaceutically acceptable salts thereof, compositions of these compounds, either alone or in combination with at least one additional therapeutic agent, processes for their preparation, their use in the treatment of diseases, their use, either alone or in combination with at least one additional therapeutic agent and optionally in combination with a pharmaceutically acceptable carrier, for the manufacture of pharmaceutical preparations, use of the pharmaceutical preparations for the treatment of diseases, and a method of treatment of said diseases, comprising administering the 2H-indazole derivatives to a mammal, especially a human.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is the protein kinase family.

Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues.

Kinases are important therapeutic targets for the development of anti-inflammatory drugs (Cohen, 2009. Current Opinion in Cell Biology 21, 1-8), for example kinases that are involved in the orchestration of adaptive and innate immune responses. Kinase targets of particular interest are members of the IRAK family. The interleukin-1 receptor-associated kinases (IRAKs) are critically involved in the regulation of intracellular signaling networks controlling inflammation (Ringwood and Li, 2008. Cytokine 42, 1-7). IRAKs are expressed in many cell types and can mediate signals from various cell receptors including toll-like receptors (TLRs). IRAK4 is thought to be the initial protein kinase activated downstream of the interleukin-1 (IL-1) receptor and all toll-like-receptors (TLRs) except TLR3, and initiates signaling in the innate immune system via the rapid activation of IRAK1 and slower activation of IRAK2. IRAK1 was first identified through biochemical purification of the IL-1 dependent kinase activity that co-immunoprecipitates with the IL-1 type 1 receptor (Cao et al., 1996. Science 271(5252): 1128-31). IRAK2 was identified by the search of the human expressed sequence tag (EST) database for sequences homologous to IRAK1 (Muzio et al., 1997. Science 278(5343): 1612-5). IRAK3 (also called IRAKM) was identified using a murine EST sequence encoding a polypeptide with significant homology to IRAK1 to screen a human phytohemagglutinin-activated peripheral blood leukocyte (PBL) cDNA library (Wesche et al., 1999. J. Biol. Chem. 274(27): 19403-10). IRAK4 was identified by database searching for IRAK-like sequences and PCR of a universal cDNA library (Li et al., 2002. Proc. Natl. Acad. Sci. USA 99(8):5567-5572). Many diseases are associated with abnormal cellular responses triggered by kinase-mediated events.

Many diseases and/or disorders are associated with abnormal cellular responses triggered by kinase-mediated events. These diseases and/or disorders include, but are not limited to, cancers, allergic diseases, autoimmune diseases, inflammatory diseases and/or disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, neurological and neurodegenerative diseases and/or disorders, chronic inflammatory demyelinating neuropathies, cardiovascular, vascular or heart diseases, epilepsy, ischemic stroke, ophthalmic diseases, ocular diseases, asthma, Alzheimer's disease, amyotrophic lateral sclerosis, Parkinson's disease, traumatic brain injury, chronic traumatic encephalopathy and hormone-related diseases.

In view of the above, IRAK4 inhibitors are considered to be of value in the treatment and/or prevention for multiple therapeutic indications over a wide range of unmet needs.

### BRIEF DESCRIPTION OF THE FIGURES

FIG. 1: A schematic of plasma and PBS buffer solution including volumes to RED plated and aliquoted to crash plates.

### SUMMARY OF THE INVENTION

Compounds of the present disclosure are potent and brain penetrant IRAK4 inhibitors. Specifically, including a cyclopropyl pyridone moiety in the compounds of the present disclosure surprisingly result in dramatic increase in potency against IRAK4 (e.g, high potency in the IRAK4 biochemical assay and longer binding half life in Surface Plasmon Resonance (SPR) binding assay as described in the Examples). The compounds of the present disclosure have the desirable potency, solubility and brain penetrating properties.

In a first aspect, the present disclosure relates to a compound of formula (I):

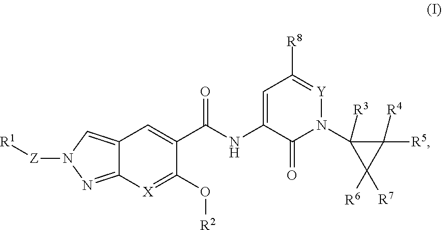

or a pharmaceutically acceptable salt thereof, wherein:
X is CH, CF or N;
Y is CH or N;
Z is ring A or —CH₂-ring A-*, wherein -* indicates the point of connection to R¹;

Ring A is

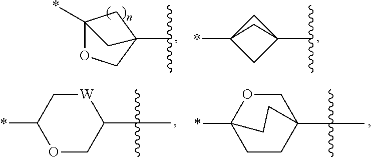

wherein n is 1 or 2; W is absent, CH₂ or O, and -* indicates the point of connection to R¹;
R¹ is H, —CN, C₁₋₃alkoxy or C₁₋₃alkyl optionally substituted with 1 to 3 substituents independently selected from halo and C₁-C₃alkoxy; or
R¹—Z is

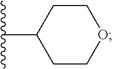 [SMILES: *C1CCOCC1]

R² is C₃₋₆cycloalkyl or C₁₋₄alkyl, wherein the C₃₋₆cycloalkyl or C₁₋₄alkyl is optionally substituted with 1 to 3 halo; and
R³, R⁴, R⁵, R⁶ and R⁷ are each independently selected from H, halo, CN, C₁₋₄alkyl, C₁₋₄haloalkyl, C₁₋₄alkoxy, and C₁₋₄alkoxyC₁₋₄alkyl, or any two of R³, R⁴, R⁵, R⁶ and R⁷ together with the carbon atoms from which they are attached form a C₃₋₆cycloalkyl or a 4 to 6 membered heterocyclyl containing one or two heteroatoms independently selected O, N, and S; and
R⁸ is H or halo.

Another aspect of the disclosure relates to pharmaceutical compositions comprising compounds of formula (I) or pharmaceutically acceptable salts thereof, and a pharmaceutical carrier. Such compositions can be administered in accordance with a method of the present disclosure, typically as part of a therapeutic regimen for the treatment or prevention of conditions and disorders related to interleukin-1 receptor-associated kinases activity. In certain embodiments, the pharmaceutical compositions may additionally comprise further one or more therapeutically active ingredients or therapeutic agents suitable for the use in combination with the compounds of the invention. In certain embodiments, the compounds or the pharmaceutical compositions of the present disclosure can be used in combination with one or more additional therapeutically active ingredients or therapeutic agents in a method of present disclosure. In some embodiments, the further or additional therapeutically active ingredient or therapeutic agent is an agent that can be used for the treatment of autoimmune diseases, inflammatory diseases, bone diseases, metabolic diseases, neurological and neurodegenerative diseases, cancer, cardiovascular diseases, allergies, asthma, Alzheimer's disease, and hormone-related diseases.

Another aspect of the present disclosure relates to the pharmaceutical combinations comprising compounds of the invention and other therapeutic agents for the use as a medicament in the treatment of patients having disorders related to interleukin-1 receptor-associated kinases activity. Such combinations can be administered in accordance with a method of the invention, typically as part of a therapeutic regiment for the treatment or prevention of autoimmune diseases, inflammatory diseases, bone diseases, metabolic diseases, neurological and neurodegenerative diseases, cancer, cardiovascular diseases, allergies, asthma, Alzheimer's disease, and hormone-related diseases. Also provided in the present disclosure are compounds or pharmaceutical compositions described herein for use in the treatment of patients having disorders related to interleukin-1 receptor-associated kinases activity. Uses of the compounds or pharmaceutical compositions described herein for the manufacture of a medicament for treating patients having disorders related to interleukin-1 receptor-associated kinases activity are also included in the present disclosure.

### DETAILED DESCRIPTION OF THE INVENTION

The present disclosure provides compounds and pharmaceutical compositions thereof that may be useful in the treatment or prevention of conditions and/or disorders through mediation of IRAK4 function. In some embodiments, the compounds of present disclosure are IRAK4 inhibitors.

In a first embodiment, the present disclosure provides a compound of formula (I):

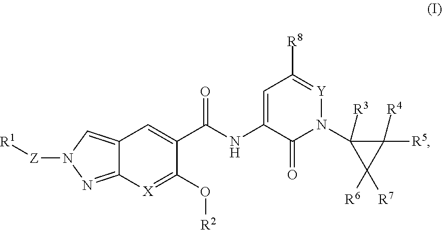

or a pharmaceutically acceptable salt thereof, wherein the variables in formula (I) are as defined in the first aspect above.

In a second embodiment, for the compound of formula (I) described in the first embodiment, or a pharmaceutically acceptable salt thereof, X is CH; and the remaining variables are as described in the first embodiment.

In a third embodiment, for the compound of formula (I) described in the first embodiment, or a pharmaceutically acceptable salt thereof, X is N; and the remaining variables are as described in the first embodiment.

In a fourth embodiment, for the compound of formula (I), or a pharmaceutically acceptable salt thereof, Y is CH; and the remaining variables are as described in the first, second or third embodiment.

In a fifth embodiment, for the compound of formula (I), or a pharmaceutically acceptable salt thereof, Y is N; and the remaining variables are as described in the first, second or third embodiment.

In a sixth embodiment, for the compound of formula (I), or a pharmaceutically acceptable salt thereof, Z is ring A, ring A is

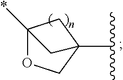

and the remaining variables are as described in the first, second, third, fourth or fifth embodiment.

In a seventh embodiment, for the compound of formula (I), or a pharmaceutically acceptable salt thereof, Z is ring A, ring A is

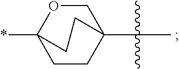 [SMILES: *C12CCC(*)(CC1)OC2]

and the remaining variables are as described in the first, second, third, fourth or fifth embodiment.

In an eighth embodiment, for the compound of formula (I), or a pharmaceutically acceptable salt thereof, ring A is

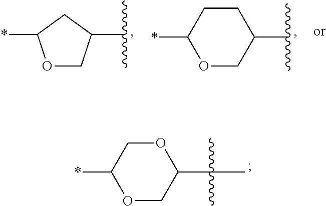

and the remaining variables are as described in the first, second, third, fourth or fifth embodiment. In some embodiments, for compounds of the eighth embodiment, Z is —CH₂-ring A-*. In some embodiments, for compounds of the eighth embodiment, Z is ring A.

In a ninth embodiment, the compound of the present disclosure is represented by Formula (II), (III), (IV) or (V):

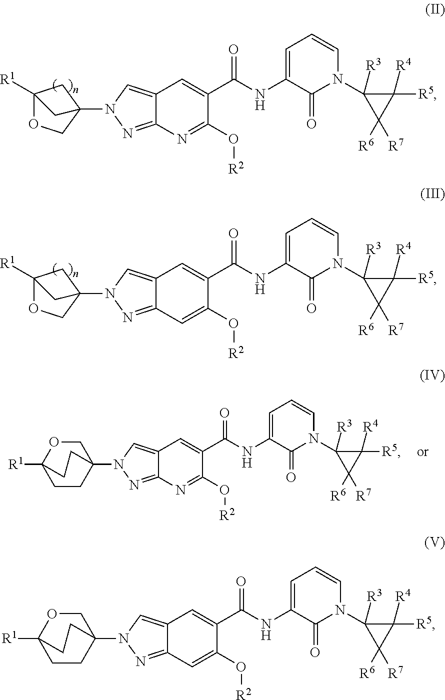

or a pharmaceutically acceptable salt thereof, wherein the variables R¹, R², R³, R⁴, R⁵, R⁶, R⁷ and n depicted in Formula (II), (III), (IV) or (V) are as described in the first embodiment.

In a tenth embodiment, the compound of the present disclosure is represented by Formula (IIA), (IIB), (IIIA), or (IIIB):

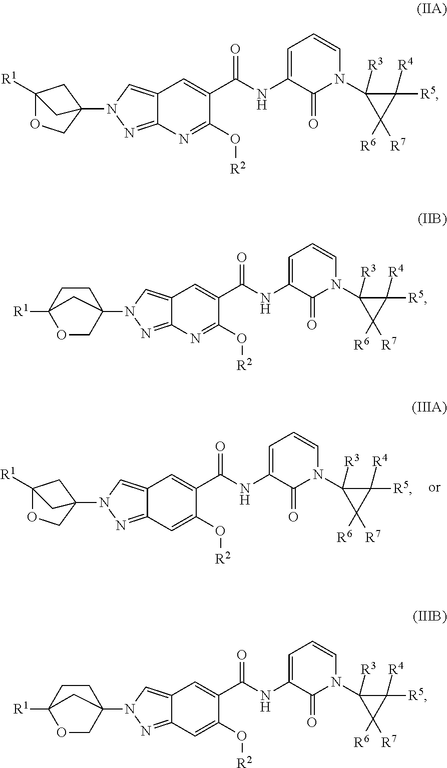

or a pharmaceutically acceptable salt thereof, the variables R¹, R², R³, R⁴, R, R⁶ and R⁷ depicted in Formula (A), (JIB), (IIA) or (IIIB) are as described in the first embodiment.

In an eleventh embodiment, for compounds of formula (I), (II), (III), (IV), (V), (IIA), (IIB), (IIIA) or (IIIB), or a pharmaceutically acceptable salt thereof, R¹ is H or C₁₋₃alkyl optionally substituted with 1 to 3 substituents independently selected from halo or C₁-C₃alkoxy; and the remaining variables are as described in the first, second, third, fourth, fifth, sixth, seventh, eighth, ninth or tenth embodiment.

In a twelfth embodiment, for compounds of formula (I), (II), (III), (IV), (V), (IIA), (IIB), (IIIA) or (IIIB), or a pharmaceutically acceptable salt thereof, R¹ is C₁₋₃alkyl; and the remaining variables are as described in the first, second, third, fourth, fifth, sixth, seventh, eighth, ninth or tenth embodiment.

In a thirteenth embodiment, for compounds of formula (I), (II), (III), (IV), (V), (IIA), (JIB), (IIIA) or (IIIB), or a pharmaceutically acceptable salt thereof, R¹ is C₁₋₃alkyl optionally substituted with 1 or 2 substituents independently selected from halo and C₁-C₃alkoxy; and the remaining variables are as described in the first, second, third, fourth, fifth, sixth, seventh, eighth, ninth or tenth embodiment.

In a fourteenth embodiment, for compounds of formula (I), (II), (III), (IV), (V), (IIA), (IIB), (IIIA) or (IIIB), or a pharmaceutically acceptable salt thereof, R¹ is H, —CH₃, —CH₂F, or —CH₂OCH₃; and the remaining variables are as described in the first, second, third, fourth, fifth, sixth, seventh, eighth, ninth or tenth embodiment.

In a fifteenth embodiment, for compounds of formula (I), (II), (III), (IV), (V), (IIA), (IIB), (IIIA) or (IIIB), or a pharmaceutically acceptable salt thereof, R¹ is —CH₃; and the remaining variables are as described in the first, second, third, fourth, fifth, sixth, seventh, eighth, ninth or tenth embodiment.

In a sixteenth embodiment, for compounds of formula (I), (II), (III), (IV), (V), (IIA), (IIB), (IIIA) or (IIIB), or a pharmaceutically acceptable salt thereof, R¹ is —CH₃, —CH₂F, or —CH₂OCH₃; and the remaining variables are as described in the first, second, third, fourth, fifth, sixth, seventh, eighth, ninth or tenth embodiment.

In a seventeenth embodiment, for compounds of formula (I), (II), (III), (IV), (V), (IIA), (IIB), (IIIA) or (IIIB), or a pharmaceutically acceptable salt thereof, R² is C₃₋₄alkyl or C₃-4cycloalkyl, wherein the C₃₋₄alkyl is optionally substituted with 1 to 3 fluoro; and the remaining variables are as described in the first, second, third, fourth, fifth, sixth, seventh, eighth, ninth, tenth, eleventh, twelfth, thirteenth, fourteenth, fifteenth, or sixteenth embodiment.

In an eighteenth embodiment, for compounds of formula (I), (II), (III), (IV), (V), (IIA), (IIB), (IIIA) or (IIIB), or a pharmaceutically acceptable salt thereof, R² is C₃₋₄alkyl; and the remaining variables are as described in the first, second, third, fourth, fifth, sixth, seventh, eighth, ninth, tenth, eleventh, twelfth, thirteenth, fourteenth, fifteenth, or sixteenth embodiment

In a ninteenth embodiment, for compounds of formula (I), (II), (III), (IV), (V), (IIA), (IIB), (IIIA) or (IIIB), or a pharmaceutically acceptable salt thereof, R² is —CH(CH₃)₂, —CH(CH₃)CH₂CH₃, or cyclobutyl; and the remaining variables are as described in the first, second, third, fourth, fifth, sixth, seventh, eighth, ninth, tenth, eleventh, twelfth, thirteenth, fourteenth, fifteenth, or sixteenth embodiment

In a twentieth embodiment, for compounds of formula (I), (II), (III), (IV), (V), (IIA), (IIB), (IIIA) or (IIIB), or a pharmaceutically acceptable salt thereof, R² is —CH(CH₃)₂, —CH(CH₃)CH₂CH₃, cycopropyl, or cyclobutyl; and the remaining variables are as described in the first, second, third, fourth, fifth, sixth, seventh, eighth, ninth, tenth, eleventh, twelfth, thirteenth, fourteenth, fifteenth, or sixteenth embodiment

In a twenty-first embodiment, for compounds of formula (I), (II), (III), (IV), (V), (IIA), (IIB), (IIIA) or (IIIB), or a pharmaceutically acceptable salt thereof, R² is —CH(CH₃)₂; and the remaining variables are as described in the first, second, third, fourth, fifth, sixth, seventh, eighth, ninth, tenth, eleventh, twelfth, thirteenth, fourteenth, fifteenth, or sixteenth embodiment.

In a twenty-second embodiment, for compounds of formula (I), (II), (III), (IV), (V), (IIA), (IIB), (IIIA) or (IIIB), or a pharmaceutically acceptable salt thereof, R¹ is H or C₁₋₃alkyl optionally substituted with 1 to 3 substituents independently selected from halo or C₁-C₃alkoxy; R² is C₃₋₄alkyl; and the remaining variables are as described in the first, second, third, fourth, fifth, sixth, seventh, eighth, ninth or tenth embodiment.

In a twenty-third embodiment, for compounds of formula (I), (II), (III), (IV), (V), (IIA), (JIB), (IIIA) or (IIIB), or a pharmaceutically acceptable salt thereof, R³, R⁴, R⁵, R⁶ and R⁷ are each independently selected from H, halo, and C₁₋₃alkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-fourth embodiment, for compounds of formula (I), (II), (III), (IV), (V), (IIA), (IIB), (IIIA) or (IIIB), or a pharmaceutically acceptable salt thereof, R³, R⁴, R⁵, R⁶ and R⁷ are each independently selected from H, F, and —CH₃; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-fifth embodiment, for compounds of formula (I), (II), (III), (IV), (V), (IIA), (JIB), (IIIA) or (IIIB), or a pharmaceutically acceptable salt thereof, R³, R⁴, R⁵, R⁶ and R⁷ are all H; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-sixth embodiment, for compounds of formula (I), (II), (III), (IV), (V), (IIA), (IIB), (IIIA) or (IIIB), or a pharmaceutically acceptable salt thereof, R³, R⁵, R⁶ and R⁷ are all H, and R⁴ is H, F, or —CH₃; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-seventh embodiment, the compound of present disclosure is represented by the following formula:

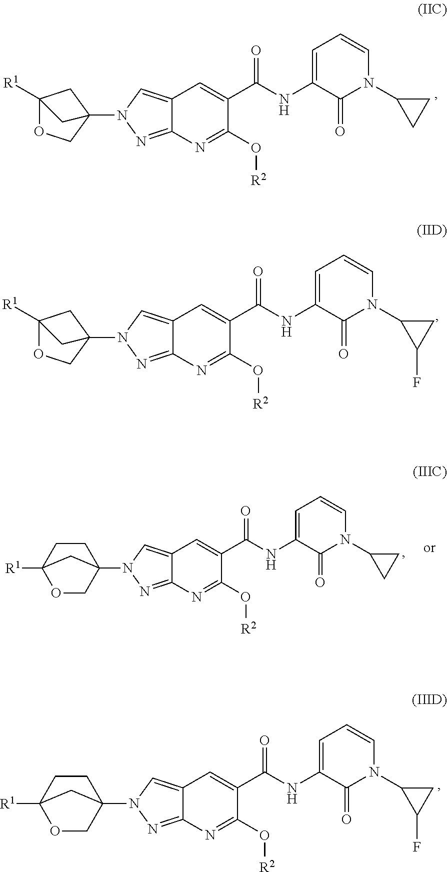

or a pharmaceutically acceptable salt thereof, wherein R¹ is C₁₋₃alkyl and R² is C₃₋₄alkyl.

In a twenty-eight embodiment, the compound of present disclosure is represented by the following formula:

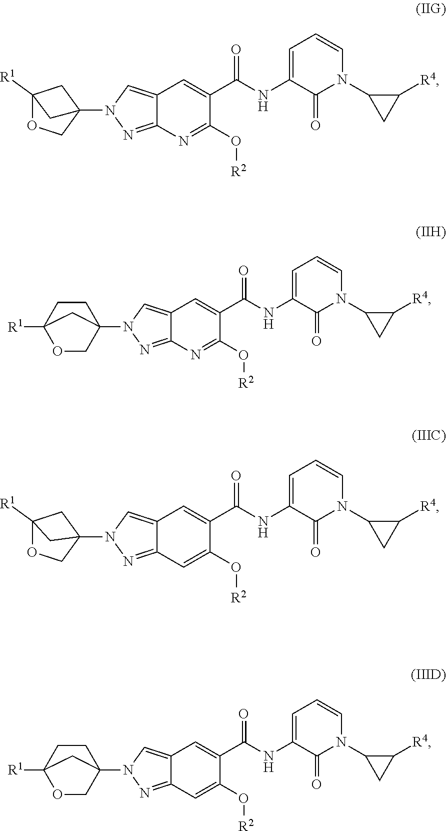

or a pharmaceutically acceptable salt thereof, wherein R¹ is C₁₋₃alkyl optionally substituted with 1 or 2 substituents independently selected from halo or C₁-C₃alkoxy; R² is C₃₋₄alkyl; and R⁴ is H, halo or C₁₋₃alkyl.

In a twenty-ninth embodiment, the compound of present disclosure is represented by the following formula:

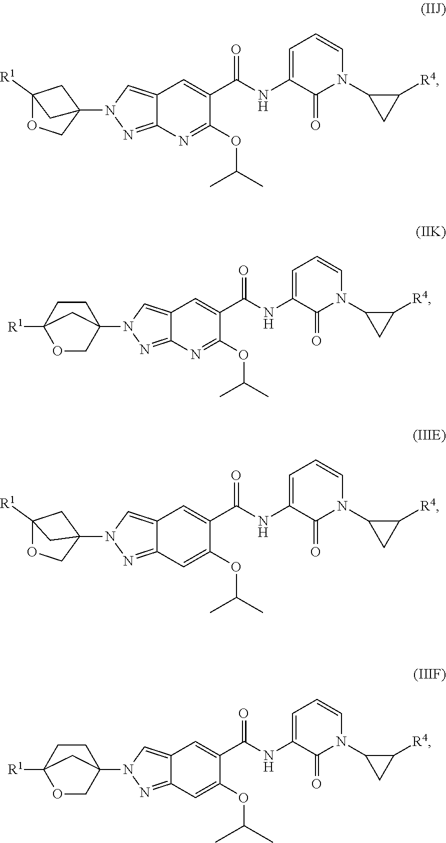

or a pharmaceutically acceptable salt thereof, wherein R¹ is C₁₋₃alkyl optionally substituted with 1 or 2 substituents independently selected from halo or C₁-C₃alkoxy and R⁴ is H, halo or C₁₋₃alkyl.

In a thirtieth embodiment, for compounds of formula (IIG), (IIH), (IIIC), (IIID), (IIJ), (IIK), (IIIE), (IIIF), or a pharmaceutically acceptable salt thereof, R¹ is —CH₃, —CH₂F, or —CH₂OCH₃; and R⁴ is H, F, or —CH₃.

In a thirty-first embodiment, the present disclosure provides a compound described herein (e.g., a compound of any one Examples 1-97) or a pharmaceutically acceptable salt thereof.

In a thirty-second embodiment, the present disclosure provides a compound selected from the group consisting of:
6-cyclobutoxy-N-(1-cyclopropyl-2-oxo-1,2-dihydropyridin-3-yl)-2-(1-methyl-2-oxabicyclo[2.1.1]hexan-4-yl)-2H-indazole-5-carboxamide;
N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide;
N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide;
(S)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide;
(R)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide;
6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-pyrazolo[3,4-b]pyridine-5-carboxamide;
6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide;
N-(1-cyclopropyl-2-oxo-1,2-dihydropyridin-3-yl)-6-isopropoxy-2-(1-methyl-2-oxabicyclo[2.1.1]hexan-4-yl)-2H-indazole-5-carboxamide;
N-(1-cyclopropyl-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(1-cyclopropyl-2-oxo-1,2-dihydropyridin-3-yl)-2-(1-(fluoromethyl)-2-oxabicyclo[2.1.1]hexan-4-yl)-6-isopropoxy-2H-pyrazolo[3,4-b]pyridine-5-carboxamide;
N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide;
N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
(R)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
(S)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
(S)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide;
(R)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide;
6-Isopropoxy-2-(1-methyl-2-oxabicyclo[2.1.1]hexan-4-yl)-N-(1-(1-methylcyclopropyl)-2-oxo-1,2-dihydropyridin-3-yl)-2H-pyrazolo[3,4-b]pyridine-5-carboxamide;
2-(1-(Fluoromethyl)-2-oxabicyclo[2.1.1]hexan-4-yl)-6-isopropoxy-N-(1-(1-methylcyclopropyl)-2-oxo-1,2-dihydropyridin-3-yl)-2H-pyrazolo[3,4-b]pyridine-5-carboxamide;
(R)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide;
(S)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide;
(R)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
(S)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
6-cyclobutoxy-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
6-cyclobutoxy-N-(1-(cis-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
6-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
6-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
2-(2-oxabicyclo[2.1.1]hexan-4-yl)-N-(1-cyclopropyl-2-oxo-1,2-dihydropyridin-3-yl)-6-isopropoxy-2H-pyrazolo[3,4-b]pyridine-5-carboxamide;
N-(1-cyclopropyl-2-oxo-1,2-dihydropyridin-3-yl)-6-isopropoxy-2-(1-(methoxymethyl)-2-oxabicyclo[2.2.1]heptan-4-yl)-2H-pyrazolo[3,4-b]pyridine-5-carboxamide;
N-(1-cyclopropyl-2-oxo-1,2-dihydropyridin-3-yl)-6-isopropoxy-2-((1S,4S)-1-(methoxymethyl)-2-oxabicyclo[2.2.1]heptan-4-yl)-2H-pyrazolo[3,4-b]pyridine-5-carboxamide;
N-(1-cyclopropyl-2-oxo-1,2-dihydropyridin-3-yl)-6-isopropoxy-2-((1R,4R)-1-(methoxymethyl)-2-oxabicyclo[2.2.1]heptan-4-yl)-2H-pyrazolo[3,4-b]pyridine-5-carboxamide;
Cis-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-[1-[(1R,2S)-2-fluorocyclopropyl]-2-oxo-3-pyridyl]-6-isopropoxy-2-(1-methyl-2-oxabicyclo[2.1.1]hexan-4-yl)pyrazolo[3,4-b]pyridine-5-carboxamide;
N-[1-[(1S,2R)-2-fluorocyclopropyl]-2-oxo-3-pyridyl]-6-isopropoxy-2-(1-methyl-2-oxabicyclo[2.1.1]hexan-4-yl)pyrazolo[3,4-b]pyridine-5-carboxamide;
Cis-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide;
N-(1-((1R,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide;
N-(1-((1S,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide;
Trans-6-isopropoxy-2-(1-methyl-2-oxabicyclo[2.1.1]hexan-4-yl)-N-(1-(2-methylcyclopropyl)-2-oxo-1,2-dihydropyridin-3-yl)-2H-pyrazolo[3,4-b]pyridine-5-carboxamide;
(Trans)-6-isopropoxy-2-(1-methyl-2-oxabicyclo[2.1.1]hexan-4-yl)-N-(1-(2-methylcyclopropyl)-2-oxo-1,2-dihydropyridin-3-yl)-2H-indazole-5-carboxamide;
6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-indazole-5-carboxamide;
6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-indazole-5-carboxamide;
(Trans)-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(1-((1S,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(1-((1R,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(2-cyclopropyl-3-oxo-2,3-dihydropyridazin-4-yl)-6-isopropoxy-2-(1-methyl-2-oxabicyclo[2.1.1]hexan-4-yl)-2H-indazole-5-carboxamide;
N-(2-cyclopropyl-3-oxo-2,3-dihydropyridazin-4-yl)-6-isopropoxy-2-(1-methyl-2-oxabicyclo[2.1.1]hexan-4-yl)-2H-pyrazolo[3,4-b]pyridine-5-carboxamide;
N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-pyrazolo[3,4-b]pyridine-5-carboxamide;
N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-pyrazolo[3,4-b]pyridine-5-carboxamide;
N-(1-((1S,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide;
N-(1-((1S,2R)-2-fluorocyclopropyl)-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide;
N-(1-((1S,2R)-2-fluorocyclopropyl)-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide;
N-(1-((1S,2R)-2-fluorocyclopropyl)-2-oxo-1,2-dihydropyridin-3-yl)-2-(1-(fluoromethyl)-2-oxabicyclo[2.1.1]hexan-4-yl)-6-isopropoxy-2H-indazole-5-carboxamide;
N-(1-((1R,2S)-2-fluorocyclopropyl)-2-oxo-1,2-dihydropyridin-3-yl)-2-(1-(fluoromethyl)-2-oxabicyclo[2.1.1]hexan-4-yl)-6-isopropoxy-2H-indazole-5-carboxamide;
N-(1-((1R,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide;
N-(1-((1R,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
N-(1-((1S,2S)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
6-cyclobutoxy-N-(1-((1R,2R)-2-fluorocyclopropyl)-2-oxo-1,2-dihydropyridin-3-yl)-2-(1-(methoxymethyl)-2-oxabicyclo[2.1.1]hexan-4-yl)-2H-indazole-5-carboxamide;
6-cyclobutoxy-N-(1-((1S,2S)-2-fluorocyclopropyl)-2-oxo-1,2-dihydropyridin-3-yl)-2-(1-(methoxymethyl)-2-oxabicyclo[2.1.1]hexan-4-yl)-2H-indazole-5-carboxamide;
N-(1-((1R,2R)-2-fluorocyclopropyl)-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide;
N-(1-((1S,2S)-2-fluorocyclopropyl)-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide;
N-(1-((1R,2R)-2-fluorocyclopropyl)-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide;
N-(1-((1S,2S)-2-fluorocyclopropyl)-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide;
N-(1-((1R,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide;
N-(1-((1S,2S)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide;
6-cyclobutoxy-N-(1-((1R,2R)-2-fluorocyclopropyl)-2-oxo-1,2-dihydropyridin-3-yl)-2-(1-(methoxymethyl)-2-oxabicyclo[2.1.1]hexan-4-yl)-2H-pyrazolo[3,4-b]pyridine-5-carboxamide;
6-cyclobutoxy-N-(1-((1S,2S)-2-fluorocyclopropyl)-2-oxo-1,2-dihydropyridin-3-yl)-2-(1-(methoxymethyl)-2-oxabicyclo[2.1.1]hexan-4-yl)-2H-pyrazolo[3,4-b]pyridine-5-carboxamide;
N-(1-((1S,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
2-(2-oxabicyclo[2.1.1]hexan-4-yl)-N-(1-((1S,2R)-2-fluorocyclopropyl)-2-oxo-1,2-dihydropyridin-3-yl)-6-isopropoxy-2H-indazole-5-carboxamide;
2-(1-(fluoromethyl)-2-oxabicyclo[2.1.1]hexan-4-yl)-6-isopropoxy-N-(1-((1R,2R)-2-methylcyclopropyl)-2-oxo-1,2-dihydropyridin-3-yl)-2H-indazole-5-carboxamide;
2-(1-(fluoromethyl)-2-oxabicyclo[2.1.1]hexan-4-yl)-6-isopropoxy-N-(1-((1S,2S)-2-methylcyclopropyl)-2-oxo-1,2-dihydropyridin-3-yl)-2H-indazole-5-carboxamide;
6-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)-2H-pyrazolo[3,4-b]pyridine-5-carboxamide;
6-isopropoxy-2-(1-(methoxymethyl)-2-oxabicyclo[2.1.1]hexan-4-yl)-N-(1-((1R,2S)-2-methylcyclopropyl)-2-oxo-1,2-dihydropyridin-3-yl)-2H-pyrazolo[3,4-b]pyridine-5-carboxamide;
6-isopropoxy-2-(1-(methoxymethyl)-2-oxabicyclo[2.1.1]hexan-4-yl)-N-(1-((1R,2S)-2-methylcyclopropyl)-2-oxo-1,2-dihydropyridin-3-yl)-2H-indazole-5-carboxamide;
2-(2-oxabicyclo[2.1.1]hexan-4-yl)-6-isopropoxy-N-(1-((1R,2S)-2-methylcyclopropyl)-2-oxo-1,2-dihydropyridin-3-yl)-2H-indazole-5-carboxamide;
2-(2-oxabicyclo[2.1.1]hexan-4-yl)-6-isopropoxy-N-(1-((1R,2S)-2-methylcyclopropyl)-2-oxo-1,2-dihydropyridin-3-yl)-2H-pyrazolo[3,4-b]pyridine-5-carboxamide;
6-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-isopropoxy-2-(1-(methoxymethyl)-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-isopropoxy-2-(1-(methoxymethyl)-2-oxabicyclo[2.1.1]hexan-4-yl)-N-(1-((1S,2R)-2-methylcyclopropyl)-2-oxo-1,2-dihydropyridin-3-yl)-2H-indazole-5-carboxamide;
2-(2-oxabicyclo[2.1.1]hexan-4-yl)-6-isopropoxy-N-(1-((1S,2R)-2-methylcyclopropyl)-2-oxo-1,2-dihydropyridin-3-yl)-2H-indazole-5-carboxamide;
2-(2-oxabicyclo[2.1.1]hexan-4-yl)-6-isopropoxy-N-(1-((1S,2R)-2-methylcyclopropyl)-2-oxo-1,2-dihydropyridin-3-yl)-2H-pyrazolo[3,4-b]pyridine-5-carboxamide;
2-(1-(fluoromethyl)-2-oxabicyclo[2.1.1]hexan-4-yl)-6-isopropoxy-N-(1-((1R,2S)-2-methylcyclopropyl)-2-oxo-1,2-dihydropyridin-3-yl)-2H-indazole-5-carboxamide;
2-(1-(fluoromethyl)-2-oxabicyclo[2.1.1]hexan-4-yl)-6-isopropoxy-N-(1-((1S,2R)-2-methylcyclopropyl)-2-oxo-1,2-dihydropyridin-3-yl)-2H-indazole-5-carboxamide;
N-(1-((1R,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(1-((1S,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
6-cyclopropoxy-N-(1-((1S,2R)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
6-cyclopropoxy-N-(1-((1S,2R)-2-fluorocyclopropyl)-2-oxo-1,2-dihydropyridin-3-yl)-2-(1-(methoxymethyl)-2-oxabicyclo[2.1.1]hexan-4-yl)-2H-pyrazolo[3,4-b]pyridine-5-carboxamide;
2-((1S,4S)-2-oxabicyclo[2.2.1]heptan-4-yl)-6-cyclopropoxy-N-(1-((1S,2R)-2-fluorocyclopropyl)-2-oxo-1,2-dihydropyridin-3-yl)-2H-pyrazolo[3,4-b]pyridine-5-carboxamide;
2-((1R,4R)-2-oxabicyclo[2.2.1]heptan-4-yl)-6-cyclopropoxy-N-(1-((1S,2R)-2-fluorocyclopropyl)-2-oxo-1,2-dihydropyridin-3-yl)-2H-pyrazolo[3,4-b]pyridine-5-carboxamide;
6-cyclopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-cyclopropoxy-2-(1-(methoxymethyl)-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
2-(2-oxabicyclo[2.1.1]hexan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-(2-oxabicyclo[2.1.1]hexan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-((1S,4S)-2-oxabicyclo[2.2.1]heptan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-((1S,4S)-2-oxabicyclo[2.2.1]heptan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
6-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-indazole-5-carboxamide; and
6-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)-2H-indazole-5-carboxamide;
6-cyclopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-cyclopropoxy-2-(1-(methoxymethyl)-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
2-(2-oxabicyclo[2.1.1]hexan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-(2-oxabicyclo[2.1.1]hexan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-((1S,4S)-2-oxabicyclo[2.2.1]heptan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-((1S,4S)-2-oxabicyclo[2.2.1]heptan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
6-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-indazole-5-carboxamide; and
6-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)-2H-indazole-5-carboxamide,
   or a pharmaceutically acceptable salt thereof.

The present disclosure also provides a pharmaceutical composition comprising a compound according to any one of the preceding embodiments, or a pharmaceutically acceptable salt thereof, and one or more pharmaceutically acceptable carriers.

In certain embodiments, the pharmaceutical composition further comprises one or more additional pharmaceutical or therapeutic agent(s).

In certain embodiments, the present disclosure provides a method of treating an IRAK4 mediated disease in a subject in need of the treatment comprising administering to the subject a compound described herein (e.g., a compound described in any one of the first to twenty-fifth embodiments) or a pharmaceutically acceptable salt thereof or a pharmaceutical composition thereof.

In certain embodiments, the present disclosure provides the use of a compound described herein (e.g., a compound described in any one of the first to twenty-fifth embodiments) or a pharmaceutically acceptable salt thereof or a pharmaceutical composition comprising a compound described herein or a pharmaceutically acceptable salt thereof for the manufacture of a medicament for the treatment of a disorder or disease mediated by IRAK4 in a subject in need of the treatment.

In certain embodiments, the present disclosure provides the use of a compound described herein (e.g., a compound described in any one of the first to twenty-fifth embodiments) or a pharmaceutically acceptable salt thereof or a pharmaceutical composition comprising a compound described herein or a pharmaceutically acceptable salt thereof for the treatment of a disorder or disease mediated by IRAK4 in a subject in need of the treatment.

In certain embodiments, the IRAK4 mediated disease is selected from an autoimmune disease, an inflammatory disease, a bone disease, a metabolic disease, a neurological and neurodegenerative disease and/or disorder, cancer, a cardiovascular disease, allergies, asthma, Alzheimer's disease, a hormone-related disease, ischemic stroke, cerebral ischemia, hypoxia, TBI (Traumatic Brain Injury), CTE (Chronic Traumatic Encephalopathy), epilepsy, Parkinson's disease (PD), multiple Sclerosis (MS) and amyotrophic lateral sclerosis (ALS).

In some embodiments, the present disclosure provides a method of treating MS selected from relapsing-remitting MS (RRMS), secondary progressive MS (SPMS), non-relapsing SPMS, primary progressive MS (PPMS), and clinically isolated syndrome (CIS). The method comprises administering to the subject a compound described herein (e.g., a compound described in any one of the first to twenty-fifth embodiments) or a pharmaceutically acceptable salt thereof or a pharmaceutical composition thereof. In certain embodiments, the present disclosure provides a method of treating a relapsing form of MS. The method comprises administering to the subject a compound described herein (e.g., a compound described in any one of the first to twenty-fifth embodiments) or a pharmaceutically acceptable salt thereof or a pharmaceutical composition thereof. As used herein, a “relapsing form of MS” includes clinically isolated syndrome (CIS), relapsing-remitting disease (RRMS), and active secondary progressive disease.

CIS is a first episode of neurologic symptoms caused by inflammation and demyelination in the central nervous system. The episode, which by definition must last for at least 24 hours, is characteristic of multiple sclerosis but does not yet meet the criteria for a diagnosis of MS because people who experience a CIS may or may not go on to develop MS. When CIS is accompanied by lesions on a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS.

RRMS, the most common disease course of MS, is characterized by clearly defined attacks of new or increasing neurologic symptoms. These attacks—also called relapses or exacerbations—are followed by periods of partial or complete recovery (remissions). During remissions, all symptoms may disappear, or some symptoms may continue and become permanent. However, there is no apparent progression of the disease during the periods of remission. RRMS can be further characterized as either active (with relapses and/or evidence of new MRI activity over a specified period of time) or not active, as well as worsening (a confirmed increase in disability following a relapse) or not worsening.

SPMS follows an initial relapsing-remitting course. Some people who are diagnosed with RRMS will eventually transition to a secondary progressive course in which there is a progressive worsening of neurologic function (accumulation of disability) over time. SPMS can be further characterized as either active (with relapses and/or evidence of new MRI activity during a specified period of time) or not active, as well as with progression (evidence of disability accumulation over time, with or without relapses or new MRI activity) or without progression.

PPMS is characterized by worsening neurologic function (accumulation of disability) from the onset of symptoms, without early relapses or remissions. PPMS can be further characterized as either active (with an occasional relapse and/or evidence of new MRI activity over a specified period of time) or not active, as well as with progression (evidence of disability accumulation over time, with or without relapse or new MRI activity) or without progression.

In certain embodiments, the IRAK4 mediated disease is selected from disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, chronic inflammatory demyelinating neuropathies, vascular or heart diseases, ophthalmic diseases and ocular diseases.

In certain embodiments, the IRAK4 mediated disease is selected from the group consisting from rheumatoid arthritis, psoriatic arthritis, osteoarthritis, systemic lupus erythematosus, lupus nephritis, neuropsychiatric lupus, ankylosing spondylitis, osteoporosis, systemic sclerosis, multiple sclerosis, neuromyelitis optica, psoriasis, type I diabetes, type II diabetes, inflammatory bowel disease, Cronh's disease, ulcerative colitis, hyperimmunoglobulinemia D, periodic fever syndrome, Cryopyrin-associated periodic syndromes, Schnitzler's syndrome, systemic juvenile idiopathic arthritis, adult's onset Still's disease, gout, pseudogout, SAPHO syndrome, Castleman's disease, sepsis, stroke, atherosclerosis, celiac disease, deficiency of IL-1 receptor antagonist, Alzheimer's disease, Parkinson's disease, and cancer.

The compounds, or pharmaceutically acceptable salts thereof described herein may be used to decrease the expression or activity of IRAK4, or to otherwise affect the properties and/or behavior of IRAK4 polypeptides or polynucleotides, e.g., stability, phosphorylation, kinase activity, interactions with other proteins, etc. in a cell.

One embodiment of the present disclosure includes a method of decreasing the expression or activity of IRAK4, or to otherwise affect the properties and/or behavior of IRAK4 polypeptides or polynucleotides in a subject comprising administering to said subject an effective amount of at least one compound described herein, or a pharmaceutically acceptable salt thereof.

One embodiment of the present disclosure includes a method for treating an inflammatory disease in a subject, the method comprising administering to the subject a therapeutically effective amount of a compound described herein, or a pharmaceutically acceptable salt thereof, thereby treating the inflammatory disease in the subject.

In one embodiment, the inflammatory disease is a pulmonary disease or a disease of the airway.

In one embodiment, the pulmonary disease and disease of the airway is selected from Adult Respiratory Disease Syndrome (ARDS), Chronic Obstructive Pulmonary Disease (COPD), pulmonary fibrosis, interstitial lung disease, asthma, chronic cough, and allergic rhinitis.

In one embodiment, the inflammatory disease is selected from transplant rejection, CD14 mediated sepsis, non-CD14 mediated sepsis, inflammatory bowel disease, Behcet's syndrome, ankylosing spondylitis, sarcoidosis, and gout.

One embodiment of the present disclosure includes a method for treating an autoimmune disease, cancer, cardiovascular disease, a disease of the central nervous system, a disease of the skin, an ophthalmic disease and condition, and bone disease in a subject, the method comprising administering to the subject a therapeutically effective amount of a compound disclosed herein, or a pharmaceutically acceptable salt thereof, thereby treating the autoimmune disease, cancer, cardiovascular disease, disease of the central nervous system, disease of the skin, ophthalmic disease and condition, and bone disease in the subject.

In one embodiment, the autoimmune disease is selected from rheumatoid arthritis, systemic lupus erythematosus, multiple sclerosis, neuromyelitis optica, diabetes, systemic sclerosis, and Sjogren's syndrome.

In one embodiment, the autoimmune disease is type 1 diabetes.

In one embodiment, the cancer is selected from Waldenstrim's macroglobulinemia, solid tumors, skin cancer, and lymphoma.

In one embodiments, the cancer is selected from lymphoma, leukemia, and Myelodysplastic Syndrome.

In one embodiment, the leukemia is Acute Myelogenous Leukemia (AML) or chronic lymphocytic leukemia (CLL), and the lymphoma is non-Hodgkin's Lymphoma (NHL), small lymphocytic lymphoma (SLL), macroglobulinemia/lymphoplasmacytic lymphoma (WM/LPL), or DLBC lymphomas.

In one embodiment, the cardiovascular disease is selected from stroke and atherosclerosis.

In one embodiment, the disease of the central nervous system is a neurodegenerative disease.

In one embodiment, the disease of the skin is selected from rash, contact dermatitis, psoriasis, and atopic dermatitis.

In one embodiment, the bone disease is selected from osteoporosis and osteoarthritis.

In one embodiment, the inflammatory bowel disease is selected from Crohn's disease and ulcerative colitis.

One embodiment of the present disclosure includes a method for treating an ischemic fibrotic disease, the method comprising administering to the subject a therapeutically effective amount of a compound described herein, or a pharmaceutically acceptable salt thereof, thereby treating the ischemic fibrotic disease in the subject. In one embodiment, the ischemic fibrotic disease is selected from stroke, acute lung injury, acute kidney injury, ischemic cardiac injury, acute liver injury, and ischemic skeletal muscle injury.

One embodiment of the present disclosure includes a method for treating post-organ transplantation fibrosis, the method comprising administering to the subject a therapeutically effective amount of a compound described herein, or a pharmaceutically acceptable salt thereof, thereby treating post-organ transplantation fibrosis in the subject.

One embodiment of the present disclosure includes a method for treating hypertensive or diabetic end organ disease in a subject, the method comprising administering to the subject a therapeutically effective amount of a compound described herein, or a pharmaceutically acceptable salt thereof, thereby treating hypertensive or diabetic end organ disease in the subject.

One embodiment of the present disclosure includes a method for treating hypertensive kidney disease in a subject, the method comprising administering to the subject a therapeutically effective amount of a compound described herein, or a pharmaceutically acceptable salt thereof, thereby treating hypertensive kidney disease in the subject.

One embodiment of the present disclosure includes a method for treating idiopathic pulmonary fibrosis (IPF) in a subject, the method comprising administering to the subject a therapeutically effective amount of a compound described herein, or a pharmaceutically acceptable salt thereof, thereby treating IPF in the subject.

One embodiment of the present disclosure includes a method for treating scleroderma or systemic sclerosis in a subject, the method comprising administering to the subject a therapeutically effective amount of a compound described herein, or a pharmaceutically acceptable salt thereof, thereby treating scleroderma or systemic sclerosis in the subject.

One embodiment of the invention includes a method for treating liver cirrhosis in a subject, the method comprising administering to the subject a therapeutically effective amount of a compound described herein, or a pharmaceutically acceptable salt thereof, thereby treating liver cirrhosis in the subject.

One embodiment of the invention includes a method for treating fibrotic diseases in a subject wherein tissue injury and/or inflammation are present, the method comprising administering to the subject a therapeutically effective amount of a compound described herein, or a pharmaceutically acceptable salt thereof, thereby treating fibrotic diseases where tissue injury and/or inflammation are present in the subject. The fibrotic diseases include, for example, pancreatitis, peritonitis, burns, glomerulonephritis, complications of drug toxicity, and scarring following infections.

Scarring of the internal organs is a major global health problem, which is the consequence of subclinical injury to the organ over a period of time or as the sequela of acute severe injury or inflammation. All organs may be affected by scarring and currently there are few therapies the specifically target the evolution of scarring. Increasing evidence indicates that scarring per se provokes further decline in organ function, inflammation and tissue ischemia. This may be directly due the deposition of the fibrotic matrix which impairs function such as in contractility and relaxation of the heart and vasculature or impaired inflation and deflation of lungs, or by increasing the space between microvasculature and vital cells of the organ that are deprived of nutrients and distorting normal tissue architecture. However recent studies have shown that myofibroblasts themselves are inflammatory cells, generating cytokines, chemokines and radicals that promote injury; and myofibroblasts appear as a result of a transition from cells that normally nurse and maintain the microvasculature, known as pericytes. The consequence of this transition of phenotype is an unstable microvasculature that leads to aberrant angiogenesis, or rarefaction.

The present disclosure relates to methods and compositions for treating, preventing, and/or reducing scarring in organs. More particularly, the present disclosure relates to methods and composition for treating, preventing, and/or reducing scarring in kidneys.

It is contemplated that the present disclosure, methods and compositions described herein can be used as an antifibrotic, or used to treat, prevent, and/or reduce the severity and damage from fibrosis.

It is additionally contemplated that the present disclosure, methods and compositions described herein can be used to treat, prevent, and/or reduce the severity and damage from fibrosis.

It is further contemplated that the present disclosure, methods and compositions described herein can used as an anti-inflammatory, used to treat inflammation.

Some non-limiting examples of organs include: kidney, hearts, lungs, stomach, liver, pancreas, hypothalamus, stomach, uterus, bladder, diaphragm, pancreas, intestines, colon, and so forth.

In certain embodiments, the present disclosure relates to the aforementioned methods, wherein said compound is administered parenterally.

In certain embodiments, the present disclosure relates to the aforementioned methods, wherein said compound is administered intramuscularly, intravenously, subcutaneously, orally, pulmonary, rectally, intrathecally, topically or intranasally.

In certain embodiments, the present disclosure relates to the aforementioned methods, wherein said compound is administered systemically.

In certain embodiments, the present disclosure relates to the aforementioned methods, wherein said subject is a mammal.

In certain embodiments, the present disclosure relates to the aforementioned methods, wherein said subject is a primate.

In certain embodiments, the present disclosure relates to the aforementioned methods, wherein said subject is a human.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”.

The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms.

As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Pharmacology and Utility

Compounds of the present disclosure have been found to modulate IRAK4 activity and may be beneficial for the treatment of neurological, neurodegenerative and other additional diseases

Another aspect of the invention provides a method for treating or lessening the severity of a disease, disorder, or condition associated with the modulation of IRAK4 in a subject, which comprises administering to the subject a compound of Formula (I) or a pharmaceutically acceptable salt thereof.

In certain embodiments, the present invention provides a method of treating a condition, disease or disorder implicated by a deficiency of IRAK4 activity, the method comprising administering a composition comprising a compound of Formula (I) to a subject, preferably a mammal (e.g., a human), in need of treatment thereof.

As used herein, an “effective amount” and a “therapeutically effective amount” can used interchangeably. It means an amount effective for treating or lessening the severity of one or more of the diseases, disorders or conditions as recited above.

The compounds and compositions, according to the methods of the present disclosure, may be administered using any amount and any route of administration effective for treating or lessening the severity of one or more of the diseases, disorders or conditions recited above.

The compounds of the present invention are typically used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations.

### Combination Therapy

The compounds of the present invention can be used, alone or in combination with other therapeutic agents, in the treatment of various conditions or disease states. The compound(s) of the present invention and other therapeutic agent(s) may be administered simultaneously (either in the same dosage form or in separate dosage forms) or sequentially.

Two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present disclosure includes the use of a combination of an IRAK inhibitor compound as provided in the compound of formula (I) and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of formula (I) or a pharmaceutically acceptable salt of the compound; (b) a second pharmaceutically active agent; and (c) a pharmaceutically acceptable carrier, vehicle or diluent.

The compounds of the present invention can be administered alone or in combination with one or more additional therapeutic agents. By “administered in combination” or “combination therapy” it is meant that a compound of the present disclosure and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect. Thus, the methods of prevention and treatment described herein include use of combination agents.

The combination agents are administered to a mammal, including a human, in a therapeutically effective amount. By “therapeutically effective amount” it is meant an amount of a compound of the present disclosure that, when administered alone or in combination with an additional therapeutic agent to a mammal, is effective to treat the desired disease/condition e.g., inflammatory condition such as systemic lupus erythematosus. See also, T. Koutsokeras and T. Healy, Systemic lupus erythematosus and lupus nephritis, Nat Rev Drug Discov, 2014, 13(3), 173-174, for therapeutic agents useful treating lupus.

In particular, it is contemplated that the compounds of the disclosure may be administered with the following therapeutic agents: Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as Tecfidera® and beta interferon (e.g., Avonex® and Rebif®), Copaxone®, and mitoxantrone; treatments for asthma such as albuterol and Singulair*; agents for treating schizophrenia such as zyprexa, risperdal, seroquel, and haloperidol; anti-inflammatory agents such as corticosteroids, T F blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3 A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein the term “stroke” has the meaning normally accepted in the art. The term can broadly refer to the development of neurological deficits associated with the impaired blood flow regardless of cause. Potential causes include, but are not limited to, thrombosis, hemorrhage and embolism. The term “ischemic stroke” refers more specifically to a type of stroke that is of limited extent and caused due to a blockage of blood flow.

As used herein, a subject is “in need of” a treatment if such subject would benefit biologically, medically or in quality of life from such treatment (preferably, a human).

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls). “Halo-substituted alkyl” or “haloalkyl” refers to an alkyl group having at least one halogen substitution.

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄alkoxyC₁₋₄ alkyl” refers to a C₁₋₄ alkyl group as defined herein, wherein at least of the hydrogen atoms is replaced by an C₁₋₄ alkoxy. The C₁₋₄alkoxyC₁₋₄ alkyl group is connected through the rest of the molecule described herein through the alkyl group.

“Halogen” or “halo” may be fluorine, chlorine, bromine or iodine (preferred halogens as substituents are fluorine and chlorine).

As used herein, the term “halo-substituted-C₁₋₄alkyl” or “C₁₋₄haloalkyl” refers to a C₁₋₄alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “carbocyclic ring” refers to a nonaromatic hydrocarbon ring that is either partially or fully saturated and may exist as a single ring, bicyclic ring (including fused, spiral or bridged carbocyclic rings) or a spiral ring. Unless specified otherwise, the carbocyclic ring generally contains 4- to 7-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a monocyclic ring which is fully saturated which has 4 to 7 ring atoms and which contains 1 to 2 heteroatoms, independently selected from sulfur, oxygen and/or nitrogen. Exemplary heterocyclyl group includes oxtanyl, tetrahydrofuranyl, dihydrofuranyl, 1,4-dioxanyl, morpholinyl, 1,4-dithianyl, piperazinyl, piperidinyl, 1,3-dioxolanyl, pyrrolinyl, pyrrolidinyl, tetrahydropyranyl, oxathiolanyl, dithiolanyl, 1,3-dioxanyl, 1,3-dithianyl, oxathianyl, thiomorpholinyl, thiomorpholinyl 1,1 dioxide, tetrahydro-thiopyran 1,1-dioxide, 1,4-diazepanyl. In some embodiments, the heterocyclyl group is a 4 to 6 membered heterocyclyl group. In some embodiments, a heterocyclyl group contains at least one oxygen ring atom. In some embodiments, a heterocyclyl group is selected from oxtanyl, tetrahydrofuranyl, 1,4-dioxanyl and tetrahydropyranyl.

As used herein the term “spiral” ring means a two-ring system wherein both rings share one common atom. Examples of spiral rings include 5-oxaspiro[2.3]hexane, oxaspiro[2.4]heptanyl, 5-oxaspiro[2.4]heptanyl, 4-oxaspiro[2.4]heptane, 4-oxaspiro[2.5]octanyl, 6-oxaspiro[2.5]octanyl, oxaspiro[2.5]octanyl, oxaspiro[3.4]octanyl, oxaspiro[bicyclo[2.1.1]hexane-2,3′-oxetan]-1-yl, oxaspiro[bicyclo[3.2.0]heptane-6,1′-cyclobutan]-7-yl, 2,6-diazaspiro[3.3]heptanyl, -oxa-6-azaspiro[3.3]heptane, 2,2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. bicyclo[1.1.1]pentane, bicyclo[2.2.1]heptane and bicyclo [3.2.1]octane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of formula (I), as well as all stereoisomers (including diastereoisomers and enantiomers), rotamers, tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the invention and does not pose a limitation on the scope of the invention otherwise claimed.

In one embodiment, the present disclosure provides a compound of the Examples as an isolated stereoisomer wherein the compound has one stereocenter and the stereoisomer is in the R configuration.

In one embodiment, the present disclosure provides a compound of the Examples as an isolated stereoisomer wherein the compound has one stereocenter and the stereoisomer is in the S configuration.

In one embodiment, the present disclosure provided a compound of the Examples as an isolated stereoisomer wherein the compound has two stereocenters and the stereoisomer is in the R R configuration.

In one embodiment, the present disclosure provided a compound of the Examples as an isolated stereoisomer wherein the compound has two stereocenters and the stereoisomer is in the R S configuration.

In one embodiment, the present disclosure provided a compound of the Examples as an isolated stereoisomer wherein the compound has two stereocenters and the stereoisomer is in the S R configuration.

In one embodiment, the present disclosure provided a compound of the Examples as an isolated stereoisomer wherein the compound has two stereocenters and the stereoisomer is in the S S configuration.

In one embodiment, the present disclosure provided a compound of the Examples, wherein the compound has one or two stereocenters, as a racemic mixture.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the formula (I) as defined herein, in free form. In another embodiment, the present disclosure relates to a compound of the formula (I) as defined herein, in salt form. In another embodiment, the present disclosure relates to a compound of the formula (I) as defined herein, in acid addition salt form. In a further embodiment, the present disclosure relates to a compound of the formula (I) as defined herein, in pharmaceutically acceptable salt form. In yet a further embodiment, the present disclosure relates to a compound of the formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Furthermore, the compounds of the present disclosure, including their salts, may also be obtained in the form of their hydrates, or include other solvents used for their crystallization. The compounds of the present disclosure may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Compounds of the present disclosure, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

The compounds of the present disclosure, including salts, hydrates and solvates thereof, may inherently or by design form polymorphs.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

The further optional reduction, oxidation or other functionalization of compounds of formula (I) may be carried out according to methods well known to those skilled in the art. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present disclosure having at least one salt-forming group may be prepared in a manner known to those skilled in the art. For example, acid addition salts of compounds of the present invention are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a commercially available chiral HPLC column.

The present disclosure further includes any variant of the present processes, in which the reaction components are used in the form of their salts or optically pure material. Compounds of the invention and intermediates can also be converted into each other according to methods generally known to those skilled in the art.

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### EXEMPLIFICATION

### Abbreviation

CO=carbon monoxide
PE=petroleum ether
EtOAc=EA=ethyl acétate
ESI=electrospray ionisation
MeOH=methanol
EtOH=ethanol
TEA=Triethylamine
T3P®=Propanephosphonic acid anhydride
DCM=dichloromethane
DEA=diethylamine
DMF=dimethylformamide
HATU=Hexafluorophosphate Azabenzotriazole Tetramethyl Uronium
HCl=hydrochloric acid
NBS=N-bromosuccinimide
LCMS=liquid chromatography mass spectrometry
HPLC=high pressure liquid chromatography
THF=tetrahydrofuran
MeCN=ACN=acetonitrile
AcOH=acetic acid
DMAP=4-dimethylaminopyridine
TFA=trifluoroacetic acid
DIPEA=diisopropylethyl amine
TLC=Thin Layer Chromatography
SFC=Supercritical Fluid Chromatography
N₂=Nitrogen
MBPR=Manual Back Pressure Regulator
ABPR=Automatic Back Pressure Regulator
RPHPLC=Reverse Phase HPLC
NH₄HCO₃=Ammonium Bicarbonate
CO₂=Carbon Dioxide
NH₄OH=Ammonium Hydroxide
Hunigs Base=N,N-diisopropylethylamine
NH₄Cl=ammonium chloride
MgSO₄=magnesium sulfate
NaH=sodium hydride
Xantphos=4,5-Bis(diphenylphosphino)-9,9-dimethylxanthene
LiOH·H₂O=lithium hydroxide hydrate
Na₂SO₄=sodium sulfate
NaHCO₃=sodium bicarbonate
Pd(OAc)₂=Palladium (II) Acetate
NaOH=Sodium Hydroxide
Pd(dppf)Cl₂=[1,1′-Bis(diphenylphosphino)ferrocene]dichloropalladium(II)
KNO₃=potassium nitrate
H₂SO₄=sulfuric acid
i-PrOH=IPA=isopropanol
Tf₂O=trifluoromethanesulfonic anhydride
DMSO=dimethyl sulfoxide
K₂CO₃=potassium carbonate
NaIO₄=sodium periodate
K₂OsO₄=potassium osmate
CDCl₃=deuterated chloroform
P(n-Bu)₃=tributylphosphine
Na₂SO₃=sodium sulfite
KHSO₄=potassium bisulfate
Cs₂CO₃=cesium carbonate
Br₂=bromine

### GENERAL METHODS

The compounds of the Examples were analyzed or purified according to one of the Purification Methods referred to below unless otherwise described.

Where preparative TLC or silica gel chromatography have been used, one skilled in the art may choose any combination of solvents to purify the desired compound. Silica gel column chromatography was performed using 20-40 M (particle size), 250-400 mesh, or 400-632 mesh silica gel using either a Teledyne ISCO Combiflash RF or a Grace Reveleris X2 with ELSD purification systems or using pressurized nitrogen (˜10-15 psi) to drive solvent through the column (“flash chromatography”).

Wherein an SCX column has been used, the eluant conditions are MeOH followed by methanolic ammonia.

Except where otherwise noted, reactions were run under an atmosphere of nitrogen. Where indicated, solutions and reaction mixtures were concentrated by rotary evaporation under vacuum.

### Analytical Methods

ESI-MS data (also reported herein as simply MS) were recorded using Waters System (Acquity HPLC and a Micromass ZQ mass spectrometer); all masses reported are the m/z of the protonated parent ions unless recorded otherwise.

### LC/MS:

A sample was dissolved in a suitable solvent such as MeCN, dimethyl sulfoxide (DMSO), or MeOH and was injected directly into the column using an automated sample handler. The analysis used one of the following methods: (1) acidic method (1.5, 2, 3.5, 4, or 7 min runs, see Acidic LCMS section for additional details vide infra: conducted on a Shimadzu 2010 Series, Shimadzu 2020 Series, or Waters Acquity UPLC BEH. (MS ionization: ESI) instrument equipped with a C18 column (2.1 mm×30 mm, 3.0 mm or 2.1 mm×50 mm, C18, 1.7 m), eluting with 1.5 mL/4 L of trifluoroacetic acid (TFA) in water (solvent A) and 0.75 mL/4 L of TFA in MeCN (solvent B) or (2) basic method (3, 3.5, 7 min runs, see Basic LCMS section for additional details vide infra: conducted on a Shimadzu 2020 Series or Waters Acquity UPLC BEH (MS ionization: ESI) instrument equipped with XBridge Shield RP18, Sum column (2.1 mm×30 mm, 3.0 mm i.d.) or 2.1 mm×50 mm, C18, 1.7 m column, eluting with 2 mL/4 L NH₃·H₂O in water (solvent A) and MeCN (solvent B).

The disclosure further includes any variant of the present processes, in which the reaction components are used in the form of their salts or optically pure material. Compounds of the disclosure and intermediates can also be converted into each other according to methods generally known to those skilled in the art.

### SFC Analytical Separation

Instrument: Waters UPC2 analytical SFC (SFC-H). Column: ChiralCel OJ, 150×4.6 mm I.D., 3 μm. Mobile phase: A for CO2 and B for Ethanol (0.05% DEA). Gradient: B 40%. Flow rate: 2.5 mL/min. Back pressure: 100 bar. Column temperature: 35° C. Wavelength: 220 nm.

Detectors: Gilson UV/VIS-156 with UV detection at 220/254 nm, Gilson 281 automatic collection, utilizing acidic, basic and neutral methods. For mass-directed peak collection, an ACQUITY QDa Mass Detector (Waters Corporation) was employed.

### Preparative SFC Purification

Instrument: MG III preparative SFC (SFC-1). Column: ChiralCel OJ, 250×30 mm I.D., 5 μm. Mobile phase: A for CO2 and B for Ethanol (0.1% NH3H2O). Gradient: B 50%.
Flow rate: 40 mL/min. Back pressure: 100 bar. Column temperature: 38° C. Wavelength: 220 nm. Cycle time: ˜8 min.
Column: Chiralpak AD-H; 250 mm×30 mm, 5 m; 40% (EtOH+0.1% DEA)/CO₂
Column: Chiralpak IA; 250 mm×30 mm, 5 m; 40% (MeOH+0.1% DEA)/CO₂
Column: Chiralpak IB; 250 mm×30 mm, 5 m; 40% (EtOH+0.1% DEA)/CO₂
Column: Chiralpak AD-H; 250 mm×30 mm, 5 m; 40% (EtOH+0.1% NH₄OH)/CO₂
Column: Chiralpak OJ-H; 250 mm×30 mm, 5 m; 30% (EtOH+0.1% NH₄OH)/CO₂
Column: Chiralpak OD; 250 mm×30 mm, 5 m; 35% (EtOH+0.1% NH₄OH)/CO₂

### ¹H-NMR

¹H nuclear magnetic resonance (NMR) spectra were in all cases consistent with the proposed structures. The 1H NMR spectra were recorded on a Bruker Avance III HD 500 MHz, Bruker Avance III 500 MHz, Bruker Avance III 400 MHz, Varian-400 VNMRS, or Varian-400 MR. Characteristic chemical shifts (6) are given in parts-per-million downfield from tetramethylsilane (for ¹H-NMR) using conventional abbreviations for designation of major peaks: e.g. s, singlet; d, doublet; t, triplet; q, quartet; dd, double doublet; dt, double triplet; m, multiplet; br, broad. The following abbreviations have been used for common solvents: CDCl₃, deuterochloroform; DMSO-d₆, hexadeuterodimethyl sulfoxide; and MeOH-d₄, deuteron-methanol. Where appropriate, tautomers may be recorded within the NMR data; and some exchangeable protons may not be visible.

Typically, the compounds of Formula (I) can be prepared according to the schemes provided below. The following examples serve to illustrate the invention without limiting the scope thereof. Methods for preparing such compounds are described hereinafter.

### GENERAL SCHEMES

Scheme 1, 2, 3, 4, 5 and 6 provide potential routes for making compounds of Formula (I).

### Scheme 1

According to a first process, compounds of Formula (I), may be prepared from compounds of Formulae (II′), (III′), (IV′), (V′), (VI′), (VII′) and (VIII′) as illustrated by Scheme 1.

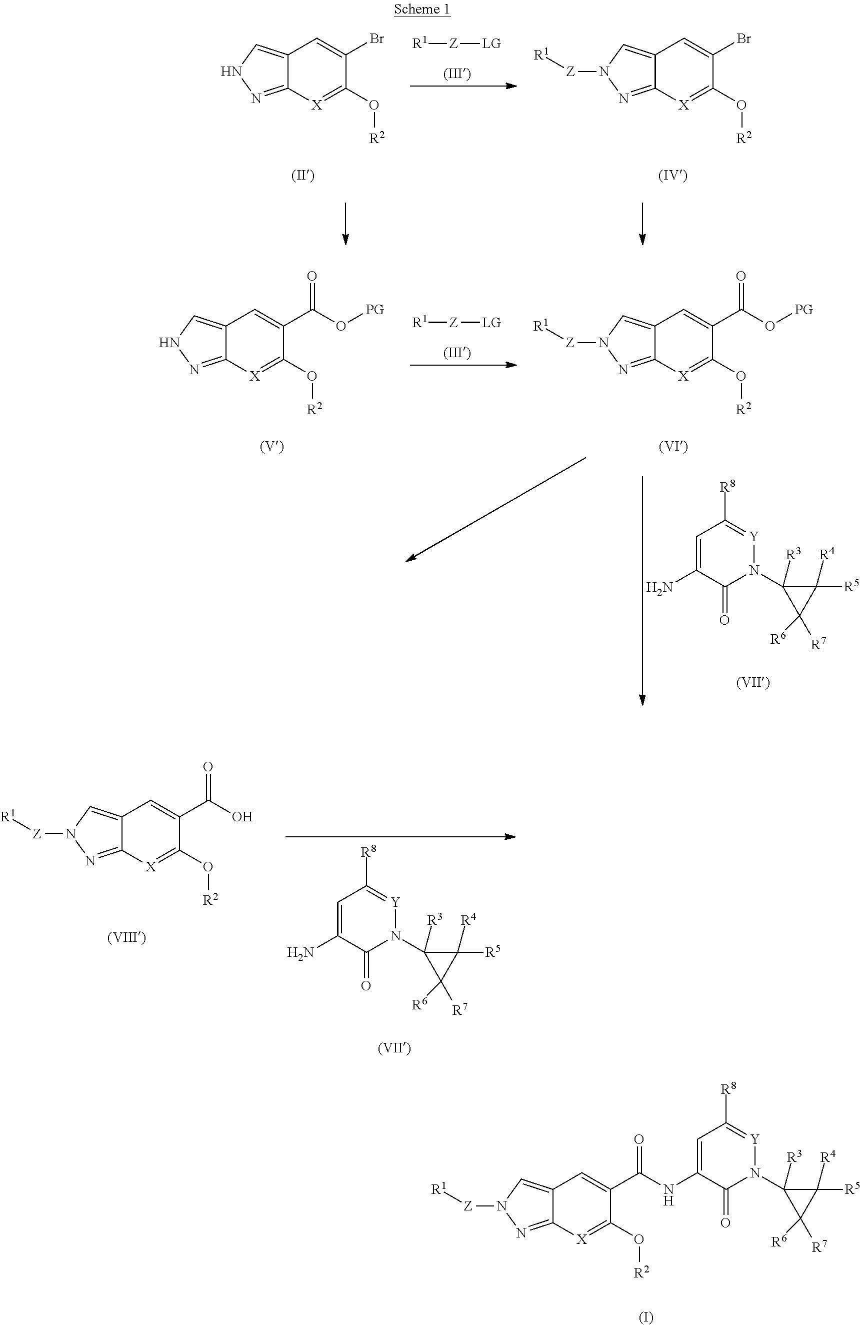

In Scheme 1, LG is a leaving group, typically mesylate, tosylate, iodo or bromo; PG is a carboxylic acid protecting group, typically C₁-C₄ alkyl or phenyl and preferably Me, Et or phenyl; and the remaining variables are as defined above for Formula (I).

Compounds of Formula (IV′) may be prepared from the compound of Formula (II′) and the compound of Formula (III′) by an alkylation reaction in the presence of a suitable inorganic base and a suitable polar aprotic solvent at between 0° C. and an elevated temperature. Preferred conditions comprise reaction of the compound of Formula (II′) with the compound of Formula (III′) in the presence of K₂CO₃ or Cs₂CO₃ in DMF at between 0° C. and 110° C.

Alternatively, compounds of Formula (IV′) may be prepared by an addition reaction of the compound of Formula (II′) with R^{1′}CH═CH₂, (wherein R^{1′}CH₂—CH₂ is an entity that may be transformed using standard chemical transformations to R¹), in the presence of a non-nucleophilic base, such as DBU in a suitable solvent, such as MeCN at between rt and 50° C., followed by a standard chemical transformation, such as a reduction of an ester, to provide the compound of Formula (IV′).

Compounds of Formula (V′) may be prepared from the bromide of Formula (II′) by a palladium catalysed carbonylation reaction, in the presence of a suitable palladium catalyst, organic base and suitable alcohol at an elevated temperature under an atmosphere of CO. When PG is methyl or ethyl, preferred conditions comprise, reaction of the bromide of Formula (II′) under an atmosphere of CO in the presence of suitable palladium catalyst such as Pd(dppf)Cl₂, an organic base such as TEA in a solvent such as MeOH or EtOH at between 8° and 100° C.

Alternatively, when PG is phenyl, compounds of Formula (V′) may be prepared from the bromide of Formula (II′) by a palladium catalyzed reaction with phenyl formate, in the presence of a suitable palladium catalyst such as Pd(OAc)₂ with a phosphine-based ligand such as BINAP or XantPhos, an organic base such as N,N-diethylethanamine, in a solvent such as MeCN at between 8° and 100° C.

Compounds of Formula (VI′) may be prepared from the compound of Formula (V′) and the compound of Formula (III′) by an alkylation reaction as described above, for the preparation of compounds of the Formula (IV′).

Alternatively compounds of Formula (VI′) may be prepared from the bromide of Formula (IV′) via a palladium catalysed carbonylation reaction as previously described above, for the preparation of compounds of the Formula (V′).

Compounds of Formula (VIII′) may be prepared by the hydrolysis of the ester of Formula (VI′) under suitable acidic or basic conditions in a suitable aqueous solvent. Preferred conditions comprise the treatment of the ester of Formula (VI′) with an alkali metal base such as LiOH, NaOH or K₂CO₃ in aqueous MeOH and/or THF at between rt and the reflux temperature of the reaction.

The compound of Formula (I) may be prepared by an amide bond formation of the acid of Formula (VIII′) and the amine of Formula (VII′) in the presence of a suitable coupling agent and organic base, optionally in a suitable polar aprotic solvent. Preferred conditions, comprise the reaction of the acid of Formula (VIII′) with the amine of Formula (VII′) in the presence of coupling agent preferably, T3P®, CDI, HATU or HOAt, in the presence of a suitable organic base such as TEA, DIPEA or pyridine, optionally in a suitable solvent, such as DMF, DMSO, EtOAc, dioxane or MeCN at between rt and the reflux temperature of the reaction.

Alternatively, compounds of Formula (I) may be prepared directly from compounds of Formula (VI′) by reaction with the amine of Formula (VII′) in the presence of DABAL-Me₃, according to the method described by Novak et al (Tet. Lett. 2006, 47, 5767). Preferred conditions comprise reaction of the ester of Formula (VI′) with the amine of Formula (VII′) in the presence of DABAL-Me₃, in a suitable solvent such as THF at rt.

### Scheme 2

According to a second process, compounds of Formula (I) can be prepared from compounds of Formulae (III′), (VII′), (IX′) and (X′) as illustrated by Scheme 2.

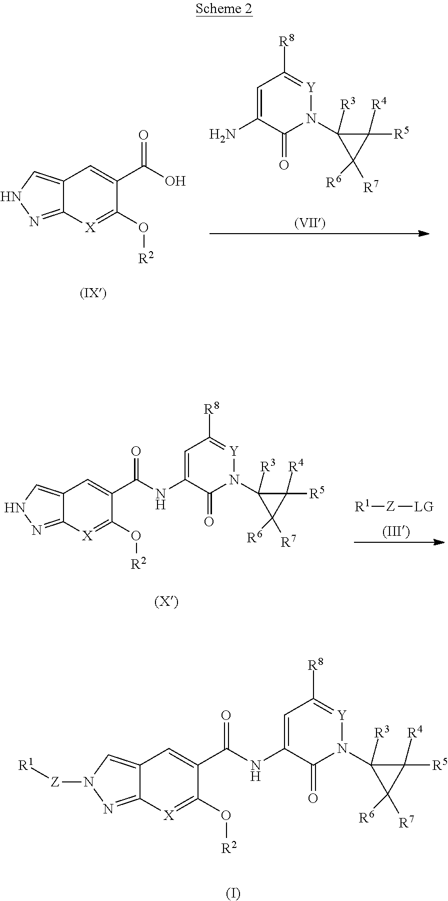

In Scheme 2, LG is as defined in Scheme 1; and the remaining variables are as defined above for Formula (I).

The compound of Formula (X′) may be prepared by an amide bond formation of the acid of Formula (IX′) and the amine of Formula (VII′) in the presence of a suitable coupling agent and organic base in a suitable polar aprotic solvent as previously described in Scheme 1.

Compounds of Formula (I) can be prepared from the compound of Formula (X′) and the compound of Formula (III′) by an alkylation reaction in the presence of a suitable inorganic base and a suitable polar aprotic solvent as previously described in Scheme 1.

### Scheme 3

According to a third process, compounds of Formula (II′) can be prepared from compounds of Formulae (XII′), (XIII′) and (XIV′) as illustrated by Scheme 3.

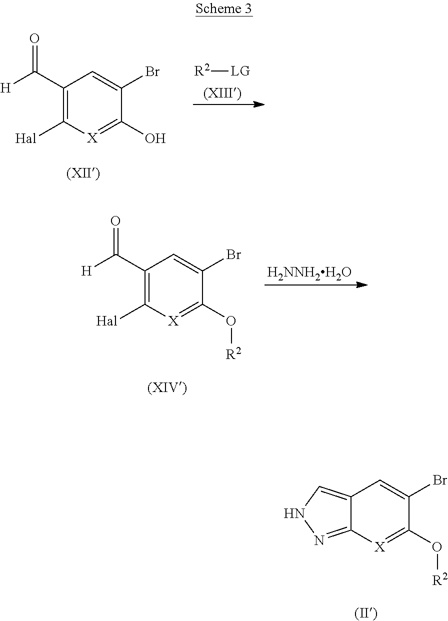

In Scheme 3, Hal is halogen, preferably fluorine; LG is as defined in Scheme 1; and the remaining variables are as defined above for Formula (I).

Compounds of Formula (XIV′) can be prepared from the compound of Formula (XII′) and the compound of Formula (XIII′) by an alkylation reaction in the presence of a suitable inorganic base and a suitable polar aprotic solvent between rt and elevated temperature. Preferred conditions comprise reaction of the compound of Formula (XII′) with the compound of Formula (XIII′) in the presence of K₂CO₃ in DMF at between 50° C. and 100° C.

Compounds of Formula (II′) can be prepared by the condensation of the compound of Formula (XIV′) with hydrazine hydrate in the presence of a suitable inorganic base such as K₂CO₃ and a suitable polar aprotic solvent, such as DMSO at elevated temperature, such as 100° C.

### Scheme 4

According to a fourth process, compounds of Formula (IV′) can be prepared from compounds of Formulae (III′), (XV′) and (XVI′) as illustrated by Scheme 4.

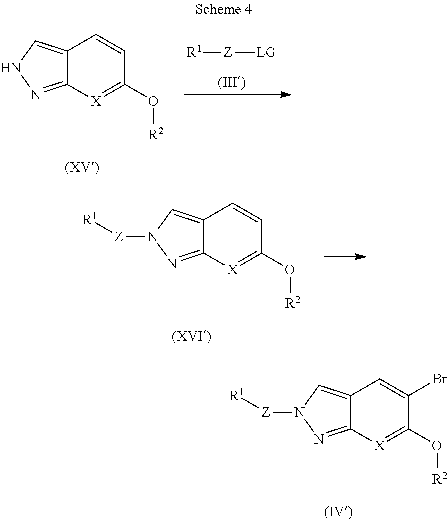

Compounds of Formula (XVI′) can be prepared from the compound of Formula (XV′) and the compound of Formula (III′) by an alkylation reaction, as previously described in Scheme 1.

Compounds of Formula (IV′) can be prepared from the compound of Formula (XVI′) by a bromination reaction, using Br₂ under acidic conditions, typically in AcOH, at about rt.

### Scheme 5

According to a fifth process, compounds of Formula (IV′), where X═CH, can be prepared from compounds of the Formulae (XVII′) and (XVIII′) as illustrated in Scheme 5.

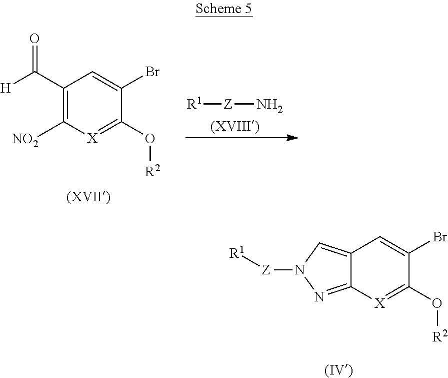

Compounds of Formula (IV′) may be prepared from the compound of Formula (XVII′) and the amine of Formula (XVIII′), by a cyclisation reaction under Cadogan like conditions. Typical conditions comprise reaction of the aldehyde of Formula (XVII′) with the amine of Formula (XVIII′) in the presence of a suitable organic base, such as TEA in a suitable alcoholic solvent, such as isopropanol, at elevated temperature, followed by treatment with a suitable phosphine ligand, such as P(n-Bu)₃ or PPh₃.

### Scheme 6

Alternatively, according to a sixth process, compounds of Formula (IV′), can be prepared starting from the compound of Formula (XIX′) as illustrated in Scheme 6.

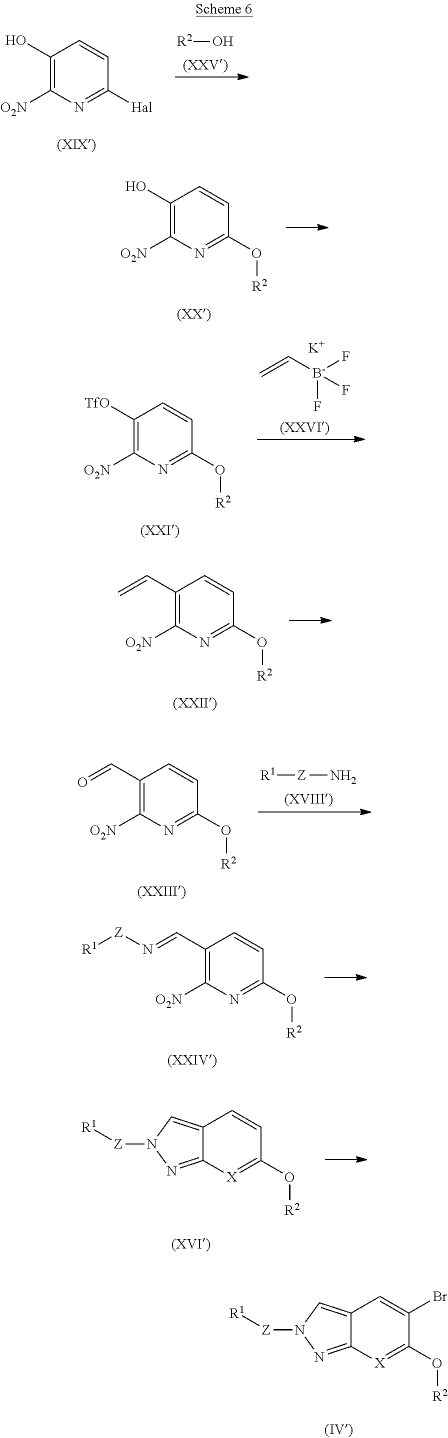

In Scheme 6, Hal is preferably F; and the remaining variables are as defined above for Formula (I).

Nucleophilic displacement of a halogen (Hal) within Formula (XIX′) with the sodium anion of R²—OH (XXV′) to give Formula (XX′). Triflation of Formula (XX′) with Tf₂O in a non-polar aprotic solvent, such as DCM in the presence of an amine base such as triethylamine or DIPEA provides compounds of Formula (XXI′). Palladium-catalyzed cross coupling (Suzuki reaction) using a catalyst such as Pd(dppf)Cl₂ or [PPh₃]₄Pd in the presence of an inorganic base such as potassium carbonate or sodium carbonate with a suitable organometallic alkene reagent such as formula (XXVI′) by heating in a polar aprotic solvent such as dioxane, 2-Methyl-THF or DMF provides compounds of Formula (XXII′). Compounds of formula (XXII′) can undergo oxidative cleavage of the alkene with sodium periodate or osmium tetroxide to provide aldehyde compounds of formula (XXIII′). Condensation of the aldehyde of formula (XXIII′) with amine compounds of formula (XVIII′) in the presence of an amine base such as triethylamine in a polar protic solvent such as isopropanol yields imine compounds of formula (XXIV′). The imine of formula (XXIV′) can be isolated and purified by silica gel chromatography, or can be isolated as a crude residue after evaporation of the solvent and used directly in the next reaction. The imine of formula (XXIV′) can undergo Cadogan cyclization similar to that described above to provide compounds of Formula (XVI′), which can undergo bromination as described above to form compounds of Formula (IV′).

It will be appreciated by those skilled in the art that the experimental conditions set forth in the schemes that follow are illustrative of suitable conditions for effecting the transformations shown, and that it may be necessary or desirable to vary the precise conditions employed for the preparation of the compound of Formula (I). It will be further appreciated that it may be necessary or desirable to carry out the transformations in a different order from that described in the schemes, or to modify one or more of the transformations, to provide the desired compound of the invention.

### INTERMEDIATE PREPARATIONS

### Preparation 1: 6-isopropoxy-2-nitronicotinaldehyde

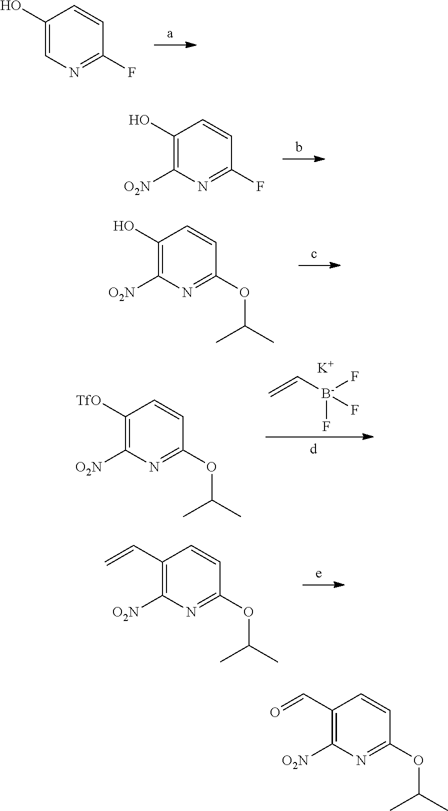

Step a: The KNO₃ (22.4 g, 221.06 mmol) was added to H₂SO₄ (300 mL) at 0° C. and then 6-fluoropyridin-3-ol (25 g, 221.06 mmol, 1.0 eq.) was added. The mixture was stirred at 25° C. for 4 h. The mixture was poured into ice water (1500 mL) and the precipitated solid was filtered, collected and dried to give 6-fluoro-2-nitropyridin-3-ol (28.0 g, 72% yield) as a yellow solid. ¹H NMR (500 MHz, CHLOROFORM-d) δ ppm=10.23 (s, 1H), 7.79 (dd, J=11.0, 7.5 Hz, 1H), 7.34 (dd, J=11.0, 4.5 Hz, 1H).

Step b: Sodium metal (8.14 g, 354 mmol) was added to i-PrOH (500 mL) at 0° C. and the mixture was heated at 60° C. under N₂ until the sodium was dissolved completely. Then the temperature was lowered to 30° C. and 6-fluoro-2-nitropyridin-3-ol (28.0 g, 177 mmol) was added, and the mixture stirred at 50° C. for 16 h. The mixture was concentrated and then water (500 mL) was added. The mixture was extracted with EtOAc (3×300 mL). The combined organic layers were washed with brine (100 mL), dried (Na₂SO₄), filtered and concentrated.

The resulting residue was purified by silica gel chromatography (5% EtOAc in PE) to give 6-isopropoxy-2-nitropyridin-3-ol (17 g, 44% yield) as a yellow oil. ¹H NMR (500 MHz, CHLOROFORM-d) δ ppm=10.14 (s, 1H), 7.51 (d, J=9.0 Hz, 1H), 7.04 (d, J=9.0 Hz, 1H), 5.35-5.27 (m, 1H), 1.37 (d, J=6.0 Hz, 6H).

Step c: To a solution of give 6-isopropoxy-2-nitropyridin-3-ol (18 g, 90.83 mmol) in DCM (300 mL) was added TEA (18.4 g, 182 mmol) and Tf₂O (30.8 g, 109 mmol) at 0° C. and the solution was maintained at 0° C. for 1 h. The solution was concentrated and then water (200 mL) was added. The mixture was extracted with DCM (2×200 mL). The combined organic layers were washed with brine (50 mL), dried (Na₂SO₄), filtered and concentrated. The resulting residue was purified by silica gel chromatography (5% EtOAc in PE) to give 6-isopropoxy-2-nitropyridin-3-yl trifluoromethanesulfonate (24 g, 72% yield) as a yellow oil. ¹H NMR (500 MHz, DMSO-d₆) δ ppm=8.27 (d, J=11.5 Hz, 1H), 7.42 (d, J=11.0 Hz, 1H), 5.26-5.18 (m, 1H), 1.35 (d, J=8.0 Hz, 6H).

Step d: To a solution of 6-isopropoxy-2-nitropyridin-3-yl trifluoromethanesulfonate (24 g, 72.7 mmol) in dioxane (500 mL) and water (60 mL) was added potassium trifluoro(vinyl)borate (14.6 g, 109 mmol), K₂CO₃ (20.1 g, 145 mmol) and Pd(dppf)Cl₂ (5.32 g, 7.27 mmol) under N₂ flow. The mixture was stirred at 80° C. for 16 h. The mixture was concentrated and then water (300 mL) was added. The mixture was extracted with EtOAc (3×200 mL). The combined organic layers were washed with brine (100 mL), dried (Na₂SO₄), filtered and concentrated. The residue was purified by silica gel chromataography (10% EtOAc in PE) to give 6-isopropoxy-2-nitro-3-vinylpyridine (14.3 g, 89.8% yield) as a yellow oil. ¹H NMR (500 MHz, CHLOROFORM-d) δ ppm=7.93 (d, J=8.5 Hz, 1H), 6.93 (d, J=9.0 Hz, 1H), 6.89 (dd, J=17.5, 10.0 Hz, 1H), 5.72 (d, J=17.0 Hz, 1H), 5.44 (d, J=11.0 Hz, 1H), 5.33-5.29 (m, 1H), 1.37 (d, J=6.0 Hz, 6H).

Step e: To a solution of 6-isopropoxy-2-nitro-3-vinylpyridine (14.3 g, 68.7 mmol) in dioxane (200 mL) and water (60 mL) was added NaIO₄ (29.4 g, 137 mmol) and K₂OsO₄ (1.27 g, 3.43 mmol). The mixture was stirred at 25° C. for 2 h and then was concentrated. Water (300 mL) was added and the mixture was extracted with EtOAc (2×200 mL). The combined organic layers were washed with brine (50 mL), dried (Na₂SO₄), filtered and concentrated. The residue was purified by silica gel chromatography (gradient 0-5% EtOAc in PE) to give title compound 6-isopropoxy-2-nitronicotinaldehyde (11.5 g, 71.7% yield) as a gray oil. ¹H NMR (500 MHz, CHLOROFORM-d) δ ppm=10.18 (s, 1H), 8.27 (d, J=8.5 Hz, 1H), 7.04 (d, J=8.5 Hz, 1H), 5.48-5.39 (m, 1H), 1.40 (d, J=6.0 Hz, 6H).

### Preparation 2: (E)-1-(6-isopropoxy-2-nitropyridin-3-yl)-N-(1-methyl-2-oxabicyclo[2.1.1]hexan-4-yl)methanimine

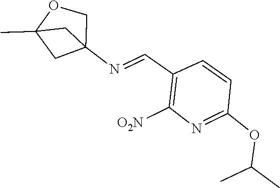 [SMILES: CC(C)Oc1ccc(/C=N/C23COC(C)(C2)C3)c([N+](=O)[O-])n1]

To a mixture of 1-methyl-2-oxabicyclo[2.1.1]hexan-4-amine (5.50 g, 36.8 mmol, HCl), TEA (10.83 g, 107.1 mmol, 14.92 mL) in i-PrOH (60 mL) was added 6-isopropoxy-2-nitronicotinaldehyde [preparation 1](4.50 g, 21.4 mmol) at 20° C. The resulting mixture was heated at 80° C. for 12 h under N₂ atmosphere. The mixture was cooled to room temperature and evaporated to give the crude product, which was purified by column chromatography on silica gel (Petroleum ether/EtOAc=3/1) to give (E)-1-(6-isopropoxy-2-nitropyridin-3-yl)-N-(1-methyl-2-oxabicyclo[2.1.1]hexan-4-yl)methanimine (4.80 g, 73.4% yield) as a yellow solid. 1H NMR: (400 MHz, CDCl₃) δ ppm 8.45 (s, 1H), 8.37 (d, J=8.4 Hz, 1H), 6.91 (dd, J=8.4, 0.8 Hz, 1H), 5.27-5.34 (m, 1H), 3.72 (s, 2H), 1.97 (dd, J=4.4, 1.6 Hz, 2H), 1.72 (dd, J=4.4, 1.6 Hz, 2H), 1.45 (s, 3H), 1.33 (s, 3H), 1.31 (s, 3H).

### Preparation 3: 6-isopropoxy-2-(1-methyl-2-oxabicyclo[2.1.1]hexan-4-yl)-2H-pyrazolo[3,4-b]pyridine

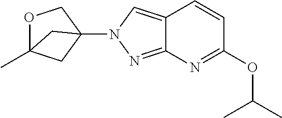 [SMILES: CC(C)Oc1ccc2cn(C34COC(C)(C3)C4)nc2n1]

A mixture of (E)-1-(6-isopropoxy-2-nitropyridin-3-yl)-N-(1-methyl-2-oxabicyclo[2.1.1]hexan-4-yl)methanimine (4.80 g, 15.7 mmol) and tributylphosphane (8.92 g, 44.1 mmol, 11.0 mL) in i-PrOH (100 mL) was stirred at 80° C. for 4 h under N₂ atmosphere. The mixture was cooled to room temperature and evaporated to give the crude product, which was purified by column chromatography on silica gel (Petroleum ether/EtOAc=1/1) to give 6-isopropoxy-2-(1-methyl-2-oxabicyclo[2.1.1]hexan-4-yl)-2H-pyrazolo[3,4-b]pyridine (1.33 g, 30.9% yield) as a yellow solid.

### Preparation 4: 5-bromo-6-isopropoxy-2-(1-methyl-2-oxabicyclo[2.1.1]hexan-4-yl)-2H-pyrazolo[3,4-b]pyridine

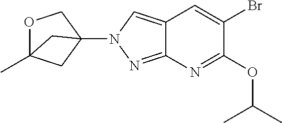 [SMILES: CC(C)Oc1nc2nn(C34COC(C)(C3)C4)cc2cc1Br]

To a mixture of 6-isopropoxy-2-(1-methyl-2-oxabicyclo[2.1.1]hexan-4-yl)-2H-pyrazolo[3,4-b]pyridine [preparation 3](460 mg, 1.68 mmol) in MeCN (30 mL) was added NBS (329 mg, 1.85 mmol) at 20° C. The resulting mixture was stirred at 20° C. for 12 h under N₂ atmosphere. The mixture was evaporated to give the crude product, which was purified by column chromatography on silica gel (Petroleum ether/EtOAc=1/1) to give 5-bromo-6-isopropoxy-2-(1-methyl-2-oxabicyclo[2.1.1]hexan-4-yl)-2H-pyrazolo[3,4-b]pyridine (1.50 g, 87.5% yield) as a yellow solid. LCMS (ESI): 352.0, 354.1 [M+H]⁺.

### Preparation 5: Methyl 6-isopropoxy-2-(1-methyl-2-oxabicyclo[2.1.1]hexan-4-yl)-2H-pyrazolo[3,4-b]pyridine-5-carboxylate

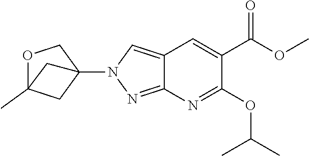

To a mixture of Pd(dppf)Cl₂ (311.6 mg, 425.8 μmol), TEA (1.29 g, 12.8 mmol, 1.78 mL) in MeOH (50 mL) was added 5-bromo-6-isopropoxy-2-(1-methyl-2-oxabicyclo[2.1.1]hexan-4-yl)-2H-pyrazolo[3,4-b]pyridine [preparation 4](1.50 g, 4.26 mmol) at 20° C. The resulting mixture stirred at 80° C. for 12 h under CO (50 psi) atmosphere. The mixture was cooled to room temperature and evaporated to give the crude product, which was purified by column chromatography on silica gel (Petroleum ether/EtOAc=1/1) to give methyl 6-isopropoxy-2-(1-methyl-2-oxabicyclo[2.1.1]hexan-4-yl)-2H-pyrazolo[3,4-b]pyridine-5-carboxylate (1.30 g, 92.1% yield) as an off-white solid. 1H NMR: (400 MHz, CDCl₃) δ ppm 8.45 (s, 1H), 7.85 (s, 1H), 5.50-5.51 (m, 1H), 4.15 (s, 2H), 3.83 (s, 3H), 2.24-2.26 (m, 4H), 1.51 (s, 3H), 1.35 (d, J=6.4 Hz, 6H).

### Preparation 6: 6-isopropoxy-2-(1-methyl-2-oxabicyclo[2.1.1]hexan-4-yl)-2H-pyrazolo[3,4-b]pyridine-5-carboxylic acid

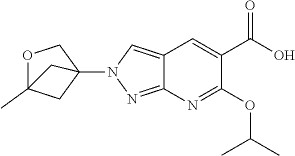 [SMILES: CC(C)Oc1nc2nn(C34COC(C)(C3)C4)cc2cc1C(=O)O]

A mixture of LiOH (412 mg, 9.81 mmol) and methyl 6-isopropoxy-2-(1-methyl-2-oxabicyclo[2.1.1]hexan-4-yl)-2H-pyrazolo[3,4-b]pyridine-5-carboxylate [preparation 5](0.390 g, 1.18 mmol) in water (6 mL) and MeOH (18 mL) was stirred at 20° C. for 6 hours under N₂ atmosphere. The mixture was evaporated to give the crude product and adjusted pH to 1-2 with 2 N HCl. The mixture was extracted with DCM (3×50 mL). The combined organic layers were washed with brine (50 mL), dried over Na₂SO₄, filtered and concentrated to give 6-isopropoxy-2-(1-methyl-2-oxabicyclo[2.1.1]hexan-4-yl)-2H-pyrazolo[3,4-b]pyridine-5-carboxylic acid (1.20 g, 96.3% yield) as an off-white solid. LCMS (ESI): 318.2 [M+H]⁺. 1H NMR: (400 MHz, DMSO-d₆) δ ppm 12.74 (s, 1H), 8.54 (s, 1H), 8.53 (s, 1H), 5.32-5.42 (m, 1H), 4.08 (s, 2H), 2.34-2.38 (m, 2H), 2.14-2.18 (m, 2H), 1.49 (s, 3H), 1.34 (d, J=6.4 Hz, 6H).

### Preparation 7: 6-cyclobutoxy-2-nitronicotinaldehyde

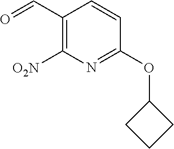 [SMILES: O=Cc1ccc(OC2CCC2)nc1[N+](=O)[O-]]

The title compound 6-cyclobutoxy-2-nitronicotinaldehyde was prepared in a similar fashion as described in the preparation for 6-isopropoxy-2-nitronicotinaldehyde (preparation 1) using cyclobutanol instead of isopropanol in Step b. LCMS (ESI): 223.2 [M+H]⁺.

### Preparation 8: 6-cyclobutoxy-2-(1-methyl-2-oxabicyclo[2.1.1]hexan-4-yl)-2H-pyrazolo[3,4-b]pyridine

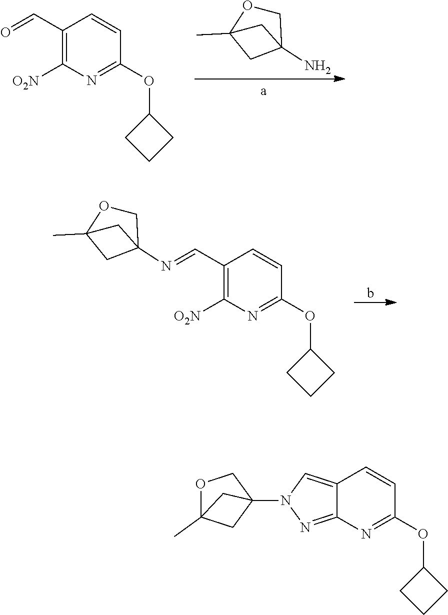

Step a: To a mixture of 1-methyl-2-oxabicyclo[2.1.1]hexan-4-amine hydrochloride (1.52 g, 10.2 mmol), TEA (2.62 g, 25.9 mmol, 3.61 mL) in i-PrOH (20 mL) was added 6-cyclobutoxy-2-nitronicotinaldehyde [preparation 7](1.15 g, 5.18 mmol) at 20° C. The resulting mixture was stirred at 80° C. for 12 h under N₂ atmosphere. The mixture was cooled to room temperature and evaporated to give the crude which was purified by column chromatography on silica gel (33% EtOAc in PE) to give (E)-1-(6-cyclobutoxy-2-nitropyridin-3-yl)-N-(1-methyl-2-oxabicyclo[2.1.1]hexan-4-yl)methanimine (1.35 g, 82.2% yield) as a yellow solid.

Step b: To a mixture of (E)-1-(6-cyclobutoxy-2-nitropyridin-3-yl)-N-(1-methyl-2-oxabicyclo[2.1.1]hexan-4-yl)methanimine (1.35 g, 4.25 mmol) in i-PrOH (15 mL) was added tributylphosphane (2.58 g, 12.7 mmol, 3.19 mL) at 20° C. The resulting mixture was stirred at 80° C. for 4 h under N₂ atmosphere. The mixture was cooled to room temperature and evaporated to give the crude which was purified by column chromatography on silica gel (Petroleum ether/EtOAc=1/1) to give 6-cyclobutoxy-2-(1-methyl-2-oxabicyclo[2.1.1]hexan-4-yl)-2H-pyrazolo[3,4-b]pyridine (0.40 g, 33% yield) as a yellow solid. 1H NMR: (400 MHz, CDCl₃) δ ppm 7.84 (s, 1H), 7.82 (s, 1H), 6.58 (d, J=9.2 Hz, 1H), 5.40-5.48 (m, 1H), 4.23 (s, 2H), 2.56-2.59 (m, 2H), 2.29-2.33 (m, 4H), 2.12-2.24 (m, 2H), 1.67-1.88 (m, 2H), 1.59 (s, 3H).

### Preparation 9: 5-Bromo-6-cyclobutoxy-2-(1-methyl-2-oxabicyclo[2.1.1]hexan-4-yl)-2H-pyrazolo[3,4-b]pyridine

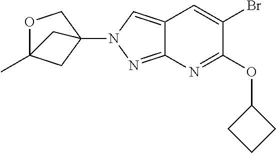 [SMILES: CC12CC(n3cc4cc(Br)c(OC5CCC5)nc4n3)(CO1)C2]

To a mixture of 6-cyclobutoxy-2-(1-methyl-2-oxabicyclo[2.1.1]hexan-4-yl)-2H-pyrazolo[3,4-b]pyridine [preparation 8](460 mg, 1.68 mmol) in MeCN (10 mL) was added NBS (329 mg, 1.85 mmol) at 20° C. The resulting mixture was stirred at 20° C. for 12 h under N₂ atmosphere. The mixture was evaporated to give the crude which was purified by column chromatography on silica gel (Petroleum ether/EtOAc=1/1) to give 5-bromo-6-cyclobutoxy-2-(1-methyl-2-oxabicyclo[2.1.1]hexan-4-yl)-2H-pyrazolo[3,4-b]pyridine (0.50 g, 82% yield) as a yellow solid. 1H NMR: (400 MHz, CDCl₃) δ ppm 8.13 (s, 1H), 7.81 (s, 1H), 5.40-5.48 (m, 1H), 4.22 (s, 2H), 2.56-2.67 (m, 2H), 2.25-2.35 (m, 6H), 1.69-1.93 (m, 2H), 1.60 (s, 3H).

### Preparation 10: Methyl 6-cyclobutoxy-2-(1-methyl-2-oxabicyclo[2.1.1]hexan-4-yl)-2H-pyrazolo[3,4-b]pyridine-5-carboxylate

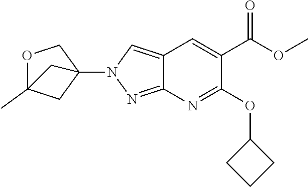

To a mixture of Pd(dppf)Cl₂ (100.4 mg, 137.3 μmol), TEA (417 mg, 4.12 mmol, 574 μL) in MeOH (30 mL) was added 5-bromo-6-cyclobutoxy-2-(1-methyl-2-oxabicyclo[2.1.1]hexan-4-yl)-2H-pyrazolo[3,4-b]pyridine [preparation 9](500 mg, 1.37 mmol) at 20° C. The resulting mixture was stirred at 80° C. for 12 h under CO (50 psi) atmosphere. The mixture was cooled to room temperature and evaporated to give the crude which was purified by column chromatography on silica gel (Petroleum ether/EtOAc=1/2) to give methyl 6-cyclobutoxy-2-(1-methyl-2-oxabicyclo[2.1.1]hexan-4-yl)-2H-pyrazolo[3,4-b]pyridine-5-carboxylate (0.41 g, 87% yield) as a yellow solid. LCMS (ESI): 344.1 [M+H]⁺. 1H NMR: (400 MHz, CDCl₃) δ ppm 8.53 (s, 1H), 7.93 (s, 1H), 5.39-5.55 (m, 1H), 4.21 (s, 2H), 3.91 (s, 3H), 2.55-2.64 (m, 2H), 2.30-2.35 (m, 4H), 2.19-2.27 (m, 2H), 1.66-1.89 (m, 2H), 1.58 (s, 3H).

### Preparation 11: 6-cyclobutoxy-2-(1-methyl-2-oxabicyclo[2.1.1]hexan-4-yl)-2H-pyrazolo[3,4-b]pyridine-5-carboxylic acid

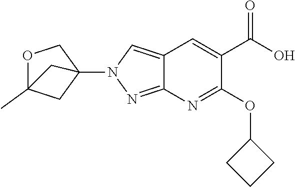 [SMILES: CC12CC(n3cc4cc(C(=O)O)c(OC5CCC5)nc4n3)(CO1)C2]

A mixture of LiOH (122 mg, 2.91 mmol) and methyl 6-cyclobutoxy-2-(1-methyl-2-oxabicyclo[2.1.1]hexan-4-yl)-2H-pyrazolo[3,4-b]pyridine-5-carboxylate [preparation 10](0.390 g, 1.18 mmol) in water (4 mL) and MeOH (12 mL) was stirred at 20° C. for 12 h under N₂ atmosphere. The mixture was evaporated to give the crude and adjusted pH to 1-2 with 2 N HCl. The mixture was extracted with DCM (3×50 mL). The combined organic layers were washed with brine (50 mL), dried over Na₂SO₄, filtered and concentrated to give 6-cyclobutoxy-2-(1-methyl-2-oxabicyclo[2.1.1]hexan-4-yl)-2H-pyrazolo[3,4-b]pyridine-5-carboxylic acid (280 mg, 73.0% yield) as an off-white solid. LCMS (ESI): 330.2 [M+H]⁺. 1H NMR: (400 MHz, DMSO-d₆) δ ppm 12.79 (br.s, 1H), 8.56 (s, 1H), 8.55 (s, 1H), 5.18-5.26 (m, 1H), 4.07 (s, 2H), 2.44-2.49 (m, 2H), 2.36-2.38 (m, 2H), 2.14-2.16 (m, 2H), 2.02-2.09 (m, 2H), 1.65-1.83 (m, 2H), 1.49 (s, 3H).

### Preparation 12: 6-Cyclobutoxy-2-(1-methyl-2-oxabicyclo[2.2.1]heptan-4-yl)-2H-pyrazolo[3,4-b]pyridine

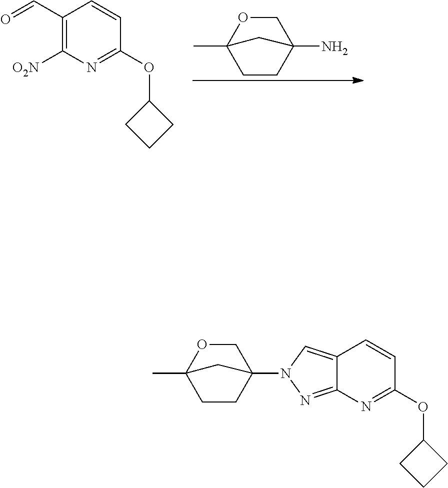

To a solution of 1-methyl-2-oxabicyclo[2.2.1]heptan-4-amine (331 mg, 2.03 mmol) and 6-cyclobutoxy-2-nitronicotinaldehyde [preparation 7](300 mg, 1.35 mmol) in IPA (10 mL) was added TEA (137 mg, 1.35 mmol, 188 μL) and stirred at 80° C. for 16 h. To a mixture cooled to 25° C. was added P(n-Bu)₃ (819 mg, 4.05 mmol) in portions under N₂ atmosphere. The reaction mixture was quenched by the addition of saturated aqueous NH₄Cl solution (20 mL), the aqueous layers were separated and extracted with EtOAc (50 mL×2). The combined organic layers were washed with brine (50 mL), dried over Na₂SO₄, filtered and concentrated in vacuo to give the residue, which was purified by Combi-Flash (PE/EtOAc=10/1 to 5/1) to give 6-Cyclobutoxy-2-(1-methyl-2-oxabicyclo[2.2.1]heptan-4-yl)-2H-pyrazolo[3,4-b]pyridine (140 mg, 31.2% yield) as yellow oil. LCMS (ESI): 300.1 [M+H]⁺.

### Preparation 13: 5-bromo-6-cyclobutoxy-2-(1-methyl-2-oxabicyclo[2.2.1]heptan-4-yl)-2H-pyrazolo[3,4-b]pyridine

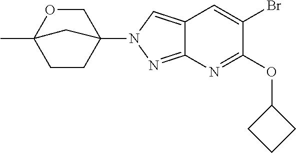 [SMILES: CC12CCC(n3cc4cc(Br)c(OC5CCC5)nc4n3)(CO1)C2]

To a solution of 6-cyclobutoxy-2-(1-methyl-2-oxabicyclo[2.2.1]heptan-4-yl)-2H-pyrazolo[3,4-b]pyridine [preparation 12](140 mg, 468 mol) in acetonitrile (10 mL) was added NBS (83.2 mg, 468 mol) and stirred at 25° C. for 16 h. The mixture was concentrated and then water (80 mL) was added. The mixture was extracted with EtOAc (50 mL×3). The combined organic layers were washed with brine (50 mL), dried over Na₂SO₄, filtered and concentrated in vacuo to give the residue, which was purified by prep-TLC (DCM/EtOAc=10/1) to give 5-bromo-6-cyclobutoxy-2-(1-methyl-2-oxabicyclo[2.2.1]heptan-4-yl)-2H-pyrazolo[3,4-b]pyridine (100 mg, 50.9% yield) as yellow solid. LCMS (ESI): 379.9 [M+H]⁺.

### Preparation 14: methyl 6-cyclobutoxy-2-(1-methyl-2-oxabicyclo[2.2.1]heptan-4-yl)-2H-pyrazolo[3,4-b]pyridine-5-carboxylate

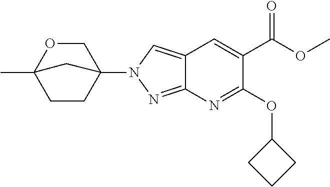

To a solution of 5-bromo-6-cyclobutoxy-2-(1-methyl-2-oxabicyclo[2.2.1]heptan-4-yl)-2H-pyrazolo[3,4-b]pyridine [preparation 13](100 mg, 264 mol) in MeOH (10 mL) was added Pd(dppf)Cl₂ (19.3 mg, 26.4 mol) and TEA (267 mg, 2.64 mmol). The reaction system was charged with CO for three times. After that, the reaction mixture was stirred under 80° C. and CO (50 psi) for 16 h. After the reaction mixture was cool down to 20° C., the mixture was filtered through celite plate, the filtrate was concentrated in vacuo to give the crude product, which was purified by silica gel chromatography (PE:EtOAc=1:1) to give methyl 6-cyclobutoxy-2-(1-methyl-2-oxabicyclo[2.2.1]heptan-4-yl)-2H-pyrazolo[3,4-b]pyridine-5-carboxylate (80.0 mg, 76.2% yield) as yellow oil. LCMS (ESI): 358.5 [M+H]⁺.

### Preparation 15: 6-cyclobutoxy-2-(1-methyl-2-oxabicyclo[2.2.1]heptan-4-yl)-2H-pyrazolo[3,4-b]pyridine-5-carboxylic acid

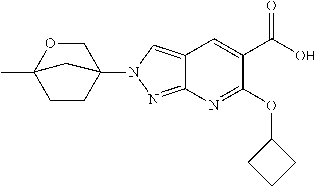 [SMILES: CC12CCC(n3cc4cc(C(=O)O)c(OC5CCC5)nc4n3)(CO1)C2]

To a solution of methyl 6-cyclobutoxy-2-(1-methyl-2-oxabicyclo[2.2.1]heptan-4-yl)-2H-pyrazolo[3,4-b]pyridine-5-carboxylate [preparation 14](80.0 mg, 224 mol) in MeOH (1 mL) and water (1 mL) was added LiOH (28.2 mg, 671 mol) at 25° C. and stirred at 25° C. for 1 h. The mixture was adjusted by HCl aq to pH=7 and concentrated in vacuo to give the residue, which was lyophilized to give 6-cyclobutoxy-2-(1-methyl-2-oxabicyclo[2.2.1]heptan-4-yl)-2H-pyrazolo[3,4-b]pyridine-5-carboxylic acid (74.0 mg, 86.6% yield) as white solid. LCMS (ESI): 344.1 [M+H]⁺. ¹H NMR: (500 MHz, DMSO-d₆) δ ppm 8.50 (s, 1H), 8.47 (s, 1H), 5.24-5.17 (m, 1H), 4.04 (d, J=6.0 Hz, 1H), 3.96 (dd, J=6.0, 3.5 Hz, 1H), 2.48-2.41 (m, 2H), 2.34-2.28 (m, 2H), 2.25-2.17 (m, 2H), 2.12-2.03 (m, 2H), 1.98-1.91 (m, 1H), 1.84-1.76 (m, 2H), 1.72-1.62 (m, 1H), 1.39 (s, 3H).

### Preparation 16: 2-(1-(Fluoromethyl)-2-oxabicyclo[2.1.1]hexan-4-yl)-6-isopropoxy-2H-pyrazolo[3,4-b]pyridine

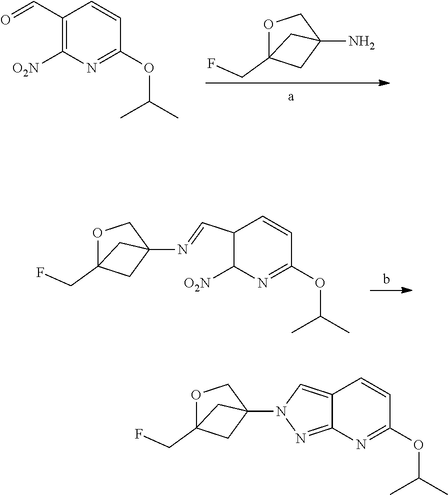

Step a: To a solution of 6-isopropoxy-2-nitronicotinaldehyde [preparation 1](502 mg, 2.39 mmol) in IPA (5 mL) was added 1-(fluoromethyl)-2-oxabicyclo[2.1.1]hexan-4-amine (400 mg, 2.39 mmol, HCl) and TEA (212 mg, 2.09 mmol) at 25° C. The mixture was stirred at 80° C. for 16 h. The reaction mixture was concentrated in vacuo to give the residue, which was purified by silica gel chromatography using a gradient (20-33% EtOAc in PE) to give (E)-N-(1-(fluoromethyl)-2-oxabicyclo[2.1.1]hexan-4-yl)-1-(6-isopropoxy-2-nitropyridin-3-yl)methanimine (570 mg, 22.0% yield) as a yellow solid, which was used immediately in the next step.

Step b: To a solution of (E)-N-(1-(fluoromethyl)-2-oxabicyclo[2.1.1]hexan-4-yl)-1-(6-isopropoxy-2-nitropyridin-3-yl)methanimine (570 mg, 1.75 mmol) in IPA (7 mL) was added tributylphosphane (1.06 g, 5.26 mmol) at 25° C., the reaction system was charged with N₂ for three times. The mixture was stirred at 80° C. for 3 h. The reaction mixture was quenched by the addition of saturated aqueous NH₄Cl solution (100 mL), the aqueous layer was separated and extracted with EtOAc (50 mL×2). The combined organic layers were washed with brine (50 mL), dried over Na₂SO₄, filtered and concentrated in vacuo to give the residue, which was purified by Combi-Flash (PE/EtOAc=5/1 to 3/1) to give 2-(1-(fluoromethyl)-2-oxabicyclo[2.1.1]hexan-4-yl)-6-isopropoxy-2H-pyrazolo[3,4-b]pyridine (110 mg, 19.4% yield) as a yellow solid. LCMS (ESI): 292.3 [M+H]⁺.

### Preparation 17: 5-bromo-2-(1-(fluoromethyl)-2-oxabicyclo[2.1.1]hexan-4-yl)-6-isopropoxy-2H-pyrazolo[3,4-b]pyridine

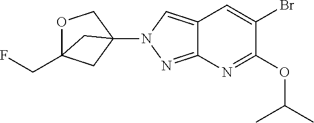 [SMILES: CC(C)Oc1nc2nn(C34COC(CF)(C3)C4)cc2cc1Br]

To a solution of 2-(1-(fluoromethyl)-2-oxabicyclo[2.1.1]hexan-4-yl)-6-isopropoxy-2H-pyrazolo[3,4-b]pyridine [preparation 16](110 mg, 377 mol) in acetonitrile (5 mL) was added NBS (67.2 mg, 377 mol) at 0° C. The mixture was stirred at 25° C. for 14 h. The mixture was diluted with saturated Na₂SO₃·aq (50 mL) and it was extracted with EtOAc (100 mL×2). The combined organic layers were washed with brine (50 mL) and dried over Na₂SO₄, filtered. The filtrate was concentrated in vacuo to give the residue, which was purified by prep-TLC (DCM/EA=20/1) to give 5-bromo-2-(1-(fluoromethyl)-2-oxabicyclo[2.1.1]hexan-4-yl)-6-isopropoxy-2H-pyrazolo[3,4-b]pyridine (130 mg, 83.7% yield) as white solid. LCMS (ESI): 372.1 [M+H]⁺.

### Preparation 18: methyl 2-(1-(fluoromethyl)-2-oxabicyclo[2.1.1]hexan-4-yl)-6-isopropoxy-2H-pyrazolo[3,4-b]pyridine-5-carboxylate

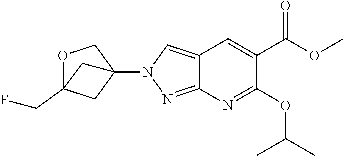 [SMILES: COC(=O)c1cc2cn(C34COC(CF)(C3)C4)nc2nc1OC(C)C]

To a solution of 5-bromo-2-(1-(fluoromethyl)-2-oxabicyclo[2.1.1]hexan-4-yl)-6-isopropoxy-2H-pyrazolo[3,4-b]pyridine [preparation 17](70.0 mg, 189 mol) in MeOH (20 mL) was added TEA (191 mg, 1.89 mmol) and Pd(dppf)Cl₂ (13.8 mg, 18.9 mol) at 25° C. under N₂. Then the mixture was stirred at 80° C. under CO (50 psi) for 24 hours. The mixture was concentrated to give the residue. The residue was purified by combi-flash (PE/EA from 3/1 to 1/1) to give methyl 2-(1-(fluoromethyl)-2-oxabicyclo[2.1.1]hexan-4-yl)-6-isopropoxy-2H-pyrazolo[3,4-b]pyridine-5-carboxylate (60.0 mg, 81.7% yield) as a yellow oil. LCMS (ESI): 350.1 [M+H]⁺.

### Preparation 19: 2-(1-(fluoromethyl)-2-oxabicyclo[2.1.1]hexan-4-yl)-6-isopropoxy-2H-pyrazolo[3,4-b]pyridine-5-carboxylic acid

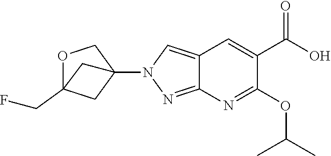 [SMILES: CC(C)Oc1nc2nn(C34COC(CF)(C3)C4)cc2cc1C(=O)O]

To a solution of methyl 2-(1-(fluoromethyl)-2-oxabicyclo[2.1.1]hexan-4-yl)-6-isopropoxy-2H-pyrazolo[3,4-b]pyridine-5-carboxylate [preparation 18](60.0 mg, 172 mol) in MeOH (3 mL) and water (3 mL) was added NaOH (13.7 mg, 343 mol) at 20° C. The mixture was stirred at 20° C. for 2 hours. MeOH was evaporated under vacuum. The mixture was acidified with HCl to pH<7 and evaporated under vacuum to give 2-(1-(fluoromethyl)-2-oxabicyclo[2.1.1]hexan-4-yl)-6-isopropoxy-2H-pyrazolo[3,4-b]pyridine-5-carboxylic acid (50.0 mg, 78.1% yield) as a white solid. LCMS (ESI): 336.2 [M+H]⁺.

### Preparation 20: 6-Isopropoxy-2-(1-(methoxymethyl)-2-oxabicyclo[2.1.1]hexan-4-yl)-2H-pyrazolo[3,4-b]pyridine

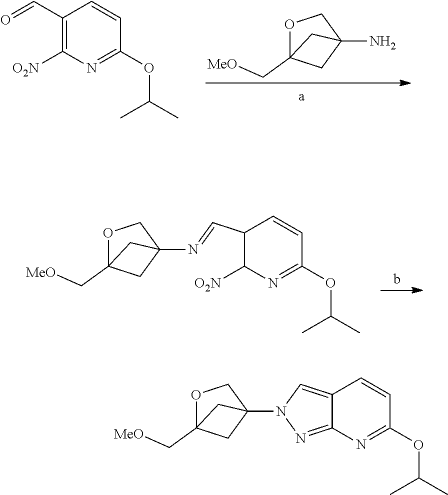

Step a: To a solution of 1-(methoxymethyl)-2-oxabicyclo[2.1.1]hexan-4-amine hydrochloride (200 mg, 1.11 mmol) in IPA (10 mL) was added 6-isopropoxy-2-nitronicotinaldehyde [preparation 1](200 mg, 951 mol) and TEA (96.3 mg, 951 mol, 133 L) at 20° C. The reaction was stirred at 80° C. for 3 hours. Solvent was evaporated under vacuum. The residue was purified by silica gel chromatography (10%-33% EtOAc in PE) to give (E)-1-(6-isopropoxy-2-nitropyridin-3-yl)-N-(1-(methoxymethyl)-2-oxabicyclo[2.1.1]hexan-4-yl)methanimine (300 mg, 84.6% yield) as a yellow solid. ¹H NMR: (500 MHz, CDCl₃) δ: 8.54 (s, 1H), 8.45 (d, J=9.0 Hz, 1H), 6.99 (d, J=8.5 Hz, 1H), 5.41-5.35 (m, 1H), 3.84 (s, 2H), 3.72 (s, 2H), 3.45 (s, 3H), 2.16-2.10 (m, 2H), 1.92-1.86 (m, 2H), 1.40 (d, J=6.0 Hz, 6H).

Step b: To a solution of (E)-1-(6-isopropoxy-2-nitropyridin-3-yl)-N-(1-(methoxymethyl)-2-oxabicyclo[2.1.1]hexan-4-yl)methanimine (300 mg, 889 mol) in IPA (5 mL) was added tributylphosphane (540 mg, 2.67 mmol, 666 L) at 20° C. The reaction system was charged with N₂ for three times. The reaction was stirred at 80° C. for 3 hours. The reaction mixture was quenched by the addition of saturated aqueous NH₄Cl solution (10 mL), extracted with EtOAc (10 mL×3). The combined organic layer was dried over Na₂SO₄; filtered and evaporated under vacuum. The combined residue was purified by prep-HPLC (Column: Welch Xtimate C18 150×25 mm×5 um, water (10 mM NH₄HCO₃)-ACN as a mobile phase, from 30% to 60%, Gradient Time (min): 10, Flow Rate (ml/min): 25) to give 6-isopropoxy-2-(1-(methoxymethyl)-2-oxabicyclo[2.1.1]hexan-4-yl)-2H-pyrazolo[3,4-b]pyridine (69.0 mg, 23.0% yield) as colorless oil. LCMS (ESI): 304.1 [M+H]⁺. ¹H NMR (400 MHz, CDCl₃) E: 7.83-7.80 (m, 2H), 6.55 (d, J=8.8 Hz, 1H), 5.64-5.54 (m, 1H), 4.28 (s, 2H), 3.76 (s, 2H), 3.47 (s, 3H), 2.42-2.36 (m, 4H), 1.39 (d, J=6.4 Hz, 6H).

### Preparation 21: 5-bromo-6-isopropoxy-2-(1-(methoxymethyl)-2-oxabicyclo[2.1.1]hexan-4-yl)-2H-pyrazolo[3,4-b]pyridine

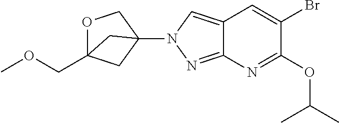 [SMILES: COCC12CC(n3cc4cc(Br)c(OC(C)C)nc4n3)(CO1)C2]

To a solution of 6-isopropoxy-2-(1-(methoxymethyl)-2-oxabicyclo[2.1.1]hexan-4-yl)-2H-pyrazolo[3,4-b]pyridine [preparation 20](69.0 mg, 227 mol) in MeCN (5 mL) was added NBS (40.5 mg, 227 mol) at 20° C. The reaction was stirred at 20° C. for 14 hours. The reaction was quenched with saturate Na₂SO₃ (15 mL), extracted with EtOAc (10 mL×3). The combined organic layer was dried over Na₂SO₄; filtered and evaporated under vacuum. The residue was purified by prep-TLC (DCM:EtOAc=10:1) to give 5-bromo-6-isopropoxy-2-(1-(methoxymethyl)-2-oxabicyclo[2.1.1]hexan-4-yl)-2H-pyrazolo[3,4-b]pyridine (50.0 mg, 51.7% yield) as yellow oil. LCMS (ESI): 381.9 [M+H]⁺.

### Preparation 22: methyl 6-isopropoxy-2-(1-(methoxymethyl)-2-oxabicyclo[2.1.1]hexan-4-yl)-2H-pyrazolo[3,4-b]pyridine-5-carboxylate

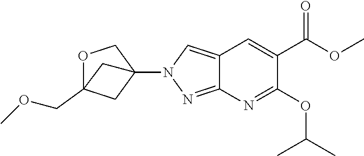

To a solution of 5-bromo-6-isopropoxy-2-(1-(methoxymethyl)-2-oxabicyclo[2.1.1]hexan-4-yl)-2H-pyrazolo[3,4-b]pyridine [preparation 21](50.0 mg, 131 mol) in MeOH (10 mL) was added Pd(dppf)Cl₂ (9.6 mg, 13 mol) and TEA (132 mg, 1.31 mmol, 182 L) at 20° C. under Argon. The mixture was stirred at 80° C. under carbon monoxide (50 psi) for 14 hours. The reaction was evaporated under vacuum to give the residue. The residue was purified by silica gel chromatography (gradient 0-30% EtOAc in PE) to give methyl 6-isopropoxy-2-(1-(methoxymethyl)-2-oxabicyclo[2.1.1]hexan-4-yl)-2H-pyrazolo[3,4-b]pyridine-5-carboxylate (47.0 mg, 89.5% yield) as yellow oil. LCMS (ESI): 362.3 [M+H]⁺.

### Preparation 23: 6-isopropoxy-2-(1-(methoxymethyl)-2-oxabicyclo[2.1.1]hexan-4-yl)-2H-pyrazolo[3,4-b]pyridine-5-carboxylic acid

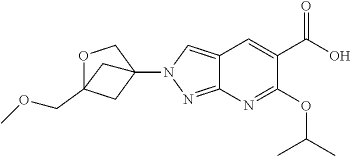 [SMILES: COCC12CC(n3cc4cc(C(=O)O)c(OC(C)C)nc4n3)(CO1)C2]

To a solution of methyl 6-isopropoxy-2-(1-(methoxymethyl)-2-oxabicyclo[2.1.1]hexan-4-yl)-2H-pyrazolo[3,4-b]pyridine-5-carboxylate [preparation 22](47.0 mg, 130 mol) in MeOH (1 mL) and water (0.5 mL) was added NaOH (10.4 mg, 260 mol) at 20° C. The reaction was stirred at 20° C. for 2 hours. MeOH was evaporated under vacuum. The mixture was acidified with aqueous KHSO₄ to pH<7 and evaporated under vacuum to give 6-isopropoxy-2-(1-(methoxymethyl)-2-oxabicyclo[2.1.1]hexan-4-yl)-2H-pyrazolo[3,4-b]pyridine-5-carboxylic acid (45.0 mg, 89.6% yield) as a brown solid. LCMS (ESI): 348.3 [M+H]⁺.

### Preparation 24: 5-bromo-4-isopropoxy-2-nitrobenzaldehyde

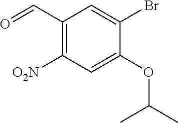 [SMILES: CC(C)Oc1cc([N+](=O)[O-])c(C=O)cc1Br]

NaH (64.4 mg, 1.61 mmol, 60% purity) was added to propan-2-ol (15.40 g, 256.3 mmol, 19.49 mL) at 0° C. The mixture was stirred at 0° C. for 30 minutes and transferred dropwise to a solution of 5-bromo-4-fluoro-2-nitro-benzaldehyde (501 mg, 2.02 mmol) in THF (10.0 mL). The reaction mixture was stirred at rt for 16 hrs. Water (10 mL) was added and the reaction mixture was extracted with EtOAc (3×50 mL). The combined organic layer was dried over Na₂SO₄ and filtered. The filtrate was concentrated in vacuo to give the residue, which was purified by silica gel column chromatography (PE:EA=20:1) to give 5-bromo-4-isopropoxy-2-nitrobenzaldehyde (120 mg, 250 μmol, 12.4% yield) as a white solid. ¹H NMR (400 MHz, CHLOROFORM-d) δ 1.51 (d, J=6.02 Hz, 6H) 4.72-4.86 (m, 1H) 7.53 (s, 1H) 8.24 (s, 1H) 10.33 (s, 1H).

### Preparation 25: 5-bromo-6-isopropoxy-2-(1-(methoxymethyl)-2-oxabicyclo[2.1.1]hexan-4-yl)-2H-indazole

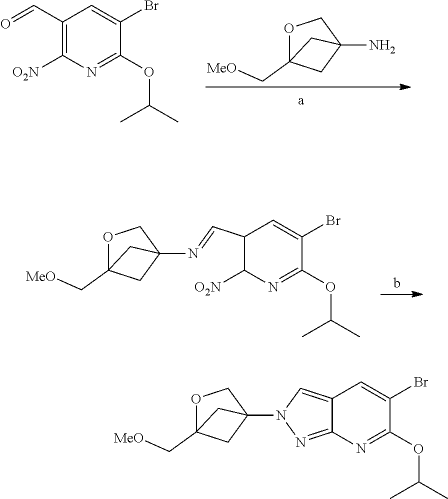

Step a: To a solution of 5-bromo-4-isopropoxy-2-nitrobenzaldehyde [preparation 24](400 mg, 1.39 mmol) and 1-(methoxymethyl)-2-oxabicyclo[2.1.1]hexan-4-amine (216 mg, 1.53 mmol) in i-PrOH (5 mL) was added TEA (140 mg, 1.39 mmol) at 25° C. The mixture was warmed to 80° C. stirred at 80° C. for 16 h. The mixture was cooled to 20° C., and then concentrated in vacuo to give the residue, which was purified by Combi Flash (PE/EtOAc=3/1) to give (E)-1-(5-bromo-4-isopropoxy-2-nitrophenyl)-N-(1-(methoxymethyl)-2-oxabicyclo[2.1.1]hexan-4-yl)methanimine (440 mg, 76.9% yield) as a yellow solid. ¹H NMR: (500 MHz, CDCl₃) δ: 8.70 (s, 1H), 8.35 (s, 1H), 7.49 (s, 1H), 4.78-4.68 (m, 1H), 3.86, (s, 2H), 3.71 (s, 2H), 3.45, (s, 3H), 2.14 (dd, J=4.5 Hz, 1.0 Hz, 2H), 1.9 (d, J=4.5 Hz, 2H), 1.45 (d, J=6.0 Hz, 6H).

Step b: To a solution of (E)-1-(5-bromo-4-isopropoxy-2-nitrophenyl)-N-(1-(methoxymethyl)-2-oxabicyclo[2.1.1]hexan-4-yl)methanimine (440 mg, 1.07 mmol) in i-PrOH (5 mL) was added P(n-Bu)₃ (649 mg, 3.21 mmol) at 20° C. The mixture was warmed to 80° C. and stirred at 80° C. for 3 h under N₂. The reaction was quenched with saturated NH4Cl aq. (20 mL) and it was extracted with EtOAc (20 mL×3). The combined organic layer was washed with brine (30 mL×2) and dried over Na₂SO₄, filtered under the vacuo. The filtrate was concentrated in vacuo to give the residue, which was purified by Combi Flash (PE:EtOAc=10:1 to 3:1) to give 5-bromo-6-isopropoxy-2-(1-(methoxymethyl)-2-oxabicyclo[2.1.1]hexan-4-yl)-2H-indazole (210 mg, 51.7% yield) as a colorless oil. ¹H NMR: (400 MHz, CDCl₃) Q: 7.84 (d, J=6.4 Hz, 2H), 7.02 (s, 1H), 4.63-4.54 (m, 1H), 4.23 (s, 2H), 3.74 (s, 2H), 3.45 (s, 3H), 2.38 (s, 4H), 1.42 (d, J=6.0 Hz, 6H).

### Preparation 26: Methyl 6-isopropoxy-2-(1-(methoxymethyl)-2-oxabicyclo[2.1.1]hexan-4-yl)-2H-indazole-5-carboxylate

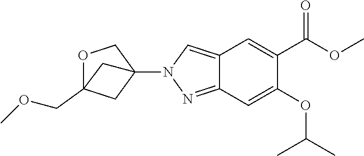

To a solution of 5-bromo-6-isopropoxy-2-(1-(methoxymethyl)-2-oxabicyclo[2.1.1]hexan-4-yl)-2H-indazole [preparation 25](210 mg, 551 mol) in MeOH (30 mL) was added Pd(dppf)Cl₂ (40.3 mg, 55.1 mol) and TEA (557 mg, 5.51 mmol) at 20° C. The mixture was degassed with CO for 3 times and it was stirred at 80° C. under CO (50 psi) for 30 h. The mixture was concentrated in vacuo to give residue which was purified by silica gel chromatography (PE:EA=1:1) to give methyl 6-isopropoxy-2-(1-(methoxymethyl)-2-oxabicyclo[2.1.1]hexan-4-yl)-2H-indazole-5-carboxylate (110 mg, 305 mol, 55.5% yield) as yellow oil. ¹H NMR: (400 MHz, CDCl₃) δ: 8.11 (s, 1H), 7.99 (s, 1H), 7.05 (s, 1H), 4.64-4.58 (m, 1H), 4.27 (s, 2H), 3.91 (s, 3H), 3.77 (s, 2H), 3.48 (s, 3H), 2.42 (s, 4H), 1.41 (t, J=5.6 Hz, 6H).

### Preparation 27: 6-isopropoxy-2-(1-(methoxymethyl)-2-oxabicyclo[2.1.1]hexan-4-yl)-2H-indazole-5-carboxylic acid

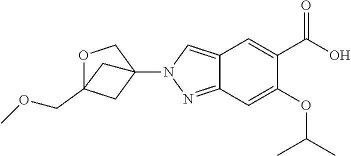 [SMILES: COCC12CC(n3cc4cc(C(=O)O)c(OC(C)C)cc4n3)(CO1)C2]

To a solution of methyl 6-isopropoxy-2-(1-(methoxymethyl)-2-oxabicyclo[2.1.1]hexan-4-yl)-2H-indazole-5-carboxylate [preparation 26](40.0 mg, 111 mol) in MeOH (6 mL) and H20 (2 mL) was added LiOH H₂O (13.9 mg, 333 mol) at 25° C. The reaction was stirred at 25° C. for 16 h. MeOH was evaporated under vacuum. The mixture was neutralized with con. HCl to pH=7 and dried to give 6-isopropoxy-2-(1-(methoxymethyl)-2-oxabicyclo[2.1.1]hexan-4-yl)-2H-indazole-5-carboxylic acid (68.0 mg, crude) as a white solid. LCMS (ESI): 346.8 [M+H]⁺.

### Preparation 28: tetrahydro-2H-pyran-4-yl 4-methylbenzenesulfonate

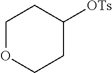 [SMILES: Cc1ccc(S(=O)(=O)OC2CCOCC2)cc1]

To a solution of tetrahydro-2H-pyran-4-ol (5.0 g, 49.0 mmol) in DCM (100 mL) was added pyridine (7.75 g, 97.92 mmol), 4-methylbenzenesulfonyl chloride (9.33 g, 49.0 mmol) and DMAP (598.1 mg, 4.90 mmol) and the reaction stirred at 50° C. for 16 hrs. The reaction mixture was diluted with water (150 mL), the layers separated and the organic phase washed with water (150 mL×2). The organic layer was concentrated in vacuo and the residue purified by silica gel chromatography with eluent (PE-EtOAc 94/6) to afford tetrahydro-2H-pyran-4-yl 4-methylbenzenesulfonate (6.17 g, 44.2% yield) as a clear oil. LCMS m/z=257.0 [M+H]⁺.

### Preparation 29: 5-bromo-6-isopropoxy-2-(1-methyl-2-oxabicyclo[2.2.1]heptan-4-yl)-2H-indazole

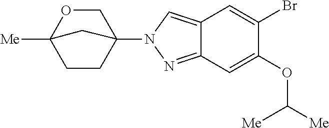 [SMILES: CC(C)Oc1cc2nn(C34CCC(C)(C3)OC4)cc2cc1Br]

1-Methyl-2-oxabicyclo[2.2.1]heptan-4-amine hydrochloride (290 mg, 1.77 mmol) was added in one portion, followed by TEA (179.3 mg, 1.77 mmol) to a solution of 5-bromo-4-isopropoxy-2-nitrobenzaldehyde [preparation 24](510.5 mg, 1.77 mmol) in isopropanol (6 mL), the vial sealed and the resulting yellow solution heated to 80° C. with stirring overnight. The mixture was cooled to rt and P(n-Bu)₃ (1.08 g, 5.32 mmol) was added in one portion. The vessel was sealed and the reaction stirred at 80° C. for an additional 16 hr. The mixture was cooled to rt, diluted with EtOAc (15 mL), washed with saturated NH₄Cl solution (10 mL), brine (10 mL) and dried over anhydrous MgSO₄. The solution was filtered, and the filtrate concentrated in vacuo. The residue was purified by silica gel chromatography (EtOAc in heptane 0/100 to 50/50) to afford 5-bromo-6-isopropoxy-2-(1-methyl-2-oxabicyclo[2.2.1]heptan-4-yl)-2H-indazole (308.2 mg, 47.7% yield) as a yellow solid.

### Preparation 30: 5-bromo-6-isopropoxy-2-(1-methyl-2-oxabicyclo[2.1.1]hexan-4-yl)-2H-indazole

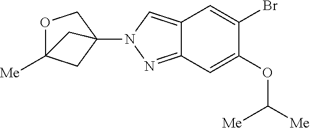 [SMILES: CC(C)Oc1cc2nn(C34COC(C)(C3)C4)cc2cc1Br]

1-Methyl-2-oxabicyclo[2.1.1]hexan-4-amine hydrochloride (1.04 g, 6.94 mmol) was added in one portion, followed by TEA (702.5 mg, 6.94 mmol) to a solution of 5-bromo-4-isopropoxy-2-nitrobenzaldehyde [preparation 24](2.0 g, 6.94 mmol) in isopropanol (15 mL), the vial sealed and the resulting yellow solution heated to 80° C. with stirring overnight. The mixture was cooled to rt and P(n-Bu)₃ (4.21 g, 20.82 mmol) was added in one portion. The vessel was sealed and the reaction stirred at 80° C. for an additional 16 hr. The mixture was cooled to rt, diluted with EtOAc (30 mL), washed with saturated NH₄Cl solution (15 mL), brine (15 mL) and dried over anhydrous MgSO₄. The solution was filtered, and the filtrate concentrated in vacuo. The residue was purified by silica gel chromatography (EtOAc in heptane 0/100 to 50/50) to afford 5-bromo-6-isopropoxy-2-(1-methyl-2-oxabicyclo[2.1.1]hexan-4-yl)-2H-indazole (901 mg, 37.0% yield) as an orange yellow solid.

### Preparation 31: 5-bromo-6-cyclobutoxy-2-(1-methyl-2-oxabicyclo[2.1.1]hexan-4-yl)-2H-indazole

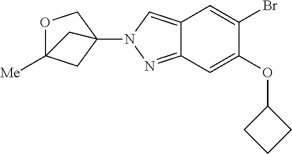 [SMILES: CC12CC(n3cc4cc(Br)c(OC5CCC5)cc4n3)(CO1)C2]

1-Methyl-2-oxabicyclo[2.1.1]hexan-4-amine hydrochloride (99.7 mg, 0.67 mmol) was added in one portion, followed by TEA (67.4 mg, 0.67 mmol) to a solution of 5-bromo-4-cyclobutoxy-2-nitrobenzaldehyde [preparation 58](200 mg, 0.67 mmol) in isopropanol (4 mL), the vial sealed and the resulting yellow solution heated to 80° C. with stirring overnight. The mixture was cooled to rt and P(n-Bu)₃ (404.5 mg, 2.0 mmol) was added in one portion. The vessel was sealed and the reaction stirred at 80° C. for an additional 16 hr. The mixture was cooled to rt, diluted with EtOAc (10 mL), washed with saturated NH₄Cl solution (10 mL), brine (10 mL) and dried over anhydrous MgSO₄. The solution was filtered, and the filtrate concentrated in vacuo. The residue was purified by silica gel chromatography (EtOAc in heptane 0/100 to 50/50) to afford 5-bromo-6-cyclobutoxy-2-(1-methyl-2-oxabicyclo[2.1.1]hexan-4-yl)-2H-indazole (216 mg, 89.4% yield) as an orange brown solid. LCMS m/z=362.9 [M+H]⁺

### Preparation 32: 6-chloro-2-((tetrahydrofuran-3-yl)methyl)-2H-pyrazolo[3,4-b]pyridine hydrochloride

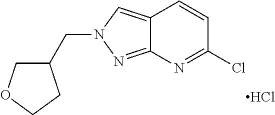 [SMILES: Clc1ccc2cn(CC3CCOC3)nc2n1 ; Cl]

To a solution of 6-chloro-2H-pyrazol[3,4-b]pyridine (2.0 g, 13.02 mmol) in DMF (15 mL) was added Cs₂CO₃ (8.49 g, 26.04 mmol) and (tetrahydrofuran-3-yl)methyl methanesulfonate (3.05 g, 16.93 mmol) and the reaction mixture stirred at 100° C. for 14 hr. The reaction was filtered and the filtrate concentrated in vacuo. The residue was purified by prep-HPLC (Phenomenex Synergi C18 150×30 μm, 4 mm; MeCN/H₂O+0.05% HCl; 24-34%) to afford 6-chloro-2-((tetrahydrofuran-3-yl)methyl)-2H-pyrazolo[3,4-b]pyridine (240 mg, 7.8% yield) as a yellow solid. LCMS m/z=238.2 [M+H]⁺

### Preparation 33: 6-chloro-2-(tetrahydro-2H-pyran-4-yl)-2H-pyrazolo[3,4-b]pyridine

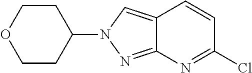 [SMILES: Clc1ccc2cn(C3CCOCC3)nc2n1]

6-Chloro-2-(tetrahydro-2H-pyran-4-yl)-2H-pyrazolo[3,4-b]pyridine was obtained as a yellow solid, 900 mg, 89.2% yield, from 6-chloro-2H-pyrazol[3,4-b]pyridine and tetrahydro-2H-pyran-4-yl 4-methylbenzenesulfonate [preparation 28], following a similar procedure to that described in Preparation 32, except the crude product was purified by prep-HPLC (Welch Xtimate C18 150×40 mm×10 μm, MeCN/H₂O+0.1% TFA; 24-44%). LCMS m/z=238.0 [M+H]⁺

### Preparation 34: 6-chloro-2-(tetrahydro-2H-pyran-2-yl)-2H-pyrazolo[3,4-b]pyridine

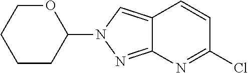 [SMILES: Clc1ccc2cn(C3CCCCO3)nc2n1]

6-Chloro-2-(tetrahydro-2H-pyran-2-yl)-2H-pyrazolo[3,4-b]pyridine was obtained as a yellow oil, 1.40 g, 90.1% yield, from 6-chloro-2H-pyrazol[3,4-b]pyridine and 3,4-dihydro-2H-pyran following the procedure described in Preparation 53. LCMS m/z=237.9 [M+H]⁺

### Preparation 35: 6-isopropoxy-2-((tetrahydrofuran-3-yl)methyl)-2H-pyrazolo[3,4-b]pyridine

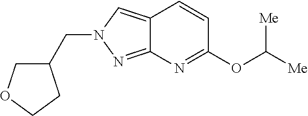 [SMILES: CC(C)Oc1ccc2cn(CC3CCOC3)nc2n1]

To a solution of 6-chloro-2-((tetrahydrofuran-3-yl)methyl)-2H-pyrazolo[3,4-b]pyridine [preparation 32](252.4 mg, 1.05 mmol) in THF (5 mL) was added NaH (168 mg, 4.20 mmol, 60% purity) and the mixture stirred at 0° C. for 30 min. Isopropanol (250 mg, 1.05 mmol) was added and the reaction stirred at 60° C. for 3 hr. The reaction was quenched with water (one drop), then concentrated in vacuo. The residue was purified by silica gel chromatography (50% EtOAC in PE) to afford 6-isopropoxy-2-((tetrahydrofuran-3-yl)methyl)-2H-pyrazolo[3,4-b]pyridine (130 mg, 47.4% yield) as a yellow oil. LCMS m/z=262.0 [M+H]⁺.

### Preparation 36: 6-isopropoxy-2-(tetrahydro-2H-pyran-2-yl)-2H-pyrazolo[3,4-b]pyridine

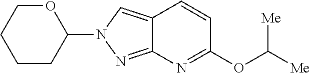 [SMILES: CC(C)Oc1ccc2cn(C3CCCCO3)nc2n1]

Starting from 6-chloro-2-(tetrahydro-2H-pyran-2-yl)-2H-pyrazolo[3,4-b]pyridine [preparation 34] and isopropanol the title compound, 6-isopropoxy-2-(tetrahydro-2H-pyran-2-yl)-2H-pyrazolo[3,4-b]pyridine, was obtained as a yellow oil (1.1 g, 68.9% yield) in a manner similar to that described in Preparation 35. LCMS m/z=262.0 [M+H]⁺

### Preparation 37: 6-Isopropoxy-2-(tetrahydro-2H-pyran-4-yl)-2H-pyrazolo[3,4-b]pyridine

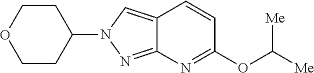 [SMILES: CC(C)Oc1ccc2cn(C3CCOCC3)nc2n1]

Starting from 6-chloro-2-(tetrahydro-2H-pyran-4-yl)-2H-pyrazolo[3,4-b]pyridine [preparation 33] and isopropanol, the title compound, 6-Isopropoxy-2-(tetrahydro-2H-pyran-4-yl)-2H-pyrazolo[3,4-b]pyridine (700 mg, 66% yield) was obtained as a yellow solid in a manner similar to that described in Preparation 35. LCMS m/z=262.0 [M+H]⁺

### Preparation 38: 5-bromo-6-isopropoxy-2-((tetrahydrofuran-3-yl)methyl)-2H-pyrazolo[3,4-b]pyridine

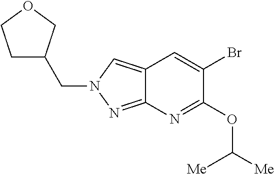 [SMILES: CC(C)Oc1nc2nn(CC3CCOC3)cc2cc1Br]

To a solution of 6-isopropoxy-2-((tetrahydrofuran-3-yl)methyl)-2H-pyrazolo[3,4-b]pyridine [preparation 35](1.96 g, 7.5 mmol) in AcOH (40 mL) was added Br₂ (1.2 g, 7.5 mmol) and the reaction stirred at 20° C. for 5 hr. The reaction was concentrated in vacuo, the residue was quenched with saturated aq. NaHCO₃ (40 mL) and extracted with EtOAc (80 mL×2). The combined organic layers were dried over Na₂SO₄, filtered and the filtrate was concentrated in vacuo. The residue was purified by Combiflash® (PE/EtOAc=34/66) to afford 5-bromo-6-isopropoxy-2-((tetrahydrofuran-3-yl)methyl)-2H-pyrazolo[3,4-b]pyridine (1.3 g, 45.9% yield) as a yellow oil. LCMS m/z=339.9 [M+H]⁺

### Preparation 40: 5-Bromo-6-isopropoxy-2H-pyrazolo[3,4-b]pyridine

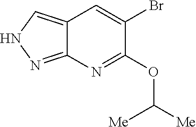 [SMILES: CC(C)Oc1nc2n[nH]cc2cc1Br]

Starting from 6-isopropoxy-2-(tetrahydro-2H-pyran-2-yl)-2H-pyrazolo[3,4-b]pyridine [Preparation 36], title compound, 5-bromo-6-isopropoxy-2H-pyrazolo[3,4-b]pyridine (280 mg, 25.9% yield) was obtained as a white solid following the procedure described i Preparation 38. LCMS m/z=257.9 [M+H]⁺

### Preparation 41: 5-Bromo-6-isopropoxy-2-(tetrahydro-2H-pyran-4-yl)-2H-pyrazolo[3,4-b]pyridine

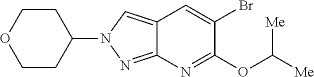 [SMILES: CC(C)Oc1nc2nn(C3CCOCC3)cc2cc1Br]

Starting from 6-isopropoxy-2-(tetrahydro-2H-pyran-4-yl)-2H-pyrazolo[3,4-b]pyridine [preparation 37], 5-bromo-6-isopropoxy-2-(tetrahydro-2H-pyran-4-yl)-2H-pyrazolo[3,4-b]pyridine was obtained as a yellow solid following the procedure described in Preparation 38. LCMS m/z=340.0 [M+H]⁺

### Preparation 42: 5-bromo-6-isopropoxy-2-(tetrahydro-2H-pyran-3-yl)-2H-pyrazolo[3,4-b]pyridine

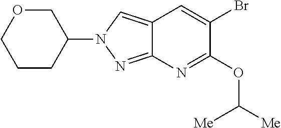 [SMILES: CC(C)Oc1nc2nn(C3CCCOC3)cc2cc1Br]

To a solution of 5-bromo-6-isopropoxy-2H-pyrazolo[3,4-b]pyridine [preparation 40](1.20 g, 4.69 mmol) in DMF (30 mL) was added K₂CO₃ (1.30 g, 9.38 mmol) and tetrahydro-2H-pyran-3-yl methanesulfonate (3.38 g, 18.76 mmol) and the reaction stirred at 100° C. for 14 hrs. The cooled mixture was concentrated in vacuo, the residue was diluted with water (100 mL) and extracted with EtOAc (40 mL×3). The combined organic layers were washed with brine (30 mL×2), dried over Na₂SO₄, filtered and evaporated under reduced pressure. The residue was purified by silica gel chromatography (25%-100% EtOAc in PE) to give 5-bromo-6-isopropoxy-2-(tetrahydro-2H-pyran-3-yl)-2H-pyrazolo[3,4-b]pyridine (150 mg, 8.5% yield) as a yellow solid. LCMS m/z=340.2 [M+H]⁺

### Preparation 43: methyl 6-isopropoxy-2-(tetrahydro-2H-pyran-3-yl)-2H-pyrazolo[3,4-b]pyridine-5-carboxylate

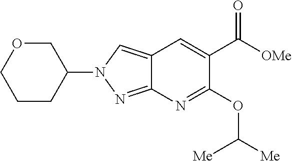 [SMILES: COC(=O)c1cc2cn(C3CCCOC3)nc2nc1OC(C)C]

To a solution of 5-bromo-6-isopropoxy-2-(tetrahydro-2H-pyran-3-yl)-2H-pyrazolo[3,4-b]pyridine [preparation 42](150 mg, 0.44 mmol) in MeOH (10 mL) was added TEA (446.2 mg, 4.41 mmol) and Pd(dppf)Cl₂ (32.3 mg, 0.044 mmol) and the reaction stirred at 80° C. under CO (50 psi) for 14 hrs. The cooled reaction was concentrated in vacuo and the residue was purified by silica gel chromatography (25%-100% EtOAc in PE) to give methyl 6-isopropoxy-2-(tetrahydro-2H-pyran-3-yl)-2H-pyrazolo[3,4-b]pyridine-5-carboxylate (70 mg, 44.7% yield) as a white solid. LCMS m/z=320.3 [M+H]⁺

### Preparation 44: Methyl 6-isopropoxy-2-((tetrahydrofuran-3-yl)methyl)-2H-pyrazolo[3,4-b]pyridine-5-carboxylate

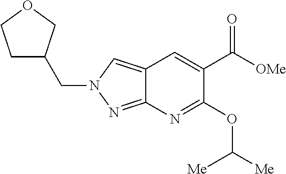 [SMILES: COC(=O)c1cc2cn(CC3CCOC3)nc2nc1OC(C)C]

To a solution of 5-bromo-6-isopropoxy-2-((tetrahydrofuran-3-yl)methyl)-2H-pyrazolo[3,4-b]pyridine [preparation 38](90 mg, 0.26 mmol) in MeOH (10 mL) was added TEA (267.7 mg, 2.65 mmol) and Pd(dppf)Cl₂ (38.7 mg, 0.053 mmol) under N₂ and the reaction mixture was stirred at 80° C. under CO (50 psi) for 14 hr. The cooled reaction was concentrated in vacuo and the residue was purified by prep-TLC (PE/EtOAc=34/66) to afford methyl 6-isopropoxy-2-((tetrahydrofuran-3-yl)methyl)-2H-pyrazolo[3,4-b]pyridine-5-carboxylate (80 mg, 93.1% yield) as a brown oil. LCMS m/z=320.0 [M+H]⁺

### Preparation 45: phenyl 6-isopropoxy-2-(1-methyl-2-oxabicyclo[2.2.1]heptan-4-yl)-2H-indazole-5-carboxylate

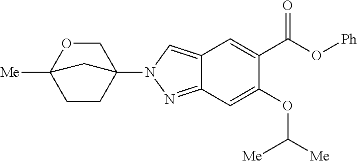 [SMILES: CC(C)Oc1cc2nn(C34CCC(C)(C3)OC4)cc2cc1C(=O)Oc1ccccc1]

TEA (213.5 mg, 2.11 mmol) was added to a mixture of 5-bromo-6-isopropoxy-2-(1-methyl-2-oxabicyclo[2.2.1]heptan-4-yl)-2H-indazole [preparation 29](308.2 mg, 0.844 mmol), Pd(OAc)₂ (18.9 mg, 0.084 mmol), Xantphos (97.6 mg, 0.169 mmol) and phenyl formate (257.6 mg, 2.11 mmol) in MeCN (6 mL) at rt. The mixture was sealed and heated at 90° C. overnight. The cooled reaction was filtered through Celite® and the filtrate was concentrated in vacuo. The residue was purified by Isco® automatic purification system (3:1 EtOAc:EtOH in heptanes 0/100 to 50/50) to afford phenyl 6-isopropoxy-2-(1-methyl-2-oxabicyclo[2.2.1]heptan-4-yl)-2H-indazole-5-carboxylate (258.3 mg, 75.3% yield) as a yellow gum. LCMS m/z=407.3 [M+H]⁺

### Preparation 46: phenyl 6-isopropoxy-2-(1-methyl-2-oxabicyclo[2.1.1]hexan-4-yl)-2H-indazole-5-carboxylate

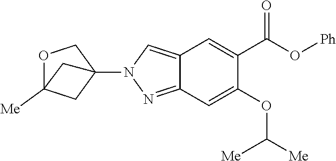 [SMILES: CC(C)Oc1cc2nn(C34COC(C)(C3)C4)cc2cc1C(=O)Oc1ccccc1]

TEA (650.2 mg, 6.42 mmol) was added to a mixture of 5-bromo-6-isopropoxy-2-(1-methyl-2-oxabicyclo[2.1.1]hexan-4-yl)-2H-indazole [preparation 30](901 mg, 2.57 mmol), Pd(OAc)₂ (57.7 mg, 0.257 mmol), Xantphos (297.4 mg, 0.514 mmol) and phenyl formate (784.6 mg, 6.42 mmol) in MeCN (9 mL) at rt. The mixture was sealed and heated at 90° C. overnight. The cooled reaction was filtered through Celite® and the filtrate was concentrated in vacuo. The residue was purified by Isco® automatic purification system (3:1 EtOAc:EtOH in heptanes 0/100 to 50/50) to afford phenyl 6-isopropoxy-2-(1-methyl-2-oxabicyclo[2.1.1]hexan-4-yl)-2H-indazole-5-carboxylate (631 mg, 62.6% yield) as an orange solid. LCMS m/z=393.3 [M+H]⁺

### Preparation 47: phenyl 6-cyclobutoxy-2-(1-methyl-2-oxabicyclo[2.1.1]hexan-4-yl)-2H-indazole-5-carboxylate

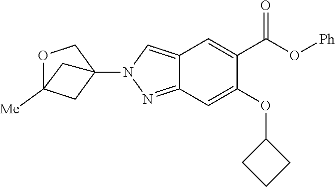 [SMILES: CC12CC(n3cc4cc(C(=O)Oc5ccccc5)c(OC5CCC5)cc4n3)(CO1)C2]

TEA (150.6 mg, 1.49 mmol) was added to a mixture of 5-bromo-6-cyclobutoxy-2-(1-methyl-2-oxabicyclo[2.1.1]hexan-4-yl)-2H-indazole [preparation 31](216.3 mg, 0.595 mmol), Pd(OAc)₂ (13.3 mg, 0.06 mmol), Xantphos (68.9 mg, 0.119 mmol) and phenyl formate (181.8 mg, 1.49 mmol) in MeCN (4 mL) at rt. The mixture was sealed and heated at 90° C. overnight. The cooled reaction was filtered through Celite® and the filtrate was concentrated in vacuo. The residue was purified by Isco® automatic purification system (3:1 EtOAc:EtOH in heptanes 0/100 to 50/50) to afford phenyl 6-cyclobutoxy-2-(1-methyl-2-oxabicyclo[2.1.1]hexan-4-yl)-2H-indazole-5-carboxylate (208 mg, 86.4% yield) as an orange yellow solid. LCMS m/z=405.2 [M+H]⁺

### Preparation 48: 6-isopropoxy-2-(1-methyl-2-oxabicyclo[2.2.1]heptan-4-yl)-2H-indazole-5-carboxylic acid

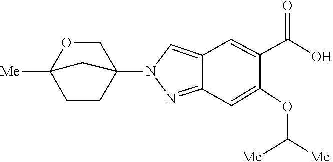 [SMILES: CC(C)Oc1cc2nn(C34CCC(C)(C3)OC4)cc2cc1C(=O)O]

To a solution of phenyl 6-isopropoxy-2-(1-methyl-2-oxabicyclo[2.2.1]heptan-4-yl)-2H-indazole-5-carboxylate [preparation 45](258.3 mg, 0.64 mmol) in H₂O (1 mL) and THF (2 mL) was added LiOH·H₂O (53.3 mg, 1.27 mmol) and the reaction stirred at rt for 16 hrs. The mixture was neutralized using 1M HCl, then extracted with EtOAc (10 mL×3). The combined organics were dried over MgSO₄, filtered and the filtrate evaporated under reduced pressure to afford 6-isopropoxy-2-(1-methyl-2-oxabicyclo[2.2.1]heptan-4-yl)-2H-indazole-5-carboxylic acid (233 mg, crude) as a yellow gum, which was used without further purification. LCMS m/z=331.1 [M+H]⁺

### Preparation 49: 6-isopropoxy-2-(1-methyl-2-oxabicyclo[2.1.1]hexan-4-yl)-2H-indazole-5-carboxylic acid

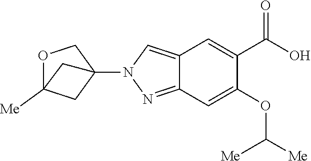 [SMILES: CC(C)Oc1cc2nn(C34COC(C)(C3)C4)cc2cc1C(=O)O]

To a solution of phenyl 6-isopropoxy-2-(1-methyl-2-oxabicyclo[2.1.1]hexan-4-yl)-2H-indazole-5-carboxylate [preparation 46](631 mg, 1.61 mmol) in H₂O (2 mL) and THF (6 mL) was added LiOH·H₂O (135.1 mg, 3.22 mmol) and the reaction stirred at rt for 16 h. The mixture was neutralized using 1M HCl, then extracted with EtOAc (20 mL×3). The combined organics were dried over MgSO₄, filtered and the filtrate evaporated under reduced pressure to afford 6-isopropoxy-2-(1-methyl-2-oxabicyclo[2.1.1]hexan-4-yl)-2H-indazole-5-carboxylic acid (765.7 mg, crude) as a brown solid, which was used without further purification. LCMS m/z=317.1 [M+H]⁺

### Preparation 50: 6-cyclobutoxy-2-(1-methyl-2-oxabicyclo[2.1.1]hexan-4-yl)-2H-indazole-5-carboxylic acid

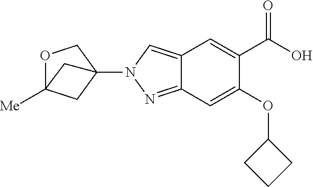 [SMILES: CC12CC(n3cc4cc(C(=O)O)c(OC5CCC5)cc4n3)(CO1)C2]

To a solution of phenyl 6-cyclobutoxy-2-(1-methyl-2-oxabicyclo[2.1.1]hexan-4-yl)-2H-indazole-5-carboxylate [preparation 47](208 mg, 0.514 mmol) in H₂O (1 mL) and THF (3 mL) was added LiOH·H₂O (43.2 mg, 1.03 mmol) and the reaction stirred at rt for 16 hrs. The mixture was neutralized using 1M HCl, then extracted with EtOAc (10 mL×3). The combined organics were dried over MgSO₄, filtered and the filtrate evaporated under reduced pressure to afford 6-cyclobutoxy-2-(1-methyl-2-oxabicyclo[2.1.1]hexan-4-yl)-2H-indazole-5-carboxylic acid (190 mg, crude), which was used without further purification. LCMS m/z=329.1 [M+H]⁺.

### Preparation 51: 6-isopropoxy-2-(tetrahydro-2H-pyran-3-yl)-2H-pyrazolo[3,4-b]pyridine-5-carboxylic acid

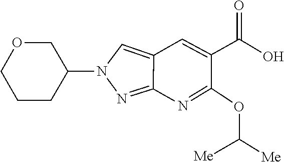 [SMILES: CC(C)Oc1nc2nn(C3CCCOC3)cc2cc1C(=O)O]

To a solution of methyl 6-isopropoxy-2-(tetrahydro-2H-pyran-3-yl)-2H-pyrazolo[3,4-b]pyridine-5-carboxylate [preparation 43](70 mg, 0.22 mmol) in MeOH (2 mL) and water (2 mL) was added NaOH (17.5 mg, 0.44 mmol) and the reaction stirred at 20° C. for 14 hrs. The reaction was concentrated in vacuo and the residue was acidified with aqueous KHSO₄ to pH<7 and evaporated under reduced pressure to afford 6-isopropoxy-2-(tetrahydro-2H-pyran-3-yl)-2H-pyrazolo[3,4-b]pyridine-5-carboxylic acid (65 mg, crude) as a white solid. LCMS m/z=306.3 [M+H]⁺

### Preparation 52: 6-isopropoxy-2-((tetrahydrofuran-3-yl)methyl)-2H-pyrazolo[3,4-b]pyridine-5-carboxylic acid

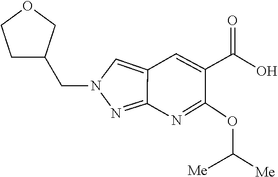 [SMILES: CC(C)Oc1nc2nn(CC3CCOC3)cc2cc1C(=O)O]

To a solution of methyl 6-isopropoxy-2-((tetrahydrofuran-3-yl)methyl)-2H-pyrazolo[3,4-b]pyridine-5-carboxylate [preparation 44](80 mg, 0.25 mmol) in MeOH (1 mL) and water (1 mL) was added NaOH (20 mg, 0.50 mmol) at 20° C. and the reaction stirred at 20° C. for 5 hr. The mixture was concentrated in vacuo to remove MeOH, the solution neutralized using aq. KHSO₄ then evaporated under reduced pressure to afford 6-isopropoxy-2-((tetrahydrofuran-3-yl)methyl)-2H-pyrazolo[3,4-b]pyridine-5-carboxylic acid (50 mg, 98.1% yield) as a white solid. ¹H NMR (400 MHz, DMSO-d₆) δ:1.33 (d, 6H), 1.58-1.67 (m, 1H), 1.88-1.97 (m, 1H), 2.81-2.88 (m, 1H), 3.47-3.53 (m, 1H), 3.61-3.70 (m, 2H), 3.75-3.81 (m, 1H), 4.35 (d, 2H), 5.35-5.42 (m, 1H), 8.45 (s, 1H), 8.51 (s, 1H).

### Preparation 53: 5-bromo-6-isopropoxy-2-(tetrahydro-2H-pyran-2-yl)-2H-pyrazolo[3,4-b]pyridine

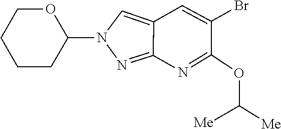 [SMILES: CC(C)Oc1nc2nn(C3CCCCO3)cc2cc1Br]

To a solution of 5-bromo-6-isopropoxy-2H-pyrazolo[3,4-b]pyridine [preparation 40](281 mg, 1.1 mmol) in DCM (10 mL) was added 3,4-dihydro-2H-pyran (139 mg, 1.65 mmol) and 4-methylbenzenesulfonic acid hydrate (41.8 mg, 0.22 mmol) and the reaction stirred at rt for 16 h. The reaction mixture was filtered and concentrated in vacuo. The residue was purified by silica gel column chromatography using silica gel chromatography eluting with PE/EtOAc (75/25) to afford 5-bromo-6-isopropoxy-2-(tetrahydro-2H-pyran-2-yl)-2H-pyrazolo[3,4-b]pyridine was obtained as a colorless oil (350 mg, 91.5% yield. LCMS m/z=339.9 [M+H]⁺.

### Preparation 54: methyl 6-isopropoxy-2-(tetrahydro-2H-pyran-2-yl)-2H-pyrazolo[3,4-b]pyridine-5-carboxylate

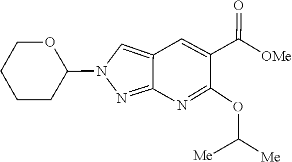 [SMILES: COC(=O)c1cc2cn(C3CCCCO3)nc2nc1OC(C)C]

The title compound, methyl 6-isopropoxy-2-(tetrahydro-2H-pyran-2-yl)-2H-pyrazolo[3,4-b]pyridine-5-carboxylate (280 mg, 90.4% yield) was obtained as a white solid from 5-bromo-6-isopropoxy-2-(tetrahydro-2H-pyran-2-yl)-2H-pyrazolo[3,4-b]pyridine [preparation 53]according to the procedure described in Preparation 43. LCMS m/z=320.0 [M+H]⁺.

### Preparation 55: 6-Isopropoxy-2-(tetrahydro-2H-pyran-2-yl)-2H-pyrazolo[3,4-b]pyridine-5-carboxylic acid

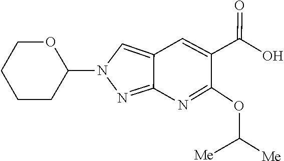 [SMILES: CC(C)Oc1nc2nn(C3CCCCO3)cc2cc1C(=O)O]

6-Isopropoxy-2-(tetrahydro-2H-pyran-2-yl)-2H-pyrazolo[3,4-b]pyridine-5-carboxylic acid was prepared as a white solid, 290 mg, crude, from methyl 6-isopropoxy-2-(tetrahydro-2H-pyran-2-yl)-2H-pyrazolo[3,4-b]pyridine-5-carboxylate [preparation 54] following the procedure described in Preparation 52. LCMS m/z=306.0 [M+H]⁺

### Preparation 56: N-(1-cyclopropyl-2-oxo-1,2-dihydropyridin-3-yl)-6-isopropoxy-2-(tetrahydro-2H-pyran-2-yl)-2H-pyrazolo[3,4-b]pyridine-5-carboxamide

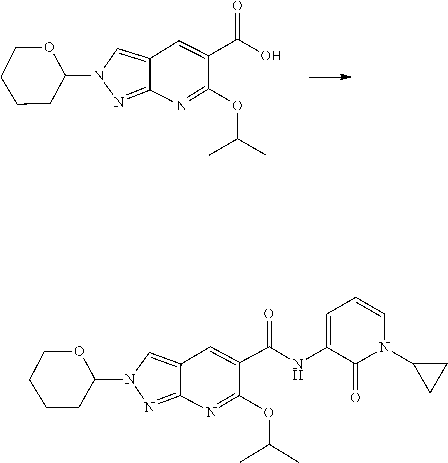 [SMILES: CC(C)Oc1nc2nn(C3CCCCO3)cc2cc1C(=O)O ; CC(C)Oc1nc2nn(C3CCCCO3)cc2cc1C(=O)Nc1cccn(C2CC2)c1=O]

To a solution of 6-isopropoxy-2-(tetrahydro-2H-pyran-2-yl)-2H-pyrazolo[3,4-b]pyridine-5-carboxylic acid [preparation 55](190 mg, 622 μmol) in Pyridine (5 mL) was added 3-amino-1-cyclopropyl-pyridin-2-one (120 mg, 643 μmol, HCl) and an EtOAc solution of T3P® (5 mL) at 20° C. The reaction mixture was stirred at 20° C. for 2 hours. The reaction was concentrated to give the residue. The residue was diluted with aqueous NaHCO₃ (30 mL), extracted with DCM (30 mL×3). The combined organic layers were dried over Na₂SO₄, filtered and concentrated to give the residue. The residue was purified by combi-flash (PE:EA from 3:1 to 0:1) to give N-(1-cyclopropyl-2-oxo-1,2-dihydropyridin-3-yl)-6-isopropoxy-2-(tetrahydro-2H-pyran-2-yl)-2H-pyrazolo[3,4-b]pyridine-5-carboxamide (200 mg, 411 μmol, 66.1% yield) as a white solid. LCMS m/z=438.3 [M+H]⁺.

### Preparation 57: N-(1-cyclopropyl-2-oxo-1,2-dihydropyridin-3-yl)-6-isopropoxy-2H-pyrazolo[3,4-b]pyridine-5-carboxamide

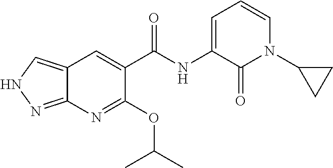 [SMILES: CC(C)Oc1nc2n[nH]cc2cc1C(=O)Nc1cccn(C2CC2)c1=O]

To a solution of N-(1-cyclopropyl-2-oxo-1,2-dihydropyridin-3-yl)-6-isopropoxy-2-(tetrahydro-2H-pyran-2-yl)-2H-pyrazolo[3,4-b]pyridine-5-carboxamide [preparation 56](200 mg, 457 μmol) in DCM (3 mL) was added TFA (3 mL) at 20° C. The mixture was stirred at 20° C. for 14 h. The mixture was concentrated to give the residue. Then the residue was diluted with H₂O (2 mL), and neutralized to pH=7 with saturated aqueous NaHCO₃. The mixture was extracted with DCM (3×20 mL), dried over Na₂SO₄, filtered and concentrated to give N-(1-cyclopropyl-2-oxo-3-pyridyl)-6-isopropoxy-2H-pyrazolo[3,4-b]pyridine-5-carboxamide (160 mg, 362 μmol, 79.2% yield) as a white solid. LCMS m/z=354.3 [M+H]⁺

### Preparation 58: 5-bromo-4-cyclobutoxy-2-nitrobenzaldehyde

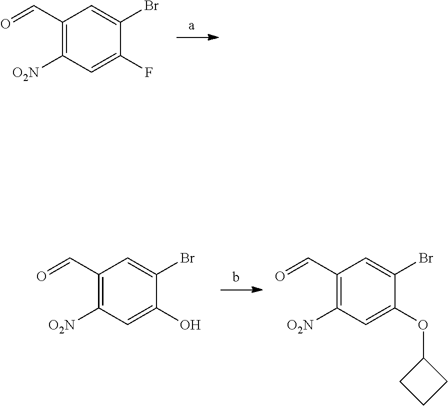 [SMILES: O=Cc1cc(Br)c(F)cc1[N+](=O)[O-] ; O=Cc1cc(Br)c(O)cc1[N+](=O)[O-] ; O=Cc1cc(Br)c(OC2CCC2)cc1[N+](=O)[O-]]

Step a. To a solution of 5-bromo-4-fluoro-2-nitro-benzaldehyde (10.0 g, 40.3 mmol) in water (20 mL) and THF (80 mL) was added NaOH (3.23 g, 80.6 mmol). The mixture was stirred at 20-25° C. for 16 h. The reaction was diluted with water (100 mL) and it was added HCl (1 M) till pH=5, and it was extracted with EtOAc (100 mL×3). The combined organic layer was washed with brine (100 mL) and dried over Na₂SO₄, filtered. The filtrate was concentrated in vacuo to give the residue, which was purified by Combi Flash (PE/EtOAc=10/1 to 3/1) to give 5-bromo-4-hydroxy-2-nitro-benzaldehyde (7.70 g, 28.2 mmol, 69.9% yield) as a yellow solid. ¹H NMR: (500 MHz, CDCl₃) δ: 10.31 (s, 1H), 8.20 (s, 1H), 7.72 (s, 1H), 6.63 (br s, 1H).

Step b. To a solution of 5-bromo-4-hydroxy-2-nitro-benzaldehyde (500 mg, 2.03 mmol) in DMF (15 mL) was added bromocyclobutane (2.74 g, 20.3 mmol, 1.91 mL) and NaHCO₃ (683 mg, 8.13 mmol, 316 L) in a microwave. The mixture was stirred at 80° C. for 3 h. The mixture was poured into ice and extracted with ethyl acetate. Then the combined organic layer was dried with Na₂SO₄. The filtrate was concentrated in vacuo to give the residue. The residue was purified by column chromatography on silica gel (from PE:EA=20:1 to 10:1) to give the 5-bromo-4-(cyclobutoxy)-2-nitro-benzaldehyde (550 mg, 1.83 mmol, 90.2% yield) as a yellow solid. ¹H NMR: (400 MHz, CDCl₃) δ: 10.32 (s, 1H), 8.21 (s, 1H), 7.37 (s, 1H), 4.89-4.80 (m, 1H), 2.61-2.58 (m, 2H), 2.35-2.30 (m, 2H), 2.00-1.97 (m, 1H), 1.85-1.80 (m, 1H).

### Preparation 59: 5-bromo-6-(cyclobutoxy)-2-(1-methyl-2-oxabicyclo[2.2.1]heptan-4-yl)indazole

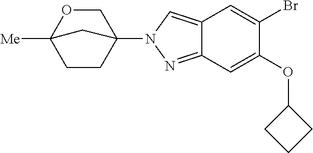 [SMILES: CC12CCC(n3cc4cc(Br)c(OC5CCC5)cc4n3)(CO1)C2]

To a 2-dram vial equipped with a stir bar was added 5-bromo-4-cyclobutoxy-2-nitrobenzaldehyde [preparation 58](917 mg, 3.06 mmol) and isopropanol (10 mL). 1-Methyl-2-oxabicyclo[2.2.1]heptan-4-amine (500 mg, 3.06 mmol, Hydrochloride) was added in one portion, followed by TEA (309 mg, 3.06 mmol, 426 μL) and the resulting solution was heated to 80° C. in a sealed 2-dram vial with stirring for overnight. The mixture was cooled to room temperature and P(n-Bu)₃ (1.85 g, 9.17 mmol, 2.29 mL) was added in one portion followed by stirring at 80° C. in a sealed 2-dram vial for overnight. The mixture was cooled to room temperature and diluted with EtOAc (10 mL). The organics were washed with ammonium chloride (10 mL), brine (10 ml), dried over MgSO₄, filtered, and concentrated in vacuo. The residue was purified by silica gel chromatography (0-50% EtOAc in heptane) to give 5-bromo-6-cyclobutoxy-2-(1-methyl-2-oxabicyclo[2.2.1]heptan-4-yl)-2H-indazole (1.04 g, 2.76 mmol, 90.2% yield) as a dark orange oil. LCMS m/z=378.8 [M+H]⁺

### Preparation 60: Phenyl 6-cyclobutoxy-2-(1-methyl-2-oxabicyclo[2.2.1]heptan-4-yl)-2H-indazole-5-carboxylate

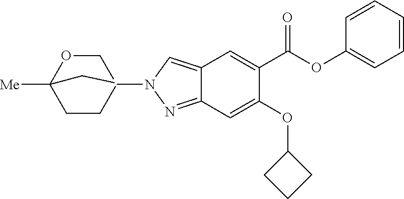

N,N-diethylethanamine (697 mg, 6.89 mmol, 960 μL) was added to a mixture of 5-bromo-6-(cyclobutoxy)-2-(1-methyl-2-oxabicyclo[2.2.1]heptan-4-yl)indazole [preparation 59](1.04 g, 2.76 mmol), diacetoxypalladium (30.9 mg, 137 μmol), (5-diphenylphosphanyl-9,9-dimethyl-xanthen-4-yl)-diphenyl-phosphane (159.5 mg, 275.6 μmol) and phenyl formate (842 mg, 6.89 mmol, 751 μL) in MeCN (10 mL) at rt. The mixture was heated at 90° C. for overnight. The reaction was cooled to room temp and filtered through a pad of celite. The filtrate was concentrated and purified by Isco automatic purification system (40 g silica gel column, 0-50% 3:1 EtOAc:EtOH in heptane) to obtain phenyl 6-(cyclobutoxy)-2-(1-methyl-2-oxabicyclo[2.2.1]heptan-4-yl)indazole-5-carboxylate (1.06 g, 2.53 mmol, 91.9% yield) as an orange oil. LCMS m/z=418.9 [M+H]⁺

### Preparation 61: 6-Cyclobutoxy-2-(1-methyl-2-oxabicyclo[2.2.1]heptan-4-yl)-2H-indazole-5-carboxylic acid

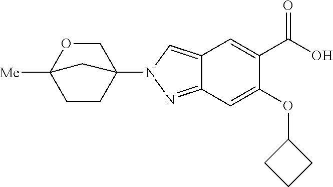 [SMILES: CC12CCC(n3cc4cc(C(=O)O)c(OC5CCC5)cc4n3)(CO1)C2]

To a solution of phenyl 6-(cyclobutoxy)-2-(1-methyl-2-oxabicyclo[2.2.1]heptan-4-yl)indazole-5-carboxylate [preparation 60](1.06 g, 2.53 mmol) in H₂O (2 mL) and THF (6 mL) was added Lithium hydroxide monohydrate (319 mg, 7.60 mmol). The mixture was stirred at rt for 16 hours. The mixture was added HCl (1M) till pH=7 and the mixture was concentrated in vacuo to give an aqueous layer. It was extracted with EtOAc (3×20 mL). The combined organic layers were dried over MgSO₄, filtered. The filtrate was concentrated in vacuo to give 6-cyclobutoxy-2-(1-methyl-2-oxabicyclo[2.2.1]heptan-4-yl)-2H-indazole-5-carboxylic acid (837 mg, 2.44 mmol, 96.5% yield, crude) as a dark brown solid. Used without further purification. LCMS m/z=342.9 [M+H]⁺-Preparation 62: 3-amino-1-(1-methylcyclopropyl)pyridin-2(1H)-one

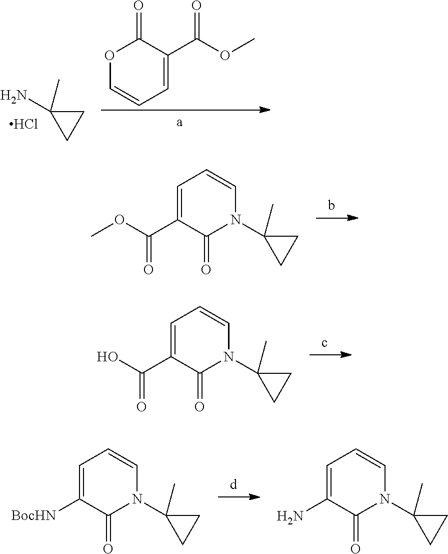

Step a: To a solution of methyl 2-oxo-2H-pyran-3-carboxylate (1.00 g, 6.49 mmol) and 1-methylcyclopropan-1-amine hydrochloride (768 mg, 7.14 mmol) in DMF (50 mL) was added TEA (1.31 g, 13.0 mmol) at 0° C. The mixture was stirred at 0° C. for 30 min and EDCI (1.62 g, 8.43 mmol) and DMAP (159 mg, 1.30 mmol) were added. The resulting mixture was stirred at 25° C. for 12 h. The mixture was diluted with water (100 mL) and the mixture was extracted with EtOAc (3×100 mL). The combined organic layers were washed with brine (100 mL), dried (Na₂SO₄) and filtered. The filtrate was concentrated and the residue was purified by silica gel chromatography (PE/EtOAc=3/1 to 0/1) to give methyl 1-(1-methylcyclopropyl)-2-oxo-1,2-dihydropyridine-3-carboxylate (220 mg, 16% yield) as a yellow oil. LCMS (ESI) m/z 207.9 (M+H)⁺. ¹HNMR (500 MHz, CHLOROFORM-d) δ ppm=8.14 (dd, J=7.0, 2.0 Hz, 1H), 7.66 (d, J=6.5 Hz, 1H), 6.21 (t, J=7.0 Hz, 1H), 3.91 (s, 3H), 1.54 (s, 3H), 1.05-0.95 (m, 4H).

Step b: To a solution of methyl 1-(1-methylcyclopropyl)-2-oxo-1,2-dihydropyridine-3-carboxylate (250 mg, 1.21 mmol) in MeOH (2 mL) and water (1 mL) was added LiOH (86.7 mg, 3.62 mmol). The mixture was stirred at 20° C. for 16 h. The reaction mixture was acidified with 1 M aqueous HCl to pH=5, and it was further diluted with water (20 mL). The mixture was extracted with EtOAc (3×20 mL). The combined organic layers were dried (Na₂SO₄) filtered and concentrated in vacuo to give 1-(1-methylcyclopropyl)-2-oxo-1,2-dihydropyridine-3-carboxylic acid (210 mg, 90% yield) as a yellow solid. ¹HNMR (400 MHz, DMSO-d₆) δ ppm=14.70 (brs, 1H), 8.33 (dd, J=7.2, 2.0 Hz, 1H), 8.23 (dd, J=6.6, 2.0 Hz, 1H), 6.66 (t, J=7.2 Hz, 1H), 1.46 (s, 3H), 1.10-1.00 (m, 2H), 0.95-0.85 (m, 2H).

Step c: To a solution of 1-(1-methylcyclopropyl)-2-oxo-1,2-dihydropyridine-3-carboxylic acid (210 mg, 1.09 mmol) in t-BuOH (10 mL) was added DPPA (449 mg, 1.63 mmol) and TEA (220 mg, 2.17 mmol). The mixture was stirred at 90° C. for 12 h. The reaction mixture was concentrated in vacuo and the residue was purified by silica gel chromatography (5%-20% PE in EtOAc) to give tert-butyl (1-(1-methylcyclopropyl)-2-oxo-1,2-dihydropyridin-3-yl)carbamate (140 mg, 48.7% yield) as a yellow oil. LCMS (ESI) m/z 265.0 (M+H)⁺.

Step d: To a solution of tert-butyl (1-(1-methylcyclopropyl)-2-oxo-1,2-dihydropyridin-3-yl)carbamate (50 mg, 190 mol) in EtOAc (1 mL) was added an EtOAc solution of HCl (4 M, 2.5 mL). The mixture was stirred at 20° C. for 1 h. The mixture was concentrated in vacuo to give 3-amino-1-(1-methylcyclopropyl)pyridin-2(1H)-one hydrochloride (35 mg, 2.2% yield) as a yellow solid. ¹H NMR (500 MHz, DMSO-d₆) δ ppm=7.50-7.40 (m, 1H), 7.20-7.10 (m, 1H), 6.20 (t, J=7.0 Hz, 1H), 1.42 (s, 3H), 1.00-0.90 (m, 4H).

### Preparation 63: 3-amino-1-(2,2-dimethylcyclopropyl)pyridin-2(1H)-one

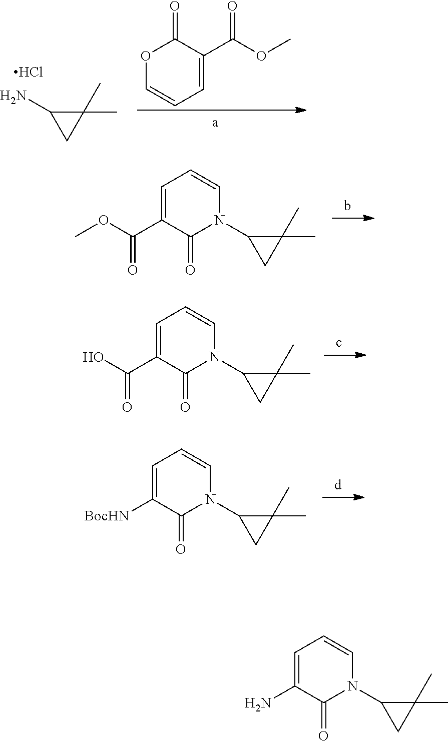

Step a: To a solution of compound methyl 2-oxo-2H-pyran-3-carboxylate (500 mg, 3.24 mmol) and compound 2,2-dimethylcyclopropan-1-amine hydrochloride (395 mg, 3.24 mmol) in DMF (5 mL) was added TEA (657 mg, 6.49 mmol (0.9 mL) at 0° C. After 30 min, DMAP (79.2 mg, 649 mol) was added, followed by and EDCI (808 mg, 4.22 mmol). The resulting mixture was stirred at rt for 12 h. The mixture was diluted with water (30 mL) and the mixture was extracted with EtOAc (3×50 mL). The combined organic layers were washed with brine (50 mL), dried (Na₂SO₄) and filtered. The filtrate was concentrated and the residue was purified by silica gel chromatography, eluting with (PE/EtOAc=3/1 to 0/1) to give methyl 1-(2,2-dimethylcyclopropyl)-2-oxo-1,2-dihydropyridine-3-carboxylate (220 mg, 30% yield) as yellow oil. LCMS (ESI) m/z 222.0 (M+H)⁺. ¹HNMR (400 MHz, CHLOROFORM-d) δ ppm=8.17 (d, J=7.0 Hz, 1H), 7.51 (d, J=7.0 Hz, 1H), 6.19 (t, J=6.5 Hz, 1H), 3.91 (s, 3H), 3.15-3.10 (m, 1H), 1.31 (s, 3H), 1.00-0.95 (m, 1H), 0.86 (s, 3H), 0.80-0.75 (m, 1H).

Step b: To a solution of compound methyl 1-(2,2-dimethylcyclopropyl)-2-oxo-1,2-dihydropyridine-3-carboxylate (220 mg, 994 mol) in MeOH (2 mL) and water (1 mL) was added LiOH (71 mg, 3.0 mmol). The mixture was stirred at 20° C. for 1 h. The reaction mixture diluted with aqueous HCl (1 M) to pH=5, and water was added (20 mL). The mixture was extracted with EtOAc (30 mL×3) and the combined organic layers were dried (Na₂SO₄) and filtered. The filtrate was concentrated in vacuo to give compound 1-(2,2-dimethylcyclopropyl)-2-oxo-1,2-dihydropyridine-3-carboxylic acid (200 mg, 97% yield) as a yellow solid, which was used without further purification. LCMS (ESI) m/z 207.9 (M+H)⁺.

Step c: To a solution of compound 1-(2,2-dimethylcyclopropyl)-2-oxo-1,2-dihydropyridine-3-carboxylic acid (150 mg, 723 mol) in t-BuOH (10 mL) was added DPPA (298 mg, 1.09 mmol, 0.2 mL) and TEA (219 mg, 2.17 mmol, 0.3 mL). The mixture was stirred at 90° C. for 12 h. The reaction mixture was concentrated in vacuo and the residue was purified by silica gel chromatography, eluting with (PE/EtOAc=I/O to 3/1) to give compound tert-butyl (1-(2,2-dimethylcyclopropyl)-2-oxo-1,2-dihydropyridin-3-yl)carbamate (70 mg, 35% yield) as yellow oil.

Step d: To a solution compound tert-butyl (1-(2,2-dimethylcyclopropyl)-2-oxo-1,2-dihydropyridin-3-yl)carbamate (80 mg, 287 mol) in EtOAc (1 mL) was added an EtOAc solution of HCl (4 M, 4.00 mL). The mixture was stirred at 20° C. for 1 h. The solution was concentrated in vacuo to give compound 3-amino-1-(2,2-dimethylcyclopropyl)pyridin-2(1H)-one (60 mg, 97% yield, HCl) as a yellow solid, which was of sufficient purity for use in the next reaction. LCMS (ESI) m/z 178.7 (M+H)⁺. ¹HNMR (500 MHz, DMSO-d₆) δ ppm=7.20-7.10 (m, 1H), 6.95-6.85 (m, 1H), 6.11 (t, J=7.0 Hz, 1H), 3.10-3.00 (m, 1H), 1.19 (s, 3H), 1.00-0.95 (m, 1H), 0.85-0.80 (m, 1H), 0.71 (s, 3H).

### Preparation 64: Cis-racemic 3-amino-1-(2-fluorocyclopropyl)pyridin-2(1H)-one

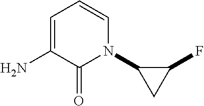

Cis-racemic 3-amino-1-(2-fluorocyclopropyl)pyridin-2(1H)-one was prepared from (cis)-2-fluorocyclopropan-1-amine in a similar fashion to that described in Preparation 65. LCMS (ESI) m/z 165.2 (M+H)⁺.

### Preparation 65: Trans-racemic 3-amino-1-(2-fluorocyclopropyl)pyridin-2(1H)-one

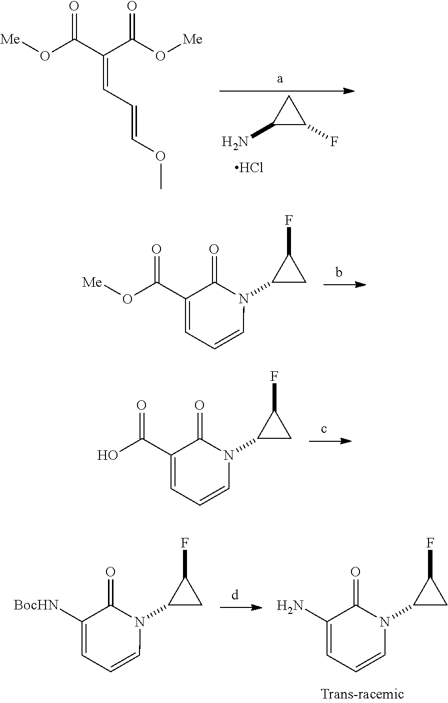

Step a: In a 30 mL vial, a mixture of racemic (trans)-2-fluorocyclopropanamine hydrochloride (279 mg, 2.50 mmol), dimethyl 2-[(E)-3-methoxyprop-2-enylidene]propanedioate (500 mg, 2.50 mmol) and triethylamine (278 mg, 2.75 mmol, 383 μL) in MeOH (3 mL) was stirred at rt for 15 h. Volatiles were evaporated under reduced pressure and the resulting residue was partitioned between dichloromethane and water. The organic layer was separated, dried over Na₂SO₄, filtered and concentrated in vacuo to obtain dimethyl racemic-(E)-2-(Trans)-(3-((2-fluorocyclopropyl)amino)allylidene)malonate. The crude material was dissolved in ethanol (3 mL) followed by the addition of solid KOH (263 mg, 4.69 mmol). The reaction mixture was stirred at rt for 1 h and then refluxed for 2 h. After that, the resulting mixture was evaporated in vacuo and the residue was dissolved in water and neutralized with conc. HCl. The aqueous solution was extracted with EtOAc (10 mL×3) and the combined organic layers were dried, filtered and concentrated to give a residual oil. It was purified by mass-directed HPLC to give methyl 1-Trans-(2-fluorocyclopropyl)-2-oxo-1,2-dihydropyridine-3-carboxylate (257 mg, 1.22 mmol, 48.6% yield) as a colorless film. LCMS m/z=211.9 [M+H]⁺; ¹H NMR (400 MHz, MeOH-d₄) δ: 1.51 (dddd, J=11.07, 8.82, 7.22, 6.27 Hz, 1H) 1.69-1.82 (m, 1H) 3.69-3.80 (m, 1H) 3.85 (s, 3H) 4.74-4.92 (m, 1H) 6.42 (t, J=7.03 Hz, 1H) 7.79 (dd, J=6.78, 2.01 Hz, 1H) 8.21 (dd, J=7.28, 2.26 Hz, 1H).

Step b: NaOH (97.2 mg, 2.43 mmol) was added to a mixture of methyl 1-Trans-(2-fluorocyclopropyl)-2-oxo-1,2-dihydropyridine-3-carboxylate (257 mg, 1.22 mmol) in THF (2 mL) and MeOH (2 mL) at rt and stirred for 5 h. The reaction mixture was dried under vacuum to give racemic 1-Trans-(2-fluorocyclopropyl)-2-oxo-1,2-dihydropyridine-3-carboxylic acid as a sodium salt. The Material was used without further purification in the next step.

Step c: To a solution of 1-Trans-(2-fluorocyclopropyl)-2-oxo-1,2-dihydropyridine-3-carboxylic acid (50.0 mg, 253 mol) in t-BuOH (3 mL) was added DPPA (105 mg, 380 mol, 82.0 L) and triethylamine (51.3 mg, 507 mol, 70.7 L). The mixture was stirred at 90° C. for 12 h. The reaction mixture was concentrated in vacuo to give the residue, which was purified by silica gel chromatography (PE/EtOAc=20/1 to 5/1) to give racemic tert-butyl (1-Trans-(2-fluorocyclopropyl)-2-oxo-1,2-dihydropyridin-3-yl)carbamate (57.8 mg, 215 mol, 84.9% yield) as a yellow oil. LCMS m/z=269.1 [M+H]⁺

Step d: To a solution of racemic tert-butyl (1-Trans-(2-fluorocyclopropyl)-2-oxo-1,2-dihydropyridin-3-yl)carbamate (142 mg, 527 mol) in dioxane (2 mL) was added HCl (4 M in dioxane, 659 L). The mixture was stirred at 22° C. for 14 h. Solvent was removed to provide rac-(Trans)-3-amino-1-(2-fluorocyclopropyl)pyridin-2(1H)-one, which was used without further purification.

### Preparation 66: Trans-racemic 3-amino-1-(2-methylcyclopropyl)pyridin-2(1H)-one hydrochloride

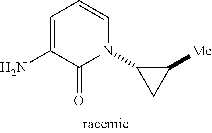

Trans-racemic 3-amino-1-(2-methylcyclopropyl)pyridin-2(1H)-one hydrochloride was prepared from Trans-2-methylcyclopropan-1-amine hydrochloride in a similar fashion to that described in Preparation 65. LCMS (ESI) m/z 169.0 (M+H)⁺.

### Preparation 67: 2-(2-oxabicyclo[2.1.1]hexan-4-yl)-6-isopropoxy-2H-pyrazolo[3,4-b]pyridine-5-carboxylic acid

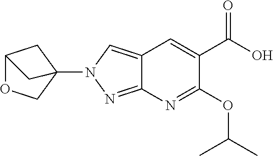 [SMILES: CC(C)Oc1nc2nn(C34COC(C3)C4)cc2cc1C(=O)O]

The title compound, 2-(2-oxabicyclo[2.1.1]hexan-4-yl)-6-isopropoxy-2H-pyrazolo[3,4-b]pyridine-5-carboxylic acid, was prepared in a similar fashion to that described in Preparations 2-6, starting with 2-oxabicyclo[2.1.1]hexan-4-amine instead of 1-methyl-2-oxabicyclo[2.1.1]hexan-4-amine in Preparation 2. LCMS m/z=342.9 [M+H]⁺

### Preparation 68: 6-(sec-butoxy)-2-(1-methyl-2-oxabicyclo[2.1.1]hexan-4-yl)-2H-pyrazolo[3,4-b]pyridine-5-carboxylic acid

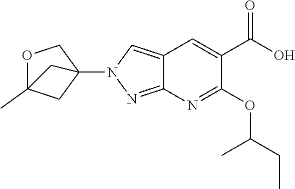 [SMILES: CCC(C)Oc1nc2nn(C34COC(C)(C3)C4)cc2cc1C(=O)O]

The title compound, 6-(sec-butoxy)-2-(1-methyl-2-oxabicyclo[2.1.1]hexan-4-yl)-2H-pyrazolo[3,4-b]pyridine-5-carboxylic acid, was prepared in a similar fashion to the preparation described for 6-isopropoxy-2-(1-methyl-2-oxabicyclo[2.1.1]hexan-4-yl)-2H-pyrazolo[3,4-b]pyridine-5-carboxylic acid [preparations 1-6] using butan-2-ol instead of isopropanol in Step b of Preparation 1. LCMS m/z=331.8 [M+H]⁺

### Preparation 69: 6-(sec-butoxy)-2-(1-(fluoromethyl)-2-oxabicyclo[2.1.1]hexan-4-yl)-2H-pyrazolo[3,4-b]pyridine-5-carboxylic acid

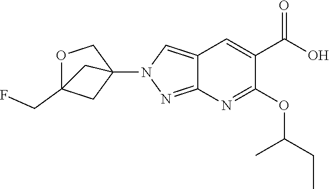 [SMILES: CCC(C)Oc1nc2nn(C34COC(CF)(C3)C4)cc2cc1C(=O)O]

The title compound, 6-(sec-butoxy)-2-(1-(fluoromethyl)-2-oxabicyclo[2.1.1]hexan-4-yl)-2H-pyrazolo[3,4-b]pyridine-5-carboxylic acid, was prepared in a similar fashion to the preparation described for 2-(1-(fluoromethyl)-2-oxabicyclo[2.1.1]hexan-4-yl)-6-isopropoxy-2H-pyrazolo[3,4-b]pyridine-5-carboxylic acid [preparation 16] using the starting material derived from butan-2-ol instead of isopropanol in Step b of Preparation 1. LCMS (ESI): 350.2 [M+H]⁺.

### Preparation 70: 6-isopropoxy-2-(1-(methoxymethyl)-2-oxabicyclo[2.2.1]heptan-4-yl)-2H-pyrazolo[3,4-b]pyridine-5-carboxylic acid

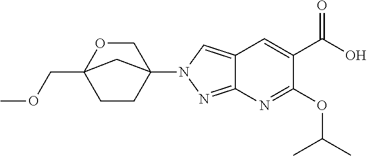 [SMILES: COCC12CCC(n3cc4cc(C(=O)O)c(OC(C)C)nc4n3)(CO1)C2]

The title compound, 6-isopropoxy-2-(1-(methoxymethyl)-2-oxabicyclo[2.2.1]heptan-4-yl)-2H-pyrazolo[3,4-b]pyridine-5-carboxylic acid, was obtained as an off-white solid in a similar fashion to that described in preparations 2-6, starting from 1-(methoxymethyl)-2-oxabicyclo[2.2.1]heptan-4-amine instead of 1-methyl-2-oxabicyclo[2.1.1]hexan-4-amine. LCMS m/z=362.2 [M+H]⁺. ¹H NMR (400 MHz, DMSO-d6) δ ppm 8.51 (s, 1H), 8.50 (s, 1H), 5.41-5.34 (m, 1H), 4.09 (d, J=6.4 Hz, 1H), 3.99-3.97 (m, 1H), 3.59 (d, J=5.6 Hz, 2H), 3.33 (s, 3H), 2.35-2.30 (m, 3H), 2.27-2.19 (m, 1H), 2.03-1.96 (m, 1H), 1.87-1.79 (m, 1H), 1.34 (s, 3H), 1.33 (s, 3H).

### Preparation 71: 6-isopropoxy-2-(1-methyl-2-oxabicyclo[2.2.1]heptan-4-yl)-2H-pyrazolo[3,4-b]pyridine-5-carboxylic acid

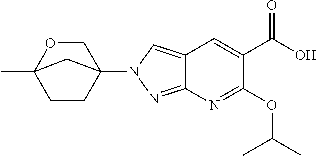 [SMILES: CC(C)Oc1nc2nn(C34CCC(C)(C3)OC4)cc2cc1C(=O)O]

The title compound, 6-isopropoxy-2-(1-methyl-2-oxabicyclo[2.2.1]heptan-4-yl)-2H-pyrazolo[3,4-b]pyridine-5-carboxylic acid, was obtained as a white solid in a similar fashion to that described in preparations 12-15, starting from 6-isopropoxy-2-nitronicotinaldehyde [preparation 1] instead of 6-cyclobutoxy-2-nitronicotinaldehyde. LCMS (ESI): 331.9 [M+H]⁺. ¹H NMR (500 MHz, MeOD) δ: 8.22 (s, 1H), 8.09 (s, 1H), 5.49-5.43 (m, 1H), 4.16 (d, J=6.0 Hz, 1H), 4.07 (dd, J₁=6.5 Hz, J₂=4.0 Hz, 1H), 2.46-2.40 (m, 1H), 2.35 (s, 2H), 2.34-2.26 (m, 1H), 2.06-1.99 (m, 1H), 1.97-1.90 (m, 1H), 1.47 (s, 3H), 1.40 (d, J=6.0 Hz, 6H).

### Preparation 72: 3-amino-1-((1R,2S)-2-methylcyclopropyl)pyridin-2(1H)-one hydrochloride

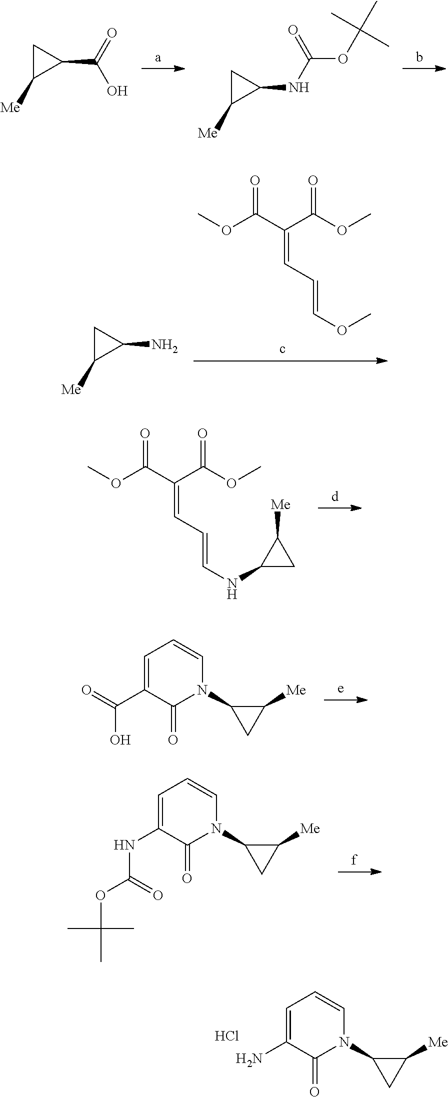

Step a: To a solution of (1R,2S)-2-methylcyclopropane-1-carboxylic acid (2.16 g, 21.58 mmol) in t-BuOH (20 mL) was added DPPA (6.53 g, 23.73 mmol) and TEA (7.20 g, 71.20 mmol) and the reaction was stirred at 90° C. for 72 h under N₂ atmosphere. Sat. aq. NaHCO₃ solution (30 mL) was added and the mixture extracted with EtOAc (30 mL×3). The combined organic layers were washed with brine (100 mL), dried over Na₂SO₄, filtered and concentrated. The crude material was purified by silica gel column chromatography (PE/EtOAc=15/1 to 5/1) to give tert-butyl ((1R,2S)-2-methylcyclopropyl)carbamate (2.7 g, 73.1% yield) as yellow solid. 1H NMR: (400 MHz, CDCl₃) δ ppm 4.56 (br s, 1H), 2.54 (br s, 1H), 1.45 (s, 9H), 1.06 (d, J=6.0 Hz, 3H), 0.97-0.82 (m, 2H), 0.10-0.02 (m, 1H).

Step b: To a solution of tert-butyl ((1R,2S)-2-methylcyclopropyl)carbamate (2.7 g, 15.77 mmol) in dioxane (10 mL) was added HCl/dioxane (4 M, 10 mL) and the reaction was stirred at 20° C. for 12 h under N₂ atmosphere. The mixture was concentrated under reduced pressure to give (1R,2S)-2-methylcyclopropan-1-amine hydrochoride (1.1 g, 64.9% yield) as yellow solid. 1H NMR: (400 MHz, DMSO-d₆) δ ppm 8.45 (br s, 2H), 2.54-2.49 (m, 1H), 1.21 (d, J=6.4 Hz, 3H), 1.10-0.99 (m, 1H), 0.93-0.85 (m, 1H), 0.57-0.50 (m, 1H).

Step c: To a solution of (1R,2S)-2-methylcyclopropan-1-amine hydrochoride (1.1 g, 10.22 mmol) in MeOH (20 mL) was added dimethyl (E)-2-(3-methoxyallylidene)malonate (3.07 g, 15.34 mmol) and TEA (3.10 g, 30.67 mmol) and the reaction was stirred at 20° C. for 2 h under N₂. The residue was purified by silica gel column chromatography (PE/EtOAc=5/1 to 1/1) to give dimethyl 2-((E)-3-(((1R,2S)-2-methylcyclopropyl)amino)allylidene)malonate (750 mg, 30.7% yield) as yellow oil. LCMS m/z=240.0 [M+H]⁺

Step d: A mixture of dimethyl 2-((E)-3-(((1R,2S)-2-methylcyclopropyl)amino)allylidene)malonate (750 mg, 3.13 mmol) in EtOH (5 mL) and KOH (299 mg, 5.33 mmol) was stirred at 25° C. for 1 h and 90° C. for a further 2 h. The resulting mixture was evaporated under reduced pressure and the residue was dissolved in water (10 mL) and the pH adjusted to 4-5 with 1M HCl. The mixture was extracted with EtOAc (10 mL×3), the organic layers were washed with brine (30 mL), dried over Na₂SO₄, filtered and concentrated to give 1-((1R,2S)-2-methylcyclopropyl)-2-oxo-1,2-dihydropyridine-3-carboxylic acid (580 mg, 95.8% yield) as a yellow solid which was used in the next step without further purification. 1H NMR (400 MHz, CDCl₃) δ ppm 14.33 (s, 1H), 8.52 (dd, J=7.2, 2.0 Hz, 1H), 7.67 (dd, J=6.8, 2.0 Hz, 1H), 6.54 (t, J=7.0 Hz, 1H), 3.52-3.46 (m, 1H), 1.56-1.50 (m, 1H), 1.38-1.31 (m, 1H), 0.88 (d, J=6.4 Hz, 3H), 0.78-0.73 (m, 1H).

Step e: To a mixture of 1-((1R,2S)-2-methylcyclopropyl)-2-oxo-1,2-dihydropyridine-3-carboxylic acid (580 mg, 3.0 mmol) in t-BuOH (3 mL) and TEA (455.67 mg, 4.50 mmol) was added DPPA (991.41 mg, 3.60 mmol) and the reaction mixture was stirred at 90° C. for 2 h. Water (20 mL) was added and the mixture was extracted with EtOAc (20 mL×3). The combined organic layers were washed with brine (50 mL), dried over Na₂SO₄, filtered and concentrated. The crude material was purified by silica gel column chromatography (PE/EtOAc=5/1 to 1/1) to give tert-butyl (1-((1R,2S)-2-methylcyclopropyl)-2-oxo-1,2-dihydropyridin-3-yl)carbamate (460 mg, 58.0% yield) as a yellow solid. LCMS m/z=265.0 [M+H]⁺

Step f: A mixture of tert-butyl (1-((1R,2S)-2-methylcyclopropyl)-2-oxo-1,2-dihydropyridin-3-yl)carbamate (600 mg, 2.27 mmol) in dioxane (5 mL) and HCl/dioxane (4 M, 10 mL) was stirred at 40° C. for 12 h. The mixture was concentrated under reduced pressure, the residue was diluted with water (9 mL) and MeCN (3 mL) then lyophilised to give 3-amino-1-((1R,2S)-2-methylcyclopropyl)pyridin-2(1H)-one hydrochloride (416 mg, 91.2% yield) as a yellow solid. LCMS m/z=165.1 [M+H]⁺

### Preparation 73: 3-amino-1-((1S,2R)-2-methylcyclopropyl)pyridin-2(1H)-one hydrochloride

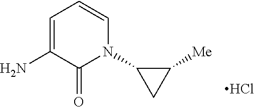 [SMILES: C[C@@H]1C[C@@H]1n1cccc(N)c1=O ; Cl]

3-Amino-1-((1S,2R)-2-methylcyclopropyl)pyridin-2(1H)-one hydrochloride was obtained as a yellow solid, from (1S,2R)-2-methylcyclopropane-1-carboxylic acid, following the steps described in Preparation 72. LCMS m/z=165.2 [M+H]⁺

### Preparations 74A and 75A. 3-amino-1-((1R,2R)-2-fluorocyclopropyl)pyridin-2(1H)-one hydrochloride and 3-amino-1-((1S,2S)-2-fluorocyclopropyl)pyridin-2(1H)-one hydrochloride

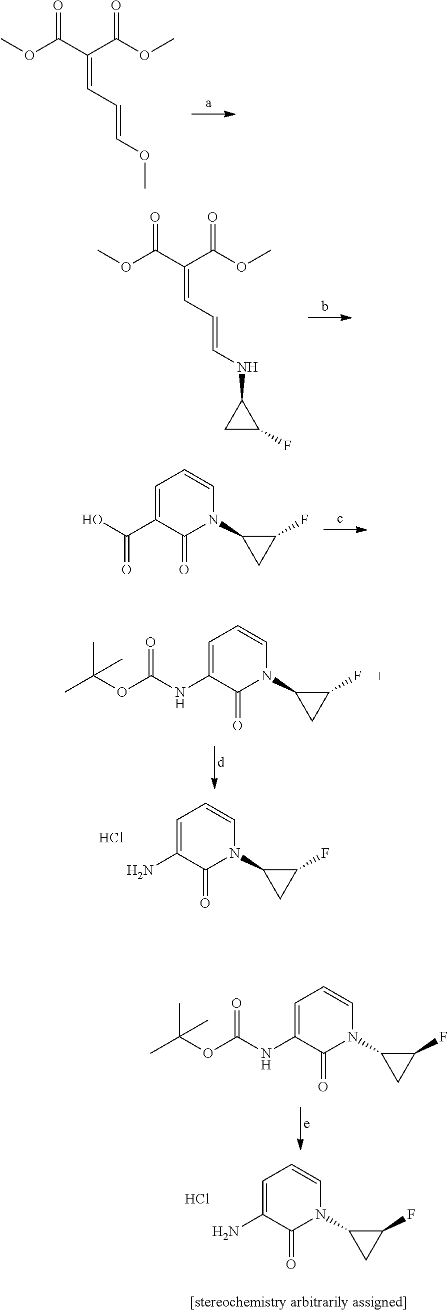

Step a. To a solution of dimethyl (E)-2-(3-methoxyallylidene)malonate (4.99 g, 24.92 mmol) in MeOH (50 mL) was added trans-2-fluorocyclopropanamine (2.78 g, 24.92 mmol), TEA (5.04 g, 49.85 mmol) and the reaction stirred at 25° C. for 16 h. The mixture was concentrated in vacuo, the residue was diluted with water (50 mL) and extracted with EtOAc (50 mL×3). The combined organic layer was washed with brine (50 mL), dried over Na₂SO₄ and filtered. The filtrate was concentrated in vacuo to give trans dimethyl 2-((E)-3-((2-fluorocyclopropyl)amino)allylidene)malonate (6.8 g, crude) as yellow oil and it was used directly in the next step.

Step b. To a solution of trans dimethyl 2-((E)-3-((2-fluorocyclopropyl)amino)allylidene)malonate (6.7 g, 27.55 mmol) in EtOH (100 mL) was added KOH (2.47 g, 44.07 mmol) and the mixture was stirred at 25° C. for 3 h. The reaction mixture was acidified to pH 5 using 1M HCl, diluted with water (300 mL) and extracted with EtOAc (200 mL×3). The combined organic layer was washed with brine (100 mL), dried over Na₂SO₄ and filtered. The filtrate was concentrated in vacuo to give trans-1-(2-fluorocyclopropyl)-2-oxo-1,2-dihydropyridine-3-carboxylic acid (4 g, crude) as brown solid. LCMS m/z=197.6 [M+H]⁺

Step c. To a solution of trans-1-(2-fluorocyclopropyl)-2-oxo-1,2-dihydropyridine-3-carboxylic acid (4 g, 20.29 mmol) in t-BuOH (100 mL) was added DPPA (8.37 g, 30.43 mmol) and TEA (6.16 g, 60.86 mmol) and the reaction stirred at 90° C. for 16 h. The mixture was concentrated and then water (300 mL) was added. The mixture was extracted with EtOAc (300 mL×3), the combined organic layers were washed with brine (200 mL), dried over Na₂SO₄, filtered and concentrated in vacuo. The residue was purified by CombiFlash® (PE/EtOAc=1/1) and the product was further purified by SFC (Column: ChiralPak AD-3 150×4.6 mm I.D., 3 um, Mobile phase: A: CO₂ B: Ethanol (0.05% DEA), Gradient: from 5% to 40% of B in 4.5 min, Flow rate: 2.5 mL/min Column temp.: 40° C.) to give tert-butyl (1-((1R,2R)-2-fluorocyclopropyl)-2-oxo-1,2-dihydropyridin-3-yl)carbamate (560 mg, 9.8% yield, stereochemistry arbitrily defined). RT=2.555 min. LCMS m/z=268.1 [M+H]⁺

and tert-butyl (1-((1S,2S)-2-fluorocyclopropyl)-2-oxo-1,2-dihydropyridin-3-yl)carbamate (560 mg, 9.8% yield) as brown solid. RT=2.842 min. LCMS m/z=268.1 [M+H]⁺

Step d. tert-Butyl (1-((1R,2R)-2-fluorocyclopropyl)-2-oxo-1,2-dihydropyridin-3-yl)carbamate (560 mg, 2.09 mmol) was dissolved in HCl/dioxane (30 mL) and the mixture was stirred at 25° C. for 16 h. The mixture was concentrated in vacuo to give 3-amino-1-((1R,2R)-2-fluorocyclopropyl)pyridin-2(1H)-one hydrochloride (Stereochemistry arbitrarily assigned), (400 mg, 93.7% yield) as white solid. LCMS m/z=168.9 [M+H]⁺

Step e. tert-Butyl (1-((1S,2S)-2-fluorocyclopropyl)-2-oxo-1,2-dihydropyridin-3-yl)carbamate (560 mg, 2.09 mmol) was dissolved in HCl/dioxane (30 mL) and the mixture was stirred at 25° C. for 16 h. The mixture was concentrated in vacuo to give 3-amino-1-((1S,2S)-2-fluorocyclopropyl)pyridin-2(1H)-one hydrochloride (400 mg, 93.7% yield) as white solid. LCMS m/z=168.9 [M+H]⁺

### Preparation 74B: 3-amino-1-((1R,2R)-2-fluorocyclopropyl)pyridin-2(1H)-one hydrochloride

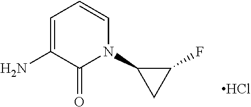 [SMILES: Nc1cccn([C@@H]2C[C@H]2F)c1=O ; Cl]

3-Amino-1-((1R,2R)-2-fluorocyclopropyl)pyridin-2(1H)-one hydrochloride was obtained from (1R,2R)-2-fluorocyclopropane-1-carboxylic acid, following the steps described in Preparation 72.

### Preparation 75B: 3-amino-1-((1S,2S)-2-fluorocyclopropyl)pyridin-2(1H)-one hydrochloride

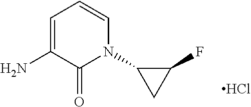 [SMILES: Nc1cccn([C@H]2C[C@@H]2F)c1=O ; Cl]

3-Amino-1-((1S,2S)-2-fluorocyclopropyl)pyridin-2(1H)-one hydrochloride was obtained from (1S,2S)-2-fluorocyclopropane-1-carboxylic acid, following the steps described in Preparation 72.

### Preparation 76: 3-amino-1-((1R,2S)-2-fluorocyclopropyl)pyridin-2(1H)-one hydrochloride

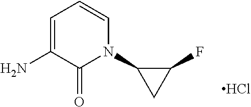 [SMILES: Nc1cccn([C@@H]2C[C@@H]2F)c1=O ; Cl]

3-Amino-1-((1R,2S)-2-fluorocyclopropyl)pyridin-2(1H)-one hydrochloride was obtained from (1R,2S)-2-fluorocyclopropane-1-carboxylic acid, following the steps described in Preparation 72.

### Preparation 77: 3-amino-1-((1S,2R)-2-fluorocyclopropyl)pyridin-2(1H)-one hydrochloride

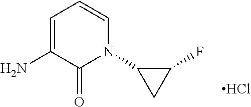

3-Amino-1-((1S,2R)-2-fluorocyclopropyl)pyridin-2(1H)-one hydrochloride was obtained from (1S,2R)-2-fluorocyclopropane-1-carboxylic acid, following the steps described in Preparation 72.

### Preparation 78: 2-(2-oxabicyclo[2.1.1]hexan-4-yl)-5-bromo-6-isopropoxy-2H-indazole

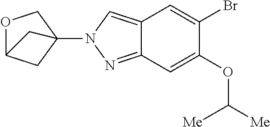 [SMILES: CC(C)Oc1cc2nn(C34COC(C3)C4)cc2cc1Br]

Part 1: To a solution of 5-bromo-4-isopropoxy-2-nitrobenzaldehyde (Preparation 24, 500 mg, 1.74 mmol) in IPA (10 mL) was added 2-oxabicyclo[2.1.1]hexan-4-amine (353 mg, 2.60 mmol) and TEA (176 mg, 1.74 mmol) and the mixture stirred at 80° C. for 16 h. The mixture was concentrated in vacuo to give the residue which was purified by Combi-Flash (PE/EtOAc=10/1 to 5/1) to give (E)-N-(2-oxabicyclo[2.1.1]hexan-4-yl)-1-(5-bromo-4-isopropoxy-2-nitrophenyl)methanimine as a yellow oil (620 mg, 87%). ¹H NMR (500 MHz, CDCl₃) δ: 8.72 (s, 1H), 8.38 (s, 1H), 7.50 (s, 1H), 4.75-4.70 (m, 1H), 4.63 (s, 1H), 3.75 (s, 2H), 2.13-2.08 (m, 2H), 1.96-1.91 (m, 2H), 1.47 (d, 6H).

Part 2: To a solution of (E)-N-(2-oxabicyclo[2.1.1]hexan-4-yl)-1-(5-bromo-4-isopropoxy-2-nitrophenyl)methanimine (Part 1, 620 mg, 1.68 mmol) in IPA (10 mL) was added P(n-Bu)₃ (1.02 g, 5.04 mmol) and the mixture was stirred at 80° C. for 16 h. The mixture was concentrated and H₂O (80 mL) was added. The mixture was extracted with EtOAc (3×50 mL). The combined organics were washed with brine (50 mL), dried (Na₂SO₄) and evaporated to dryness in vacuo to give a residue which was purified by Combi-Flash (PE/EtOAc=10/1 to 3/1) to give 2-(2-oxabicyclo[2.1.1]hexan-4-yl)-5-bromo-6-isopropoxy-2H-indazole as a yellow solid (500 mg, 79%). LCMS m/z=337.1 [M+H]⁺.

### Preparation 79: 5-bromo-2-(1-(fluoromethyl)-2-oxabicyclo[2.1.1]hexan-4-yl)-6-isopropoxy-2H-indazole

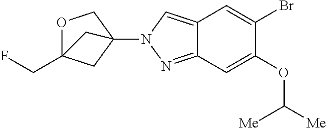 [SMILES: CC(C)Oc1cc2nn(C34COC(CF)(C3)C4)cc2cc1Br]

Part 1: To a solution of 5-bromo-4-isopropoxy-2-nitrobenzaldehyde (Preparation 24, 2 g, 6.94 mmol) in IPA (10 mL) was added 1-(fluoromethyl)-2-oxabicyclo[2.1.1]hexan-4-amine (1.09 g, 8.33 mmol) and TEA (702 mg, 6.94 mmol) and the mixture stirred at 80° C. for 16 h. The mixture was concentrated in vacuo to give the residue which was purified by Combi-Flash (PE/EtOAc=10/1 to 5/1) to give (E)-1-(5-bromo-4-isopropoxy-2-nitrophenyl)-N-(1-(fluoromethyl)-2-oxabicyclo[2.1.1]hexan-4-yl)methanimine as a yellow oil (2.5 mg, 80%). LCMS m/z=402.6 [M+H]⁺.

Part 2: To a solution of (E)-1-(5-bromo-4-isopropoxy-2-nitrophenyl)-N-(1-(fluoromethyl)-2-oxabicyclo[2.1.1]hexan-4-yl)methanimine (Part 1, 2.5 mg, 6.23 mmol) in IPA (10 mL) was added P(n-Bu)₃ (3.78 g, 18.7 mmol) and the mixture was stirred at 80° C. for 16 h. The mixture was concentrated and H₂O (80 mL) was added. The mixture was extracted with EtOAc (3×50 mL). The combined organics were washed with brine (50 mL), dried (Na₂SO₄) and evaporated to dryness in vacuo to give a residue which was purified by Combi-Flash (PE/EtOAc=10/1 to 3/1) to give 5-bromo-2-(1-(fluoromethyl)-2-oxabicyclo[2.1.1]hexan-4-yl)-6-isopropoxy-2H-indazole as a yellow solid (2 mg, 78%). ¹H NMR: (400 MHz, CDCl₃) δ: 7.87 (d, J=6.0 Hz, 1H), 7.28 (s, 1H), 7.04 (s, 1H), 4.78 (d, J=47.2 Hz, 2H), 4.65-4.58 (m, 1H), 4.28 (s, 2H), 2.49-2.44 (m, 4H), 1.44 (d, J=6.4 Hz, 6H).

### Preparation 80: 5-bromo-6-isopropoxy-2-(1-methyl-2-oxabicyclo[2.2.1]heptan-4-yl)-2H-indazole

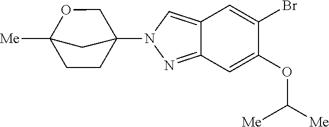 [SMILES: CC(C)Oc1cc2nn(C34CCC(C)(C3)OC4)cc2cc1Br]

To a solution of 5-bromo-4-isopropoxy-2-nitrobenzaldehyde (Preparation 24, 1 g, 3.47 mmol, 1.0 eq.) in IPA (20 mL) was added 1-methyl-2-oxabicyclo[2.2.1]heptan-4-amine (441 mg, 3.47 mmol) and TEA (351 mg, 3.47 mmol) and stirred at 80° C. for 16 h. The reaction mixture was cooled down to 20° C. and P(nBu)₃ (2.11 g, 10.41 mmol) was added and the mixture stirred at 80° C. for 16 h. The reaction mixture was quenched by the addition of saturated aqueous NH4Cl solution (100 mL), the aqueous layer was separated and extracted with EtOAc (2×100 mL). The combined organics were washed with brine (50 mL), dried (Na₂SO₄) and evaporated to dryness in vacuo. The residue was purified by Combi-Flash (PE/EtOAc=10/1 to 5/1) to give 5-bromo-6-isopropoxy-2-(1-methyl-2-oxabicyclo[2.2.1]heptan-4-yl)-2H-indazole as a yellow oil (700 mg, 49%). LCMS m/z=366.8 [M+H]⁺.

### Preparation 81: 5-bromo-6-cyclobutoxy-2-(1-(methoxymethyl)-2-oxabicyclo[2.1.1]hexan-4-yl)-2H-indazole

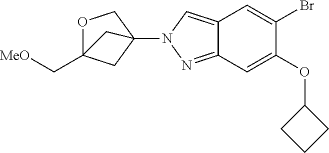 [SMILES: COCC12CC(n3cc4cc(Br)c(OC5CCC5)cc4n3)(CO1)C2]

Part 1. 1-(methoxymethyl)-2-oxabicyclo[2.1.1]hexan-4-amine (286 mg, 2.00 mmol) was added to a solution of 5-bromo-4-cyclobutoxy-2-nitrobenzaldehyde (Preparation 58, 600 mg, 2.00 mmol) in IPA (20 mL) and the mixture was stirred at 80° C. for 16 h. The mixture was cooled to room temperature and diluted with EtOAc (10 mL). The organics were washed with saturated ammonium chloride solution (10 mL), brine (10 ml), dried (Na₂SO₄) and evaporated to dryness in vacuo. The residue was purified by silica gel chromatography (PE:EA=10:1 to 3:1) to give (E)-1-(5-bromo-4-cyclobutoxy-2-nitrophenyl)-N-(1-(methoxymethyl)-2-oxabicyclo[2.1.1]hexan-4-yl)methanimine as a yellow oil (360 mg, 42%) which was used directly in Part 2.

Part 2. To a solution of (E)-1-(5-bromo-4-cyclobutoxy-2-nitrophenyl)-N-(1-(methoxymethyl)-2-oxabicyclo[2.1.1]hexan-4-yl)methanimine (360 mg, 0.8465 mmol) in IPA (20 mL) was added P(nBu)₃ (514 mg, 2.54 mmol) at 20° C. and the mixture stirred at 80° C. for 16 h. The mixture was cooled to room temperature and diluted with EtOAc (10 mL). The organics were washed with saturated ammonium chloride solution (10 mL), brine (10 ml), dried (Na₂SO₄) and evaporated to dryness in vacuo. The residue was purified by silica gel chromatography (PE:EA=10:1 to 3:1) to give 5-bromo-6-cyclobutoxy-2-(1-(methoxymethyl)-2-oxabicyclo[2.1.1]hexan-4-yl)-2H-indazole as a yellow solid (300 mg, 82%). ¹H NMR: (400 MHz, CDCl₃) δ: 7.86 (d, J=2.4 Hz, 2H), 6.88 (s, 1H), 4.78-4.63 (m, 1H), 4.25 (s, 2H), 3.76 (s, 2H), 3.47 (s, 3H), 2.56-2.54 (m, 2H), 2.41-2.38 (m, 4H), 2.28-2.27 (m, 2H), 1.93-1.91 (m, 1H), 1.76-1.73 (m, 1H).

### Preparation 82: methyl 2-(2-oxabicyclo[2.1.1]hexan-4-yl)-6-isopropoxy-2H-indazole-5-carboxylate

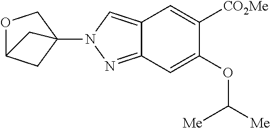 [SMILES: COC(=O)c1cc2cn(C34COC(C3)C4)nc2cc1OC(C)C]

RBr: 2-(2-oxabicyclo[2.1.1]hexan-4-yl)-5-bromo-6-isopropoxy-2H-indazole (Preparation 78); Pd(^{t}Bu₃P)₂

To a solution of 2-(2-oxabicyclo[2.1.1]hexan-4-yl)-5-bromo-6-isopropoxy-2H-indazole (Preparation 78, 240 mg, 0.712 mmol) in MeOH (50 mL) was added Pd(t-Bu₃P)₂ (36.4 mg, 0.072 mmol) and TEA (720 mg, 7.12 mmol). The reaction system was purged with CO (3×) and the reaction mixture was stirred under 80° C. and CO (50 psi) for 16 h. The mixture was filtered through a pad of celite and the filtrate evaporated to dryness in vacuo. The reside was purified by Combi-Flash (PE/EtOAc=1/1) to give methyl 2-(2-oxabicyclo[2.1.1]hexan-4-yl)-6-isopropoxy-2H-indazole-5-carboxylate as a yellow oil (170 mg, 68%). ¹H NMR: (400 MHz, CDCl₃) δ: 8.12 (s, 1H), 8.03 (s, 1H), 7.11 (s, 1H), 4.73 (s, 1H), 4.68-4.63 (m, 1H), 4.19 (s, 2H), 3.91 (s, 3H), 2.58-2.54 (m, 2H), 2.43-2.40 (m, 2H), 1.42 (d, J=6.4 Hz, 6H).

### Preparation 83: methyl 2-(1-(fluoromethyl)-2-oxabicyclo[2.1.1]hexan-4-yl)-6-isopropoxy-2H-indazole-5-carboxylate

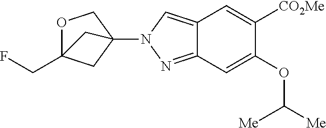 [SMILES: COC(=O)c1cc2cn(C34COC(CF)(C3)C4)nc2cc1OC(C)C]

To a solution of 5-bromo-2-(1-(fluoromethyl)-2-oxabicyclo[2.1.1]hexan-4-yl)-6-isopropoxy-2H-indazole (Preparation 79, 2 g, 5.42 mmol) in MeOH (50 mL) was added Pd(dppf)Cl₂ (396 mg, 0.542 mmol) and TEA (5.48 g, 54.2 mmol). The reaction mixture was purged with CO (3×) the reaction mixture stirred under 80° C. and CO (50 psi) for 48 h. The mixture was filtered through a pad of celite and the filtrate evaporated to dryness in vacuo. The residue was purified by Combi-Flash (PE/EtOAc=1/1) to give methyl 2-(1-(fluoromethyl)-2-oxabicyclo[2.1.1]hexan-4-yl)-6-isopropoxy-2H-indazole-5-carboxylate as a yellow oil (1.5 g, 76%). ¹H NMR: (500 MHz, CDCl₃) δ: 8.11 (s, 1H), 7.99 (s, 1H), 7.05 (s, 1H), 4.79-4.69 (m, 2H), 4.65-4.59 (m, 1H), 4.29 (s, 2H), 3.91 (s, 3H), 2.52-2.49 (m, 2H), 2.46-2.43 (m, 2H), 1.42 (d, J=6.0 Hz, 6H).

### Preparation 84: methyl 6-isopropoxy-2-(1-methyl-2-oxabicyclo[2.2.1]heptan-4-yl)-2H-indazole-5-carboxylate

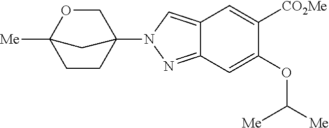 [SMILES: COC(=O)c1cc2cn(C34CCC(C)(C3)OC4)nc2cc1OC(C)C]

To a solution of 5-bromo-6-isopropoxy-2-(1-methyl-2-oxabicyclo[2.2.1]heptan-4-yl)-2H-indazole (Preparation 80, 700 mg, 1.92 mmol) in MeOH (20 mL) was added TEA (1.94 g, 19.2 mmol) and Pd(dppf)Cl₂ (140 mg, 0.192 mmol). The reaction system was purged with CO (3×) and the reaction mixture stirred under 80° C. and CO (50 psi) for 16 h. The mixture was filtered through a pad of celite and the filtrate was concentrated in vacuo. The residue was purified by Combi-Flash (PE/EtOAc=1/1) to give methyl 6-isopropoxy-2-(1-methyl-2-oxabicyclo[2.2.1]heptan-4-yl)-2H-indazole-5-carboxylate as a yellow solid (550 mg, 75%). LCMS m/z=345.2 [M+H]⁺.

### Preparation 85: methyl 6-cyclobutoxy-2-(1-(methoxymethyl)-2-oxabicyclo[2.1.1]hexan-4-yl)-2H-indazole-5-carboxylate

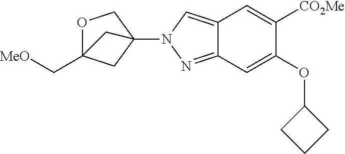 [SMILES: COCC12CC(n3cc4cc(C(=O)OC)c(OC5CCC5)cc4n3)(CO1)C2]

To a solution of 5-bromo-6-cyclobutoxy-2-(1-(methoxymethyl)-2-oxabicyclo[2.1.1]hexan-4-yl)-2H-indazole (Preparation 81, 300 mg, 0.763 mmol) in MeOH (50 mL) was added TEA (772 mg, 7.63 mmol) and Pd(dppf)Cl₂ (112 mg, 0.153 mmol). The mixture was degassed with CO (3×) and then stirred at 80° C. under CO (50 Psi) for 48 h. The mixture was concentrated in vacuo and the residue was purified by Combi Flash (PE/EA=1/1) to give the methyl 6-cyclobutoxy-2-(1-(methoxymethyl)-2-oxabicyclo[2.1.1]hexan-4-yl)-2H-indazole-5-carboxylate as a white solid (250 mg, 88%). LCMS m/z=373.1 [M+H]⁺.

### Preparation 86: 2-(1-(fluoromethyl)-2-oxabicyclo[2.1.1]hexan-4-yl)-6-isopropoxy-2H-indazole-5-carboxylic acid

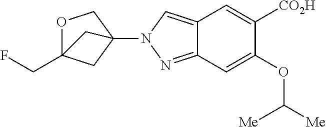 [SMILES: CC(C)Oc1cc2nn(C34COC(CF)(C3)C4)cc2cc1C(=O)O]

To a solution of methyl 2-(1-(fluoromethyl)-2-oxabicyclo[2.1.1]hexan-4-yl)-6-isopropoxy-2H-indazole-5-carboxylate (Preparation 83, 1.5 g, 4.31 mmol) in H₂O (5 mL) and MeOH (5 mL) was added LiOH (542 mg, 12.9 mmol) and the mixture was stirred at 25° C. for 16 h. The mixture was adjusted by 1 N HCl to pH=7 and concentrated in vacuo and the residue was lyophilized to afford 2-(1-(fluoromethyl)-2-oxabicyclo[2.1.1]hexan-4-yl)-6-isopropoxy-2H-indazole-5-carboxylic acid as a brown solid (1.96 g, crude). LCMS m/z=335.1 [M+H]⁺.

### Preparation 87-88

The title compounds were prepared from the appropriate ester (RCO₂Me) using an analogous method to that described for Preparation 86.

| Prepara- | |
|---|---|
| tion | |
| Number | Name/Structure/RCO₂Me/Data |
| 87 | 2-(2-oxabicyclo[2.1.1]hexan-4-yl)-6-isopropoxy-2H-indazole-5- |
| | carboxylic acid |
| | 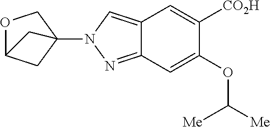 [SMILES: CC(C)Oc1cc2nn(C34COC(C3)C4)cc2cc1C(=O)O] |
| | RCO₂Me: methyl 2-(2-oxabicyclo[2.1.1]hexan-4-yl)-6- |
| | isopropoxy-2H-indazole-5-carboxylate (Preparation 82) |
| | Brown solid (210 mg, crude); LCMS m/z = 303.2 [M + H]⁺. |
| 88 | 6-cyclobutoxy-2-(1-(methoxymethyl)-2-oxabicyclo[2.1.1]hexan-4- |
| | yl)-2H-indazole-5-carboxylic acid |
| | 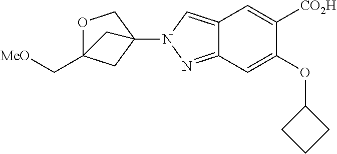 [SMILES: COCC12CC(n3cc4cc(C(=O)O)c(OC5CCC5)cc4n3)(CO1)C2] |
| | RCO₂Me: methyl 6-cyclobutoxy-2-(1-(methoxymethyl)-2- |
| | oxabicyclo[2.1.1]hexan-4-yl)-2H-indazole-5-carboxylate |
| | (Preparation 85) |
| | Yellow solid (300 mg, 99%); LCMS m/z = 359.1 [M + H]⁺. |

### Preparation 89 and 90: Methyl 6-isopropoxy-2-((1S,4S)-1-methyl-2-oxabicyclo[2.2.1]heptan-4-yl)-2H-indazole-5-carboxylate and methyl 6-isopropoxy-2-((1R,4R)-1-methyl-2-oxabicyclo[2.2.1]heptan-4-yl)-2H-indazole-5-carboxylate

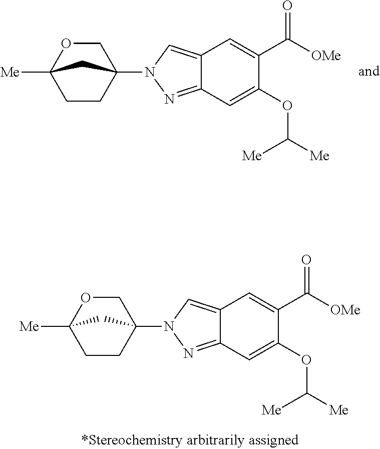 [SMILES: COC(=O)c1cc2cn([C@@]34CC[C@@](C)(C3)OC4)nc2cc1OC(C)C ; COC(=O)c1cc2cn([C@]34CC[C@](C)(C3)OC4)nc2cc1OC(C)C]

Methyl 6-isopropoxy-2-(1-methyl-2-oxabicyclo[2.2.1]heptan-4-yl)-2H-indazole-5-carboxylate (100 mg, 0.2904 mmol) was purified by prep-SFC (Diacel Chiralpak AY-H, 250×30 mm, 5 mm); 40% of IPA (0.05% DEA) in C₀₂ to give the title compounds.

*Peak 1, Preparation 89, methyl 6-isopropoxy-2-((1S,4S)-1-methyl-2-oxabicyclo[2.2.1]heptan-4-yl)-2H-indazole-5-carboxylate or methyl 6-isopropoxy-2-((1R,4R)-1-methyl-2-oxabicyclo[2.2.1]heptan-4-yl)-2H-indazole-5-carboxylate (white solid, 50 mg, 50%). LCMS m/z=345.1 [M+H]⁺.

*Peak 2, Preparation 90, methyl 6-isopropoxy-2-((1S,4S)-1-methyl-2-oxabicyclo[2.2.1]heptan-4-yl)-2H-indazole-5-carboxylate or methyl 6-isopropoxy-2-((1R,4R)-1-methyl-2-oxabicyclo[2.2.1]heptan-4-yl)-2H-indazole-5-carboxylate (white solid, 50 mg, 50%). LCMS m/z=345.1 [M+H]⁺.

### Preparation 91: 6-Isopropoxy-2-((1S,4S)-1-methyl-2-oxabicyclo[2.2.1]heptan-4-yl)-2H-indazole-5-carboxylic acid

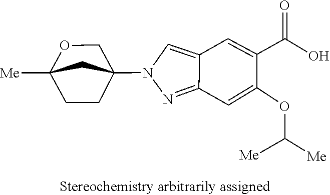 [SMILES: CC(C)Oc1cc2nn([C@@]34CC[C@@](C)(C3)OC4)cc2cc1C(=O)O]

NaOH (17.4 mg, 0.435 mmol) was added to a solution of methyl 6-isopropoxy-2-((1S,4S)-1-methyl-2-oxabicyclo[2.2.1]heptan-4-yl)-2H-indazole-5-carboxylate (Peak 1, Preparation 89, 50 mg, 0.145 mmol) in H₂O (2 mL) and MeOH (2 mL) and the mixture stirred at 15° C. for 16 h. The reaction mixture was concentrated in vacuo and the residue diluted with water (10 mL) and the pH adjusted 3 by addition of 1M HCl (aq.). The mixture was lyophilized to give 6-isopropoxy-2-((1S,4S)-1-methyl-2-oxabicyclo[2.2.1]heptan-4-yl)-2H-indazole-5-carboxylic acid as a yellow solid (50 mg, 94%) as a yellow solid. LCMS m/z=331.0 [M+H]⁺.

### Preparation 92: 6-isopropoxy-2-((1R,4R)-1-methyl-2-oxabicyclo[2.2.1]heptan-4-yl)-2H-indazole-5-carboxylic acid

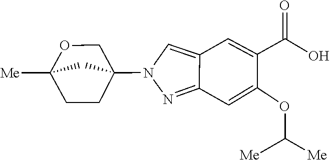 [SMILES: CC(C)Oc1cc2nn([C@]34CC[C@](C)(C3)OC4)cc2cc1C(=O)O]

The title compound was prepared as a yellow solid (50 mg, 94%) using an analogous method to that described for Preparation 91 from methyl 6-isopropoxy-2-((1R,4R)-1-methyl-2-oxabicyclo[2.2.1]heptan-4-yl)-2H-indazole-5-carboxylate (Peak 2, Preparation 90). LCMS m/z=331.0 [M+H]⁺.

### Preparation 93: 6-cyclopropoxy-2-nitronicotinaldehyde

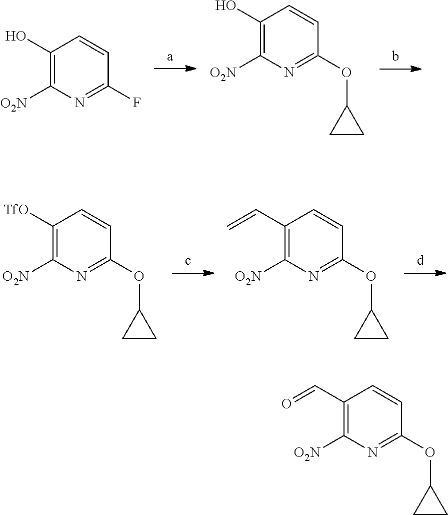 [SMILES: O=[N+]([O-])c1nc(F)ccc1O ; O=[N+]([O-])c1nc(OC2CC2)ccc1O ; O=[N+]([O-])c1nc(OC2CC2)ccc1OS(=O)(=O)C(F)(F)F ; C=Cc1ccc(OC2CC2)nc1[N+](=O)[O-] ; O=Cc1ccc(OC2CC2)nc1[N+](=O)[O-]]

Step a: To a solution of cyclopropanol (16.16 g, 278.33 mmol) in THF (200 mL) was added NaH (5.57 g, 139.16 mmol) at 0° C. and stirred at 0° C. for 0.5 h. 6-fluoro-2-nitropyridin-3-ol (11 g, 69.58 mmol, 1.0 eq.) was added and the mixture stirred at 25° C. for 4 h. The reaction mixture was concentrated and adjusted by 1N HCl to pH=5 and extracted with EtOAc (3×200 mL). The combined organics were washed with brine (200 mL), dried (Na₂SO₄) and concentrated in vacuo. The residue was purified by Combi-Flash (PE/EtOAc=10/1) to give 6-cyclopropoxy-2-nitropyridin-3-ol as a yellow solid (2.1 g, 15.4%). ¹H NMR (500 MHz, CDCl₃) δ: 10.17 (s, 1H), 7.55 (d, J=8.5 Hz, 1H), 7.09 (d, J=9.0 Hz, 1H), 4.35 (t, J=3.0 Hz, 1H), 0.86-0.83 (m, 2H), 0.79-0.75 (m, 2H).

Step b: To a solution of 6-cyclopropoxy-2-nitropyridin-3-ol (Part a, 2.3 g, 11.73 mmol) in DCM (100 mL) was added TEA (2.37 g, 23.45 mmol) and Tf₂O (3.97 g, 14.07 mmol) at 0° C. and stirred at 0° C. for 1 h. The mixture was concentrated and water (200 mL) added. The mixture was extracted with DCM (2×200 mL) and the combined organics washed with brine (50 mL), dried (Na₂SO₄) and concentrated in vacuo to give a residue which was purified by combi-Flash (PE/EtOAc=20/1) to give 6-cyclopropoxy-2-nitropyridin-3-yl trifluoromethanesulfonate as a yellow oil (3.5 g, 91%). ¹H NMR (500 MHz, DMSO-d₆) δ: 8.35 (d, J=9.0 Hz, 1H), 7.55 (d, J=9.0 Hz, 1H), 4.35-4.30 (m, 1H), 0.86-0.82 (m, 2H), 0.81-0.77 (m, 2H).

Step c: To a solution of 6-cyclopropoxy-2-nitropyridin-3-yl trifluoromethanesulfonate (Part b, 3.5 g, 10.66 mmol) in dioxane (50 mL) and water (6 mL) was added K₂CO₃ (2.95 g, 21.33 mmol) and Pd(dppf)Cl₂ (780.26 mg, 1.07 mmol) under N₂ and stirred at 80° C. for 16 h. The mixture was concentrated and water (200 mL) added and the mixture extracted with EtOAc (3×100 mL). The combined organics were washed with brine (100 mL), dried (Na₂SO₄) and concentrated in vacuo and the residue purified by Combi-Flash (PE/EtOAc=10/1) to give 6-cyclopropoxy-2-nitro-3-vinylpyridine as a yellow oil (1.6 g, 65.5%). ¹H NMR (500 MHz, CDCl₃) δ: 7.96 (d, J=8.5 Hz, 1H), 7.00 (d, J=8.5 Hz, 1H), 6.90 (dd, J=17.0 Hz, 11.0 Hz, 1H), 5.74 (d, J=17.5 Hz, 1H), 5.48 (d, J=11.0 Hz, 1H), 4.35-4.30 (m, 1H), 0.87-0.82 (m, 2H), 0.81-0.78 (m, 2H).

Step d: To a solution of 6-cyclopropoxy-2-nitro-3-vinylpyridine (Part c, 1.6 g, 7.76 mmol) in dioxane (20 mL) and water (6 mL) was added K₂OsO₄ (143 mg, 0.388 mmol) and NaIO₄ (3.32 g, 0.388 mmol) and stirred at 25° C. for 2 h. The mixture was concentrated and then water (50 mL) was added. The mixture was extracted with EtOAc (3×20 mL) and the combined organics washed with brine (50 mL), dried (Na₂SO₄) and concentrated in vacuo to give a residue which was purified by Combi-Flash (PE/EtOAc=I/O to 20/1) to give 6-cyclopropoxy-2-nitronicotinaldehyde as a grey oil (800 mg, 44.6%). ¹H NMR (500 MHz, CDCl₃) δ: 10.22 (s, 1H), 8.32 (d, J=8.5 Hz, 1H), 7.12 (d, J=8.5 Hz, 1H), 4.49-4.45 (m, 1H), 0.93-0.88 (m, 2H), 0.87-0.83 (m, 2H).

### Preparation 94: (E)-1-(6-cyclopropoxy-2-nitropyridin-3-yl)-N-(1-methyl-2-oxabicyclo[2.1.1]hexan-4-yl)methanimine

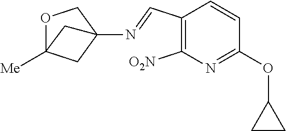 [SMILES: CC12CC(/N=C/c3ccc(OC4CC4)nc3[N+](=O)[O-])(CO1)C2]

To a solution of 6-cyclopropoxy-2-nitronicotinaldehyde (Preparation 93, 500 mg, 2.40 mmol) in IPA (30 mL) was added 1-methyl-2-oxabicyclo[2.1.1]hexan-4-amine (431.24 mg, 2.88 mmol) and TEA (243 mg, 2.40 mmol) and the mixture stirred at 80° C. for 16 h. The reaction mixture was concentrated in vacuo and the residue purified by Combi-Flash (PE/EtOAc=10/1 to 5/1) to give (E)-1-(6-cyclopropoxy-2-nitropyridin-3-yl)-N-(1-methyl-2-oxabicyclo[2.1.1]hexan-4-yl)methanimine as a yellow oil (700 mg, 96%). ¹H NMR (500 MHz, CDCl₃) δ: 8.54 (s, 1H), 8.49 (d, J=8.5 Hz, 1H), 7.06 (d, J=9.0 Hz, 1H), 4.43-4.38 (m, 1H), 2.06-2.04 (m, 2H), 1.83-1.77 (m, 2H), 1.53 (s, 3H), 0.88-0.86 (m, 2H), 0.86-0.82 (m, 2H).

### Preparation 95-98

The title compounds were prepared from 6-cyclopropoxy-2-nitronicotinaldehyde (Preparation 93) or 6-cyclobutoxy-2-nitronicotinaldehyde (Preparation 7) and the appropriate amine (RNH₂) using an analogous method to that described for Preparation 94.

| Preparation | |
|---|---|
| Number | Name/Structure/RNH₂/Data |
| 95 | (E)-1-(6-cyclopropoxy-2-nitropyridin-3-yl)-N-(1-(methoxymethyl)-2- |
| | oxabicyclo[2.1.1]hexan-4-yl)methanimine |
| | 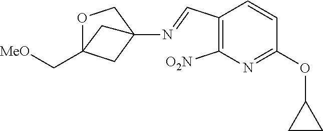 [SMILES: COCC12CC(/N=C/c3ccc(OC4CC4)nc3[N+](=O)[O-])(CO1)C2] |
| | RNH₂: 1-(methoxymethyl)-2-oxabicyclo[2.1.1]hexan-4-amine |
| | ¹H NMR (400 MHz, CDCl₃) δ: 8.57 (s, 1H), 8.53 (d, J = 8.8 Hz, 1H), 7.06 (d, |
| | J = 8.4 Hz, 1), 4.44-4.38 (m, 1H), 3.86 (s, 2H), 3.72 (s, 2H), 3.46 (s, 3H), 2.15- |
| | 2.13 (m, 2H), 1.93-1.90 (m, 2H), 0.91-0.86 (m, 2H), 0.86-0.82 (m, 2H). |
| 96 | (E)-N-(2-oxabicyclo[2.1.1]hexan-4-yl)-1-(6-cyclopropoxy-2-nitropyridin-3- |
| | yl)methanimine |
| | 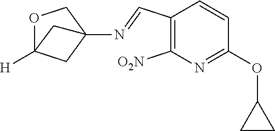 [SMILES: O=[N+]([O-])c1nc(OC2CC2)ccc1/C=N/C12COC(C1)C2] |
| | RNH₂: 2-oxabicyclo[2.1.1]hexan-4-amine |
| | ¹H NMR (400 MHz, CDCl₃) δ: = 8.57 (s, 1H), 8.52 (d, J = 8.5 Hz, 1H), 7.07 |
| | (d, J = 8.5 Hz, 1H), 4.63 (s, 1H), 4.42-4.39 (m, 1H), 3.74 (s, 2H), 2.12-2.08 |
| | (m, 2H), 1.95-1.90 (m, 2H), 0.89-0.87 (m, 2H), 0.86-0.82 (m, 2H). |
| 97 | (E)-N-(2-oxabicyclo[2.2.1]heptan-4-yl)-1-(6-cyclopropoxy-2-nitropyridin-3- |
| | yl)methanimine |
| | 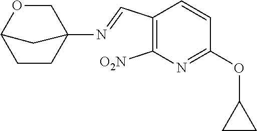 [SMILES: O=[N+]([O-])c1nc(OC2CC2)ccc1/C=N/C12CCC(C1)OC2] |
| | RNH₂: 2-oxabicyclo[2.2.1]heptan-4-amine |
| | ¹H NMR (400 MHz, CDCl₃) δ: 8.59 (s, 1H), 8.52 (d, J = 9.0 Hz, 1H), 7.06 (d, |
| | J = 8.5 Hz, 1H), 4.48-4.46 (m, 1H), 4.42-4.38 (m, 1H), 3.80-3.78 (m, 1H), |
| | 3.77-3.74 (m, 1H), 2.10-2.06 (m, 1H), 1.97-1.94 (m, 2H), 1.92-1.83 (m, 2H), |
| | 1.78 (d, J = 9.5 Hz, 1H), 0.92-0.85 (m, 4H). |
| 98 | (E)-1-(6-cyclopropoxy-2-nitropyridin-3-yl)-N-(1-(fluoromethyl)-2- |
| | oxabicyclo[2.1.1]hexan-4-yl)methanimine |
| | 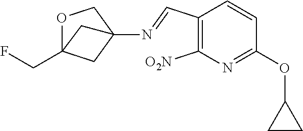 [SMILES: O=[N+]([O-])c1nc(OC2CC2)ccc1/C=N/C12COC(CF)(C1)C2] |
| | RNH₂: 1-(fluoromethyl)-2-oxabicyclo[2.1.1]hexan-4-amine |
| | ¹H NMR (400 MHz, CDCl₃) δ: 8.58 (s, 1H), 8.52 (d, J = 8.4 Hz, 1H), 7.09 (d, |
| | J = 8.4 Hz, 1H), 4.80-4.70 (m, 2H), 4.45-4.35 (m, 1H), 3.89 (s, 2H), 2.25-2.15 |
| | (m, 2H), 2.00-1.90 (m, 2H), 0.90-0.70 (m, 4H). |
| 99 | (E)-1-(6-cyclobutoxy-2-nitro-2,3-dihydropyridin-3-yl)-N-(1- |
| | (methoxymethyl)-2-oxabicyclo[2.1.1]hexan-4-yl)methanimine |
| | 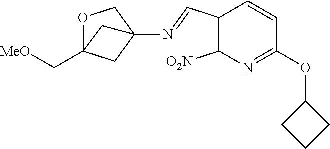 [SMILES: COCC12CC(/N=C/C3C=CC(OC4CCC4)=NC3[N+](=O)[O-])(CO1)C2] |
| | RNH₂: 1-(methoxymethyl)-2-oxabicyclo[2.1.1]hexan-4-amine |
| | Yellow oil (1.5 g, 86%); LCMS m/z = 348.2 [M + H]⁺. |

### Preparation 100: methyl 6-cyclopropoxy-2-(1-methyl-2-oxabicyclo[2.1.1]hexan-4-yl)-2H-pyrazolo[3,4-b]pyridine-5-carboxylate

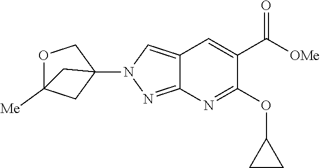 [SMILES: COC(=O)c1cc2cn(C34COC(C)(C3)C4)nc2nc1OC1CC1]

Part a): To a solution of (E)-1-(6-cyclopropoxy-2-nitropyridin-3-yl)-N-(1-methyl-2-oxabicyclo[2.1.1]hexan-4-yl)methanimine (Preparation 94, 700 mg, 2.31 mmol) in IPA (30 mL) was added P(Cy)₃ (1.94 g, 6.92 mmol) and stirred at 70° C. for 16 h. The reaction mixture was quenched by the addition of saturated aqueous NH₄Cl solution (100 mL), the aqueous layer was separated and extracted with EtOAc (2×50 mL). The combined organics were washed with brine (50 mL), dried (Na₂SO₄) and concentrated in vacuo to give a residue which was purified by Combi-Flash (PE/EtOAc=10/1 to 5/1) to give 6-cyclopropoxy-2-(1-methyl-2-oxabicyclo[2.1.1]hexan-4-yl)-2H-pyrazolo[3,4-b]pyridine as a yellow oil (430 mg, 68.7%).

Part b): To a solution of 6-cyclopropoxy-2-(1-methyl-2-oxabicyclo[2.1.1]hexan-4-yl)-2H-pyrazolo[3,4-b]pyridine (Part a, 430 mg, 1.58 mmol) in acetonitrile (10 mL) was added NBS (226 mg, 1.27 mmol) and stirred at 25° C. for 16 h. The mixture was concentrated and water (80 mL) added. The mixture was extracted with EtOAc (50 mL×3) and the combined organics were washed with brine (50 mL), dried over Na₂SO₄, filtered and concentrated in vacuo to give a residue which was purified by Combi-Flash (PE/EtOAc=3/1) to give 5-bromo-6-cyclopropoxy-2-(1-methyl-2-oxabicyclo[2.1.1]hexan-4-yl)-2H-pyrazolo[3,4-b]pyridine as a yellow solid (300 mg, 48%).

Part c): To a solution of 5-bromo-6-cyclopropoxy-2-(1-methyl-2-oxabicyclo[2.1.1]hexan-4-yl)-2H-pyrazolo[3,4-b]pyridine (Part b, 300 mg, 0.857 mmol) in MeOH (20 mL) was added Pd(dppf)Cl₂ (62.68 mg, 0.086 mmol) and TEA (867 mg, 8.57 mmol). The reaction system was charged with CO for three times and then stirred under 80° C. and CO (50 psi) for 16 h. The reaction mixture was filtered through a pad of celite and the filtrate was concentrated in vacuo to give a residue which was purified by Combi-Flash (PE/EtOAc 1/1) to give methyl 6-cyclopropoxy-2-(1-methyl-2-oxabicyclo[2.1.1]hexan-4-yl)-2H-pyrazolo[3,4-b]pyridine-5-carboxylate as a yellow oil (210 mg, 70.7%). LCMS m/z=330.1 [M+H].

### Preparation 101-104

The title compounds were prepared from the appropriate nitropyridine (RNO₂) using an analogous 3-part procedure as described for Preparation 100.

| Preparation | |
|---|---|
| Number | Name/Structure/RNO₂/Data |
| 101 | methyl 6-cyclopropoxy-2-(1-(methoxymethyl)-2-oxabicyclo[2.1.1]hexan-4- |
| | yl)-2H-pyrazolo[3,4-b]pyridine-5-carboxylate |
| | 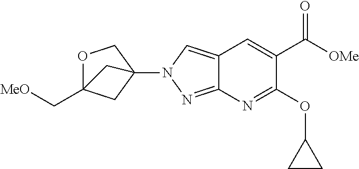 [SMILES: COCC12CC(n3cc4cc(C(=O)OC)c(OC5CC5)nc4n3)(CO1)C2] |
| | RNO₂: (E)-1-(6-cyclopropoxy-2-nitropyridin-3-yl)-N-(1-(methoxymethyl)-2- |
| | oxabicyclo[2.1.1]hexan-4-yl)methanimine (Preparation 95) |
| | White solid (120 mg, 96%); LCMS m/z = 360.1 [M + H]⁺. |
| 102 | methyl 2-(2-oxabicyclo[2.1.1]hexan-4-yl)-6-cyclopropoxy-2H-pyrazolo[3,4- |
| | b]pyridine-5-carboxylate |
| | 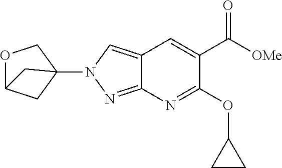 [SMILES: COC(=O)c1cc2cn(C34COC(C3)C4)nc2nc1OC1CC1] |
| | RNO₂: (E)-N-(2-oxabicyclo[2.1.1]hexan-4-yl)-1-(6-cyclopropoxy-2- |
| | nitropyridin-3-yl)methanimine (Preparation 96) |
| | Yellow oil (110 mg, 65%); LCMS m/z = 316.1 [M + H]⁺. |
| 103 | methyl 2-(2-oxabicyclo[2.2.1]heptan-4-yl)-6-cyclopropoxy-2H-pyrazolo[3,4- |
| | b]pyridine-5-carboxylate |
| | 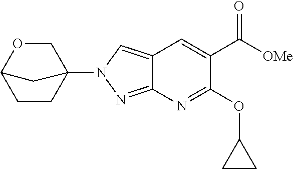 [SMILES: COC(=O)c1cc2cn(C34CCC(C3)OC4)nc2nc1OC1CC1] |
| | RNO₂: (E)-N-(2-oxabicyclo[2.2.1]heptan-4-yl)-1-(6-cyclopropoxy-2- |
| | nitropyridin-3-yl)methanimine (Preparation 97) |
| | Yellow oil (350 mg, 98%); LCMS m/z = 330.1 [M + H]⁺. |
| 104 | methyl 6-cyclopropoxy-2-(1-(fluoromethyl)-2-oxabicyclo[2.1.1]hexan-4-yl)- |
| | 2H-pyrazolo[3,4-b]pyridine-5-carboxylate |
| | 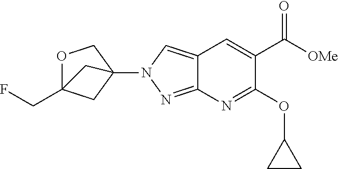 [SMILES: COC(=O)c1cc2cn(C34COC(CF)(C3)C4)nc2nc1OC1CC1] |
| | RNO₂: (E)-1-(6-cyclopropoxy-2-nitropyridin-3-y1)-N-(1-(fluoromethyl)-2- |
| | oxabicyclo[2.1.1]hexan-4-yl)methanimine (Preparation 98) |
| | Yellow solid (120 mg, 82%); LCMS m/z = 348.1 [M + H]⁺. |
| 105 | methyl 6-cyclobutoxy-2-(1-(methoxymethyl)-2-oxabicyclo[2.1.1]hexan-4- |
| | yl)-2H-pyrazolo[3,4-b]pyridine-5-carboxylate |
| | 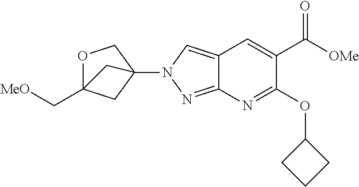 [SMILES: COCC12CC(n3cc4cc(C(=O)OC)c(OC5CCC5)nc4n3)(CO1)C2] |
| | RNO₂: (E)-1-(6-cyclobutoxy-2-nitro-2,3-dihydropyridin-3-yl)-N-(1- |
| | (methoxymethyl)-2-oxabicyclo[2.1.1]hexan-4-yl)methanimine (Preparation |
| | 99) |
| | Yellow oil (70 mg, 27%); LCMS m/z = 374.0 [M + H]⁺. |

### Preparation 106: 6-cyclopropoxy-2-(1-methyl-2-oxabicyclo[2.1.1]hexan-4-yl)-2H-pyrazolo[3,4,b]pyridine-5-carboxylic acid

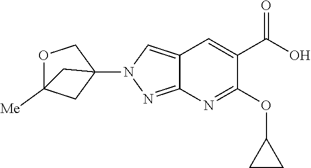 [SMILES: CC12CC(n3cc4cc(C(=O)O)c(OC5CC5)nc4n3)(CO1)C2]

To a solution of methyl 6-cyclopropoxy-2-(1-methyl-2-oxabicyclo[2.1.1]hexan-4-yl)-2H-pyrazolo[3,4-b]pyridine-5-carboxylate (Preparation 100, 200 mg, 0.607 mmol) in MeOH (1 mL) and water (1 mL) was added LiOH (76.45 mg, 1.82 mmol) and the mixture stirred at 25° C. for 2 h. The mixture was adjusted by HCl aq. (1 mol/L) to pH=7 and concentrated in vacuo to give the residue which was lyophilized to give 6-cyclopropoxy-2-(1-methyl-2-oxabicyclo[2.1.1]hexan-4-yl)-2H-pyrazolo[3,4-b]pyridine-5-carboxylic acid as a white solid (190 mg, 99%). LCMS m/z=316.0 [M+H]⁺.

### Preparation 107-110

The title compounds were prepared from the appropriate methyl ester (RC₂Me) using an analogous method to that described for Preparation 106.

| Preparation | |
|---|---|
| Number | Name/Structure/RCO₂Me/Data |
| 107 | 6-cyclopropoxy-2-(1-(methoxymethyl)-2-oxabicyclo[2.1.1]hexan-4-yl)-2H- |
| | pyrazolo[3,4-b]pyridine-5-carboxylic acid |
| | 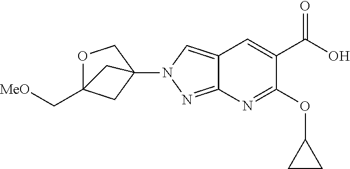 [SMILES: COCC12CC(n3cc4cc(C(=O)O)c(OC5CC5)nc4n3)(CO1)C2] |
| | RCO₂Me: methyl 6-cyclopropoxy-2-(1-(methoxymethyl)-2- |
| | oxabicyclo[2.1.1]hexan-4-yl)-2H-pyrazolo[3,4-b]pyridine-5-carboxylate |
| | (Preparation 101) |
| | White solid (120 mg, 96%); LCMS m/z = 346.1 [M + H]⁺. |
| 108 | 2-(2-oxabicyclo[2.1.1]hexan-4-yl)-6-cyclopropoxy-2H-pyrazolo[3,4- |
| | b]pyridine-5-carboxylic acid |
| | 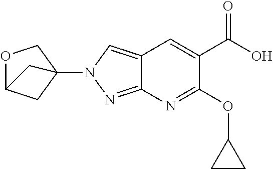 [SMILES: O=C(O)c1cc2cn(C34COC(C3)C4)nc2nc1OC1CC1] |
| | RCO₂Me: methyl 2-(2-oxabicyclo[2.1.1]hexan-4-yl)-6-cyclopropoxy-2H- |
| | pyrazolo[3,4-b]pyridine-5-carboxylate (Preparation 102) |
| | White solid (100 mg, 95%); LCMS m/z = 302.1 [M + H]⁺. |
| 109 | 2-(2-oxabicyclo[2.2.1]heptan-4-yl)-6-cyclopropoxy-2H-pyrazolo[3,4- |
| | b]pyridine-5-carboxylic acid |
| | 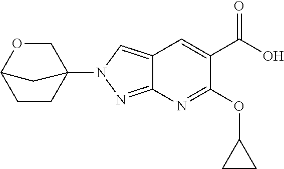 [SMILES: O=C(O)c1cc2cn(C34CCC(C3)OC4)nc2nc1OC1CC1] |
| | RCO₂Me: methyl 2-(2-oxabicyclo[2.2.1]heptan-4-yl)-6-cyclopropoxy-2H- |
| | pyrazolo[3,4-b]pyridine-5-carboxylate (Preparation 103) |
| | White solid (330 mg, 98%); LCMS m/z = 316.1 [M + H]⁺. |
| 110 | 6-cyclopropoxy-2-(1-(fluoromethyl)-2-oxabicyclo[2.1.1]hexan-4-yl)-2H- |
| | pyrazolo[3,4-b]pyridine-5-carboxylic acid |
| | 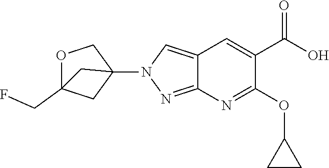 [SMILES: O=C(O)c1cc2cn(C34COC(CF)(C3)C4)nc2nc1OC1CC1] |
| | RCO₂Me: methyl 6-cyclopropoxy-2-(1-(fluoromethyl)-2- |
| | oxabicyclo[2.1.1]hexan-4-yl)-2H-pyrazolo[3,4-b]pyridine-5-carboxylate |
| | (Preparation 104) |
| | White solid (110 mg, 95%); LCMS m/z = 334.1 [M + H]⁺. |
| 111 | 6-cyclobutoxy-2-(1-(methoxymethyl)-2-oxabicyclo[2.1.1]hexan-4-yl)-2H- |
| | pyrazolo[3,4-b]pyridine-5-carboxylic acid |
| | 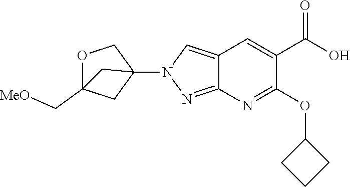 [SMILES: COCC12CC(n3cc4cc(C(=O)O)c(OC5CCC5)nc4n3)(CO1)C2] |
| | RCO₂Me: methyl 6-cyclobutoxy-2-(1-(methoxymethyl)-2- |
| | oxabicyclo[2.1.1]hexan-4-yl)-2H-pyrazolo[3,4-b]pyridine-5-carboxylate |
| | (Preparation 105) |
| | White solid (110 mg, 95%); LCMS m/z = 334.1 [M + H]⁺. |

### Preparation 112 and 113: 2-((1S,4S)-2-oxabicyclo[2.2.1]heptan-4-yl)-6-cyclopropoxy-2H-pyrazolo[3,4-b]pyridine-5-carboxylic acid and 2-((1R,4R)-2-oxabicyclo[2.2.1]heptan-4-yl)-6-cyclopropoxy-2H-pyrazolo[3,4-b]pyridine-5-carboxylic acid

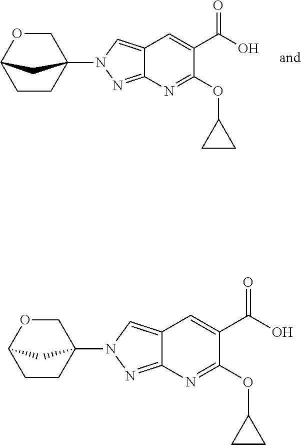 [SMILES: O=C(O)c1cc2cn([C@@]34CC[C@@H](C3)OC4)nc2nc1OC1CC1 ; O=C(O)c1cc2cn([C@]34CC[C@H](C3)OC4)nc2nc1OC1CC1]

*Stereochemistry arbitrarily assigned

2-(2-oxabicyclo[2.2.1]heptan-4-yl)-6-cyclopropoxy-2H-pyrazolo[3,4-b]pyridine-5-carboxylic acid (Preparation 109, 330 mg, 1.05 mmol) was further purified by prep-SFC (Cellulose-2 100×4.6 mm, 3 mm, 50% EtOH (0.05% DEA) in CO₂) to give the title compounds.

*Peak 1, Preparation 112 (White solid; 120 mg, 36%): 2-((1S,4S)-2-oxabicyclo[2.2.1]heptan-4-yl)-6-cyclopropoxy-2H-pyrazolo[3,4-b]pyridine-5-carboxylic acid; LCMS m/z=316.1 [M+H]⁺.

*Peak 2, Preparation 113 (White solid; 110 mg, 33%): 2-((1R,4R)-2-oxabicyclo[2.2.1]heptan-4-yl)-6-cyclopropoxy-2H-pyrazolo[3,4-b]pyridine-5-carboxylic acid; LCMS m/z=316.1 [M+H]⁺.

The following examples below were purified using the following prep-HPLC method unless otherwise noted. Prep-HPLC-A: Phenomenex Synergi C18 150×30 mm, 4 mm; 49-69% MeCN/H₂O (0.05% (NH₄HCO₃)-ACN); Prep-HPLC-B: Welch Xtimate C18 150×25 mm, 5 μm; 42-72% MeCN/H₂O (10 mm NH₄HCO₃): Prep-HPLC-C-Waters Sunfire OBD 100×50 mm, 5 mm; 5-75% MeCN/H₂O (+0.1% TFA).

### EXAMPLES

### Example 1: 6-Cyclobutoxy-N-(1-cyclopropyl-2-oxo-1,2-dihydropyridin-3-yl)-2-(1-methyl-2-oxabicyclo[2.1.1]hexan-4-yl)-2H-indazole-5-carboxamide

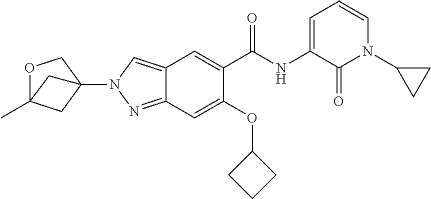 [SMILES: CC12CC(n3cc4cc(C(=O)Nc5cccn(C6CC6)c5=O)c(OC5CCC5)cc4n3)(CO1)C2]

To a solution of 6-cyclobutoxy-2-(1-methyl-2-oxabicyclo[2.1.1]hexan-4-yl)-2H-indazole-5-carboxylic acid [preparation 50](50.0 mg, 152 mol) and 3-amino-1-cyclopropylpyridin-2(1H)-one (91.5 mg, 609 mol) in Pyridine (2 mL) was added T₃P (2 mL). The mixture was stirred at 20° C. for 16 hours. The mixture was concentrated in vacuo to give the residue, which was diluted with saturated NaHCO₃ aq. till pH=7. And this mixture was extracted with EtOAc (50 mL×3). The combined organic layer was washed with brine (50 mL) and dried over Na₂SO₄, filtered. The filtrate was concentrated in vacuo to give the residue, which was purified by prep-HPLC (Column: Agela DuraShell C18 150×25 mm×5 um, water (0.05% NH₃H₂O+10 mM NH₄HCO₃)-ACN as a mobile phase, from 27% to 57%, Gradient Time=10 minutes, Flow Rate (ml/min): 25) to give 6-cyclobutoxy-N-(1-cyclopropyl-2-oxo-1,2-dihydropyridin-3-yl)-2-(1-methyl-2-oxabicyclo[2.1.1]hexan-4-yl)-2H-indazole-5-carboxamide (18.0 mg, 25.7% yield) as a white solid. LCMS m/z=461.0 [M+H]⁺. ¹H NMR: (400 MHz, CHLOROFORM-d) δ ppm 10.95 (s, 1H), 8.70 (s, 1H), 8.65-8.61 (m, 1H), 8.05 (s, 1H), 7.04-7.01 (m, 1H), 6.95 (s, 1H), 6.25-6.20 (m, 1H), 4.93-4.87 (m, 1H), 4.23 (s, 2H), 3.48-3.42 (m, 1H), 2.69-2.62 (m, 4H), 2.37-2.31 (m, 4H), 2.05-2.01 (m, 1H), 1.84-1.76 (m, 1H), 1.60 (s, 3H), 1.19-1.14 (m, 2H), 0.95-0.90 (m, 2H).

### Examples 2 and 3: N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide and N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide

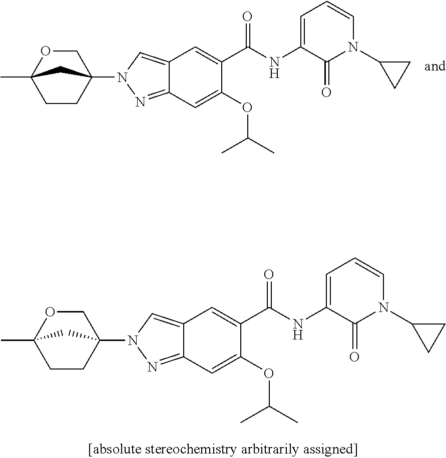 [SMILES: CC(C)Oc1cc2nn([C@@]34CC[C@@](C)(C3)OC4)cc2cc1C(=O)Nc1cccn(C2CC2)c1=O ; CC(C)Oc1cc2nn([C@]34CC[C@](C)(C3)OC4)cc2cc1C(=O)Nc1cccn(C2CC2)c1=O]

To a solution of 3-amino-1-cyclopropylpyridin-2(1H)-one (81.8 mg, 545 mol) in Pyridine (3.00 mL) was added 6-isopropoxy-2-(1-methyl-2-oxabicyclo[2.2.1]heptan-4-yl)-2H-indazole-5-carboxylic acid [preparation 48](90.0 mg, 272 mol) and T3P (3.00 mL) at 25° C. The reaction was stirred at 25° C. for 16 hours. The reaction was evaporated under vacuum to give the residue. The residue was diluted with aqueous aq. NaHCO₃ (30 mL), extracted with EtOAc (30 mL×3). The combined organic layer was dried over Na₂SO₄; filtered and concentrated to give the residue. The residue was purified by combi-flash (PE/EA from 1/1 to 0/1) to give N-(1-cyclopropyl-2-oxo-1,2-dihydropyridin-3-yl)-6-isopropoxy-2-(1-methyl-2-oxabicyclo[2.2.1]heptan-4-yl)-2H-indazole-5-carboxamide (100 mg, 71.4% yield) as a white solid. A solution of this racemic compound (120.0 mg, 259.4 mol) was purified by prep-SFC (Column: DAICEL CHIRALCEL OD-H (250 mm×30 mm, 5 m); Mobile Phase: from 60% to 60% of 0.1% NH₃H₂O MEOH; Flow Rate (ml/min): 80) to give Peak 1, Example 2, N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide (46.3 mg) and Peak 2, Example 3, N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide (43.3 mg) both as a white solid. Example 2: N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide (46.3 mg, 100% ee) LCMS: m/z=463.4 [M+H]⁺. ¹H NMR: (400 MHz, CDCl₃) δ:10.89 (s, 1H), 8.67 (s, 1H), 8.62 (dd, J=7.2, 1.6 Hz, 1H), 8.04 (s, 1H), 7.13 (s, 1H), 7.02 (dd, J=6.8, 1.6 Hz, 1H), 6.22 (t, J=14.4, 7.2 Hz, 1H), 4.89-4.82 (m, 1H), 4.23 (d, J=6.4 Hz, 1H), 4.20-4.16 (m, 1H), 3.48-3.41 (m, 1H), 2.48-2.40 (m, 2H), 2.34-2.28 (m, 2H), 2.05-1.98 (m, 2H), 1.63 (d, J=6.4 Hz, 6H), 1.52 (s, 3H), 1.19-1.13 (m, 2H), 0.94-0.89 (m, 2H).

Example 3: N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide (43.3 mg, 100% ee) LCMS: m/z=463.3 [M+H]⁺. ¹H NMR: (400 MHz, CDCl₃) δ: 10.89 (s, 1H), 8.67 (s, 1H), 8.62 (dd, J=7.2, 1.6 Hz, 1H), 8.04 (s, 1H), 7.13 (s, 1H), 7.02 (dd, J=6.8, 1.6 Hz, 1H), 6.22 (t, J=14.4, 7.2 Hz, 1H), 4.89-4.82 (m, 1H), 4.23 (d, J=6.4 Hz, 1H), 4.20-4.16 (m, 1H), 3.48-3.41 (m, 1H), 2.50-2.40 (m, 2H), 2.36-2.28 (m, 2H), 2.07-1.98 (m, 2H), 1.63 (d, J=6.4 Hz, 6H), 1.52 (s, 3H), 1.19-1.13 (m, 2H), 0.94-0.89 (m, 2H).

### Examples 4 and 5: (S)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide and (R)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide

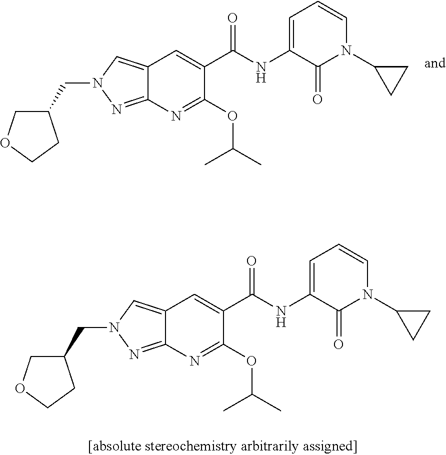 [SMILES: CC(C)Oc1nc2nn(C[C@@H]3CCOC3)cc2cc1C(=O)Nc1cccn(C2CC2)c1=O ; CC(C)Oc1nc2nn(C[C@H]3CCOC3)cc2cc1C(=O)Nc1cccn(C2CC2)c1=O]

To a solution of 6-isopropoxy-2-((tetrahydrofuran-3-yl)methyl)-2H-pyrazolo[3,4-b]pyridine-5-carboxylic acid [preparation 52](80.0 mg, 262 mol) in pyridine (3.00 mL) was added 3-amino-1-cyclopropylpyridin-2(1H)-one (78.7 mg, 524 mol) and T3P (3.00 mL) at 25° C. The reaction was stirred at 60° C. for 14 hours. The reaction was evaporated under vacuum to give the residue. The residue was diluted with aqueous aq. NaHCO₃ (30 mL), extracted with EtOAc (30 mL×3). The combined organic layer was dried over Na₂SO₄, filtered and concentrated to give the residue. The residue was purified by prep-HPLC (Column: Welch Xtimate C18 150×25 mm×5 m; Mobile Phase: from 29% to 59% of water (10 mM NH₄HCO₃)-ACN Gradient Time (10 min)) to give N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide (80.0 mg, 62.8% yield) as a white solid. A solution of the racemic sample (80.0 mg, 183 mol) was purified by prep-SFC (Column: Phenomenex Lux Cellulose-4 (250 mm×30 mm, 5 m); Mobile Phase: from 45% to 45% of 0.1% NH₃H₂O EtOH; Flow Rate (ml/min): 60) to give Peak 1, Example 4: (S)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide (28.9 mg, 66.1 mol) and Peak 2, Example 5: (R)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide (26.9 mg, 61.5 mol) both as white solids.

Example 4: (S)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide (28.9 mg, >99% ee) LCMS: m/z=438.3 [M+H]⁺. ¹H NMR: (400 MHz, CDCl₃) δ 10.98 (s, 1H), 8.98 (s, 1H), 8.60 (dd, J=7.2, 1.6 Hz, 1H), 7.96 (s, 1H), 7.03 (dd, J=6.8, 1.6 Hz, 1H), 6.22 (t, J=14.4, 7.2 Hz, 1H), 5.93-5.86 (m, 1H), 4.33 (d, J=7.6 Hz, 2H), 4.00-3.93 (m, 1H), 3.83-3.77 (m, 2H), 3.64 (dd, J=9.2, 4.8 Hz, 1H), 3.50-3.43 (m, 1H), 3.15-3.08 (m, 1H), 2.15-2.06 (m, 1H), 1.77-1.69 (m, 1H), 1.64 (d, J=6.4 Hz, 6H), 1.20-1.14 (m, 2H), 0.95-0.89 (m, 2H).

Example 5: (R)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide (26.9 mg, >99% ee) LCMS: m/z=438.3 [M+H]⁺. ¹H NMR: (400 MHz, CDCl₃) δ 10.98 (s, 1H), 8.98 (s, 1H), 8.60 (dd, J=7.2, 1.6 Hz, 1H), 7.96 (s, 1H), 7.03 (dd, J=6.8, 1.6 Hz, 1H), 6.22 (t, J=14.4, 7.2 Hz, 1H), 5.93-5.86 (m, 1H), 4.34 (d, J=7.6 Hz, 2H), 4.00-3.93 (m, 1H), 3.82-3.77 (m, 2H), 3.64 (dd, J=8.8, 4.4 Hz, 1H), 3.50-3.43 (m, 1H), 3.15-3.08 (m, 1H), 2.15-2.05 (m, 1H), 1.77-1.69 (m, 1H), 1.64 (d, J=6.0 Hz, 6H), 1.20-1.14 (m, 2H), 0.95-0.90 (m, 2H).

### Examples 6 and 7: 6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-pyrazolo[3,4-b]pyridine-5-carboxamide and 6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide

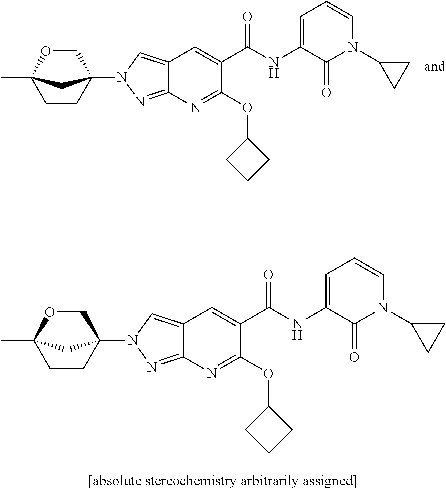 [SMILES: C[C@@]12CC[C@@](n3cc4cc(C(=O)Nc5cccn(C6CC6)c5=O)c(OC5CCC5)nc4n3)(CO1)C2 ; C[C@]12CC[C@](n3cc4cc(C(=O)Nc5cccn(C6CC6)c5=O)c(OC5CCC5)nc4n3)(CO1)C2]

To a solution of 6-cyclobutoxy-2-(1-methyl-2-oxabicyclo[2.2.1]heptan-4-yl)-2H-pyrazolo[3,4-b]pyridine-5-carboxylic acid [preparation 15](37.0 mg, 108 μmol) in pyridine (1 mL) was added 3-amino-1-cyclopropylpyridin-2(1H)-one (31.2 mg, 167 μmol) and T3P (1 mL) and stirred at 50° C. for 16 h. The mixture was adjusted by NaHCO₃ aq to pH=7 and the mixture was extracted with EtOAc (20 mL×3). The combined organic layers were washed with brine (20 mL), dried over Na₂SO₄, filtered and concentrated in vacuo to give the residue, which was purified by prep-TLC (PE/EtOAc=1/2) to give 6-cyclobutoxy-N-(1-cyclopropyl-2-oxo-1,2-dihydropyridin-3-yl)-2-(1-methyl-2-oxabicyclo[2.2.1]heptan-4-yl)-2H-pyrazolo[3,4-b]pyridine-5-carboxamide (40.0 mg, 78.1% yield) as white solid. The racemic sample (40.0 mg, 84.1 μmol) was further purified by SFC (Column: Chiralpak AD-3 50×4.6 mm I.D., 3 um, Mobile phase: A: CO2 B: ethanol (0.05% DEA), Isocratic: 40% B, Flow rate: 4 mL/min, Column temp.: 35° C., ABPR: 1500 psi) to give Peak 1, Example 6: 6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-pyrazolo[3,4-b]pyridine-5-carboxamide (12.6 mg, 31.5% yield) and Peak 2, Example 7: 6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide (21.3 mg, 53.2% yield) both as off-white solids.

Example 6: 6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-pyrazolo[3,4-b]pyridine-5-carboxamide (12.6 mg, >99% ee) LCMS: m/z=476.2 [M+H]⁺. ¹H NMR: (500 MHz, METHANOL-d₄) δ ppm 9.00 (s, 1H), 8.60 (dd, J=7.5, 1.5 Hz, 1H), 8.50 (s, 1H), 7.34 (dd, J=7.0, 1.0 Hz, 1H), 6.38 (t, J=6.5 Hz, 1H), 5.61-5.54 (m, 1H), 4.19 (d, J=6.5 Hz, 1H), 4.10 (dd, J=6.0, 3.5 Hz, 1H), 3.49-3.44 (m, 1H), 2.65-2.56 (m, 4H), 2.49-2.43 (m, 1H), 2.39 (s, 2H), 2.37-2.29 (m, 1H), 2.09-1.95 (m, 3H), 1.87-1.76 (m, 1H), 1.48 (s, 3H), 1.20-1.16 (m, 2H), 1.00-0.96 (m, 2H).

Example 7: 6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide (21.3 mg, >99% ee) LCMS: m/z=476.2 [M+H]⁺. ¹H NMR: (400 MHz, METHANOL-d₄) δ ppm 9.00 (s, 1H), 8.60 (dd, J=7.2, 1.2 Hz, 1H), 8.47 (s, 1H), 7.34 (dd, J=6.8, 1.6 Hz, 1H), 6.38 (t, J=7.2 Hz, 1H), 5.61-5.53 (m, 1H), 4.18 (d, J=6.4 Hz, 1H), 4.09 (dd, J=6.4, 3.6 Hz, 1H), 3.49-3.43 (m, 1H), 2.65-2.57 (m, 4H), 2.49-2.42 (m, 1H), 2.38 (s, 2H), 2.36-2.29 (m, 1H), 2.06-1.95 (m, 3H), 1.87-1.74 (m, 1H), 1.48 (s, 3H), 1.21-1.15 (m, 2H), 1.00-0.94 (m, 2H).

### Example 8: N-(1-cyclopropyl-2-oxo-1,2-dihydropyridin-3-yl)-6-isopropoxy-2-(1-methyl-2-oxabicyclo[2.1.1]hexan-4-yl)-2H-indazole-5-carboxamide

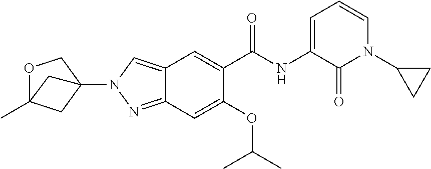 [SMILES: CC(C)Oc1cc2nn(C34COC(C)(C3)C4)cc2cc1C(=O)Nc1cccn(C2CC2)c1=O]

To a solution of 3-amino-1-cyclopropylpyridin-2(1H)-one (28.5 mg, 189 mol) in pyridine (2 mL) was added 6-isopropoxy-2-(1-methyl-2-oxabicyclo[2.1.1]hexan-4-yl)-2H-indazole-5-carboxylic acid [preparation 49](30.0 mg, 94.8 mol) and T3P (2 mL) at 25° C. The reaction was stirred at 25° C. for 14 hours. Solvent was evaporated under vacuum. The residue was diluted with aqueous NaHCO₃ (30 mL), extracted with EtOAc (30 mL×3). The organic layer was dried over Na₂SO₄; filtered and evaporated under vacuum. The residue was purified by prep-HPLC (Column: Welch Xtimate C18 150×25 mm×5 um, water (10 mM NH₄HCO₃)-ACN as a mobile phase, a mobile phase, from 34% to 64%, Gradient Time (min): 10, Flow Rate (ml/min): 25) to give N-(1-cyclopropyl-2-oxo-1,2-dihydropyridin-3-yl)-6-isopropoxy-2-(1-methyl-2-oxabicyclo[2.1.1]hexan-4-yl)-2H-indazole-5-carboxamide (18.1 mg, 42.6% yield) as a white solid. LCMS: m/z=449.0 [M+H]⁺. ¹H NMR: (500 MHz, CDCl₃) δ:10.87 (brs, 1H), 8.67 (s, 1H), 8.62 (dd, J₁=7.5 Hz, J₂=1.5 Hz, 1H), 8.04 (s, 1H), 7.13 (s, 1H), 7.01 (dd, J₁=7.0 Hz, J₂=1.5 Hz, 1H), 6.23-6.19 (m, 1H), 4.88-4.82 (m, 1H), 4.23 (s, 2H), 3.47-3.42 (m, 1H), 2.37-2.31 (m, 4H), 1.62 (d, J=6.0 Hz, 6H), 1.60 (s, 3H), 1.18-1.13 (m, 2H), 0.93-0.89 (m, 2H).

### Example 9: N-(1-cyclopropyl-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide

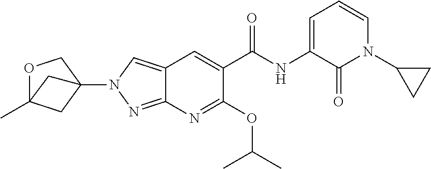 [SMILES: CC(C)Oc1nc2nn(C34COC(C)(C3)C4)cc2cc1C(=O)Nc1cccn(C2CC2)c1=O]

To a solution of 6-isopropoxy-2-(1-methyl-2-oxabicyclo[2.1.1]hexan-4-yl)-2H-pyrazolo[3,4-b]pyridine-5-carboxylic acid [preparation 6](20 mg, 0.063 mmol) and 3-amino-1-cyclopropylpyridin-2(1H)-one (14 mg, 0.094 mmol) in Pyridine (1 mL) was added T3P (1 mL, 50% in EtOAc). The mixture was stirred at 25° C. for 1 h. The mixture was diluted with saturated NaHCO₃ aq. (30 mL) and it was extracted with EtOAc (30 mL×3). The combined organic layer was washed with brine (30 mL) and dried over Na₂SO₄, filtered. The filtrate was concentrated in vacuo to give the residue, which was purified by prep-HPLC (Column: Welch Xtimate C18 150×25 mm×5 μm; Condition: water (10 mm NH₄HCO₃)-ACN; Begin B: 42; End B: 72; Gradient Time (min): 10; 100% B Hold Time (min): 2; Flow Rate (mL/min): 25) to give N-(1-cyclopropyl-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide (17.2 mg, 60.7% yield) was a white solid. LCMS: m/z=472.0 [M+Na]+. ¹HNMR: (500 MHz, CHLOROFORM-d) δ ppm 10.97 (s, 1H), 8.99 (s, 1H), 8.58 (d, J=7.5 Hz, 1H), 8.01 (s, 1H), 7.04 (d, J=7.0 Hz, 1H), 6.22 (t, J=7.5 Hz, 1H), 6.00-5.90 (m, 1H), 4.24 (s, 2H), 3.50-3.40 (m, 1H), 2.40-2.30 (m, 4H), 1.64 (d, J=6.5 Hz, 6H), 1.58 (s, 3H), 1.20-1.10 (m, 2H), 1.00-0.90 (m, 2H).

### Example 10: N-(1-cyclopropyl-2-oxo-1,2-dihydropyridin-3-yl)-2-(1-(fluoromethyl)-2-oxabicyclo[2.1.1]hexan-4-yl)-6-isopropoxy-2H-pyrazolo[3,4-b]pyridine-5-carboxamide

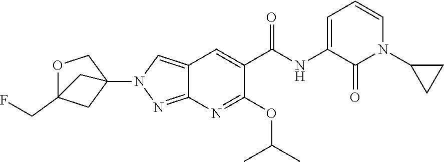 [SMILES: CC(C)Oc1nc2nn(C34COC(CF)(C3)C4)cc2cc1C(=O)Nc1cccn(C2CC2)c1=O]

To a solution of 2-(1-(fluoromethyl)-2-oxabicyclo[2.1.1]hexan-4-yl)-6-isopropoxy-2H-pyrazolo[3,4-b]pyridine-5-carboxylic acid [preparation 19](33.0 mg, 98.4 mol) in pyridine (3 mL) was added 3-amino-1-cyclopropylpyridin-2(1H)-one (40.0 mg, 214 mol, HCl) and T3P (3 mL) at 20° C. The reaction mixture was stirred at 20° C. for 14 h. The reaction was concentrated to give the residue. The residue was diluted with aqueous NaHCO₃ (30 mL), extracted with EtOAc (30 mL×3). The combined organic layers were dried over Na₂SO₄, filtered and concentrated to give the residue. The residue was purified by prep-HPLC (Column: Boston Prime C18 150×30 mm×5 m; Mobile Phase: from 42% to 72% of water (10 mM NH₄HCO₃)-ACN Gradient Time (10 min)) to give N-(1-cyclopropyl-2-oxo-1,2-dihydropyridin-3-yl)-2-(1-(fluoromethyl)-2-oxabicyclo[2.1.1]hexan-4-yl)-6-isopropoxy-2H-pyrazolo[3,4-b]pyridine-5-carboxamide (27.7 mg, 60.2% yield) as a white solid. LCMS: m/z=468.1 [M+H]⁺. ¹H NMR: (400 MHz, CDCl₃) □: 10.97 (s, 1H), 9.00 (s, 1H), 8.59 (dd, J=7.6, 2.0 Hz, 1H), 8.04 (s, 1H), 7.04 (dd, J=6.8, 1.6 Hz, 1H), 6.22 (t, J=14.8, 7.2 Hz, 1H), 5.95-5.87 (m, 1H), 4.80 (s, 1H), 4.68 (s, 1H), 4.32 (s, 2H), 3.50-3.44 (m, 1H), 2.52 (d, J=4.8 Hz, 2H), 2.46 (d, J=5.2 Hz, 2H), 1.64 (d, J=6.0 Hz, 6H), 1.21-1.15 (m, 2H), 0.95-0.90 (m, 2H).

### Example 11: N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide

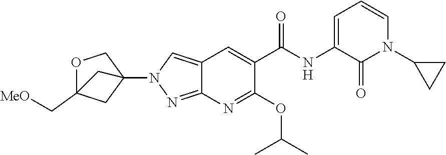 [SMILES: COCC12CC(n3cc4cc(C(=O)Nc5cccn(C6CC6)c5=O)c(OC(C)C)nc4n3)(CO1)C2]

To a solution of 6-isopropoxy-2-(1-(methoxymethyl)-2-oxabicyclo[2.1.1]hexan-4-yl)-2H-pyrazolo[3,4-b]pyridine-5-carboxylic acid [preparation 23](20.0 mg, 57.6 mol) in pyridine (1 mL) was added 3-amino-1-cyclopropylpyridin-2(1H)-one (12.0 mg, 64.3 mol, HCl) and T3P (1 mL) at 20° C. The reaction was stirred at 20° C. for 1 hour. Solvent was evaporated under vacuum. The residue was diluted with aqueous NaHCO₃ (30 mL), extracted with EtOAc (30 mL×3). The organic layer was dried over Na₂SO₄; filtered and evaporated under vacuum. The residue was purified by prep-HPLC (Column: Welch Xtimate C18 150×25 mm×5 um, water (10 mM NH₄HCO₃)-ACN as a mobile phase, from 31% to 61%, Gradient Time (min): 10, Flow Rate (ml/min): 25) to give N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide (23.5 mg, 85.1% yield) as a white solid. LCMS: m/z=480.0 [M+H]⁺. ¹H NMR: (500 MHz, CDCl₃) □: 10.97 (brs, 1H), 8.99 (s, 1H), 8.57 (dd, J₁=7.5 Hz, J₂=2.0 Hz, 1H), 8.03 (s, 1H), 7.04 (dd, J₁=7.5 Hz, J₂=1.5 Hz, 1H), 6.22 (t, J=7.5 Hz, 1H), 5.94-5.88 (m, 1H), 4.30 (s, 2H), 3.77 (s, 2H), 3.49-3.45 (m, 4H), 2.46-2.41 (m, 4H), 1.64 (d, J=6.0 Hz, 6H), 1.20-1.15 (m, 2H), 0.94-0.90 (m, 2H).

### Example 12: N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-indazole-5-carboxamide

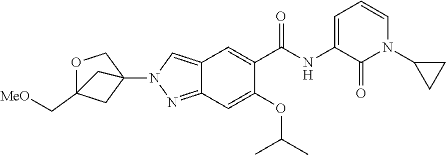 [SMILES: COCC12CC(n3cc4cc(C(=O)Nc5cccn(C6CC6)c5=O)c(OC(C)C)cc4n3)(CO1)C2]

To a solution of 6-isopropoxy-2-(1-(methoxymethyl)-2-oxabicyclo[2.1.1]hexan-4-yl)-2H-indazole-5-carboxylic acid [preparation 27](50.0 mg, 144 mol) in pyridine (1.5 mL) was added 3-amino-1-cyclopropylpyridin-2(1H)-one (22.0 mg, 146 mol) and T3P (1.5 mL) at 20° C. The reaction was stirred at 20° C. for 3 h. The reaction was evaporated under vacuum. The residue was diluted with aqueous NaHCO₃ (10 mL) to pH=7, extracted with EtOAc (30 mL×3). The combined organic layers was washed with brine (50 mL), dried over Na₂SO₄. The filtrate was concentrated in vacuum to give the residue, which was purified by prep-HPLC (Column: Welch Xtimate C18 150×25 mm×5 μm; Condition: water (10 mm NH₄HCO₃)-ACN; Begin B: 42; End B: 72; Gradient Time (min): 10; 100% B Hold Time (min): 2; Flow Rate (mL/min): 25) to give N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-indazole-5-carboxamide (26.0 mg, 18.8% yield) as a white solid. LCMS: m/z=479.2 [M+H]⁺. ¹H NMR: (400 MHz, CDCl₃) □: 10.85 (s, 1H), 8.65 (s, 1H), 8.60 (d, J=7.2 Hz, 1H), 8.04 (s, 1H), 7.10 (s, 1H), 7.00 (d, J=7.2 Hz, 1H), 6.19 (d, J=7.2 Hz, 1H), 4.86-4.80 (m, 1H), 4.26 (s, 2H), 3.75 (s, 2H), 3.46-3.41 (m, 4H), 2.40-2.30 (m, 4H), 1.60 (d, J=6.0 Hz, 6H), 1.10-1.00 (m, 2H), 0.90-0.80 (m, 2H).

### Example 13: N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-indazole-5-carboxamide

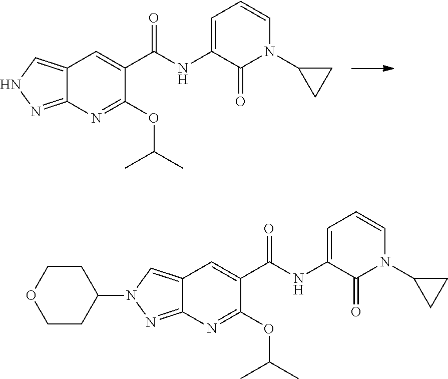 [SMILES: CC(C)Oc1nc2n[nH]cc2cc1C(=O)Nc1cccn(C2CC2)c1=O ; CC(C)Oc1nc2nn(C3CCOCC3)cc2cc1C(=O)Nc1cccn(C2CC2)c1=O]

To a solution of N-(1-cyclopropyl-2-oxo-1,2-dihydropyridin-3-yl)-6-isopropoxy-2H-pyrazolo[3,4-b]pyridine-5-carboxamide [preparation 57](80.0 mg, 226 mol) in DMF (3.00 mL) was added K₂CO₃ (93.8 mg, 679 mol) and 4-bromotetrahydro-2H-pyran (56.0 mg, 339 mol) at 20° C. The reaction mixture was heated to 100° C. and stirred for 16 hours. The reaction mixture was filtered and the filtrate was purified by prep-HPLC (Column: Welch Xtimate Welch Xtimate C18 150×25 mm×5 m; Mobile Phase: from 39% to 69% of water (10 mM NH₄HCO₃)-ACN Gradient Time (10 min)) to give N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-indazole-5-carboxamide (13.0 mg, 12.6% yield) as a yellow solid. LCMS: m/z=438.2 [M+H]⁺. ¹H NMR: (400 MHz, CDCl₃) δ:10.99 (s, 1H), 8.99 (s, 1H), 8.59 (dd, J=7.6, 1.6 Hz, 1H), 8.01 (s, 1H), 7.03 (dd, J=7.2, 1.6 Hz, 1H), 6.22 (t, J=7.2 Hz, 1H), 5.94-5.87 (m, 1H), 4.62-4.53 (m, 1H), 4.20-4.15 (m, 2H), 3.64-3.57 (m, 2H), 3.50-3.43 (m, 1H), 2.37-2.25 (m, 4H), 1.64 (d, J=6.0 Hz, 6H), 1.20-1.14 (m, 2H), 0.95-0.91 (m, 2H).

### Examples 14 and 15: (R)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide and (S)-6-(sec-butoxy)-N-(1-cyclopropyl-2-oxo-1,2-dihydropyridin-3-yl)-2-(1-methyl-2-oxabicyclo[21.1]hexan-4-yl)-2H-pyrazolo[3,4-h]pyridine-5-carboxamide

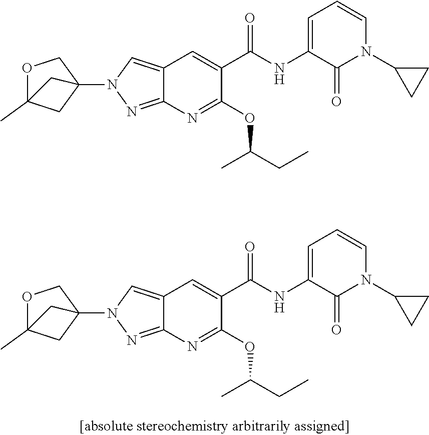 [SMILES: CC[C@@H](C)Oc1nc2nn(C34COC(C)(C3)C4)cc2cc1C(=O)Nc1cccn(C2CC2)c1=O ; CC[C@H](C)Oc1nc2nn(C34COC(C)(C3)C4)cc2cc1C(=O)Nc1cccn(C2CC2)c1=O]

To a solution of 6-(sec-butoxy)-2-(1-methyl-2-oxabicyclo[2.1.1]hexan-4-yl)-2H-pyrazolo[3,4-b]pyridine-5-carboxylic acid [preparation 68](50.0 mg, 151 mol) in pyridine (1 mL) was added 3-amino-1-cyclopropylpyridin-2(1H)-one (30.0 mg, 161 mol) and T3P (1 mL) and stirred at 25° C. for 1 h. The mixture was adjusted by NaHCO₃ aq. to pH=7 and the mixture was extracted with EtOAc (10 mL×3). The combined organic layer was washed with brine (10 mL), dried over Na₂SO₄, filtered and concentrated in vacuo to give the residue, which was purified by prep-TLC (PE/EtOAc=1/1) to give 6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide (30.0 mg, 42.9% yield) as white solid. The racemic sample (30.0 mg, 64.7 mol) was further purified by SFC (Column: Chiralpak AD-3 150; A4.6 mm I.D., 3 um, Mobile phase: A: CO₂ B: ethanol (0.05% DEA), Gradient: from 5% to 40% of B in 5 min and hold 40% for 2.5 min, then 5% of B for 2.5 min, Flow rate: 2.5 mL/min, Column temp.: 35 [symbol] æ, ABPR: 1500 psi) to give Peak 1, Example 14: (R)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide (10.1 mg, 32.5% yield) and Peak 2, Example 15: (S)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide (11.2 mg, 36.0% yield) as white solid.

Example 14: (R)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide (10.1 mg, 98% ee) LCMS: m/z=464.2 [M+H]⁺. ¹H NMR: (500 MHz, METHONAL-d4) δ ppm 9.02 (s, 1H), 8.60 (dd, J=7.5, 1.5 Hz, 1H), 8.50 (s, 1H), 7.35-7.32 (m, 1H), 6.37 (t, J=7.0 Hz, 1H), 5.69-5.61 (m, 1H), 4.17 (s, 2H), 3.47-3.42 (m, 1H), 2.45-2.39 (m, 2H), 2.31-2.26 (m, 2H), 2.22-2.15 (m, 1H), 1.95-1.85 (m, 1H), 1.59 (d, J=6.0 Hz, 3H), 1.56 (s, 3H), 1.20-1.16 (m, 2H), 1.03 (t, J=7.5 Hz, 3H), 0.99-0.94 (m, 2H).

Example 15: (S)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide (10.1 mg, 99% ee) LCMS: m/z=464.2 [M+H]⁺. ¹H NMR: (500 MHz, METHONAL-d4) δ ppm 9.02 (s, 1H), 8.60 (dd, J=7.5, 1.5 Hz, 1H), 8.50 (s, 1H), 7.35-7.32 (m, 1H), 6.37 (t, J=7.0 Hz, 1H), 5.69-5.61 (m, 1H), 4.17 (s, 2H), 3.47-3.42 (m, 1H), 2.43-2.39 (m, 2H), 2.30-2.25 (m, 2H), 2.22-2.15 (m, 1H), 1.95-1.85 (m, 1H), 1.59 (d, J=6.0 Hz, 3H), 1.56 (s, 3H), 1.20-1.16 (m, 2H), 1.03 (t, J=7.5 Hz, 3H), 0.99-0.95 (m, 2H).

### Examples 16 and 17: (S)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide and (R)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide

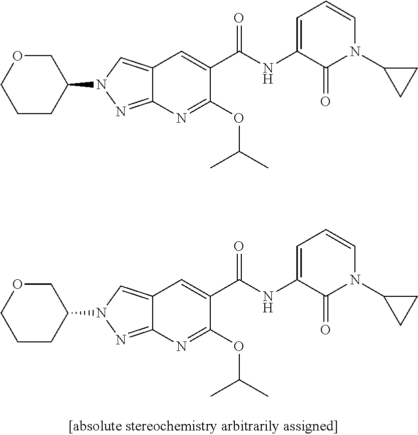 [SMILES: CC(C)Oc1nc2nn([C@H]3CCCOC3)cc2cc1C(=O)Nc1cccn(C2CC2)c1=O ; CC(C)Oc1nc2nn([C@@H]3CCCOC3)cc2cc1C(=O)Nc1cccn(C2CC2)c1=O]

To a solution of 6-isopropoxy-2-(tetrahydro-2H-pyran-3-yl)-2H-pyrazolo[3,4-b]pyridine-5-carboxylic acid [preparation 51](80.0 mg, 0.262 mmol) and 3-amino-1-cyclopropylpyridin-2(1H)-one (59.0 mg, 0.393 mmol) in Pyridine (4.00 mL) was added T3P (4.00 mL). The mixture was stirred at 20° C. for 2 h. The reaction mixture was concentrated to give the residue. The residue was diluted with water (10 mL) and adjusted by aqueous NaHCO₃ (10 mL) and extracted with EA (20 mL×3). The combined organic layer was washed with brine (30 mL), dried over Na₂SO₄, The mixture was filtered and the filtrate was purified by prep-HPLC (Column: Phenomenex Synergi C18 150*30 mm*4 um; Mobile Phase: from 49% to 69% of water (0.05% (NH₄HCO₃)-ACN) to give N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide (80.0 mg, 0.183 mmol, 69.8% yield) as a white solid. The racemic mixture (80.0 mg, 0.183 mmol) was purified by SFC (Column: Phenomenex-Cellulose-2 (250 mm*30 mm, 5 um); Mobile phase: A: CO₂ B: iso-propanol (0.05% DEA); Isocratic: 60% B; Flow rate: 2.8 mL/min; Column temp.: 35° C.; Back pressure: 1500 psi) to give Peak 1, Example 16: (S)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide (22.3 mg, 27.9% yield) and Peak 2, Example 17: (R)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide (22.6 mg, 28.3% yield) both as white solid.

Example 16: (S)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide (22.3 mg, 100% ee) LCMS: m/z=438.3 [M+H]⁺. ¹H NMR: (500 MHz, METHANOL-d₄) δ ppm 9.00 (s, 1H), 8.60 (dd, J=7.5, 1.5 Hz, 1H), 8.52 (s, 1H), 7.34 (d, J=7.0 Hz, 1H), 6.43-6.33 (m, 1H), 5.81 (dt, J=12.5, 6.0 Hz, 1H), 4.61 (s, 1H), 4.17 (dd, J=11.0, 4.0 Hz, 1H), 4.00-3.84 (m, 2H), 3.70-3.56 (m, 1H), 3.50-3.40 (m, 1H), 2.42-2.38 (m, 2H), 1.91-1.74 (m, 2H), 1.63 (d, J=6.5 Hz, 6H), 1.21-1.16 (m, 2H), 1.00-0.95 (m, 2H).

Example 17: (R)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide (22.3 mg, 96% ee) LCMS: m/z=438.3 [M+H]⁺. ¹H NMR: (500 MHz, METHANOL-d₄) δ ppm 8.99 (s, 1H), 8.59 (dd, J=7.5, 1.5 Hz, 1H), 8.51 (s, 1H), 7.33 (dd, J=7.0, 1.5 Hz, 1H), 6.37 (t, J=7.5 Hz, 1H), 5.95-5.76 (m, 1H), 4.61 (s, 1H), 4.16 (dd, J=11.5, 4.0 Hz, 1H), 3.98-3.83 (m, 2H), 3.73-3.56 (m, 1H), 3.47-3.43 (m, 1H), 2.36-2.24 (m, 2H), 1.89-1.77 (m, 2H), 1.62 (d, J=6.5 Hz, 6H), 1.18 (q, J=7.0 Hz, 2H), 1.00-0.95 (m, 2H).

### Example 18: 6-Isopropoxy-2-(1-methyl-2-oxabicyclo[2.1.1]hexan-4-yl)-N-(1-(1-methylcyclopropyl)-2-oxo-1,2-dihydropyridin-3-yl)-2H-pyrazolo[3,4-b]pyridine-5-carboxamide

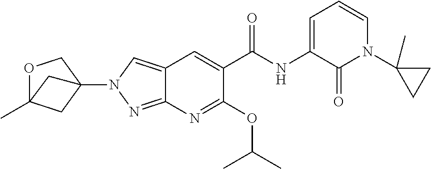 [SMILES: CC(C)Oc1nc2nn(C34COC(C)(C3)C4)cc2cc1C(=O)Nc1cccn(C2(C)CC2)c1=O]

To a solution of 6-isopropoxy-2-(1-methyl-2-oxabicyclo[2.1.1]hexan-4-yl)-2H-pyrazolo[3,4-b]pyridine-5-carboxylic acid [preparation 6](30.0 mg, 0.0945 mmol) and 3-amino-1-(1-methylcyclopropyl)pyridin-2(1H)-one [preparation 62](45.1 mg, 0.189 mmol) in Pyridine (1.00 mL) was added T3P (1.00 mL). The mixture was stirred at 20° C. for 2 h. The reaction mixture was concentrated to give the residue. The residue was diluted with water (10 mL) and adjusted by aqueous NaHCO₃ (10 mL) and extracted with EA (20 mL×3). The combined organic layer was washed with brine (30 mL), dried over Na₂SO₄, The mixture was filtered and the filtrate was purified by prep-HPLC (Column: Phenomenex Synergi C18 150*30 mm*4 um; Mobile Phase: from 49% to 69% of water (0.05% HCl)-ACN) to give 6-Isopropoxy-2-(1-methyl-2-oxabicyclo[2.1.1]hexan-4-yl)-N-(1-(1-methylcyclopropyl)-2-oxo-1,2-dihydropyridin-3-yl)-2H-pyrazolo[3,4-b]pyridine-5-carboxamide (37.3 mg, 0.0773 mmol, 80.2% yield) as a white solid. LCMS: m/z=464.3 [M+H]⁺. ¹H NMR: (500 MHz, METHANOL-d₄) δ ppm 8.98 (s, 1H), 8.54 (dd, J=7.5, 1.5 Hz, 1H), 8.49 (s, 1H), 7.42 (dd, J=7.0, 1.5 Hz, 1H), 6.37 (t, J=7.0 Hz, 1H), 5.78 (dt, J=12.5, 6.0 Hz, 1H), 4.17 (s, 2H), 2.41 (d, J=4.5 Hz, 2H), 2.28 (dd, J=4.50, 1.5 Hz, 2H), 1.63 (d, J=6.0 Hz, 6H), 1.55 (d, J=4.0 Hz, 6H), 1.15-1.10 (m, 2H), 1.07-1.05 (m, 2H).

### Example 19: 2-(1-(Fluoromethyl)-2-oxabicyclo[2.1.1]hexan-4-yl)-6-isopropoxy-N-(1-(1-methylcyclopropyl)-2-oxo-1,2-dihydropyridin-3-yl)-2H-pyrazolo[3,4-b]pyridine-5-carboxamide

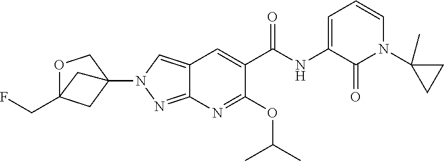 [SMILES: CC(C)Oc1nc2nn(C34COC(CF)(C3)C4)cc2cc1C(=O)Nc1cccn(C2(C)CC2)c1=O]

To a solution of 3-amino-1-(1-methylcyclopropyl)pyridin-2(1H)-one [preparation 62](30.0 mg, 149 mol, HCl) and 2-(1-(fluoromethyl)-2-oxabicyclo[2.1.1]hexan-4-yl)-6-isopropoxy-2H-pyrazolo[3,4-b]pyridine-5-carboxylic acid [preparation 19](50.1 mg, 149 mol) in pyridine (1 mL) was added T3P (1 mL, 50% in EtOAc). The mixture was stirred at 20° C. for 1 h. The reaction mixture was diluted with saturated NaHCO₃ aq. (30 mL) and it was extracted with EtOAc (20 mL×3). The combined organic layer was washed with brine (30 mL) and dried over Na₂SO₄, filtered. The filtrate was concentrated in vacuo to give the residue, which was purified by prep-HPLC (Column: Welch Xtimate C18 150×25 mm×5 m; Condition: water (10 mm NH₄HCO₃)-ACN; Begin B: 42; End B: 72; Gradient Time (min): 10; 100% B Hold Time (min): 2; Flow Rate (mL/min): 25) to give 2-(1-(fluoromethyl)-2-oxabicyclo[2.1.1]hexan-4-yl)-6-isopropoxy-N-(1-(1-methylcyclopropyl)-2-oxo-1,2-dihydropyridin-3-yl)-2H-pyrazolo[3,4-b]pyridine-5-carboxamide (15.9 mg, 22.1% yield) as a white solid. LCMS: m/z=482.1 [M+H]⁺. ¹HNMR: (500 MHz, CHLOROFORM-d) δ ppm 10.91 (s, 1H), 8.97 (s, 1H), 8.52 (d, J=7.0 Hz, 1H), 8.04 (s, 1H), 7.15 (d, J=7.0 Hz, 1H), 6.21 (t, J=7.0 Hz, 1H), 5.90-5.80 (m, 1H), 4.74 (d, J=47.0 Hz, 2H), 4.32 (s, 2H), 2.60-2.55 (m, 2H), 2.50-2.45 (m, 2H), 1.63 (d, J=6.0 Hz, 6H), 1.56 (s, 3H), 1.20-1.10 (m, 2H), 1.00-0.90 (m, 2H).

### Examples 20 and 21: (R)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide and (S)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide

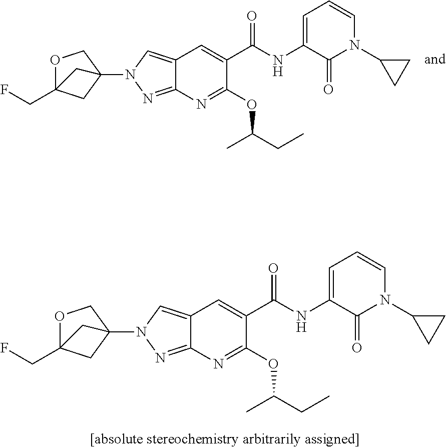 [SMILES: CC[C@@H](C)Oc1nc2nn(C34COC(CF)(C3)C4)cc2cc1C(=O)Nc1cccn(C2CC2)c1=O ; CC[C@H](C)Oc1nc2nn(C34COC(CF)(C3)C4)cc2cc1C(=O)Nc1cccn(C2CC2)c1=O]

To a solution of 6-(sec-butoxy)-2-(1-(fluoromethyl)-2-oxabicyclo[2.1.1]hexan-4-yl)-2H-pyrazolo[3,4-b]pyridine-5-carboxylic acid [preparation 69]70.0 mg, 200 mol) and 3-amino-1-cyclopropylpyridin-2(1H)-one (45.1 mg, 300 mol) in pyridine (2 mL) was added T₃P (2 mL). The mixture was stirred at 20° C. for 4 hours. The mixture was diluted with saturated NaHCO₃ aq., extracted with EtOAc (50 mL×3). The combined organic layer was washed with brine (50 mL) and dried over Na₂SO₄, filtered. The filtrate was concentrated in vacuo to give the residue, The residue was purified by prep-TLC to give 6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide (75.0 mg, 77.7% yield) as a brown solid. The racemic sample (75.0 mg, 156 mol) was purified by SFC(Column: Chiralpak AD-3 50; A4.6 mm I.D., 3 m; Mobile phase: A: CO₂ B: ethanol (0.05% DEA); Gradient: from 5% to 40% of B in 2 min and hold 40% for 1.2 min, then 5% of B for 0.8 min; Flow rate: 4 mL/min; Column temp.: 35° C.; ABPR: 1500 psi) to give Peak 1, Example 20: (R)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide (36.1 mg, 48.1% yield) and Peak 2, Example 21: (S)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide (37.9 mg, 50.5% yield) both as brown solid.

Example 20: (R)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide (36.1 mg, 100% ee) LCMS: m/z=482.3 [M+H]⁺. ¹H NMR: (500 MHz, CHLOROFORM-d) δ ppm 10.97 (s, 1H), 9.01 (s, 1H), 8.60-8.57 (m, 1H), 8.04 (s, 1H), 7.05-7.02 (m, 1H), 6.24-6.20 (m, 1H), 5.79-5.74 (m, 1H), 4.74 (d, J=47.5 Hz, 2H), 4.32 (s, 2H), 3.50-3.44 (m, 1H), 2.53-2.45 (m, 4H), 2.21-1.89 (m, 2H), 1.60 (d, J=6.5 Hz, 3H), 1.20-1.15 (m, 2H), 1.04-0.99 (m, 3H), 0.94-0.90 (m, 2H).

Example 21: (S)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide (37.9 mg, 100% ee) LCMS: m/z=482.3 [M+H]⁺. ¹H NMR: (500 MHz, CHLOROFORM-d) δ ppm 10.97 (s, 1H), 9.01 (s, 1H), 8.60-8.58 (m, 1H), 8.04 (s, 1H), 7.05-7.02 (m, 1H), 6.23-6.20 (m, 1H), 5.78-5.75 (m, 1H), 4.74 (d, J=47.5 Hz, 2H), 4.32 (s, 2H), 3.50-3.44 (m, 1H), 2.53-2.45 (m, 4H), 2.21-1.87 (m, 2H), 1.60 (d, J=8.0 Hz, 3H), 1.20-1.15 (m, 2H), 1.04-0.99 (m, 3H), 0.94-0.90 (m, 2H)

### Examples 22 and 23: (R)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide and (S)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide

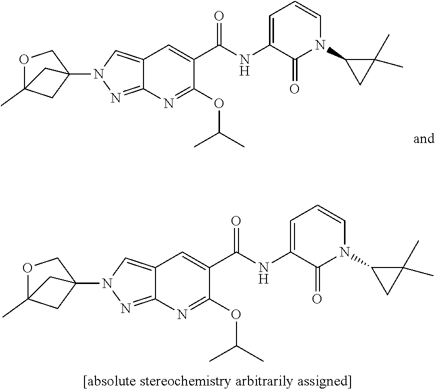

To a solution of 6-isopropoxy-2-(1-methyl-2-oxabicyclo[2.1.1]hexan-4-yl)-2H-pyrazolo[3,4-b]pyridine-5-carboxylic acid [preparation 6](60.2 mg, 189 μmol) in Pyridine (3 mL) added 3-amino-1-(2,2-dimethylcyclopropyl)pyridin-2(1H)-one [preparation 63](40.0 mg, 224 μmol) and T₃P (3 mL) at 25° C. The reaction was stirred at 25° C. for 4 hr. The mixture was tilled PH to 7 with NaHCO₃ aq. and extracted with EA (30 mL×3). The combined organic layers was washed with brine (20 mL), dried over Na₂SO₄, filtered and concentrated in vacuo to give residue. The residue was purified with preparative TLC (PE/EA=1/1) to afford compound 3 (50.0 mg, 55.2% yield) as a yellow solid. The racemic sample (50.0 mg, 104 μmol) was purified by prep SFC (Column: Chiralpak AD-3 50×4.6 mm I.D., 3 um Mobile phase: A: CO₂ B: ethanol (0.05% DEA) Gradient: from 5% to 40% of B in 2 min and hold 40% for 1.2 min, then 5% of B for 0.8 min Flow rate: 4 mL/min Column temp.: 35° C. ABPR: 1500 psi) to give Peak 1, Example 22: (R)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide (23.9 mg, 47.8% yield) and Peak 2, Example 23: (S)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide (22.1 mg, 44.2% yield) as white solid.

Example 22: (R)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide (23.9 mg, 100% ee) LCMS: m/z=478.3 [M+H]⁺. ¹H NMR: (500 MHz, CHLOROFORM-d) δ ppm 11.03 (s, 1H), 8.98 (s, 1H), 8.56 (dd, J=1.5, 7.5 Hz, 1H), 8.01 (s, 1H), 7.00 (dd, J=1.5, 7.0 Hz, 1H), 6.20 (t, J=7.0 Hz, 1H), 5.91-5.86 (m, 1H), 4.25 (s, 2H), 3.18 (dd, J=5.0, 7.5 Hz, 1H), 2.35-2.32 (m, 4H), 1.64 (d, J=6.5 Hz, 3H), 1.61-1.60 (m, 6H), 1.33 (s, 3H), 1.01 (t, J=7.0 Hz, 1H), 0.88 (s, 3H), 0.87-0.85 (m, 1H).

Example 23: (S)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide (22.1 mg, 100% ee) LCMS: m/z=478.1 [M+H]⁺. ¹H NMR: (500 MHz, CHLOROFORM-d) δ ppmfd 11.03 (s, 1H), 8.98 (s, 1H), 8.56 (dd, J=2.0, 7.5 Hz, 1H), 8.01 (s, 1H), 7.00 (dd, J=2.0, 7.0 Hz, 1H), 6.20 (t, J=7.0 Hz, 1H), 5.90-5.87 (m, 1H), 4.24 (s, 2H), 3.18 (dd, J=4.5, 7.5 Hz, 1H), 2.35-2.32 (m, 4H), 1.64 (d, J=6.5 Hz, 3H), 1.61-1.59 (m, 6H), 1.33 (s, 3H), 1.02-0.99 (m, 1H), 0.88 (s, 3H), 0.87-0.85 (m, 1H).

### Example 24: 6-cyclobutoxy-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridin-5-carboxamide

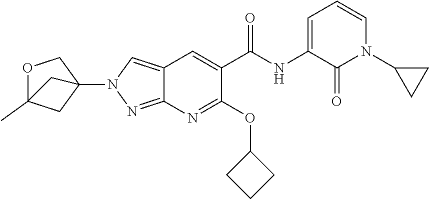 [SMILES: CC12CC(n3cc4cc(C(=O)Nc5cccn(C6CC6)c5=O)c(OC5CCC5)nc4n3)(CO1)C2]

To a mixture of 6-cyclobutoxy-2-(1-methyl-2-oxabicyclo[2.1.1]hexan-4-yl)-2H-pyrazolo[3,4-b]pyridine-5-carboxylic acid [preparation 11](32.93 mg, 0.1 mmol), 3-amino-1-cyclopropyl-pyridin-2-one HCl salt (20.53 mg, 0.11 mmol), HATU (41.94 mg, 0.11 mmol) in DMF (0.8 mL) was added Hunig's base (69.67 uL, 0.4 mmol). The mixture was stirred at rt overnight. It was partitioned between EtOAc and water. The aqueous layer was extracted with EtOAc. The combined organic phases were concentrated and purified by normal phase silica gel column (24 g, EtOAc in heptane 50-100%) to get 6-cyclobutoxy-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide as an off-white solid (39.4 mg, 85.4% yield). LCMS m/z=462.0 [M+H]⁺. ¹H NMR (METHANOL-d4, 400 MHz) δ 9.03 (s, 1H), 8.62 (dd, 1H, J=1.8, 7.5 Hz), 8.52 (s, 1H), 7.36 (dd, 1H, J=1.8, 7.0 Hz), 6.3-6.4 (m, 1H), 5.5-5.7 (m, 1H), 4.19 (s, 2H), 3.4-3.5 (m, 1H), 2.6-2.8 (m, 4H), 2.4-2.5 (m, 2H), 2.2-2.3 (m, 2H), 2.0-2.1 (m, 1H), 1.8-1.9 (m, 1H), 1.58 (s, 3H), 1.1-1.3 (m, 2H), 0.9-1.1 (m, 2H).

### Example 25: Racemic 6-cyclobutoxy-N-(1-(cis-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide

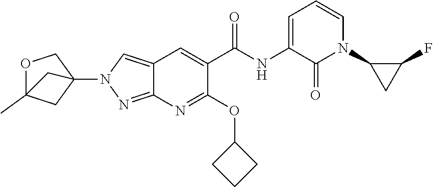 [SMILES: CC12CC(n3cc4cc(C(=O)Nc5cccn([C@@H]6C[C@@H]6F)c5=O)c(OC5CCC5)nc4n3)(CO1)C2]

To a mixture of 6-cyclobutoxy-2-(1-methyl-2-oxabicyclo[2.1.1]hexan-4-yl)-2H-pyrazolo[3,4-b]pyridine-5-carboxylic acid [preparation 11](50 mg, 152 μmol), 3-amino-1-[(cis-2-fluorocyclopropyl]pyridin-2-one [preparation 64](30.6 mg, 182 μmol), HATU (63.67 mg, 167 μmol) in DMF (1 mL) was added Hunig base (93 μL, 531 μmol). The mixture was stirred at rt overnight. It was partitioned between EtOAc/water. The aqueous layer was extracted with EtOAc. The combined organic phases were concentrated and purified by normal phase column (24 g, EtOAc in heptane 50-100%) to provide racemic 6-cyclobutoxy-N-(1-(cis-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide as a light pink solid (59 mg, 81% yield). LCMS m/z=480.2 [M+H]⁺. ¹H NMR (METHANOL-d4, 400 MHz) δ 9.03 (s, 1H), 8.65 (dd, 1H, J=1.8, 7.5 Hz), 8.52 (s, 1H), 7.4-7.5 (m, 1H), 6.43 (t, 1H, J=7.3 Hz), 5.5-5.7 (m, 1H), 4.19 (s, 2H), 3.45 (br dd, 1H, J=1.8, 6.5 Hz), 2.6-2.7 (m, 4H), 2.4-2.5 (m, 2H), 2.3-2.3 (m, 2H), 2.0-2.1 (m, 1H), 1.8-1.9 (m, 1H), 1.5-1.7 (m, 5H)

### Examples 26 and 27: 6-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide and 6-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide

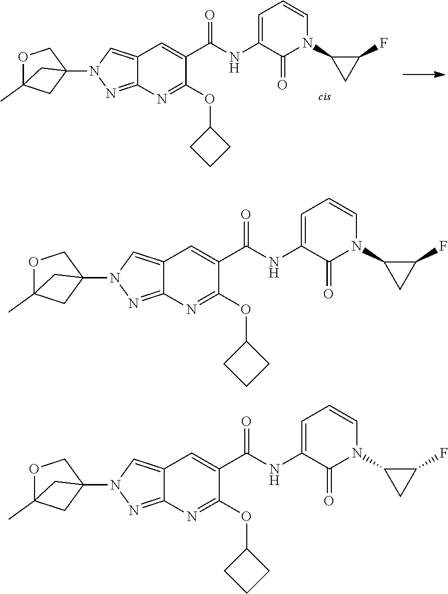

Racemic 6-cyclobutoxy-N-(1-(cis-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide [Example 25](51 mg, 0.106 mmol) was separated by SFC: CHIRALPAK AD-H 30×250 mm, 5 μm. Method: 50% MeOH w/No Modifier in CO2 (flow rate: 100 mL/min, ABPR 120 bar, MBPR 40 psi, column temp 40 deg C.) column temp 40 deg C.) to provide:

Peak 1, Example 26: 6-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide (15.1 mg, 30% yield); LCMS (ESI) m/z 480.2 (M+H)⁺; 1H NMR (METHANOL-d4, 400 MHz) δ 9.04 (s, 1H), 8.65 (dd, 1H, J=2.0, 7.5 Hz), 8.52 (s, 1H), 7.42 (dd, 1H, J=1.4, 7.4 Hz), 6.43 (t, 1H, J=7.3 Hz), 5.6-5.7 (m, 1H), 4.6-4.6 (m, 1H), 4.19 (s, 2H), 3.4-3.5 (m, 1H), 2.6-2.7 (m, 4H), 2.4-2.5 (m, 2H), 2.3-2.3 (m, 2H), 2.0-2.1 (m, 1H), 1.8-1.9 (m, 1H), 1.5-1.6 (m, 5H).

Peak 2, Example 27: 6-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide (20.1 mg. 39% yield). LCMS (ESI) m/z 480.2 (M+H)⁺; 1H NMR (METHANOL-d4, 400 MHz) δ 9.04 (s, 1H), 8.65 (dd, 1H, J=1.8, 7.5 Hz), 8.52 (s, 1H), 7.42 (dd, 1H, J=2.0, 6.8 Hz), 6.4-6.5 (m, 1H), 5.61 (s, 1H), 4.6-4.7 (m, 1H), 4.19 (s, 2H), 3.4-3.5 (m, 1H), 2.6-2.7 (m, 4H), 2.43 (dd, 2H, J=1.8, 4.5 Hz), 2.30 (dd, 2H, J=1.8, 4.5 Hz), 2.1-2.1 (m, 1H), 1.8-1.9 (m, 1H), 1.58 (s, 5H).

### Example 28: 2-(2-oxabicyclo[2.1.1]hexan-4-yl)-N-(1-cyclopropyl-2-oxo-1,2-dihydropyridin-3-yl)-6-isopropoxy-2H-pyrazolo[3,4-b]pyridine-5-carboxamide

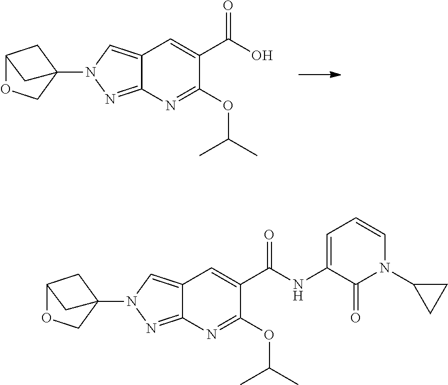 [SMILES: CC(C)Oc1nc2nn(C34COC(C3)C4)cc2cc1C(=O)O ; CC(C)Oc1nc2nn(C34COC(C3)C4)cc2cc1C(=O)Nc1cccn(C2CC2)c1=O]

The title compound, 2-(2-oxabicyclo[2.1.1]hexan-4-yl)-N-(1-cyclopropyl-2-oxo-1,2-dihydropyridin-3-yl)-6-isopropoxy-2H-pyrazolo[3,4-b]pyridine-5-carboxamide, was prepared in a similar fashion to that described for Example 9, starting with 2-(2-oxabicyclo[2.1.1]hexan-4-yl)-6-isopropoxy-2H-pyrazolo[3,4-b]pyridine-5-carboxylic acid [preparation 67]. LCMS (ESI) m/z 436.1 (M+H)⁺; ¹H NMR (400 MHz, DMSO-d₆) δ ppm=10.89 (s, 1H), 8.96 (s, 1H), 8.71 (s, 1H), 8.50-8.42 (m, 1H), 7.31 (dd, J=1.3, 7.0 Hz, 1H), 6.29 (t, J=7.2 Hz, 1H), 5.72-5.63 (m, 1H), 4.69 (s, 1H), 4.05 (s, 2H), 3.54-3.46 (m, 1H), 2.49-2.46 (m, 2H), 2.21-2.16 (m, 2H), 1.55 (d, J=6.1 Hz, 6H), 1.08-1.01 (m, 2H), 0.94-0.88 (m, 2H).

### Example 29: N-(1-cyclopropyl-2-oxo-1,2-dihydropyridin-3-yl)-6-isopropoxy-2-(1-(methoxymethyl)-2-oxabicyclo[2.2.1]heptan-4-yl)-2H-pyrazolo[3,4-b]pyridine-5-carboxamide

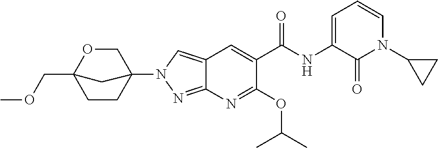 [SMILES: COCC12CCC(n3cc4cc(C(=O)Nc5cccn(C6CC6)c5=O)c(OC(C)C)nc4n3)(CO1)C2]

To a mixture of 6-isopropoxy-2-(1-(methoxymethyl)-2-oxabicyclo[2.2.1]heptan-4-yl)-2H-pyrazolo[3,4-b]pyridine-5-carboxylic acid [preparation 70](70.7 mg, 195.63 umol), 3-amino-1-cyclopropyl-pyridin-2-one HCl (40.16 mg, 215.20 umol), HATU (78.31 mg, 205.41 umol) in DMF (1 mL) was added Hunigs base (136.30 uL, 782.53 umol). The mixture was stirred at rt over weekend. It was partitioned between EtOAc/water. The aqueous layer was extracted with EtOAc. The combined organic phases were concentrated. The residue was triturated with small amount of EtOAc to get N-(1-cyclopropyl-2-oxo-1,2-dihydropyridin-3-yl)-6-isopropoxy-2-(1-(methoxymethyl)-2-oxabicyclo[2.2.1]heptan-4-yl)-2H-pyrazolo[3,4-b]pyridine-5-carboxamide as an off-white solid (27 mg, yield 28%). LCMS m/z=494.2 [M+H]⁺. ¹H NMR (METHANOL-d4, 400 MHz) δ 9.02 (s, 1H), 8.61 (dd, 1H, J=1.8, 7.5 Hz), 8.51 (s, 1H), 7.35 (dd, 1H, J=1.8, 7.0 Hz), 6.39 (t, 1H, J=7.3 Hz), 5.82 (quin, 1H, J=6.3 Hz), 4.25 (d, 1H, J=6.5 Hz), 4.13 (dd, 1H, J=3.6, 6.4 Hz), 3.7-3.7 (m, 2H), 3.5-3.5 (m, 1H), 3.46 (s, 3H), 2.5-2.6 (m, 2H), 2.3-2.4 (m, 2H), 2.13 (dt, 1H, J=4.3, 12.7 Hz), 1.9-2.0 (m, 1H), 1.64 (d, 6H, J=6.3 Hz), 1.1-1.2 (m, 2H), 0.9-1.0 (m, 2H).

The mother liquid was collected and purified by normal phase column (24 g, EtOAc in heptane 100%) to get a pale-yellow oil (68 mg, yield 70%), which was submitted to chiral separation.

### Examples 30 and 31: N-(1-cyclopropyl-2-oxo-1,2-dihydropyridin-3-yl)-6-isopropoxy-2-((1S,4S)-1-(methoxymethyl)-2-oxabicyclo[2.2.1]heptan-4-yl)-2H-pyrazolo[3,4-b]pyridine-5-carboxamide and N-(1-cyclopropyl-2-oxo-1,2-dihydropyridin-3-yl)-6-isopropoxy-2-((1R,4R)-1-(methoxymethyl)-2-oxabicyclo[2.2.1]heptan-4-yl)-2H-pyrazolo[3,4-b]pyridine-5-carboxamide

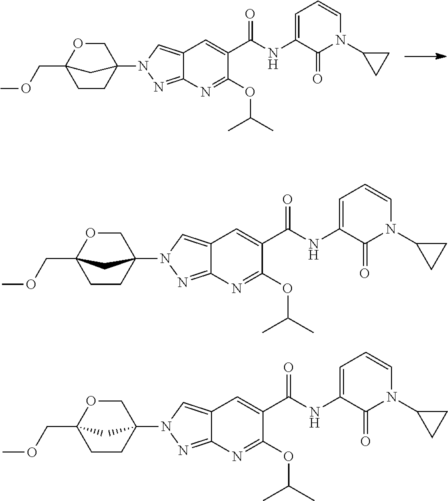 [SMILES: COCC12CCC(n3cc4cc(C(=O)Nc5cccn(C6CC6)c5=O)c(OC(C)C)nc4n3)(CO1)C2 ; COC[C@@]12CC[C@@](n3cc4cc(C(=O)Nc5cccn(C6CC6)c5=O)c(OC(C)C)nc4n3)(CO1)C2 ; COC[C@]12CC[C@](n3cc4cc(C(=O)Nc5cccn(C6CC6)c5=O)c(OC(C)C)nc4n3)(CO1)C2]

N-(1-cyclopropyl-2-oxo-1,2-dihydropyridin-3-yl)-6-isopropoxy-2-(1-(methoxymethyl)-2-oxabicyclo[2.2.1]heptan-4-yl)-2H-pyrazolo[3,4-b]pyridine-5-carboxamide [Example 29](68 mg, 0.137 mmol) was chiral separated by SFC CHIRALPAK IB CHIRALPAK IB 30×250 mm, 5 um. Method: 50% MeOH w/No Modifier in CO2 (flow rate: 100 mL/min, ABPR 120 bar, MBPR 40 psi, column temp 40 deg C.) to provide:

Peak 1, Example 30: N-(1-cyclopropyl-2-oxo-1,2-dihydropyridin-3-yl)-6-isopropoxy-2-((1S,4S)-1-(methoxymethyl)-2-oxabicyclo[2.2.1]heptan-4-yl)-2H-pyrazolo[3,4-b]pyridine-5-carboxamide (22.4 mg, 33% yield); LCMS (ESI) m/z 494.2 (M+H)⁺; 1H NMR (METHANOL-d4, 400 MHz) δ 9.02 (s, 1H), 8.61 (dd, 1H, J=1.8, 7.5 Hz), 8.51 (s, 1H), 7.3-7.4 (m, 1H), 6.39 (t, 1H, J=7.2 Hz), 5.82 (t, 1H, J=6.3 Hz), 4.25 (d, 1H, J=6.5 Hz), 4.14 (dd, 1H, J=3.5, 6.5 Hz), 3.7-3.8 (m, 2H), 3.5-3.5 (m, 1H), 3.46 (s, 3H), 2.5-2.6 (m, 2H), 2.3-2.4 (m, 2H), 2.1-2.2 (m, 1H), 1.9-2.1 (m, 1H), 1.64 (d, 6H, J=6.0 Hz), 1.2-1.2 (m, 2H), 0.9-1.0 (m, 2H)

Peak 2, Example 31: N-(1-cyclopropyl-2-oxo-1,2-dihydropyridin-3-yl)-6-isopropoxy-2-((1R,4R)-1-(methoxymethyl)-2-oxabicyclo[2.2.1]heptan-4-yl)-2H-pyrazolo[3,4-b]pyridine-5-carboxamide (20.8 mg. 31% yield). LCMS (ESI) m/z 494.2 (M+H)⁺; 1H NMR (METHANOL-d4, 400 MHz) δ 9.02 (s, 1H), 8.61 (dd, 1H, J=1.8, 7.5 Hz), 8.51 (s, 1H), 7.35 (dd, 1H, J=1.8, 7.0 Hz), 6.39 (t, 1H, J=7.2 Hz), 5.82 (t, 1H, J=6.3 Hz), 4.25 (d, 1H, J=6.5 Hz), 4.14 (dd, 1H, J=3.8, 6.5 Hz), 3.7-3.8 (m, 2H), 3.5-3.5 (m, 1H), 3.46 (s, 3H), 2.4-2.6 (m, 2H), 2.3-2.4 (m, 2H), 2.13 (d, 1H, J=4.3 Hz), 1.9-2.1 (m, 1H), 1.64 (d, 6H, J=6.0 Hz), 1.2-1.2 (m, 2H), 0.99 (dd, 2H, J=1.8, 4.0 Hz).

### Example 32: (rac)-Cis-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide

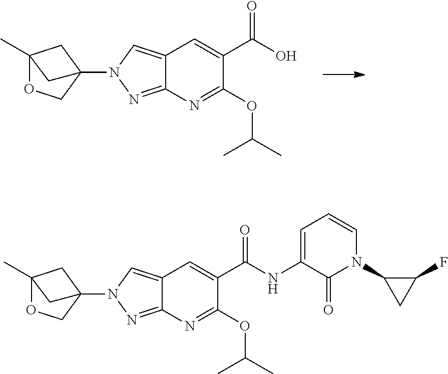

6-isopropoxy-2-(1-methyl-2-oxabicyclo[2.1.1]hexan-4-yl)-2H-pyrazolo[3,4-b]pyridine-5-carboxylic acid [preparation 6] was dissolved in DMF (1 mL), HATU (31 mg, 81 μmol) and DIPEA (38 μL, 220 μmol) were added and stirred for 1 minute at room temperature. Cis-3-amino-1-(2-fluorocyclopropyl)pyridin-2(1H)-one [preparation 64] was then added and the mixture was stirred at rt overnight. The reaction was then diluted with water, extracted with EtOAc (2×5 mL), concentrated, and the residue purified by reverse-phase HPLC eluting with 5-60% ACN/water, 0.1% TFA, (column; Waters SunFire Prep, C18 Sum, OBD 19×100 mm) to obtain (rac)-Cis-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide (29 mg, 84% yield). LCMS (ESI) m/z 464.4 (M+H)⁺. ¹H NMR (500 MHz, DMSO-d6) δ 10.88 (s, 1H), 8.96 (s, 1H), 8.68 (s, 1H), 8.50 (dd, J=1.68, 7.48 Hz, 1H), 7.43 (d, J=7.02 Hz, 1H), 6.34 (t, J=7.17 Hz, 1H), 5.67 (quin, J=6.18 Hz, 1H), 4.97-5.16 (m, 1H), 4.09 (s, 2H), 3.44-3.50 (m, 1H), 2.39 (dd, J=1.60, 4.35 Hz, 2H), 2.17 (dd, J=1.60, 4.20 Hz, 2H), 1.58-1.70 (m, 1H), 1.54 (dd, J=3.28, 6.18 Hz, 6H), 1.49 (s, 3H), 1.40-1.48 (m, 1H).

### Examples 33 and 34: N-[1-[(1R,2S)-2-fluorocyclopropyl]-2-oxo-3-pyridyl]-6-isopropoxy-2-(1-methyl-2-oxabicyclo[2.1.1]hexan-4-yl)pyrazolo[3,4-b]pyridine-5-carboxamide and N-[1-[(1S,2R)-2-fluorocyclopropyl]-2-oxo-3-pyridyl]-6-isopropoxy-2-(1-methyl-2-oxabicyclo[2.1.1]hexan-4-yl)pyrazolo[3,4-b]pyridine-5-carboxamide

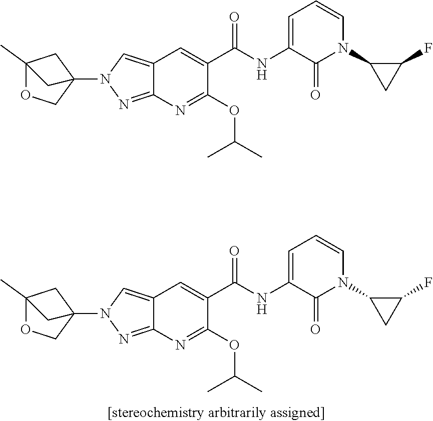 [SMILES: CC(C)Oc1nc2nn(C34COC(C)(C3)C4)cc2cc1C(=O)Nc1cccn([C@@H]2C[C@@H]2F)c1=O ; CC(C)Oc1nc2nn(C34COC(C)(C3)C4)cc2cc1C(=O)Nc1cccn([C@H]2C[C@H]2F)c1=O]

The enantiomers of racemic (Cis)-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide were separated using CHIRALPAK AD-H 30×250 mm, 5 um. Method: 40% MeOH w/0.1% DEA in CO2 (flow rate: 100 mL/min, ABPR 120 bar, MBPR 40 psi, column temp 40 deg C.) to afford as Peak 1, Example 33: N-[1-[(1R,2S)-2-fluorocyclopropyl]-2-oxo-3-pyridyl]-6-isopropoxy-2-(1-methyl-2-oxabicyclo[2.1.1]hexan-4-yl)pyrazolo[3,4-b]pyridine-5-carboxamide (7.4 mg, 60% yield, >99% ee), stereochemistry arbitrarily assigned and Peak 2, Example 34: N-[1-[(1S,2R)-2-fluorocyclopropyl]-2-oxo-3-pyridyl]-6-isopropoxy-2-(1-methyl-2-oxabicyclo[2.1.1]hexan-4-yl)pyrazolo[3,4-b]pyridine-5-carboxamide (9.1 mg, 73% yield, 98% ee), setereochemistry arbitrarily assigned.

Peak 1, Example 33: LCMS (ESI) m/z 464.4 (M+H)⁺. ¹H NMR (500 MHz, METHANOL-d4) δ 9.03 (s, 1H), 8.64 (dd, J=1.68, 7.48 Hz, 1H), 8.52 (s, 1H), 7.41 (d, J=6.56 Hz, 1H), 6.42 (t, J=7.25 Hz, 1H), 5.82 (quin, J=6.26 Hz, 1H), 5.02-5.09 (m, 1H), 4.19 (s, 2H), 3.39-3.45 (m, 1H), 2.40-2.46 (m, 2H), 2.30 (dd, J=1.60, 4.50 Hz, 2H), 1.64 (dd, J=1.45, 6.18 Hz, 6H), 1.53-1.61 (m, 5H).

Peak 2, Example 34: LCMS (ESI) m/z 464.4 (M+H)⁺. ¹H NMR (500 MHz, METHANOL-d4) Shift 9.03 (s, 1H), 8.64 (dd, J=1.75, 7.40 Hz, 1H), 8.52 (s, 1H), 7.41 (d, J=5.80 Hz, 1H), 6.42 (t, J=7.25 Hz, 1H), 5.77-5.87 (m, 1H), 5.03-5.09 (m, 1H), 4.19 (s, 2H), 3.40-3.45 (m, 1H), 2.44 (dd, J=1.45, 4.50 Hz, 2H), 2.27-2.35 (m, 2H), 1.64 (dd, J=1.45, 6.18 Hz, 6H), 1.53-1.61 (m, 5H).

### Example 35: (racemic)-Cis-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide

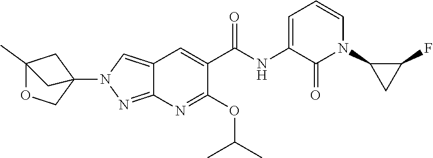 [SMILES: CC(C)Oc1nc2nn(C34COC(C)(C3)C4)cc2cc1C(=O)Nc1cccn([C@@H]2C[C@@H]2F)c1=O]

Synthesized in a similar manner as Example 32 but starting from 6-isopropoxy-2-(1-methyl-2-oxabicyclo[2.1.1]hexan-4-yl)-2H-indazole-5-carboxylic acid [preparation 49]. LCMS (ESI) m/z 467.4 (M+H)⁺. ¹H NMR (500 MHz, METHANOL-d4) δ 8.60-8.69 (m, 2H), 8.51 (s, 1H), 7.39 (br d, J=6.87 Hz, 1H), 7.17 (s, 1H), 6.42 (t, J=7.17 Hz, 1H), 4.91-5.02 (m, 2H), 4.20 (s, 2H), 3.39-3.45 (m, 1H), 2.44 (br d, J=4.43 Hz, 2H), 2.31 (dd, J=1.45, 4.50 Hz, 2H), 1.62 (d, J=6.10 Hz, 6H), 1.51-1.60 (m, 5H).

### Examples 36 and 37: N-(1-((1R,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide and N-(1-((1S,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide

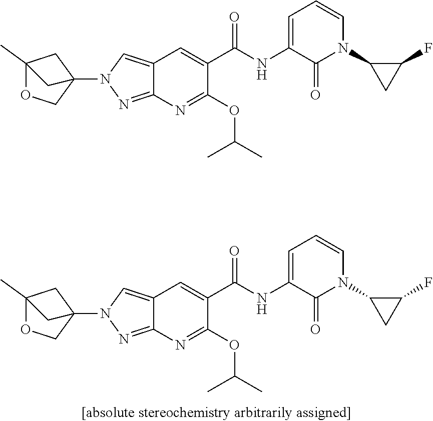 [SMILES: CC(C)Oc1nc2nn(C34COC(C)(C3)C4)cc2cc1C(=O)Nc1cccn([C@@H]2C[C@@H]2F)c1=O ; CC(C)Oc1nc2nn(C34COC(C)(C3)C4)cc2cc1C(=O)Nc1cccn([C@H]2C[C@H]2F)c1=O]

The enantiomers of racemic Cis-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide [Example 35] were separated by SFC (Daicel Chiralpak AD-H; 250×30 mm, 5 μm; 50% MeOH+0.1% Et₂NH in CO₂) to afford Peak 1, Example 36: N-(1-((1R,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide (4.7 mg, >99% ee, stereochemistry arbitrarily assigned); LCMS (ESI) m/z 467.4 (M+H)⁺. ¹H NMR (500 MHz, METHANOL-d₄) δ 8.66 (s, 1H), 8.64 (dd, J=1.68, 7.48 Hz, 1H), 8.51 (s, 1H), 7.39 (d, J=7.02 Hz, 1H), 7.17 (s, 1H), 6.42 (t, J=7.25 Hz, 1H), 4.95-5.08 (m, 2H), 4.20 (s, 2H), 3.40-3.45 (m, 1H), 2.44 (dd, J=1.60, 4.50 Hz, 2H), 2.31 (dd, J=1.60, 4.50 Hz, 2H), 1.62 (d, J=6.10 Hz, 6H), 1.52-1.60 (m, 5H).

Peak 2, Example 37: N-(1-((1S,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide (5.8 mg, >99% ee, stereochemistry arbitrarily assigned); LCMS (ESI) m/z 467.5 (M+H)⁺. ¹H NMR (500 MHz, METHANOL-d₄) δ 8.66 (s, 1H), 8.64 (dd, J=1.75, 7.40 Hz, 1H), 8.51 (d, J=0.76 Hz, 1H), 7.39 (d, J=6.26 Hz, 1H), 7.17 (s, 1H), 6.42 (t, J=7.25 Hz, 1H), 4.96-5.08 (m, 2H), 4.20 (s, 2H), 3.39-3.45 (m, 1H), 2.44 (dd, J=1.45, 4.50 Hz, 2H), 2.31 (dd, J=1.60, 4.50 Hz, 2H), 1.62 (d, J=6.10 Hz, 6H), 1.52-1.60 (m, 5H)

### Example 38: (rac)-Trans-6-isopropoxy-2-(1-methyl-2-oxabicyclo[2.1.1]hexan-4-yl)-N-(1-(2-methylcyclopropyl)-2-oxo-1,2-dihydropyridin-3-yl)-2H-pyrazolo[3,4-b]pyridine-5-carboxamide

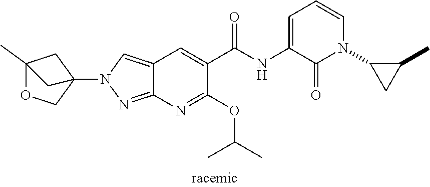

6-isopropoxy-2-(1-methyl-2-oxabicyclo[2.1.1]hexan-4-yl)-2H-pyrazolo[3,4-b]pyridine-5-carboxylic acid [preparation 6](200 mg, 630 umol) was dissolved in DMF (3 mL). DIPEA (244 mg, 1.9 mmol, 329 uL) and HATU (252 mg, 661 umol) were added. Racemic-(Trans)-3-amino-1-(2-methylcyclopropyl)pyridin-2(1H)-one hydrochloride [preparation 66](103 mg, 630 umol) was added and the mixture stirred at rt overnight. The resulting was purified by acid-modified reverse phase HPLC eluting with 05-60% ACN/water, 0.1% TFA, column; Waters SunFire Prep, C18 Sum, OBD 19×100 mm. LCMS (ESI) m/z 464.5 (M+H)⁺. ¹H NMR (500 MHz, DMSO-d6) Shift 10.91 (s, 1H), 8.95 (s, 1H), 8.67 (s, 1H), 8.44 (dd, J=1.75, 7.40 Hz, 1H), 7.30 (dd, J=1.83, 7.02 Hz, 1H), 6.27 (t, J=7.17 Hz, 1H), 5.61-5.74 (m, 1H), 4.09 (s, 2H), 3.18-3.22 (m, 1H), 2.39 (dd, J=1.53, 4.27 Hz, 2H), 2.17 (dd, J=1.53, 4.27 Hz, 2H), 1.55 (dd, J=2.75, 6.26 Hz, 6H), 1.49 (s, 3H), 1.18-1.28 (m, 4H), 1.06-1.12 (m, 1H), 0.82-0.90 (m, 1H).

### Example 39: Racemic-(Trans)-6-isopropoxy-2-(1-methyl-2-oxabicyclo[2.1.1]hexan-4-yl)-N-(1-(2-methylcyclopropyl)-2-oxo-1,2-dihydropyridin-3-yl)-2H-indazole-5-carboxamide

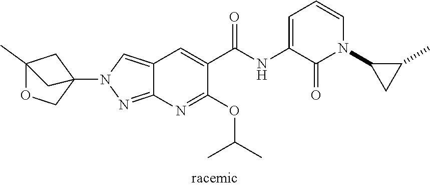

The title compound was synthesized in a similar manner to Example 38 but starting from 6-isopropoxy-2-(1-methyl-2-oxabicyclo[2.1.1]hexan-4-yl)-2H-indazole-5-carboxylic acid [preparation 49]. LCMS (ESI) m/z 463.5 (M+H)⁺. ¹H NMR (500 MHz, DMSO-d₆) δ 10.88 (s, 1H), 8.67 (s, 1H), 8.58 (s, 1H), 8.45 (dd, J=1.60, 7.40 Hz, 1H), 7.23-7.32 (m, 2H), 6.26 (t, J=7.17 Hz, 1H), 5.00 (quin, J=6.14 Hz, 1H), 4.10 (s, 2H), 3.15-3.22 (m, 1H), 2.38-2.40 (m, 2H), 2.17 (dd, J=1.45, 4.20 Hz, 2H), 1.52 (dd, J=3.13, 6.03 Hz, 6H), 1.49 (s, 3H), 1.16-1.24 (m, 4H), 1.05-1.11 (m, 1H), 0.85 (q, J=5.80 Hz, 1H).

### Examples 40 and 41: 6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-indazole-5-carboxamide and 6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-indazole-5-carboxamide

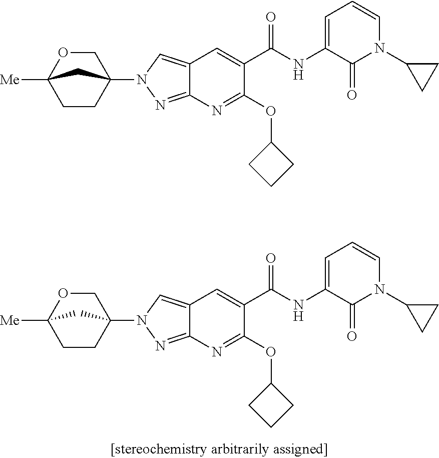 [SMILES: C[C@@]12CC[C@@](n3cc4cc(C(=O)Nc5cccn(C6CC6)c5=O)c(OC5CCC5)nc4n3)(CO1)C2 ; C[C@]12CC[C@](n3cc4cc(C(=O)Nc5cccn(C6CC6)c5=O)c(OC5CCC5)nc4n3)(CO1)C2]

To a mixture of 6-cyclobutoxy-2-(1-methyl-2-oxabicyclo[2.2.1]heptan-4-yl)-2H-indazole-5-carboxylic acid [preparation 61](100 mg, 292 μmol), 3-amino-1-cyclopropyl-pyridin-2-one (65.8 mg, 438 μmol) in pyridine (1 mL) was added T3P® (929.3 mg, 1.460 mmol, 869.3 μL, 50% purity) at room temperature. The vial contained this reaction mixture was capped and stirred at rt for 2 h. The mixture was diluted with EtOAc and water. The aqueous phase was extracted with EtOAc (3×5 mL). The combined organic layers were dried over anhydrous MgSO₄ and filtered. The filtrate was evaporated in vacuo to afford a crude residue, which was purified by reverse phase HPLC (C18 column, 5-60% acetonitrile in water with 0.1% ammonia) to afford 6-(cyclobutoxy)-N-(1-cyclopropyl-2-oxo-3-pyridyl)-2-[(1S,4S)-1-methyl-2-oxabicyclo[2.2.1]heptan-4-yl]indazole-5-carboxamide (42.1 mg, 88.7 μmol, 30.4% yield) as an off white solid. LCMS m/z=475.2 [M+H]⁺. The enantiomers were separated by SFC (Daicel Chiralpak AD-H; 250×30 mm, 5 μm; 55% EtOH+0.1% Et₂NH in CO₂) to afford

Peak 1, Example 40: 6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-indazole-5-carboxamide, stereochemistry arbitrarily assigned (16 mg, 11%, >99% ee); LCMS m/z=474.9 [M+H]⁺; ¹H NMR (400 MHz, MeOH-d₄) δ: 0.87-1.03 (m, 2H) 1.10-1.22 (m, 2H) 1.40-1.52 (m, 3H) 1.80-2.12 (m, 4H) 2.26-2.52 (m, 4H) 2.52-2.73 (m, 4H) 3.46 (td, J=7.40, 4.02 Hz, 1H) 4.11 (dd, J=6.40, 3.39 Hz, 1H) 4.14-4.26 (m, 1H) 4.94-5.07 (m, 1H) 6.27-6.45 (m, 1H) 6.83-7.05 (m, 1H) 7.33 (dd, J=7.03, 1.76 Hz, 1H) 8.39-8.50 (m, 1H) 8.56-8.70 (m, 2H).

Peak 2, Example 41: 6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-indazole-5-carboxamide, stereochemistry arbitrarily assigned (16.3 mg, 11.8%, >99% ee); LCMS m/z=474.9 [M+H]⁺; ¹H NMR (400 MHz, MeOH-d₄) δ: 0.91-1.02 (m, 2H) 1.18 (q, J=6.86 Hz, 2H) 1.48 (s, 3H) 1.81-2.11 (m, 4H) 2.25-2.50 (m, 4H) 2.57-2.75 (m, 4H) 3.40-3.55 (m, 1H) 4.09 (dd, J=6.53, 3.51 Hz, 1H) 4.19 (d, J=6.27 Hz, 1H) 4.94-5.06 (m, 1H) 6.33-6.39 (m, 1H) 6.94 (s, 1H) 7.31 (dd, J=6.90, 1.63 Hz, 1H) 8.45 (s, 1H) 8.56-8.70 (m, 2H).

### Example 42: racemic-(Trans)-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide

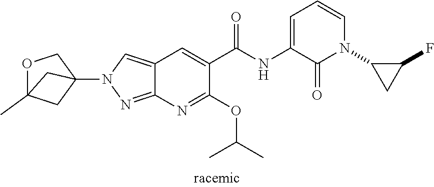

A vial was charged with 6-isopropoxy-2-(1-methyl-2-oxabicyclo[2.1.1]hexan-4-yl)-2H-pyrazolo[3,4-b]pyridine-5-carboxylic acid [preparation 6](59.0 mg, 186 μmol), racemic (Trans)-3-amino-1-(2-fluorocyclopropyl)pyridin-2(1H)-one [preparation 65](41.9 mg, 204 μmol, Hydrochloride) and pyridine (1 mL). An EtOAc solution of T3P® (592 mg, 930 μmol, 553.4 μL, 50% w/w) was added at rt. The vial was sealed and maintained at rt for 2 h. The mixture was diluted with EtOAc and water. The aqueous phase was extracted with EtOAc (3×5 mL). The combined organic layers were dried over anhydrous MgSO₄ and filtered. The filtrate was evaporated in vacuo and the residue was purified by reverse phase HPLC (C18 column, 5-60% acetonitrile in water with 0.1% ammonia) to afford racemic-(Trans)-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide (46.7 mg, 99.9 μmol, 53.7% yield) as an off white solid. LCMS m/z=468.1 [M+H]⁺; ¹H NMR (400 MHz, CHLOROFORM-d) δ 1.33-1.46 (m, 1H) 1.61 (s, 3H) 1.65 (dd, J=6.27, 1.51 Hz, 6H) 1.75-1.87 (m, 1H) 2.26-2.43 (m, 4H) 3.72-3.87 (m, 1H) 4.25 (s, 2H) 4.71-4.95 (m, 1H) 5.93 (spt, J=6.32 Hz, 1H) 6.25 (t, J=7.15 Hz, 1H) 6.94 (dd, J=7.03, 1.76 Hz, 1H) 8.03 (s, 1H) 8.59 (dd, J=7.28, 1.76 Hz, 1H) 9.00 (s, 1H) 11.02 (s, 1H).

### Examples 43 and 44: N-(1-((1S,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide and N-(1-((1R,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide

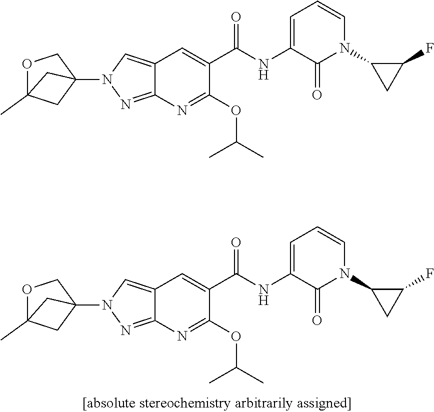 [SMILES: CC(C)Oc1nc2nn(C34COC(C)(C3)C4)cc2cc1C(=O)Nc1cccn([C@H]2C[C@@H]2F)c1=O ; CC(C)Oc1nc2nn(C34COC(C)(C3)C4)cc2cc1C(=O)Nc1cccn([C@@H]2C[C@H]2F)c1=O]

The enantiomers of racemic-(Trans)-N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide [Example 42] were separated by SFC (Daicel Chiralpak AD-H; 250×30 mm, 5 μm; 40% EtOH+0.1% Et₂NH in CO₂) to provide Peak1, Example 43: N-(1-((1S,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide, stereochemistry arbitrarily assigned (10.5 mg, 12.1%, >99% ee) LCMS m/z=468.2 [M+H]⁺; ¹H NMR (400 MHz, MeOH-d₄) δ: 1.46-1.55 (m, 1H) 1.58 (s, 3H) 1.65 (dd, J=6.27, 1.76 Hz, 6H) 1.72-1.86 (m, 1H) 2.30 (dd, J=4.52, 1.76 Hz, 2H) 2.39-2.46 (m, 2H) 3.84 (br d, J=10.29 Hz, 1H) 4.19 (s, 2H) 4.76-5.00 (m, 1H) 5.82 (dt, J=12.55, 6.27 Hz, 1H) 6.39 (t, J=7.15 Hz, 1H) 7.25 (dd, J=7.15, 1.88 Hz, 1H) 8.51 (s, 1H) 8.59 (dd, J=7.53, 1.76 Hz, 1H) 9.03 (s, 1H) and Peak 2, Example 44: N-(1-((1R,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide, stereochemistry arbitrarily assigned_(10.2 mg, 11.7%, 94% ee) LCMS m/z=468.2 [M+H]⁺; ¹H NMR (400 MHz, MeOH-d₄) δ: 1.47-1.54 (m, 1H) 1.58 (s, 3H) 1.65 (dd, J=6.15, 1.63 Hz, 6H) 1.75-1.84 (m, 1H) 2.25-2.32 (m, 2H) 2.40-2.47 (m, 2H) 3.82 (br d, J=8.78 Hz, 1H) 4.19 (s, 2H) 4.77-5.01 (m, 1H) 5.82 (t, J=6.15 Hz, 1H) 6.39 (t, J=7.15 Hz, 1H) 7.25 (dd, J=6.90, 1.63 Hz, 1H) 8.51 (s, 1H) 8.60 (dd, J=7.53, 1.76 Hz, 1H) 9.03 (s, 1H).

### Example 45: N-(2-cyclopropyl-3-oxo-2,3-dihydropyridazin-4-yl)-6-isopropoxy-2-(1-methyl-2-oxabicyclo[2.1.1]hexan-4-yl)-2H-indazole-5-carboxamide

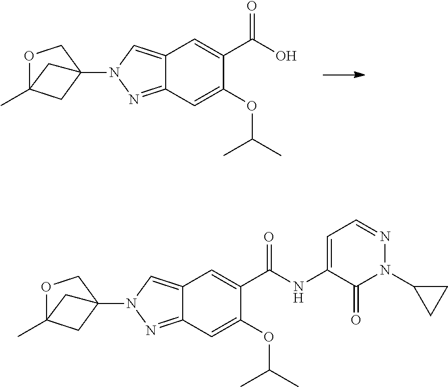

An EtOAc solution of T3P® (758 μmol, 451 μL, 50% w/w) was added to 6-isopropoxy-2-(1-methyl-2-oxabicyclo[2.1.1]hexan-4-yl)-2H-indazole-5-carboxylic acid [preparation 49](60 mg, 189 μmol) and 4-amino-2-cyclopropyl-pyridazin-3-one (46 mg, 246 μmol, Hydrochloride) in Pyridine (1.3 mL) at rt. After stirring for 4 h, the mixture was diluted with water and extracted with DCM and then EtOAc, dried over MgSO₄, filtered and concentrated. The crude material was purified by mass-directed reverse-phase HPLC (Column: Sunfire C18 100×19 mm, 5 mm; Mobile phase A: MeCN; Mobile phase B: H2O; Modifier: 0.1% TFA) to provide N-(2-cyclopropyl-3-oxo-pyridazin-4-yl)-6-isopropoxy-2-(1-methyl-2-oxabicyclo[2.1.1]hexan-4-yl)indazole-5-carboxamide (31.8 mg, 36% yield). LCMS (ESI) m/z 449.9 (M+H)⁺. 1H NMR (500 MHz, DMSO-d6) δ ppm 1.00 (dd, J=7.32, 2.44 Hz, 2H) 1.06 (br d, J=3.66 Hz, 2H) 1.50 (s, 3H) 1.53 (d, J=6.10 Hz, 6H) 2.18 (dd, J=4.27, 1.22 Hz, 2H) 2.40 (dd, J=4.27, 1.22 Hz, 2H) 4.11 (s, 2H) 4.17 (dt, J=7.78, 3.74 Hz, 1H) 5.04 (quin, J=6.10 Hz, 1H) 7.34 (s, 1H) 7.92 (d, J=4.88 Hz, 1H) 8.19 (d, J=4.27 Hz, 1H) 8.64 (s, 1H) 8.73 (s, 1H) 11.18 (s, 1H).

### Example 46: N-(2-cyclopropyl-3-oxo-2,3-dihydropyridazin-4-yl)-6-isopropoxy-2-(1-methyl-2-oxabicyclo[2.1.1]hexan-4-yl)-2H-pyrazolo[3,4-b]pyridine-5-carboxamide

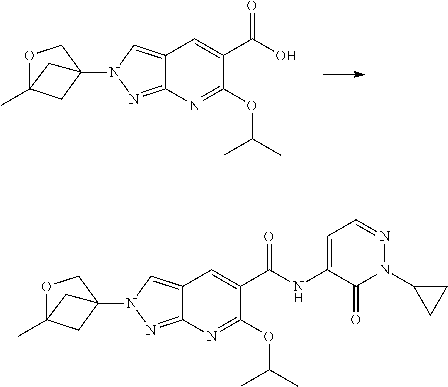

An EtOAc solution of T3P® (504 μmol, 300 μL, 50% w/w) was added to 6-isopropoxy-2-(1-methyl-2-oxabicyclo[2.1.1]hexan-4-yl)-2H-pyrazolo[3,4-b]pyridine-5-carboxylic acid [preparation 6](40 mg, 126 μmol) and 4-amino-2-cyclopropyl-pyridazin-3-one (30 mg, 163 μmol, HCl) in Pyridine (1.3 mL) at rt. After stirring for 4 h, the mixture was diluted with water and extracted with DCM and then EtOAc, dried over MgSO₄, filtered and concentrated. The crude material was purified by mass-directed reverse-phase HPLC (Column: Sunfire C18 100×19 mm, 5 mm; Mobile phase A: MeCN; Mobile phase B: H2O; Modifier: 0.1% TFA) to provide N-(2-cyclopropyl-3-oxo-pyridazin-4-yl)-6-isopropoxy-2-(1-methyl-2-oxabicyclo[2.1.1]hexan-4-yl)pyrazolo[3,4-b]pyridine-5-carboxamide (4.9 mg, 8% yield). LCMS (ESI) m/z 450.9 [M+H]⁺. ¹H NMR (500 MHz, DMSO-d6) δ ppm 0.98-1.04 (m, 2H) 1.05-1.10 (m, 2H) 1.50 (s, 3H) 1.56 (d, J=6.10 Hz, 6H) 2.18 (dd, J=4.27, 1.83 Hz, 2H) 2.41 (dd, J=4.27, 1.83 Hz, 2H) 4.10 (s, 2H) 4.13-4.22 (m, 1H) 5.61-5.76 (m, 1H) 7.93 (d, J=4.27 Hz, 1H) 8.17 (d, J=4.88 Hz, 1H) 8.72 (s, 1H) 9.01 (s, 1H) 11.17 (s, 1H).

### Examples 47 and 48: N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-pyrazolo[3,4-b]pyridine-5-carboxamide and N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-pyrazolo[3,4-b]pyridine-5-carboxamide

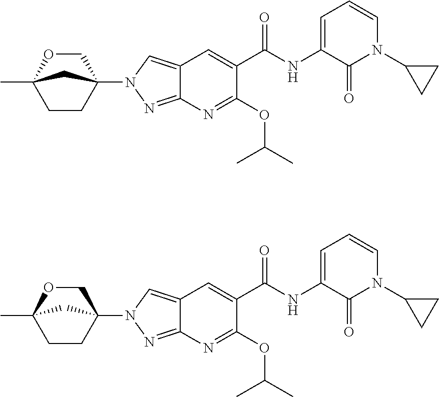 [SMILES: CC(C)Oc1nc2nn([C@@]34CC[C@@](C)(C3)OC4)cc2cc1C(=O)Nc1cccn(C2CC2)c1=O ; CC(C)Oc1nc2nn([C@]34CC[C@](C)(C3)OC4)cc2cc1C(=O)Nc1cccn(C2CC2)c1=O]

The title compounds were obtained in a manner similar to that described for Examples 40 and 41, starting from 6-isopropoxy-2-(1-methyl-2-oxabicyclo[2.2.1]heptan-4-yl)-2H-pyrazolo[3,4-b]pyridine-5-carboxylic acid [preparation 71]. The racemic product, N-(1-cyclopropyl-2-oxo-1,2-dihydropyridin-3-yl)-6-isopropoxy-2-(1-methyl-2-oxabicyclo[2.2.1]heptan-4-yl)-2H-pyrazolo[3,4-b]pyridine-5-carboxamide, was obtained as a white solid (60 mg, 84% yield) by prep-HPLC (Column: Welch Xtimate C18 150×30 mm×5 μm; Condition: water (0.05% NH₃H₂O+10 mm NH₄HCO₃)-ACN; Begin B: 49; End B: 79; Gradient Time (min): 10; 100% B Hold Time (min): 2; Flow Rate (mL/min): 25). The enantiomers were separated by SFC (Method Comments Column: Chiralpak AD-3 150×4.6 mm I.D., 3 um, Mobile phase: 40% of ethanol (0.05% DEA) in CO2, Flow rate: 2.5 mL/min, Column temp.: 35° C., ABPR: 1500 psi) to give Peak 1, Example 47: N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-pyrazolo[3,4-b]pyridine-5-carboxamide, stereochemistry arbitrarily assigned (15.8 mg, 26% yield, >99% ee) as a white solid. LCMS (ESI) m/z 464.3 [M+H]⁺. ¹H NMR (500 MHz, CHLOROFORM-d) δ ppm=10.98 (s, 1H), 8.98 (s, 1H), 8.60-8.58 (m, 1H), 8.00 (s, 1H), 7.05-7.02 (m, 1H), 6.22 (t, J=7.0 Hz, 1H), 5.95-5.89 (m, 1H), 4.24-4.19 (m, 1H), 4.19-4.18 (m, 1H), 3.49-3.44 (m, 1H), 2.51-2.45 (m, 1H), 2.44-2.41 (m, 1H), 2.37-2.28 (m, 2H), 2.07-1.95 (m, 2H), 1.63 (d, J=6.5 Hz, 6H), 1.51 (s, 3H), 1.20-1.15 (m, 2H), 0.94-0.90 (m, 2H).

Peak 2, Example 48: N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-pyrazolo[3,4-b]pyridine-5-carboxamide (stereochemistry arbitrarily assigned) was obtained as a white solid (17.4 mg, 29% yield, >99% ee). LCMS (ESI) m/z 464.3 [M+H]⁺. ¹H NMR (500 MHz, CHLOROFORM-d) δ ppm 10.98 (s, 1H), 8.98 (s, 1H), 8.60-8.58 (m, 1H), 8.00 (s, 1H), 7.04-7.02 (m, 1H), 6.22 (t, J=7.0 Hz, 1H), 5.95-5.89 (m, 1H), 4.24-4.19 (m, 1H), 4.19-4.18 (m, 1H), 3.49-3.45 (m, 1H), 2.51-2.46 (m, 1H), 2.44-2.41 (m, 1H), 2.37-2.28 (m, 2H), 2.07-1.95 (m, 2H), 1.63 (d, J=6.5 Hz, 6H), 1.51 (s, 3H), 1.20-1.15 (m, 2H), 0.94-0.90 (m, 2H).

### Example 49: N-(1-((1S,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide

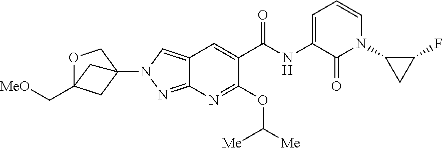 [SMILES: COCC12CC(n3cc4cc(C(=O)Nc5cccn([C@H]6C[C@H]6F)c5=O)c(OC(C)C)nc4n3)(CO1)C2]

To a solution of 6-isopropoxy-2-(1-(methoxymethyl)-2-oxabicyclo[2.1.1]hexan-4-yl)-2H-pyrazolo[3,4-b]pyridine-5-carboxylic acid (Preparation 23, 50 mg, 0.144 mmol) in pyridine (1 mL) added 3-amino-1-((1S,2R)-2-fluorocyclopropyl)pyridin-2(1H)-one (Preparation 77, 29.1 mg, 0.173 mmol) and T3P (1 mL) at 25° C. and the reaction was stirred at 25° C. for 16 h. The mixture was concentrated in vacuo and the residue diluted with saturated NaHCO₃ aq. to pH=8 and extracted with EtOAc (3×50 mL). The combined organics were washed with brine (50 mL) and dried (Na₂SO₄) and evaporated to dryness in vacuo. The residue was purified by Prep-HPLC-A to afford N-(1-((1S,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide as a white solid (50 mg, 70% yield). LCMS m/z=498.2 [M+H]⁺. ¹H NMR (MeOH-d₄, 400 MHz) δ: 8.99 (s, 1H), 8.60 (dd, J=1.6, 7.6 Hz, 1H), 8.50 (s, 1H), 7.37 (d, J=6.4 Hz, 1H), 6.38 (t, J=7.2 Hz, 1H), 5.81-5.75 (m, 1H), 5.0-4.88 (m, 1H), 4.20 (s, 2H), 3.76 (s, 2H), 3.44 (s, 3H), 3.43-3.36 (m, 1H), 2.53-2.46 (m, 2H), 2.37-2.29 (m, 2H), 1.61 (d, J=6.0 Hz, 6H), 1.59-1.50 (m, 2H).

### Example 50: N-(1-((1S,2R)-2-fluorocyclopropyl)-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide

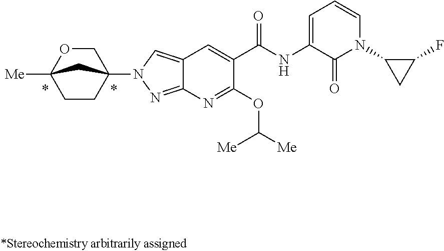 [SMILES: CC(C)Oc1nc2nn([C@@]34CC[C@@](C)(C3)OC4)cc2cc1C(=O)Nc1cccn([C@H]2C[C@H]2F)c1=O]

To a solution of 6-isopropoxy-2-((1S,4S)-1-methyl-2-oxabicyclo[2.2.1]heptan-4-yl)-2H-indazole-5-carboxylic acid (Preparation 91, 45 mg, 0.136 mmol) and 3-amino-1-((1S,2R)-2-fluorocyclopropyl)pyridin-2(1H)-one (Preparation 77, 27.5 mg, 0.164 mmol) in pyridine (2 mL) was added T3P (2 mL) and the mixture stirred at 20° C. for 2 h. The reaction mixture was evaporated to dryness in vacuo and the residue diluted with water (10 mL) and aq. NaHCO₃ (10 mL) and extracted with EtOAc (3×20 mL). The combined organics were washed with brine (30 mL), dried (Na₂SO₄) and evaporated to dryness. The residue was purified by Prep-HPLC-A to give N-(1-((1S,2R)-2-fluorocyclopropyl)-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide as a white solid (34.8 mg, 53%). LCMS m/z=481.3 [M+H]⁺. ¹H NMR (500 MHz, MeOH-d₄) δ: 8.62-8.60 (m, 2H), 8.46 (d, J=3.0 Hz, 1H), 7.36 (d, J=7.5 Hz, 1H), 7.14 (s, 1H), 6.44-6.38 (m, 1H), 5.03-5.02 (m, 1H), 4.91-4.89 (m, 1H), 4.19 (dd, J=6.0, 2.0 Hz, 1H), 4.12-4.10 (m, 1H), 3.40-3.39 (m, 1H), 2.46-2.44 (m, 1H), 2.39 (s, 2H), 2.34-2.32 (m, 1H), 2.04-1.96 (m, 2H), 1.60 (d, J=6.0 Hz, 6H), 1.58-1.51 (m, 2H), 1.48 (d, J=2.0 Hz, 3H).

### Example 51: N-(1-((1S,2R)-2-fluorocyclopropyl)-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide

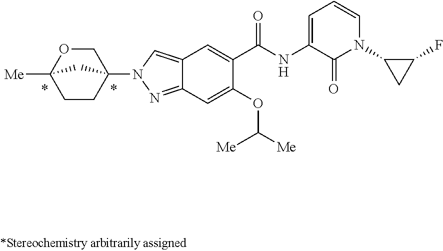 [SMILES: CC(C)Oc1cc2nn([C@]34CC[C@](C)(C3)OC4)cc2cc1C(=O)Nc1cccn([C@H]2C[C@H]2F)c1=O]

The title compound was prepared as a white solid (30.7 mg, 50%) from 6-isopropoxy-2-((1R,4R)-1-methyl-2-oxabicyclo[2.2.1]heptan-4-yl)-2H-indazole-5-carboxylic acid (Preparation 92) and 3-amino-1-((1S,2R)-2-fluorocyclopropyl)pyridin-2(1H)-one (Preparation 77) using an analogous method to that described for Example 50. LCMS m/z=481.3 [M+H]⁺. ¹H NMR (500 MHz, MeOH-d₄) δ: 8.62-8.60 (m, 2H), 8.46 (d, J=3.0 Hz, 1H), 7.36 (d, J=7.5 Hz, 1H), 7.14 (s, 1H), 6.44-6.38 (m, 1H), 5.03-5.02 (m, 1H), 4.91-4.89 (m, 1H), 4.19 (dd, J=6.0, 2.0 Hz, 1H), 4.12-4.10 (m, 1H), 3.40-3.39 (m, 1H), 2.46-2.44 (m, 1H), 2.39 (s, 2H), 2.34-2.32 (m, 1H), 2.04-1.96 (m, 2H), 1.60 (d, J=6.0 Hz, 6H), 1.58-1.51 (m, 2H), 1.48 (d, J=2.0 Hz, 3H).

### Example 52: N-(1-((1S,2R)-2-fluorocyclopropyl)-2-oxo-1,2-dihydropyridin-3-yl)-2-(1-(fluoromethyl)-2-oxabicyclo[2.1.1]hexan-4-yl)-6-isopropoxy-2H-indazole-5-carboxamide

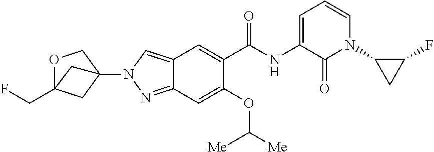 [SMILES: CC(C)Oc1cc2nn(C34COC(CF)(C3)C4)cc2cc1C(=O)Nc1cccn([C@H]2C[C@H]2F)c1=O]

To a solution of 2-(1-(fluoromethyl)-2-oxabicyclo[2.1.1]hexan-4-yl)-6-isopropoxy-2H-indazole-5-carboxylic acid (Preparation 86, 30 mg, 0.087 mmol) and 3-amino-1-((1S,2R)-2-fluorocyclopropyl)pyridin-2(1H)-one (Preparation 77, 29.1 mg, 0.173 mmol) in pyridine (3 mL) was added T3P® (3 mL) and the mixture stirred at 20° C. for 2 h. The reaction mixture was concentrated in vacuo and the residue diluted with water (10 mL) and aq. NaHCO₃ (10 mL) and extracted with EtOAc (3×20 mL). The combined organics were washed with brine (30 mL), dried (Na₂SO₄) and evaporated to dryness in vacuo. The residue was purified by Prep-HPLC-A to give N-(1-((1S,2R)-2-fluorocyclopropyl)-2-oxo-1,2-dihydropyridin-3-yl)-2-(1-(fluoromethyl)-2-oxabicyclo[2.1.1]hexan-4-yl)-6-isopropoxy-2H-indazole-5-carboxamide as a white solid (35.2 mg, 81% yield). LCMS m/z=485.2 [M+H]⁺. ¹H NMR (500 MHz, MeOH-d₄) δ: 8.64 (s, 1H), 8.62 (d, J=6.0 Hz, 1H), 8.53 (s, 1H), 7.37 (d, J=7.0 Hz, 1H), 7.16 (s, 1H), 6.41-6.38 (m, 1H), 5.03-5.02 (m, 1H), 4.98-4.95 (m, 1H), 4.78 (s, 1H), 4.68 (s, 1H), 4.25 (s, 2H), 3.42-3.38 (m, 1H), 2.59-2.56 (m, 2H), 2.42-2.39 (m, 2H), 1.60 (d, J=6.0 Hz, 6H), 1.57-1.56 (m, 1H), 1.55-1.52 (m, 1H).

### Example 53: N-(1-((1R,2S)-2-fluorocyclopropyl)-2-oxo-1,2-dihydropyridin-3-yl)-2-(1-(fluoromethyl)-2-oxabicyclo[2.1.1]hexan-4-yl)-6-isopropoxy-2H-indazole-5-carboxamide

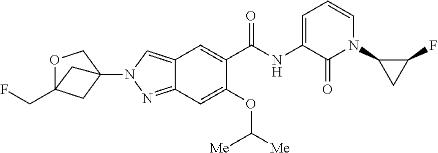 [SMILES: CC(C)Oc1cc2nn(C34COC(CF)(C3)C4)cc2cc1C(=O)Nc1cccn([C@@H]2C[C@@H]2F)c1=O]

The title compound was prepared as a white solid (27.7 mg, 66%) from 2-(1-(fluoromethyl)-2-oxabicyclo[2.1.1]hexan-4-yl)-6-isopropoxy-2H-indazole-5-carboxylic acid (Preparation 86, 30 mg, 0.087 mmol) and 3-amino-1-((1R,2S)-2-fluorocyclopropyl)pyridin-2(1H)-one (Preparation 76) using an analogous method to that described for Example 52. LCMS m/z=485.2 [M+H]⁺. ¹H NMR (500 MHz, MeOH-d₄) δ: 8.64 (s, 1H), 8.62 (d, J=6.0 Hz, 1H), 8.53 (s, 1H), 7.37 (d, J=7.0 Hz, 1H), 7.16 (s, 1H), 6.41-6.38 (m, 1H), 5.03-5.02 (m, 1H), 4.98-4.95 (m, 1H), 4.78 (s, 1H), 4.68 (s, 1H), 4.25 (s, 2H), 3.42-3.38 (m, 1H), 2.59-2.56 (m, 2H), 2.42-2.39 (m, 2H), 1.60 (d, J=6.0 Hz, 6H), 1.57-1.56 (m, 1H), 1.55-1.52 (m, 1H).

### Examples 54-94

The title compounds were prepared from the appropriate carboxylic acid (RCO₂H) and the appropriate aminopyridone (RNH₂) using an analogous method to that described for Example 53.

| Example | |
|---|---|
| No. | Name/Structure/Reactants/Data |
| 54 | N-(1-((1R,2R)-2-fluorocyclopropyl)-2-oxo-1,2-dihydropyridin-3-yl)-6- |
| | isopropoxy-2-(1-methyl-2-oxabicyclo[2.1.1]hexan-4-yl)-2H-indazole-5- |
| | carboxamide |
| | 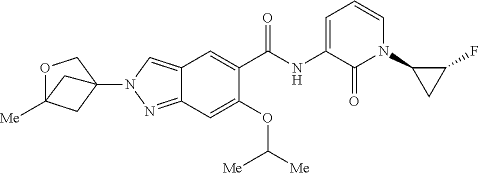 [SMILES: CC(C)Oc1cc2nn(C34COC(C)(C3)C4)cc2cc1C(=O)Nc1cccn([C@@H]2C[C@H]2F)c1=O] |
| | Absolute stereochemistry arbitrarily assigned |
| | RCO₂H: 6 isopropoxy-2-(1-methyl-2-oxabicyclo[2.1.1]hexan-4-yl)-2H- |
| | indazole-5-carboxylic acid (Preparation 49) |
| | RNH₂: 3-amino-1-((1R,2R)-2-fluorocyclopropyl)pyridin-2(1H)-one |
| | (Preparation 74A) |
| | Prep-HPLC-B; LCMS m/z = 467.1 [M + H]⁺. ¹H NMR (400 MHz, MeOH- |
| | d₄) δ: 8.64 (s, 1H), 8.58 (dd, J = 7.2, 1.6 Hz, 1H), 8.49 (s, 1H), 7.22 (d, J = |
| | 6.8 Hz, 1H), 6.37 (t, J = 6.8 Hz, 1H), 5.00-4.90 (m, 1H), 4.80-4.70 (m, |
| | 1H), 4.18 (s, 2H), 3.85-3.75 (m, 1H), 2.40-2.30 (m, 2H), 2.25-2.15 (m, |
| | 2H), 1.80-1.70 (m, 1H), 1.62 (d, J = 6.4 Hz, 6H), 1.49 (s, 1H), 1.50-1.40 |
| | (m, 1H). |
| 55 | N-(1-((1R,2R)-2-fluorocyclopropyl)-2-oxo-1,2-dihydropyridin-3-yl)-6- |
| | isopropoxy-2-(1-(methoxymethyl)-2-oxabicyclo[2.1.1]hexan-4-yl)-2H- |
| | indazole-5-carboxamide |
| | 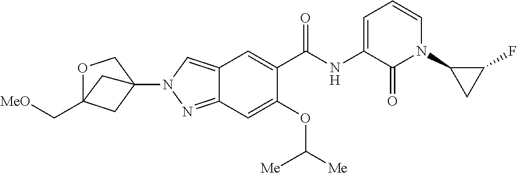 [SMILES: COCC12CC(n3cc4cc(C(=O)Nc5cccn([C@@H]6C[C@H]6F)c5=O)c(OC(C)C)cc4n3)(CO1)C2] |
| | Absolute stereochemistry arbitrarily assigned |
| | RCO₂H: 6-isopropoxy-2-(1-(methoxymethyl)-2- |
| | oxabicyclo[2.1.1]hexan-4-yl)-2H-indazole-5-carboxylic acid |
| | (Preparation 27). |
| | RNH₂: 3-amino-1-((1R,2R)-2-fluorocyclopropyl)pyridin-2(1H)-one |
| | (Preparation 74A) |
| | Prep-HPLC-B; White solid (20.4 mg, 23%); LCMS m/z = 497.2 [M + H]⁺. |
| | ¹H NMR (400 MHz, MeOH-d₄) δ: 8.64 (s, 1H), 8.57 (dd, J = 7.2, 1.2 Hz, |
| | 1H), 8.51 (s, 1H), 7.22 (d, J = 6.8 Hz, 1H), 7.16 (s, 1H), 6.37 (t, J = 7.5 |
| | Hz, 1H), 5.02-4.96 (m, 1H), 4.84-4.81 (m, 1H), 4.21 (s, 1H), 3.85-3.80 |
| | (m, 1H), 3.78 (s, 2H), 3.45 (s, 3H), 2.51-2.47 (m, 2H), 2.38-2.34 (m, 2H), |
| | 1.81-1.70 (m, 1H), 1.61 (dd, J = 6.0, 1.6 Hz, 6H), 1.54-1.46 (m, 1H). |
| 56 | N-(1-((1S,2S)-2-fluorocyclopropyl)-2-oxo-1,2-dihydropyridin-3-yl)-6- |
| | isopropoxy-2-(1-(methoxymethyl)-2-oxabicyclo[2.1.1]hexan-4-yl)-2H- |
| | indazole-5-carboxamide |
| | 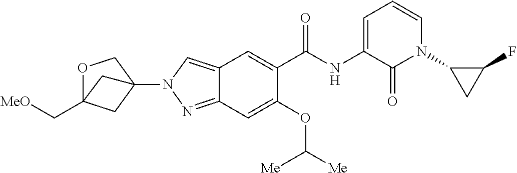 [SMILES: COCC12CC(n3cc4cc(C(=O)Nc5cccn([C@H]6C[C@@H]6F)c5=O)c(OC(C)C)cc4n3)(CO1)C2] |
| | Absolute stereochemistry arbitrarily assigned |
| | RCO₂H: 6-isopropoxy-2-(1-(methoxymethyl)-2- |
| | oxabicyclo[2.2.1]hexan-4-yl)-2H-indazole-5-carboxylic acid |
| | (Preparation 27). |
| | RNH₂: 3-amino-1-((1S,2S)-2-fluorocyclopropyl)pyridin-2(1H)-one |
| | (Preparation 75A) |
| | Prep-HPLC-B; White solid (22.2 mg, 25%); LCMS m/z = 497.2 [M + H]⁺. |
| | ¹H NMR (400 MHz, MeOH-d₄) δ: 8.64 (s, 1H), 8.57 (dd, J = 7.2, 1.2 Hz, |
| | 1H), 8.51 (s, 1H), 7.22 (d, J = 6.8 Hz, 1H), 7.16 (s, 1H), 6.37 (t, J = 7.5 |
| | Hz, 1H), 5.00-4.96 (m, 1H), 4.83-4.81 (m, 1H), 4.21 (s, 1H), 3.85-3.80 |
| | (m, 1H), 3.78 (s, 2H), 3.45 (s, 3H), 2.51-2.47 (m, 2H), 2.38-2.34 (m, 2H), |
| | 1.81-1.70 (m, 1H), 1.61 (dd, J = 6.0, 1.6 Hz, 6H), 1.54-1.46 (m, 1H). |
| 57 | 6-cyclobutoxy-N-(1-((1R,2R)-2-fluorocyclopropyl)-2-oxo-1,2- |
| | dihydropyridin-3-yl)-2-(1-(methoxymethyl)-2-oxabicyclo[2.1.1]hexan- |
| | 4-yl)-2H-indazole-5-carboxamide |
| | 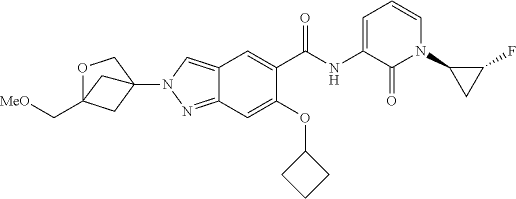 [SMILES: COCC12CC(n3cc4cc(C(=O)Nc5cccn([C@@H]6C[C@H]6F)c5=O)c(OC5CCC5)cc4n3)(CO1)C2] |
| | RCO₂H: 6-cyclobutoxy-2-(1-(methoxymethyl)-2- |
| | oxabicyclo[2.1.1]hexan-4-yl)-2H-indazole-5-carboxylic acid |
| | (Preparation 88) |
| | RNH₂: 3-amino-1-((1R,2R)-2-fluorocyclopropyl)pyridin-2(1H)-one |
| | (Preparation 74A) |
| | Prep-HPLC-A; White solid (14 mg, 33%); LCMS m/z = 509.2 [M + H]⁺. |
| | ¹H NMR (400 MHz, MeOH-d₄) δ: 8.62 (s, 1 H), 8.56 (d, J = 7.2 Hz, 1 H), |
| | 8.48 (s, 1 H), 7.20 (d, J = 6.0 Hz, 1 H), 6.94 (s, 1 H), 6.37-6.34 (m, 1 |
| | H), 5.02-4.94 (m, 2 H), 4.19 (s, 2 H), 3.82-3.75 (m, 1 H), 3.74 (s, 2 H), |
| | 3.44 (s, 3 H), 2.66-2.64 (m, 4 H), 2.49-2.48 (m, 2 H), 2.34-2.32 (m, 2 |
| | H), 2.05-1.97 (m, 1 H), 1.89-1.71 (m, 2 H), 1.51-1.48 (m, 1 H). |
| 58 | 6-cyclobutoxy-N-(1-((1S,2S)-2-fluorocyclopropyl)-2-oxo-1,2- |
| | dihydropyridin-3-yl)-2-(1-(methoxymethyl)-2-oxabicyclo[2.1.1]hexan- |
| | 4-yl)-2H-indazole-5-carboxamide |
| | 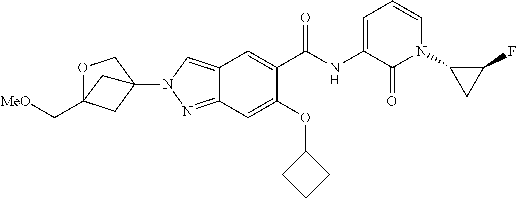 [SMILES: COCC12CC(n3cc4cc(C(=O)Nc5cccn([C@H]6C[C@@H]6F)c5=O)c(OC5CCC5)cc4n3)(CO1)C2] |
| | RCO₂H: 6-cyclobutoxy-2-(1-(methoxymethyl)-2- |
| | oxabicyclo[2.1.1]hexan-4-yl)-2H-indazole-5-carboxylic acid |
| | (Preparation 88) |
| | RNH₂: 3-amino-1-((1S,2S)-2-fluorocyclopropyl)pyridin-2(1H)-one |
| | (Preparation 75A) |
| | Prep-HPLC-A; White solid (12.4 mg, 29%); LCMS m/z = 509.2 [M + H]⁺. |
| | ¹H NMR (400 MHz, MeOH-d₄) δ: 8.62 (s, 1 H), 8.56 (d, J = 7.2 Hz, 1 H), |
| | 8.48 (s, 1 H), 7.20 (d, J = 6.0 Hz, 1 H), 6.94 (s, 1 H), 6.37-6.34 (m, 1 H), |
| | 5.02-4.94 (m, 2 H), 4.19 (s, 2 H), 3.82-3.75 (m, 1 H), 3.74 (s, 2 H), 3.44 |
| | (s, 3 H), 2.66-2.64 (m, 4 H), 2.49-2.48 (m, 2 H), 2.34-2.32 (m, 2 H), 2.05- |
| | 1.97 (m, 1 H), 1.89-1.71 (m, 2 H), 1.51-1.48 (m, 1 H). |
| 59 | N-(1-((1R,2R)-2-fluorocyclopropyl)-2-oxo-1,2-dihydropyridin-3-yl)-6- |
| | isopropoxy-2-((1S,4S)-1-methyl-2-oxabicyclo[2.2.1]heptan-4-yl)-2H- |
| | indazole-5-carboxamide |
| | 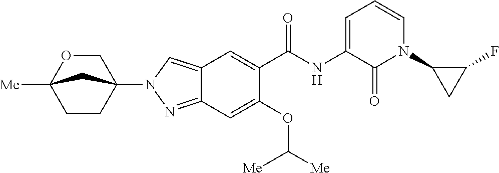 [SMILES: CC(C)Oc1cc2nn([C@@]34CC[C@@](C)(C3)OC4)cc2cc1C(=O)Nc1cccn([C@@H]2C[C@H]2F)c1=O] |
| | Absolute stereochemistry arbitrarily assigned |
| | RCO₂H: 6-isopropoxy-2-((1S,4S)-1-methyl-2-oxabicyclo[2.2.1]heptan- |
| | 4-yl)-2H-indazole-5-carboxylic acid (Preparation 91) |
| | RNH₂: 3-amino-1-((1R,2R)-2-fluorocyclopropyl)pyridin-2(1H)-one |
| | (Preparation 74A) |
| | Prep-HPLC-A; White solid (29.1 mg, 44%); LCMS m/z = 481.2 [M + H]⁺. |
| | ¹H NMR (400 MHz, MeOH-d₄) δ: 8.62 (s, 1 H), 8.56 (dd, J = 7.2, 1.6 Hz, |
| | 1 H), 8.45 (s, 1 H), 7.20 (d, J = 7.2 Hz, 1 H), 7.14 (s, 1 H), 6.36 (t, J = 7.2 |
| | Hz, 1 H), 4.99-4.97 (m, 1 H), 4.96-4.93 (m, 1 H), 4.19 (d, J = 6.0 Hz, 1 |
| | H), 4.10 (dd, J = 6.0, 4.0 Hz, 1 H), 3.81-3.78 (m, 1 H), 2.46-2.43 (m, 1 |
| | H), 2.39 (s, 2 H), 2.33-2.31 (m, 1 H), 2.05-2.01 (m, 2 H), 1.78-1.75 (m, |
| | 1 H), 1.62 (d, J = 6.4 Hz, 6 H), 1.54-1.49 (m, 1 H), 1.48 (s, 3 H). |
| 60 | N-(1-((1S,2S)-2-fluorocyclopropyl)-2-oxo-1,2-dihydropyridin-3-yl)-6- |
| | isopropoxy-2-((1S,4S)-1-methyl-2-oxabicyclo[2.2.1]heptan-4-yl)-2H- |
| | indazole-5-carboxamide |
| | 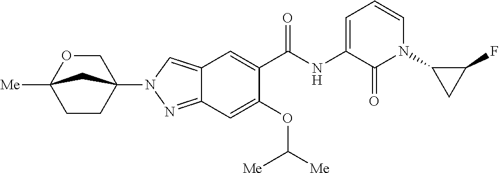 [SMILES: CC(C)Oc1cc2nn([C@@]34CC[C@@](C)(C3)OC4)cc2cc1C(=O)Nc1cccn([C@H]2C[C@@H]2F)c1=O] |
| | RCO₂H: 6-isopropoxy-2-((1S,4S)-1-methyl-2-oxabicyclo[2.2.1]heptan- |
| | 4-yl)-2H-indazole-5-carboxylic acid (Preparation 91) |
| | RNH₂: 3-amino-1-((1S,2S)-2-fluorocyclopropyl)pyridin-2(1H)-one |
| | (Preparation 75A) |
| | Prep-HPLC-A; White solid (21.1 mg, 35%); LCMS m/z = 481.2 [M + H]⁺. |
| | ¹H NMR (400 MHz, MeOH-d₄) δ: 8.62 (s, 1 H), 8.56 (dd, J = 7.2, 1.6 Hz, |
| | 1 H), 8.45 (s, 1 H), 7.20 (d, J = 7.2 Hz, 1 H), 7.14 (s, 1 H), 6.36 (t, J = 7.2 |
| | Hz, 1 H), 4.99-4.97 (m, 1 H), 4.96-4.93 (m, 1 H), 4.19 (d, J = 6.0 Hz, 1 |
| | H), 4.10 (dd, J = 6.0, 4.0 Hz, 1 H), 3.81-3.78 (m, 1 H), 2.46-2.43 (m, 1 |
| | H), 2.39 (s, 2 H), 2.33-2.31 (m, 1 H), 2.05-2.01 (m, 2 H), 1.78-1.75 (m, |
| | 1 H), 1.62 (d, J = 6.4 Hz, 6 H), 1.54-1.49 (m, 1 H), 1.48 (s, 3 H). |
| 61 | N-(1-((1R,2R)-2-fluorocyclopropyl)-2-oxo-1,2-dihydropyridin-3-yl)-6- |
| | isopropoxy-2-((1R,4R)-1-methyl-2-oxabicyclo[2.2.1]heptan-4-yl)-2H- |
| | indazole-5-carboxamide |
| | 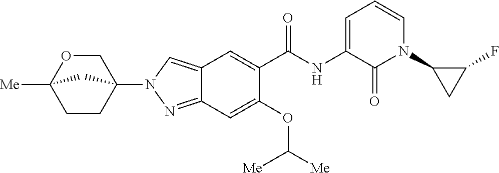 [SMILES: CC(C)Oc1cc2nn([C@]34CC[C@](C)(C3)OC4)cc2cc1C(=O)Nc1cccn([C@@H]2C[C@H]2F)c1=O] |
| | RCO₂H: 6-isopropoxy-2-((1R,4R)-1-methyl-2-oxabicyclo[2.2.1]heptan- |
| | 4-yl)-2H-indazole-5-carboxylic acid (Preparation 92) |
| | RNH₂: 3-amino-1-((1R,2R)-2-fluorocyclopropyl)pyridin-2(1H)-one |
| | (Preparation 74A) |
| | Prep-HPLC-A; White solid (11.5 mg, 16%); LCMS m/z = 481.2 [M + H]⁺. |
| | ¹H NMR (400 MHz, MeOH-d₄) δ: 8.62 (s, 1 H), 8.56 (dd, J = 7.2, 1.6 Hz, |
| | 1 H), 8.45 (s, 1 H), 7.20 (d, J = 7.2 Hz, 1 H), 7.14 (s, 1 H), 6.36 (t, J = 7.2 |
| | Hz, 1 H), 4.99-4.97 (m, 1 H), 4.96-4.93 (m, 1 H), 4.19 (d, J = 6.0 Hz, 1 |
| | H), 4.10 (dd, J = 6.0, 4.0 Hz, 1 H), 3.81-3.78 (m, 1 H), 2.46-2.43 (m, 1 |
| | H), 2.39 (s, 2 H), 2.33-2.31 (m, 1 H), 2.05-2.01 (m, 2 H), 1.78-1.75 (m, |
| | 1 H), 1.62 (d, J = 6.4 Hz, 6 H), 1.54-1.49 (m, 1 H), 1.48 (s, 3 H). |
| 62 | N-(1-((1S,2S)-2-fluorocyclopropyl)-2-oxo-1,2-dihydropyridin-3-yl)-6- |
| | isopropoxy-2-((1R,4R)-1-methyl-2-oxabicyclo[2.2.1]heptan-4-yl)-2H- |
| | indazole-5-carboxamide |
| | 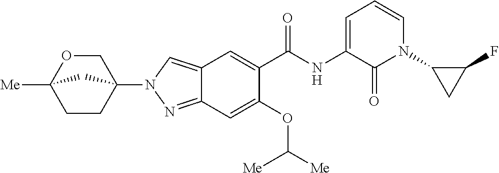 [SMILES: CC(C)Oc1cc2nn([C@]34CC[C@](C)(C3)OC4)cc2cc1C(=O)Nc1cccn([C@H]2C[C@@H]2F)c1=O] |
| | Absolute stereochemistry arbitrarily assigned |
| | RCO₂H: 6-isopropoxy-2-((1R,4R)-1-methyl-2-oxabicyclo[2.2.1]heptan- |
| | 4-yl)-2H-indazole-5-carboxylic acid (Preparation 92) |
| | RNH₂: 3-amino-1-((1S,2S)-2-fluorocyclopropyl)pyridin-2(1H)-one |
| | (Preparation 75A) |
| | Prep-HPLC-A; White solid (10 mg, 14%); LCMS m/z = 481.2 [M + H]⁺. |
| | ¹H NMR (400 MHz, MeOH-d₄) δ: 8.62 (s, 1 H), 8.56 (dd, J = 7.2, 1.6 Hz, |
| | 1 H), 8.45 (s, 1 H), 7.20 (d, J = 7.2 Hz, 1 H), 7.14 (s, 1 H), 6.36 (t, J = 7.2 |
| | Hz, 1 H), 4.99-4.97 (m, 1 H), 4.96-4.93 (m, 1 H), 4.19 (d, J = 6.0 Hz, 1 |
| | H), 4.10 (dd, J = 6.0, 4.0 Hz, 1 H), 3.81-3.78 (m, 1 H), 2.46-2.43 (m, 1 |
| | H), 2.39 (s, 2 H), 2.33-2.31 (m, 1 H), 2.05-2.01 (m, 2 H), 1.78-1.75 (m, |
| | 1 H), 1.62 (d, J = 6.4 Hz, 6 H), 1.54-1.49 (m, 1 H), 1.48 (s, 3 H). |
| 63 | N-(1-((1R,2R)-2-fluorocyclopropyl)-2-oxo-1,2-dihydropyridin-3-yl)-6- |
| | isopropoxy-2-(1-(methoxymethyl)-2-oxabicyclo[2.1.1]hexan-4-yl)-2H- |
| | pyrazolo[3,4-b]pyridine-5-carboxamide |
| | 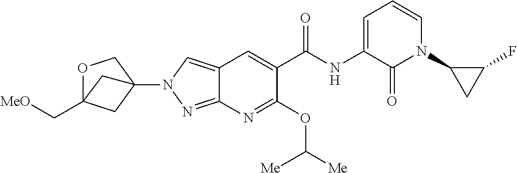 [SMILES: COCC12CC(n3cc4cc(C(=O)Nc5cccn([C@@H]6C[C@H]6F)c5=O)c(OC(C)C)nc4n3)(CO1)C2] |
| | Absolute stereochemistry arbitrarily assigned |
| | RCO₂H: 6-isopropoxy-2-(1-(methoxymethyl)-2- |
| | oxabicyclo[2.1.1]hexan-4-yl)-2H-pyrazolo[3,4-b]pyridine-5-carboxylic |
| | acid (Preparation 23) |
| | RNH₂: 3-amino-1-((1R,2R)-2-fluorocyclopropyl)pyridin-2(1H)-one |
| | (Preparation 74A) |
| | Prep-HPLC-A; White solid (14.8 mg, 21%); LCMS m/z = 498.2 [M + H]⁺. |
| | ¹H NMR (400 MHz, MeOH-d₄) δ: 9.00 (s, 1H), 8.57 (dd, J = 1.6, 7.6 Hz, |
| | 1H), 8.51 (s, 1H), 7.22 (dd, J = 1.6, 7.2 Hz, 1H), 6.36 (t, J = 7.2 Hz, 1H), |
| | 5.83-5.76 (m, 1H), 5.02-4.87 (m, 1H), 4.21 (s, 2H), 3.86-3.78 (m, 1H), |
| | 3.76 (s, 2H), 3.45 (s, 3H), 2.50 (dd, J = 1.2, 4.4 Hz, 2H), 2.34 (dd, J = 1.6, |
| | 4.4 Hz, 2H), 1.82-1.71 (m, 1H), 1.63 (dd, J = 1.6, 6.2 Hz, 6H), 1.53-1.50 |
| | (m, 1H). |
| 64 | N-(1-((1S,2S)-2-fluorocyclopropyl)-2-oxo-1,2-dihydropyridin-3-yl)-6- |
| | isopropoxy-2-(1-(methoxymethyl)-2-oxabicyclo[2.1.1]hexan-4-yl)-2H- |
| | pyrazolo[3,4-b]pyridine-5-carboxamide |
| | 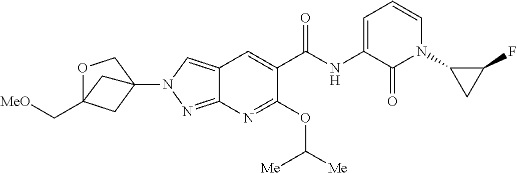 [SMILES: COCC12CC(n3cc4cc(C(=O)Nc5cccn([C@H]6C[C@@H]6F)c5=O)c(OC(C)C)nc4n3)(CO1)C2] |
| | Absolute stereochemistry arbitrarily assigned |
| | RCO₂H: 6-isopropoxy-2-(1-(methoxymethyl)-2- |
| | oxabicyclo[2.1.1]hexan-4-yl)-2H-pyrazolo[3,4-b]pyridine-5-carboxylic |
| | acid (Preparation 23) |
| | RNH₂: 3-amino-1-((1S,2S)-2-fluorocyclopropyl)pyridin-2(1H)-one |
| | (Preparation 75A) |
| | Prep-HPLC-A; White solid (14.2 mg, 20%); LCMS m/z = 481.2 [M + H]⁺. |
| | ¹H NMR (400 MHz, MeOH-d₄) δ: 9.00 (s, 1H), 8.56 (dd, J = 1.6, 7.2 Hz, |
| | 1H), 8.51 (s, 1H), 7.22 (d, J = 1.6, 7.2 Hz, 1H), 6.36 (t, J = 7.2 Hz, 1H), |
| | 5.82-5.76 (m, 1H), 5.00-4.90 (m, 1H), 5.00-4.88 (m, 1H), 4.21 (s, 2H), |
| | 3.87-3.78 (m, 1H), 3.76 (s, 2H), 3.45 (s, 3H), 2.50 (dd, J = 1.2, 4.4 Hz, |
| | 2H), 2.34 (dd, J = 1.6, 4.4 Hz, 2H), 1.83-1.72 (m, 1H), 1.63 (dd, J = 1.6, |
| | 6.0 Hz, 6H), 1.52 (s, 1H). |
| 65 | 6-cyclobutoxy-N-(1-((1R,2R)-2-fluorocyclopropyl)-2-oxo-1,2- |
| | dihydropyridin-3-yl)-2-(1-(methoxymethyl)-2-oxabicyclo[2.1.1]hexan- |
| | 4-yl)-2H-pyrazolo[3,4-b]pyridine-5-carboxamide |
| | 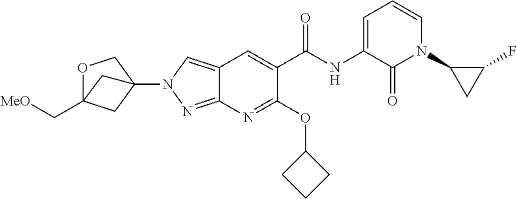 [SMILES: COCC12CC(n3cc4cc(C(=O)Nc5cccn([C@@H]6C[C@H]6F)c5=O)c(OC5CCC5)nc4n3)(CO1)C2] |
| | Absolute stereochemistry arbitrarily assigned |
| | RCO₂H: 6-cyclobutoxy-2-(1-(methoxymethyl)-2- |
| | oxabicyclo[2.1.1]hexan-4-yl)-2H-pyrazolo[3,4-b]pyridine-5-carboxylic |
| | acid (Preparation 111). |
| | RNH₂: 3-amino-1-((1R,2R)-2-fluorocyclopropyl)pyridin-2(1H)-one |
| | (Preparation 74A) |
| | Prep-HPLC-B; White solid (27.3 mg, 43%); LCMS m/z = 510.2 [M + H]⁺. |
| | ¹H NMR (500 MHz, MeOH-d₄) δ: = 9.02 (s, 1H), 8.59 (dd, J = 7.5, 1.5 |
| | Hz, 1H), 8.52 (s, 1H), 7.24 (dd, J = 7.0, 1.5 Hz, 1H), 6.38 (t, J = 7.0 Hz, |
| | 1H), 5.62-5.56 (m, 1H), 5.09-4.96 (m, 1H), 4.21 (s, 2H), 3.86-3.79 (m, |
| | 1H), 3.76 (s, 2H), 3.45 (s, 3H), 2.67-2.62 (m, 4H), 2.51-2.49 (m, 2H), |
| | 2.35-2.33 (m, 2H), 2.03-1.96 (m, 1H), 1.85-1.72 (m, 2H), 1.55-1.47 (m, |
| | 1H). |
| 66 | 6-cyclobutoxy-N-(1-((1S,2S)-2-fluorocyclopropyl)-2-oxo-1,2- |
| | dihydropyridin-3-yl)-2-(1-(methoxymethyl)-2-oxabicyclo[2.1.1]hexan- |
| | 4-yl)-2H-pyrazolo[3,4-b]pyridine-5-carboxamide |
| | 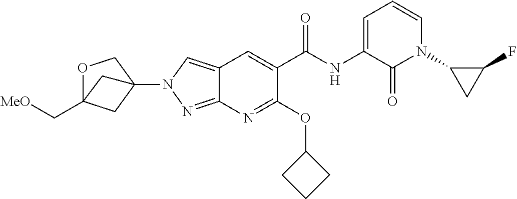 [SMILES: COCC12CC(n3cc4cc(C(=O)Nc5cccn([C@H]6C[C@@H]6F)c5=O)c(OC5CCC5)nc4n3)(CO1)C2] |
| | Absolute stereochemistry arbitrarily assigned |
| | RCO₂H: 6-cyclobutoxy-2-(1-(methoxymethyl)-2- |
| | oxabicyclo[2.1.1]hexan-4-yl)-2H-pyrazolo[3,4-b]pyridine-5-carboxylic |
| | acid (Preparation 111) |
| | RNH₂: 3-amino-1-((1S,2S)-2-fluorocyclopropyl)pyridin-2(1H)-one |
| | (Preparation 75A) |
| | Prep-HPLC-B; White solid (27.3 mg, 43%); LCMS m/z = 510.2 [M + H]⁺. |
| | ¹H NMR (500 MHz, MeOH-d₄) δ: 9.02 (s, 1H), 8.59 (dd, J = 7.5, 1.5 Hz, |
| | 1H), 8.52 (s, 1H), 7.24 (dd, J = 7.0, 1.5 Hz, 1H), 6.38 (t, J = 7.0 Hz, 1H), |
| | 5.62-5.56 (m, 1H), 5.00-4.95 (m, 1H), 4.21 (s, 2H), 3.84-3.79 (m, 1H), |
| | 3.76 (s, 2H), 3.45 (s, 3H), 2.67-2.62 (m, 4H), 2.51-2.49 (m, 2H), 2.35- |
| | 2.33 (m, 2H), 2.03-1.96 (m, 1H), 1.85-1.72 (m, 2H), 1.52-1.50 (m, 1H). |
| 67 | N-(1-((1S,2R)-2-fluorocyclopropyl)-2-oxo-1,2-dihydropyridin-3-yl)-6- |
| | isopropoxy-2-(1-(methoxymethyl)-2-oxabicyclo[2.1.1]hexan-4-yl)-2H- |
| | indazole-5-carboxamide |
| | 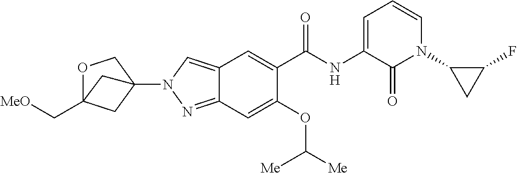 [SMILES: COCC12CC(n3cc4cc(C(=O)Nc5cccn([C@H]6C[C@H]6F)c5=O)c(OC(C)C)cc4n3)(CO1)C2] |
| | RCO₂H: 6-isopropoxy-2-(1-(methoxymethyl)-2- |
| | oxabicyclo[2.1.1]hexan-4-yl)-2H-pyrazolo[3,4-b]pyridine-5-carboxylic |
| | acid (Preparation 23) |
| | RNH₂: 3-amino-1-((1S,2R)-2-fluorocyclopropyl)pyridin-2(1H)-one |
| | hydrochloride (Preparation 77) |
| | Prep-HPLC-B; White solid (17 mg, 40%); LCMS m/z = 519.1 [M + H]⁺. |
| | ¹H NMR (400 MHz, MeOH-d₄) δ: 8.70-8.60 (m, 2H), 8.51 (s, 1H), 7.37 |
| | (d, J = 7.2 Hz, 1H), 7.15 (s, 1H), 6.39 (t, J = 7.2 Hz, 1H), 5.10-5.00 (m, |
| | 1H), 5.00-4.90 (m, 1H), 4.21 (s, 2H), 3.76 (s, 2H), 3.44 (s, 3H), 3.30-3.20 |
| | (m, 1H), 2.50-2.40 (m, 2H), 2.40-2.30 (m, 2H), 1.59 (d, J = 6.4 Hz, 6H), |
| | 1.55-1.50 (m, 1H), 1.40-1.30 (m, 1H). |
| 68 | 2-(2-oxabicyclo[2.1.1]hexan-4-yl)-N-(1-((1S,2R)-2-fluorocyclopropyl)- |
| | 2-oxo-1,2-dihydropyridin-3-yl)-6-isopropoxy-2H-indazole-5- |
| | carboxamide |
| | 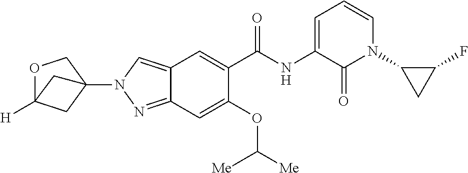 [SMILES: CC(C)Oc1cc2nn(C34COC(C3)C4)cc2cc1C(=O)Nc1cccn([C@H]2C[C@H]2F)c1=O] |
| | RCO₂H: 2-(2-oxabicyclo[2.1.1]hexan-4-yl)-6-isopropoxy-2H-indazole- |
| | 5-carboxylic acid (Preparation 87) |
| | RNH₂: 3-amino-1-((1S,2R)-2-fluorocyclopropyl)pyridin-2(1H)-one |
| | (Preparation 77) |
| | Prep-HPLC-B; White solid (25.4 mg, 17%); LCMS m/z = 453.2 [M + H]⁺. |
| | ¹H NMR (400 MHz, MeOH-d₄) δ: 8.65 (s, 1H), 8.62 (dd, J = 7.6, 1.6 Hz, |
| | 1H), 8.52 (s, 1H), 7.38 (d, J = 6.8 Hz, 1H), 7.16 (s, 1H), 6.40 (t, J = 7.2 |
| | Hz, 1H), 5.07-4.98 (m, 1H), 4.98-4.94 (m, 1H), 4.71 (s, 1H), 4.13 (s, 2H), |
| | 3.44-3.39 (m, 1H), 2.55-2.52 (m, 2H), 2.36-2.31 (m, 2H), 1.60 (d, J = 6.0 |
| | Hz, 6H), 1.58-1.54 (m, 1H), 1.54-1.50 (m, 1H). |
| 69 | 2-(1-(fluoromethyl)-2-oxabicyclo[2.1.1]hexan-4-yl)-6-isopropoxy-N-(1- |
| | ((1R,2R)-2-methylcyclopropyl)-2-oxo-1,2-dihydropyridin-3-yl)-2H- |
| | indazole-5-carboxamide |
| | 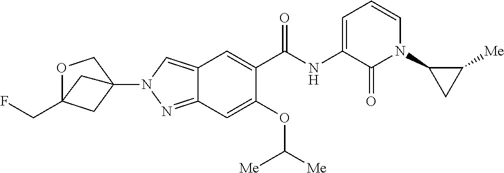 [SMILES: CC(C)Oc1cc2nn(C34COC(CF)(C3)C4)cc2cc1C(=O)Nc1cccn([C@@H]2C[C@H]2C)c1=O] |
| | Absolute stereochemistry arbitrarily assigned |
| | RCO₂H: 2-(1-(fluoromethyl)-2-oxabicyclo[2.1.1]hexan-4-yl)-6- |
| | isopropoxy-2H-indazole-5-carboxylic acid (Preparation 86) |
| | RNH₂: 3-amino-1-((1R,2R)-2-fluorocyclopropyl)pyridin-2(1H)-one |
| | (Preparation 74A) |
| | Prep-HPLC-B; White solid (34.9 mg, 61%); LCMS m/z = 481.2 [M + H]⁺. |
| | ¹H NMR (500 MHz, MeOH-d₄) δ: 8.64 (s, 1H), 8.56 (dd, J = 7.5, 2.0 Hz, |
| | 1H), 8.53 (s, 1H), 7.29 (dd, J = 6.5, 1.5 Hz, 1H), 7.16 (s, 1H), 6.35 (t, J = |
| | 7.5 Hz, 1H), 5.05-4.95 (m, 1H), 4.79-4.68 (m, 2H), 4.25 (s, 1H), 3.14- |
| | 3.10 (m, 1H), 2.58-2.54 (m, 2H), 2.43-2.37 (m, 2H), 1.61 (d, J = 6.0 Hz, |
| | 6H), 1.28-1.24 (m, 4H), 1.16-1.12 (m, 1H), 0.97-0.93 (m, 1H). |
| 70 | 2-(1-(fluoromethyl)-2-oxabicyclo[2.1.1]hexan-4-yl)-6-isopropoxy-N-(1- |
| | ((1S,2S)-2-methylcyclopropyl)-2-oxo-1,2-dihydropyridin-3-yl)-2H- |
| | indazole-5-carboxamide |
| | 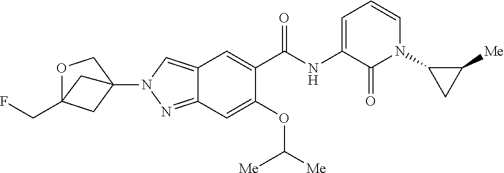 [SMILES: CC(C)Oc1cc2nn(C34COC(CF)(C3)C4)cc2cc1C(=O)Nc1cccn([C@H]2C[C@@H]2C)c1=O] |
| | Absolute stereochemistry arbitrarily assigned |
| | RCO₂H: 2-(1-(fluoromethyl)-2-oxabicyclo[2.1.1]hexan-4-yl)-6- |
| | isopropoxy-2H-indazole-5-carboxylic acid (Preparation 86) |
| | RNH₂: 3-amino-1-((1S,2S)-2-fluorocyclopropyl)pyridin-2(1H)-one |
| | (Preparation 75A) |
| | Prep-HPLC-B; White solid (33.3 mg, 58%); LCMS m/z = 481.2 [M + H]⁺. |
| | ¹H NMR (500 MHz, MeOH-d₄) δ: 8.64 (s, 1H), 8.56 (dd, J = 7.5, 1.5 Hz, |
| | 1H), 8.52 (s, 1H), 7.29 (dd, J = 7.0, 1.5 Hz, 1H), 7.16 (s, 1H), 6.35 (t, J = |
| | 7.5 Hz, 1H), 5.01-4.97 (m, 1H), 4.79-4.68 (m, 2H), 4.25 (s, 1H), 3.14- |
| | 3.10 (m, 1H), 2.60-2.54 (m, 2H), 2.43-2.37 (m, 2H), 1.61 (d, J = 6.0 Hz, |
| | 6H), 1.28-1.24 (m, 4H), 1.16-1.11 (m, 1H), 0.96-0.93 (m, 1H). |
| 71 | 6-isopropoxy-2-(1-methyl-2-oxabicyclo[2.1.1]hexan-4-yl)-N-(1- |
| | ((1R,2S)-2-methylcyclopropyl)-2-oxo-1,2-dihydropyridin-3-yl)-2H- |
| | pyrazolo[3,4-b]pyridine-5-carboxamide |
| | 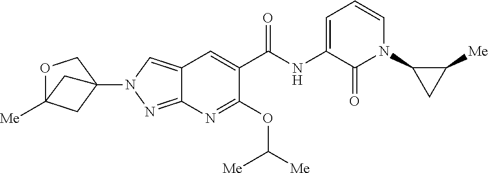 [SMILES: CC(C)Oc1nc2nn(C34COC(C)(C3)C4)cc2cc1C(=O)Nc1cccn([C@@H]2C[C@@H]2C)c1=O] |
| | RCO₂H: 6-isopropoxy-2-(1-methyl-2-oxabicyclo[2.1.1]hexan-4-yl)-2H- |
| | pyrazolo[3,4-b]pyridine-5-carboxylic acid (Preparation 6) |
| | RNH₂: 3-amino-1-((1R,2S)-2-methylcyclopropyl)pyridin-2(1H)-one |
| | hydrochloride (Preparation 72) |
| | Prep-HPLC-A; White solid (26.3 mg, 60%); LCMS m/z = 464.2 [M + H]⁺. |
| | ¹H NMR (400 MHz, MeOH-d₄) δ: 8.99 (s, 1 H), 8.61 (d, J = 6.0 Hz, 1 H), |
| | 8.48 (s, 1 H), 7.31 (d, J = 7.2 Hz, 1 H), 6.40-6.36 (m, 1 H), 5.83-5.74 |
| | (m, 1 H), 4.17 (s, 2 H), 3.48-3.43 (m, 1 H), 2.42-2.40 (m, 2 H), 2.29-2.27 |
| | (m, 2 H), 1.61 (d, J = 6.0 Hz, 6 H), 1.56 (s, 3 H), 1.51-1.43 (m, 1 H), 1.30- |
| | 1.24 (m, 1 H), 0.88-0.85 (m, 1 H), 0.84 (d, J = 6.4 Hz, 3 H). |
| 72 | 6-isopropoxy-2-(1-(methoxymethyl)-2-oxabicyclo[2.1.1]hexan-4-yl)-N- |
| | (1-((1R,2S)-2-methylcyclopropyl)-2-oxo-1,2-dihydropyridin-3-yl)-2H- |
| | pyrazolo[3,4-b]pyridine-5-carboxamide |
| | 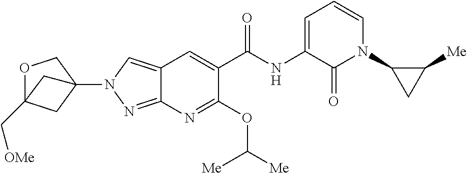 [SMILES: COCC12CC(n3cc4cc(C(=O)Nc5cccn([C@@H]6C[C@@H]6C)c5=O)c(OC(C)C)nc4n3)(CO1)C2] |
| | RCO₂H: 6-isopropoxy-2-(1-(methoxymethyl)-2- |
| | oxabicyclo[2.1.1]hexan-4-yl)-2H-pyrazolo[3,4-b]pyridine-5-carboxylic |
| | acid (Preparation 23) |
| | RNH₂: 3-amino-1-((1R,2S)-2-methylcyclopropyl)pyridin-2(1H)-one |
| | hydrochloride (Preparation 72) |
| | Prep-HPLC-A; White solid (13.1 mg, 26%); LCMS m/z = 492.2 [M + H]⁺. |
| | ¹H NMR (400 MHz, MeOH-d₄) δ: 8.97 (s, 1 H), 8.59 (d, J = 7.2 Hz, 1 H), |
| | 8.49 (s, 1 H), 7.30 (d, J = 6.0 Hz, 1 H), 6.39-6.35 (m, 1 H), 5.82-5.72 (m, |
| | 1 H), 4.20 (s, 2 H), 3.75 (s, 2 H), 3.47 (s, 1 H), 3.44 (s, 3 H), 2.50-2.48 |
| | (m, 2 H), 2.34-2.32 (m, 2 H), 1.61 (d, J = 6.0 Hz, 6 H), 1.48-1.46 (m, 1 |
| | H), 1.30-1.26 (m, 1 H), 0.91-0.86 (m, 1 H), 0.83 (d, J = 6.4 Hz, 3 H). |
| 73 | 6-isopropoxy-2-(1-(methoxymethyl)-2-oxabicyclo[2.1.1]hexan-4-yl)-N- |
| | (1-((1R,2S)-2-methylcyclopropyl)-2-oxo-1,2-dihydropyridin-3-yl)-2H- |
| | indazole-5-carboxamide |
| | 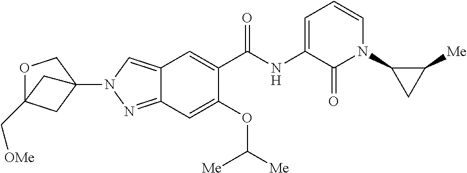 [SMILES: COCC12CC(n3cc4cc(C(=O)Nc5cccn([C@@H]6C[C@@H]6C)c5=O)c(OC(C)C)cc4n3)(CO1)C2] |
| | RCO₂H: 6-isopropoxy-2-(1-(methoxymethyl)-2- |
| | oxabicyclo[2.1.1]hexan-4-yl)-2H-indazole-5-carboxylic acid |
| | (Preparation 27) |
| | RNH₂: 3-amino-1-((1R,2S)-2-methylcyclopropyl)pyridin-2(1H)-one |
| | hydrochloride (Preparation 72) |
| | Prep-HPLC-B; White solid (15 mg, 26%); LCMS m/z = 493.1 [M + H]⁺. |
| | ¹H NMR (400 MHz, MeOH-d₄) δ: 8.30-8.40 (m, 2H), 8.51 (s, 1H), 7.31 |
| | (d, J = 6.4 Hz, 1H), 7.16 (s, 1H), 6.39 (t, J = 7.2 Hz, 1H), 5.00-4.90 (m, |
| | 1H), 4.21 (s, 2H), 3.76 (s, 2H), 3.50-3.40 (m, 4H), 2.60-2.50 (m, 2H), |
| | 2.40-2.30 (m, 2H), 1.65-1.60 (m, 6H), 1.50-1.45 (m, 1H), 1.30-1.20 (m, |
| | 2H), 0.84 (d, J = 6.4 Hz, 3H). |
| 74 | 2-(2-oxabicyclo[2.1.1]hexan-4-yl)-6-isopropoxy-N-(1-((1R,2S)-2- |
| | methylcyclopropyl)-2-oxo-1,2-dihydropyridin-3-yl)-2H-indazole-5- |
| | carboxamide |
| | 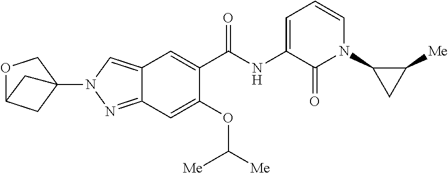 [SMILES: CC(C)Oc1cc2nn(C34COC(C3)C4)cc2cc1C(=O)Nc1cccn([C@@H]2C[C@@H]2C)c1=O] |
| | RCO₂H: 2-(2-oxabicyclo[2.1.1]hexan-4-yl)-6-isopropoxy-2H-indazole- |
| | 5-carboxylic acid (Preparation 87) |
| | RNH₂: 3-amino-1-((1R,2S)-2-methylcyclopropyl)pyridin-2(1H)-one |
| | hydrochloride (Preparation 72) |
| | Prep-HPLC-A; White solid (19.9 mg, 45%); LCMS m/z = 449.1 [M + H]⁺. |
| | ¹H NMR (400 MHz, MeOH-d₄) δ: 8.63-8.60 (m, 2 H), 8.51 (s, 1 H), 7.31 |
| | (d, J = 6.0 Hz, 1 H), 7.16 (s, 1 H), 6.40-6.37 (m, 1 H), 4.99-4.92 (m, 1 H), |
| | 4.70 (s, 1 H), 4.13 (s, 2 H), 3.46-3.41 (m, 1 H), 2.53-2.52 (m, 2 H), 2.33- |
| | 2.31 (m, 2 H), 1.60 (d, J = 6.0 Hz, 6 H), 1.50-1.48 (m, 1 H), 1.46-1.44 |
| | (m, 1 H), 0.91-0.86 (m, 1 H), 0.84 (d, J = 6.4 Hz, 3 H). |
| 75 | 2-(2-oxabicyclo[2.1.1]hexan-4-yl)-6-isopropoxy-N-(1-((1R,2S)-2- |
| | methylcyclopropyl)-2-oxo-1,2-dihydropyridin-3-yl)-2H-pyrazolo[3,4- |
| | b]pyridine-5-carboxamide |
| | 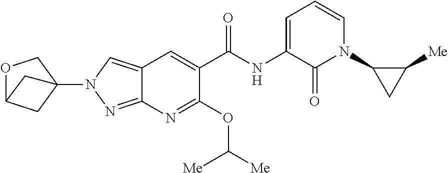 [SMILES: CC(C)Oc1nc2nn(C34COC(C3)C4)cc2cc1C(=O)Nc1cccn([C@@H]2C[C@@H]2C)c1=O] |
| | RCO₂H: 2-(2-oxabicyclo[2.1.1]hexan-4-yl)-6-isopropoxy-2H- |
| | pyrazolo[3,4-b]pyridine-5-carboxylic acid (Preparation 67) |
| | RNH₂: 3-amino-1-((1R,2S)-2-methylcyclopropyl)pyridin-2(1H)-one |
| | (Preparation 72) |
| | Prep-HPLC-B; White solid (19 mg, 32%), LCMS m/z = 450.2 [M + H]⁺. |
| | ¹H NMR (500 MHz, MeOH-d₄) δ: 9.01 (s, 1H), 8.62 (dd, J = 7.5, 1.5 Hz, |
| | 1H), 8.52 (s, 1H), 7.33 (dd, J = 7.0, 1.5 Hz, 1H), 6.39 (t, J = 7.5 Hz, 1H), |
| | 5.84-5.76 (m, 1H), 4.71 (s, 1H), 4.12 (s, 2H), 3.48-3.43 (m, 1H), 2.56- |
| | 2.50 (m, 2H), 2.35-2.29 (m, 2H), 1.62 (dd, J = 6.0, 4.5 Hz, 6H), 1.52-1.43 |
| | (m, 1H), 1.31-1.25 (m, 1H), 0.88-0.84 (m, 4H). |
| 76 | 6-isopropoxy-2-(1-methyl-2-oxabicyclo[2.1.1]hexan-4-yl)-N-(1- |
| | ((1S,2R)-2-methylcyclopropyl)-2-oxo-1,2-dihydropyridin-3-yl)-2H- |
| | pyrazolo[3,4-b]pyridine-5-carboxamide |
| | 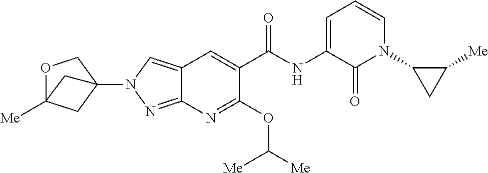 [SMILES: CC(C)Oc1nc2nn(C34COC(C)(C3)C4)cc2cc1C(=O)Nc1cccn([C@H]2C[C@H]2C)c1=O] |
| | RCO₂H: 6-isopropoxy-2-(1-methyl-2-oxabicyclo[2.1.1]hexan-4-yl)-2H- |
| | pyrazolo[3,4-b]pyridine-5-carboxylic acid (Preparation 6) |
| | RNH₂: 3-amino-1-((1S,2R)-2-methylcyclopropyl)pyridin-2(1H)-one |
| | hydrochloride (Preparation 73) |
| | Prep-HPLC-A; White solid (15 mg, 34%); LCMS m/z = 464.2 [M + H]⁺. |
| | ¹H NMR (400 MHz, MeOH-d₄) δ: 8.96 (s, 1 H), 8.58 (d, J = 6.0 Hz, 1 H), |
| | 8.46 (s, 1 H), 7.30 (d, J = 7.2 Hz, 1 H), 6.38-6.34 (m, 1 H), 5.81-5.72 (m, |
| | 1 H), 4.16 (s, 2 H), 3.46-3.41 (m, 1 H), 2.40-2.39 (m, 2 H), 2.27-2.26 |
| | (m, 2 H), 1.61 (d, J = 6.0 Hz, 6 H), 1.55 (s, 3 H), 1.51-1.46 (m, 1 H), 1.27- |
| | 1.25 (m, 1 H), 0.86-0.84 (m, 1 H), 0.83 (d, J = 6.4 Hz, 3 H). |
| 77 | 6-isopropoxy-2-(1-(methoxymethyl)-2-oxabicyclo[2.1.1]hexan-4-yl)-N- |
| | (1-((1S,2R)-2-methylcyclopropyl)-2-oxo-1,2-dihydropyridin-3-yl)-2H- |
| | pyrazolo[3,4-b]pyridine-5-carboxamide |
| | 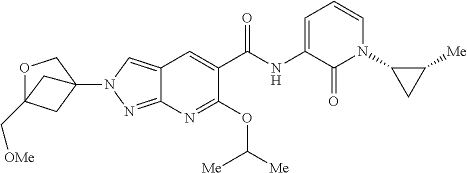 [SMILES: COCC12CC(n3cc4cc(C(=O)Nc5cccn([C@H]6C[C@H]6C)c5=O)c(OC(C)C)nc4n3)(CO1)C2] |
| | RCO₂H: 6-isopropoxy-2-(1-(methoxymethyl)-2- |
| | oxabicyclo[2.1.1]hexan-4-yl)-2H-pyrazolo[3,4-b]pyridine-5-carboxylic |
| | acid (Preparation 23) |
| | RNH₂: 3-amino-1-((1S,2R)-2-methylcyclopropyl)pyridin-2(1H)-one |
| | hydrochloride (Preparation 73) |
| | Prep-HPLC-A; White solid (15 mg, 30%); LCMS m/z = 464.2 [M + H]⁺. |
| | ¹H NMR (400 MHz, MeOH-d₄) δ: 8.99 (s, 1 H), 8.60 (d, J = 7.2 Hz, 1 H), |
| | 8.51 (s, 1 H), 7.32 (d, J = 6.0 Hz, 1 H), 6.40-6.36 (m, 1 H), 5.83-5.73 |
| | (m, 1 H), 4.20 (s, 2 H), 3.76 (s, 2 H), 3.46 (s, 1 H), 3.42 (s, 3 H), 2.51- |
| | 2.49 (m, 2 H), 2.34-2.32 (m, 2 H), 1.61 (d, J = 6.0 Hz, 6 H), 1.47-1.45 |
| | (m, 1 H), 1.30-1.26 (m, 1 H), 0.91-0.86 (m, 1 H), 0.83 (d, J = 6.4 Hz, 3 |
| | H). |
| 78 | 6-isopropoxy-2-(1-(methoxymethyl)-2-oxabicyclo[2.1.1]hexan-4-yl)-N- |
| | (1-((1S,2R)-2-methylcyclopropyl)-2-oxo-1,2-dihydropyridin-3-yl)-2H- |
| | indazole-5-carboxamide |
| | 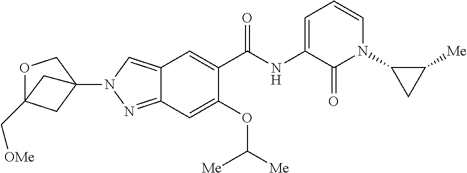 [SMILES: COCC12CC(n3cc4cc(C(=O)Nc5cccn([C@H]6C[C@H]6C)c5=O)c(OC(C)C)cc4n3)(CO1)C2] |
| | RCO₂H: 6-isopropoxy-2-(1-(methoxymethyl)-2- |
| | oxabicyclo[2.1.1]hexan-4-yl)-2H-indazole-5-carboxylic acid |
| | (Preparation 27) |
| | RNH₂: 3-amino-1-((1S,2R)-2-methylcyclopropyl)pyridin-2(1H)-one |
| | hydrochloride hydrochloride (Preparation 73) |
| | Prep-HPLC-B; White solid (20.9 mg, 37%); LCMS m/z = 493.1 [M + H]⁺. |
| | ¹H NMR (400 MHz, MeOH-d₄) δ: 8.30-8.40 (m, 2H), 8.51 (s, 1H), 7.31 |
| | (d, J = 6.4 Hz, 1H), 7.16 (s, 1H), 6.39 (t, J = 7.2 Hz, 1H), 5.00-4.90 (m, |
| | 1H), 4.21 (s, 2H), 3.76 (s, 2H), 3.50-3.40 (m, 4H), 2.60-2.50 (m, 2H), |
| | 2.40-2.30 (m, 2H), 1.65-1.60 (m, 6H), 1.50-1.45 (m, 1H), 1.30-1.20 (m, |
| | 2H), 0.84 (d, J = 6.4 Hz, 3H). |
| 79 | 2-(2-oxabicyclo[2.1.1]hexan-4-yl)-6-isopropoxy-N-(1-((1S,2R)-2- |
| | methylcyclopropyl)-2-oxo-1,2-dihydropyridin-3-yl)-2H-indazole-5- |
| | carboxamide |
| | 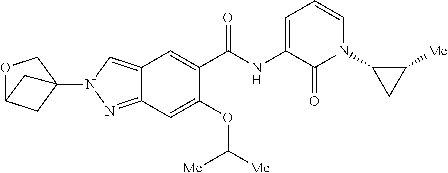 [SMILES: CC(C)Oc1cc2nn(C34COC(C3)C4)cc2cc1C(=O)Nc1cccn([C@H]2C[C@H]2C)c1=O] |
| | RCO₂H: 2-(2-oxabicyclo[2.1.1]hexan-4-yl)-6-isopropoxy-2H-indazole- |
| | 5-carboxylic acid (Preparation 87) |
| | RNH₂: 3-amino-1-((1S,2R)-2-methylcyclopropyl)pyridin-2(1H)-one |
| | hydrochloride (Preparation 73) |
| | Prep-HPLC-A; White solid (16 mg, 36%); LCMS m/z = 449.2 [M + H]⁺. |
| | ¹H NMR (400 MHz, MeOH-d₄) δ: 8.63-8.61 (m, 2H), 8.51 (s, 1H), 7.30 |
| | (d, J = 6.0 Hz, 1H), 7.16 (s, 1H), 6.40-6.37 (m, 1H), 5.01-4.92 (m, 1H), |
| | 4.70 (s, 1H), 4.13 (s, 2H), 3.47-3.42 (m, 1H), 2.54-2.53 (m, 2H), 2.33- |
| | 2.31 (m, 2H), 1.60 (d, J = 6.0 Hz, 6H), 1.50-1.48 (m, 1H), 1.46-1.42 (m, |
| | 1H), 0.91-0.86 (m, 1H), 0.84 (d, J = 6.4 Hz, 3H). |
| 80 | 2-(2-oxabicyclo[2.1.1]hexan-4-yl)-6-isopropoxy-N-(1-((1S,2R)-2- |
| | methylcyclopropyl)-2-oxo-1,2-dihydropyridin-3-yl)-2H-pyrazolo[3,4- |
| | b]pyridine-5-carboxamide |
| | 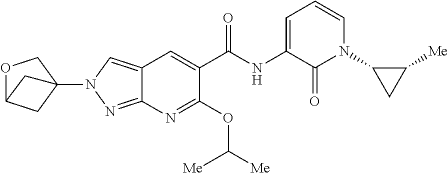 [SMILES: CC(C)Oc1nc2nn(C34COC(C3)C4)cc2cc1C(=O)Nc1cccn([C@H]2C[C@H]2C)c1=O] |
| | RCO₂H: 2-(2-oxabicyclo[2.1.1]hexan-4-yl)-6-isopropoxy-2H- |
| | pyrazolo[3,4-b]pyridine-5-carboxylic acid (Preparation 67) |
| | RNH₂: 3-amino-1-((1S,2R)-2-methylcyclopropyl)pyridin-2(1H)-one |
| | hydrochloride (Preparation 73) |
| | Prep-HPLC-B; White solid (21 mg, 35%); LCMS m/z = 450.2 [M + H]⁺. |
| | ¹H NMR (500 MHz, MeOH-d₄) δ: 9.01 (s, 1H), 8.62 (dd, J = 7.5, 1.5 Hz, |
| | 1H), 8.53 (s, 1H), 7.33 (dd, J = 7.0, 1.5 Hz, 1H), 6.39 (t, J = 7.0 Hz, 1H), |
| | 5.83-5.76 (m, 1H), 4.71 (s, 1H), 4.12 (s, 2H), 3.49-3.43 (m, 1H), 2.57- |
| | 2.50 (m, 2H), 2.35-2.28 (m, 2H), 1.62 (dd, J = 6.0, 4.5 Hz, 6H), 1.52-1.43 |
| | (m, 1H), 1.31-1.25 (m, 1H), 0.89-0.84 (m, 4H). |
| 81 | 2-(1-(fluoromethyl)-2-oxabicyclo[2.1.1]hexan-4-yl)-6-isopropoxy-N-(1- |
| | ((1R,2S)-2-methylcyclopropyl)-2-oxo-1,2-dihydropyridin-3-yl)-2H- |
| | indazole-5-carboxamide |
| | 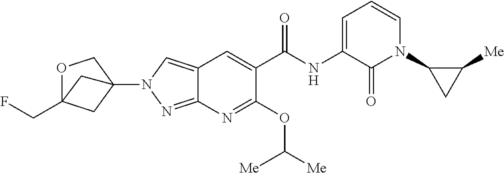 [SMILES: CC(C)Oc1nc2nn(C34COC(CF)(C3)C4)cc2cc1C(=O)Nc1cccn([C@@H]2C[C@@H]2C)c1=O] |
| | RCO₂H: 2-(1-(fluoromethyl)-2-oxabicyclo[2.1.1]hexan-4-yl)-6- |
| | isopropoxy-2H-indazole-5-carboxylic acid (Preparation 86) |
| | RNH₂: 3-amino-1-((1R,2S)-2-methylcyclopropyl)pyridin-2(1H)-one |
| | (Preparation 72) |
| | Prep-HPLC-B; White solid (35 mg, 60%); LCMS m/z = 481.2 [M + H]⁺. |
| | ¹H NMR (500 MHz, MeOH-d₄) δ: 8.64-8.61 (m, 2H), 8.53 (s, 1H), 7.32 |
| | (dd, J = 7.0, 1.5 Hz, 1H), 7.16 (s, 1H), 6.39 (t, J = 7.0 Hz, 1H), 5.00-4.94 |
| | (m, 1H), 4.79-4.68 (m, 2H), 4.25 (s, 2H), 3.47-3.42 (m, 1H), 2.58-2.54 |
| | (m, 2H), 2.43-2.38 (m, 2H), 1.60 (dd, J = 8.0, 6.0 Hz, 6H), 1.49-1.43 (m, |
| | 1H), 1.29-1.25 (m, 1H), 0.88-0.83 (m, 4H). |
| 82 | 2-(1-(fluoromethyl)-2-oxabicyclo[2.1.1]hexan-4-yl)-6-isopropoxy-N-(1- |
| | ((1S,2R)-2-methylcyclopropyl)-2-oxo-1,2-dihydropyridin-3-yl)-2H- |
| | indazole-5-carboxamide |
| | 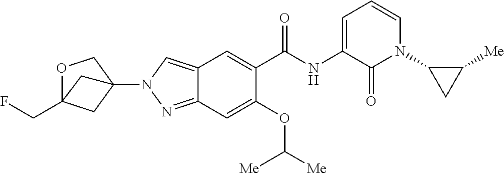 [SMILES: CC(C)Oc1cc2nn(C34COC(CF)(C3)C4)cc2cc1C(=O)Nc1cccn([C@H]2C[C@H]2C)c1=O] |
| | RCO₂H: 2-(1-(fluoromethyl)-2-oxabicyclo[2.1.1]hexan-4-yl)-6- |
| | isopropoxy-2H-indazole-5-carboxylic acid (Preparation 86) |
| | RNH₂: 3-amino-1-((1S,2R)-2-methylcyclopropyl)pyridin-2(1H)-one |
| | hydrochloride (Preparation 73). |
| | Prep-HPLC-B; White solid (26 mg, 45%); LCMS m/z = 481.2 [M + H]⁺. |
| | ¹H NMR (500 MHz, MeOH-d₄) δ: 8.64-8.61 (m, 2H), 8.53 (s, 1H), 7.32 |
| | (dd, J = 7.0, 1.5 Hz, 1H), 7.16 (s, 1H), 6.39 (t, J = 7.0 Hz, 1H), 5.00-4.94 |
| | (m, 1H), 4.79-4.68 (m, 2H), 4.25 (s, 2H), 3.47-3.42 (m, 1H), 2.58-2.54 |
| | (m, 2H), 2.43-2.38 (m, 2H), 1.60 (dd, J = 8.0, 6.0 Hz, 6H), 1.49-1.43 (m, |
| | 1H), 1.29-1.26 (m, 1H), 0.88-0.83 (m, 4H). |
| 83 | N-(1-((1R,2S)-2-fluorocyclopropyl)-2-oxo-1,2-dihydropyridin-3-yl)-6- |
| | isopropoxy-2-(1-methyl-2-oxabicyclo[2.1.1]hexan-4-yl)-2H- |
| | pyrazolo[3,4-b]pyridine-5-carboxamide |
| | 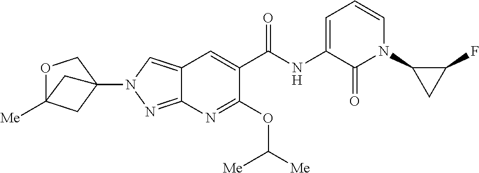 [SMILES: CC(C)Oc1nc2nn(C34COC(C)(C3)C4)cc2cc1C(=O)Nc1cccn([C@@H]2C[C@@H]2F)c1=O] |
| | RCO₂H: 6-isopropoxy-2-(1-methyl-2-oxabicyclo[2.1.1]hexan-4-yl)-2H- |
| | pyrazolo[3,4-b]pyridine-5-carboxylic acid (Preparation 6) |
| | RNH₂: 3-amino-1-((1S,2S)-2-fluorocyclopropyl)pyridin-2(1H)-one |
| | Prep-HPLC-B; White solid (54.8 mg, 74%); LCMS m/z = 468.1 [M + H]⁺. |
| | ¹H NMR (500 MHz, MeOH-d₄) δ: 8.99 (s, 1H), 8.60 (d, J = 6.0 Hz, 1H), |
| | 8.47 (s, 1H), 7.37 (d, J = 6.5 Hz, 1H), 6.38 (t, J = 7.0 Hz, 1H), 5.81-5.75 |
| | (m, 1H), 5.05-4.85 (m, 1H), 4.17 (s, 2H), 3.43-3.38 (m, 1H), 2.42-2.40 |
| | (m, 2H), 2.28-2.26 (m, 2H), 1.61 (d, J = 6.5 Hz, 6H), 1.56-1.52 (m, 5H). |
| 84 | N-(1-((1S,2R)-2-fluorocyclopropyl)-2-oxo-1,2-dihydropyridin-3-yl)-6- |
| | isopropoxy-2-(1-methyl-2-oxabicyclo[2.1.1]hexan-4-yl)-2H- |
| | pyrazolo[3,4-b]pyridine-5-carboxamide |
| | 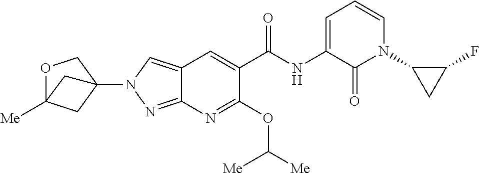 [SMILES: CC(C)Oc1nc2nn(C34COC(C)(C3)C4)cc2cc1C(=O)Nc1cccn([C@H]2C[C@H]2F)c1=O] |
| | RCO₂H: 6-isopropoxy-2-(1-methyl-2-oxabicyclo[2.1.1]hexan-4-yl)-2H- |
| | pyrazolo[3,4-b]pyridine-5-carboxylic acid (Preparation 6) |
| | RNH₂: 3-amino-1-((1S,2R)-2-fluorocyclopropyl)pyridin-2(1H)-one |
| | (Preparation 77). |
| | Prep-HPLC-B; White solid (66.2 mg, 90%); LCMS m/z = 468.2 [M + H]⁺. |
| | ¹H NMR (500 MHz, MeOH-d₄) δ: 8.99 (s, 1H), 8.60 (d, J = 6.0 Hz, 1H), |
| | 8.47 (s, 1H), 7.37 (d, J = 6.5 Hz, 1H), 6.39 (t, J = 7.0 Hz, 1H), 5.81-5.75 |
| | (m, 1H), 5.06-4.85 (m, 1H), 4.17 (s, 2H), 3.43-3.38 (m, 1H), 2.24-2.40 |
| | (m, 2H), 2.28-2.26 (m, 2H), 1.61 (d, J = 6.5 Hz, 6H), 1.56-1.52 (m, 5H). |
| 85 | 6-cyclopropoxy-N-(1-((1S,2R)-2-fluorocyclopropyl)-2-oxo-1,2- |
| | dihydropyridin-3-yl)-2-(1-methyl-2-oxabicyclo[2.1.1]hexan-4-yl)-2H- |
| | pyrazolo[3,4-b]pyridine-5-carboxamide |
| | 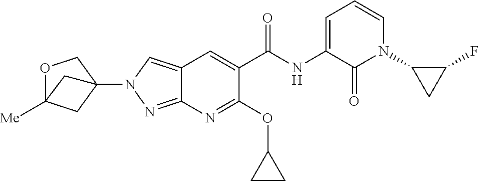 [SMILES: CC12CC(n3cc4cc(C(=O)Nc5cccn([C@H]6C[C@H]6F)c5=O)c(OC5CC5)nc4n3)(CO1)C2] |
| | RCO₂H: 6-cyclopropoxy-2-(1-methyl-2-oxabicyclo[2.1.1]hexan-4-yl)- |
| | 2H-pyrazolo[3,4-b]pyridine-5-carboxylic acid (Preparation 106) |
| | RNH₂: 3-amino-1-((1S,2R)-2-fluorocyclopropyl)pyridin-2(1H)-one |
| | (Preparation 77). |
| | Prep-HPLC-B; White solid (40.6 mg, 68%); LCMS m/z = 466.1 [M + H]⁺. |
| | ¹H NMR (400 MHz, MeOH-d₄) δ: 9.01 (s, 1H), 8.59 (dd, J = 7.6, 1.6 Hz, |
| | 1H), 8.52 (s, 1H), 7.38 (d, J = 6.0 Hz, 1H), 6.39 (t, J = 7.2 Hz, 1H), 5.08- |
| | 4.94 (m, 1H), 4.73-4.61 (m, 1H), 4.18 (s, 2H), 3.46-3.38 (m, 1H), 2.46- |
| | 2.39 (m, 2H), 2.30-2.27 (m, 2H), 1.59-1.51 (m, 5H), 1.26-1.21 (m, 2H), |
| | 0.97-0.91 (m, 2H). |
| 86 | 6-cyclopropoxy-N-(1-((1S,2R)-2-fluorocyclopropyl)-2-oxo-1,2- |
| | dihydropyridin-3-yl)-2-(1-(methoxymethyl)-2-oxabicyclo[2.1.1]hexan- |
| | 4-yl)-2H-pyrazolo[3,4-b]pyridine-5-carboxamide |
| | 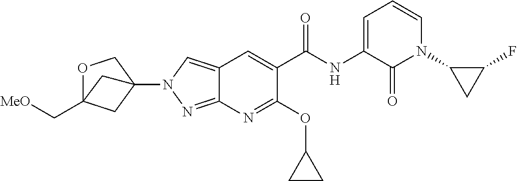 [SMILES: COCC12CC(n3cc4cc(C(=O)Nc5cccn([C@H]6C[C@H]6F)c5=O)c(OC5CC5)nc4n3)(CO1)C2] |
| | RCO₂H: 6-cyclopropoxy-2-(1-(methoxymethyl)-2- |
| | oxabicyclo[2.1.1]hexan-4-yl)-2H-pyrazolo[3,4-b]pyridine-5-carboxylic |
| | acid (Preparation 107) |
| | RNH₂: 3-amino-1-((1S,2R)-2-fluorocyclopropyl)pyridin-2(1H)-one |
| | (Preparation 77). |
| | Prep-HPLC-B; White solid (13.4 mg, 23%); LCMS m/z = 496.1 [M + H]⁺. |
| | ¹H NMR (500 MHz, MeOH-d₄) δ: = 9.02 (s, 1H), 8.60 (d, J = 6.0 Hz, |
| | 1H), 8.55 (s, 1H), 7.38 (d, J = 6.5 Hz, 1H), 6.40 (t, J = 6.40 (t, J = 7.0 Hz, 1H), 5.05- |
| | 4.93 (m, 1H), 4.73-4.70 (m, 1H), 4.22 (s, 2H), 3.77 (s, 2H), 3.45 (s, 3H), |
| | 3.43-3.40 (m, 1H), 2.53-2.50 (m, 2H), 2.36-2.34 (m, 2H), 1.59-1.52 (m, |
| | 2H), 1.24-1.22 (m, 2H), 0.96-0.93 (m, 2H). |
| 87 | 2-((1S,4S)-2-oxabicyclo[2.2.1]heptan-4-yl)-6-cyclopropoxy-N-(1- |
| | ((1S,2R)-2-fluorocyclopropyl)-2-oxo-1,2-dihydropyridin-3-yl)-2H- |
| | pyrazolo[3,4-b]pyridine-5-carboxamide |
| | 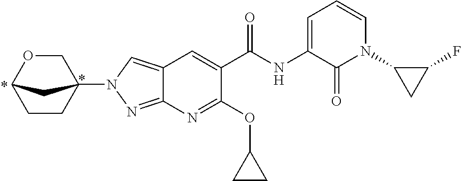 [SMILES: O=C(Nc1cccn([C@H]2C[C@H]2F)c1=O)c1cc2cn([C@@]34CC[C@@H](C3)OC4)nc2nc1OC1CC1] |
| | *absolute stereochemistry arbitrarily assigned |
| | RCO₂H: 2-((1S,4S)-2-oxabicyclo[2.2.1]heptan-4-yl)-6-cyclopropoxy- |
| | 2H-pyrazolo[3,4-b]pyridine-5-carboxylic acid (Preparation 112, |
| | stereochemistry arbitrarily assigned) |
| | RNH₂: 3-amino-1-((1S,2R)-2-fluorocyclopropyl)pyridin-2(1H)-one |
| | (Preparation 77). |
| | Prep-HPLC-B; White solid (14.7 mg, 25%); LCMS m/z = 466.1 [M + H]⁺. |
| | ¹H NMR (500 MHz, MeOH-d₄) δ: 9.02 (s, 1H), 8.59 (dd, J = 7.5, 1.5 Hz, |
| | 1H), 8.53 (s, 1H), 7.38 (d, J = 7.0 Hz, 1H), 6.39 (t, J = 7.0 Hz, 1H), 5.06- |
| | 4.92 (m, 1H), 4.73-4.69 (m, 1H), 4.54 (s, 1H), 4.15-4.13 (m, 1H), 4.03 |
| | (dd, J = 6.5 Hz, 3.5 Hz, 1H), 3.45-3.39 (m, 1H), 2.55-2.52 (m, 1H), 2.39- |
| | 2.34 (m, 2H), 2.29-2.27 (m, 1H), 2.11-2.08 (m, 1H), 2.09-2.01 (m, 1H), |
| | 1.59-1.51 (m, 2H), 1.26-1.21 (m, 2H), 0.97-0.92 (m, 2H). |
| 88 | 2-((1R,4R)-2-oxabicyclo[2.2.1]heptan-4-yl)-6-cyclopropoxy-N-(1- |
| | ((1S,2R)-2-fluorocyclopropyl)-2-oxo-1,2-dihydropyridin-3-yl)-2H- |
| | pyrazolo[3,4-b]pyridine-5-carboxamide |
| | 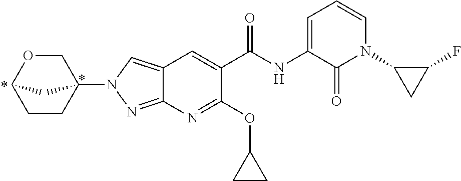 [SMILES: O=C(Nc1cccn([C@H]2C[C@H]2F)c1=O)c1cc2cn([C@]34CC[C@H](C3)OC4)nc2nc1OC1CC1] |
| | *absolute stereochemistry arbitrarily assigned |
| | RCO₂H: 2-((1R,4R)-2-oxabicyclo[2.2.1]heptan-4-yl)-6-cyclopropoxy- |
| | 2H-pyrazolo[3,4-b]pyridine-5-carboxylic acid (Preparation 113, |
| | stereochemistry arbitrarily assigned) |
| | RNH₂: 3-amino-1-((1S,2R)-2-fluorocyclopropyl)pyridin-2(1H)-one |
| | (Preparation 77). |
| | Prep-HPLC-B; White solid (10.1 mg, 17%); LCMS m/z = 466.1 [M + H]⁺. |
| | ¹H NMR (500 MHz, MeOH-d₄) δ: 9.02 (s, 1H), 8.59 (d, J = 7.5 Hz, 1H), |
| | 8.54 (s, 1H), 7.38 (d, J = 7.0 Hz, 1H), 6.40 (t, J = 7.5 Hz, 1H), 5.06-4.92 |
| | (m, 1H), 4.73-4.69 (m, 1H), 4.54 (s, 1H), 4.15-4.13 (m, 1H), 4.05-4.02 |
| | (m, 1H), 3.45-3.39 (m, 1H), 2.55-2.52 (m, 1H), 2.39-2.34 (m, 2H), 2.29- |
| | 2.25 (m, 1H), 2.10-2.06 (m, 1H), 2.05-2.01 (m, 1H), 1.59-1.51 (m, 2H), |
| | 1.26-1.21 (m, 2H), 0.67-0.82 (m, 2H). |
| 89 | 6-cyclopropoxy-2-(1-methyl-2-oxabicyclo[2.1.1]hexan-4-yl)-N-(1- |
| | ((1S,2R)-2-methylcyclopropyl)-2-oxo-1,2-dihydropyridin-3-yl)-2H- |
| | pyrazolo[3,4-b]pyridine-5-carboxamide |
| | 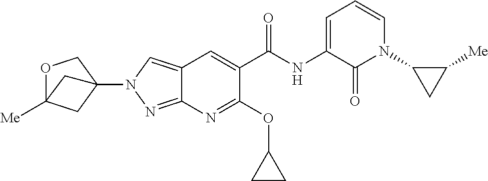 [SMILES: C[C@@H]1C[C@@H]1n1cccc(NC(=O)c2cc3cn(C45COC(C)(C4)C5)nc3nc2OC2CC2)c1=O] |
| | RCO₂H: 6-cyclopropoxy-2-(1-methyl-2-oxabicyclo[2.1.1]hexan-4-yl)- |
| | 2H-pyrazolo[3,4-b]pyridine-5-carboxylic acid (Preparation 106) |
| | RNH₂: 3-amino-1-((1S,2R)-2-methylcyclopropyl)pyridin-2(1H)-one |
| | hydrochloride (Preparation 73). |
| | Prep-HPLC-B; White solid (36 mg, 48%); LCMS m/z = 448.1 [M + H]⁺. |
| | ¹H NMR (500 MHz, MeOH-d₄) δ: 9.02 (s, 1H), 8.60 (d, J = 7.0 Hz, 1H), |
| | 8.55 (s, 1H), 7.32 (d, J = 6.0 Hz, 1H), 6.39 (t, J = 7.0 Hz, 1H), 4.73-4.70 |
| | (m, 2H), 4.13 (s, 2H), 3.48-3.43 (m, 1H), 2.55-2.52 (m, 2H), 2.33-2.31 |
| | (m, 1H), 1.51-1.44 (m, 1H), 1.30-1.25 (m, 2H), 1.23-1.16 (m, 2H), 0.98- |
| | 0.93 (m, 2H), 0.85 (d, J = 6.0 Hz, 3H). |
| 90 | 6-cyclopropoxy-2-(1-(methoxymethyl)-2-oxabicyclo[2.1.1]hexan-4-yl)- |
| | N-(1-((1S,2R)-2-methylcyclopropyl)-2-oxo-1,2-dihydropyridin-3-yl)- |
| | 2H-pyrido[3,4-b]pyridine-5-carboxamide |
| | 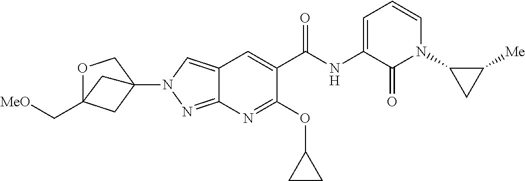 [SMILES: COCC12CC(n3cc4cc(C(=O)Nc5cccn([C@H]6C[C@H]6C)c5=O)c(OC5CC5)nc4n3)(CO1)C2] |
| | RCO₂H: 6-cyclopropoxy-2-(1-(methoxymethyl)-2- |
| | oxabicyclo[2.1.1]hexan-4-yl)-2H-pyrazolo[3,4-b]pyridine-5-carboxylic |
| | acid (Preparation 107) |
| | RNH₂: 3-amino-1-((1S,2R)-2-methylcyclopropyl)pyridin-2(1H)-one |
| | (Preparation 73). |
| | Prep-HPLC-B; White solid (33.8 mg, 48%); LCMS m/z = 492.1 [M + H]⁺. |
| | ¹H NMR (500 MHz, MeOH-d₄) δ: 9.01 (s, 1H), 8.59 (dd, J = 7.5, 2.0 Hz, |
| | 1H), 8.54 (s, 1H), 7.32 (dd, J = 7.0, 1.5 Hz, 1H), 6.38 (t, J = 7.5 Hz, 1H), |
| | 4.73-4.69 (m, 1H), 4.22 (s, 2H), 3.76 (s, 2H), 3.48-3.44 (m, 4H), 2.54- |
| | 2.48 (m, 2H), 2.37-2.33 (m, 2H), 1.51-1.45 (m, 1H), 1.30-1.26 (m, 1H), |
| | 1.24-1.20 (m, 2H), 0.98-0.92 (m, 2H), 0.88-0.86 (m, 1H), 0.84 (d, J = 6.5 |
| | Hz, 3H). |
| 91 | 2-(2-oxabicyclo[2.1.1]hexan-4-yl)-6-cyclopropoxy-N-(1-((1S,2R)-2- |
| | methylcycloproyl)-2-oxo-1,2-dihydropyridin-3-yl)-2H-pyrazolo[3,4- |
| | b]pyridine-5-carboxamide |
| | 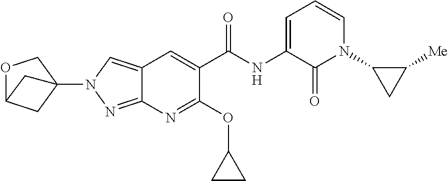 [SMILES: C[C@@H]1C[C@@H]1n1cccc(NC(=O)c2cc3cn(C45COC(C4)C5)nc3nc2OC2CC2)c1=O] |
| | RCO₂H: 2-(2-oxabicyclo[2.1.1]hexan-4-yl)-6-cyclopropoxy-2H- |
| | pyrazolo[3,4-b]pyridine-5-carboxylic acid (Preparation 108) |
| | RNH₂: 3-amino-1-((1S,2R)-2-methylcyclopropyl)pyridin-2(1H)-one |
| | (Preparation 73). |
| | Prep-HPLC-B; White solid (36 mg, 48%); LCMS m/z = 448.1 [M + H]⁺. |
| | ¹H NMR (500 MHz, MeOH-d₄) δ: 9.02 (s, 1H), 8.60 (d, J = 6.5 Hz, 1H), |
| | 8.55 (s, 1H), 7.32 (d, J = 6.0 Hz, 1H), 6.39 (t, J = 7.0 Hz, 1H), 4.73-4.70 |
| | (m, 2H), 4.13 (s, 2H), 3.48-3.43 (m, 1H), 2.55-2.53 (m, 2H), 2.33-2.31 |
| | (m, 2H), 1.51-1.44 (m, 1H), 1.30-1.25 (m, 2H), 1.24-1.17 (m, 2H), 0.98- |
| | 0.93 (m, 2H), 0.85 (d, J = 6.0 Hz, 3H). |
| 92 | 2-(2-oxabicyclo[2.1.1]hexan-4-yl)-6-cyclopropoxy-N-(1-((1S,2R)-2- |
| | methylcyclopropyl)-2-oxo-1,2-dihydropyridin-3-yl)-2H-pyrazolo[3,4- |
| | b]pyridine-5-carboxamide |
| | 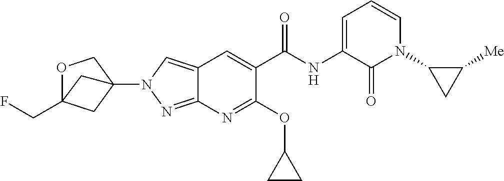 [SMILES: C[C@@H]1C[C@@H]1n1cccc(NC(=O)c2cc3cn(C45COC(CF)(C4)C5)nc3nc2OC2CC2)c1=O] |
| | RCO₂H: 6-cyclopropoxy-2-(1-(fluoromethyl)-2- |
| | oxabicyclo[2.1.1]hexan-4-yl)-2H-pyrazolo[3,4-b]pyridine-5-carboxylic |
| | acid (Preparation 110) |
| | RNH₂: 3-amino-1-((1S,2R)-2-methylcyclopropyl)pyridin-2(1H)-one |
| | (Preparation 73). |
| | Prep-HPLC-B; White solid (18 mg, 31%); LCMS m/z = 480.2 [M + H]⁺. |
| | ¹H NMR (400 MHz, MeOH-d₄) δ: 9.02 (s, 1H), 8.60 (dd, J = 7.6, 2.4 Hz, |
| | 1H), 8.57 (s, 1H), 7.32 (d, J = 6.8 Hz, 1H), 6.39 (t, J = 7.2 Hz, 1H), 4.80- |
| | 4.70 (m, 3H), 4.25 (s, 2H), 3.50-3.40 (m, 1H), 2.60-2.50 (m, 2H), 2.40- |
| | 2.30 (m, 2H), 1.50-1.40 (m, 1H), 1.30-1.20 (m, 4H), 1.00-0.90 (m, 2H), |
| | 0.84 (d, J = 6.0 Hz, 3H). |
| 93 | 2-((1S,4S)-2-oxabicyclo[2.2.1]heptan-4-yl)-6-cyclopropoxy-N-(1- |
| | ((1S,2R)-2-methylcyclopropyl)-2-oxo-1,2-dihydropyridin-3-yl)-2H- |
| | pyrazolo[3,4-b]pyridine-5-carboxamide |
| | 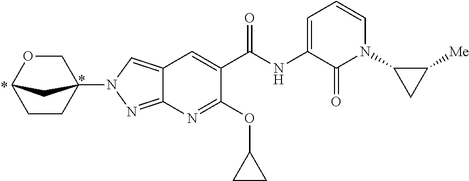 [SMILES: C[C@@H]1C[C@@H]1n1cccc(NC(=O)c2cc3cn([C@@]45CC[C@@H](C4)OC5)nc3nc2OC2CC2)c1=O] |
| | *absolute stereochemistry arbitrarily assigned |
| | RCO₂H: 2-((1S,4S)-2-oxabicyclo[2.2.1]heptan-4-yl)-6-cyclopropoxy- |
| | 2H-pyrazolo[3,4-b]pyridine-5-carboxylic acid (Preparation 112, |
| | stereochemistry arbitrarily assigned) |
| | RNH₂: 3-amino-1-((1S,2R)-2-methylcyclopropyl)pyridin-2(1H)-one |
| | (Preparation 73). |
| | Prep-HPLC-B; White solid (12.5 mg, 21%); LCMS m/z = 462.2 [M + H]⁺. |
| | ¹H NMR (400 MHz, MeOH-d₄) δ: 9.03 (s, 1H), 8.61 (m, J = 7.6 Hz, 1H), |
| | 8.55 (s, 1H), 7.34 (d, J = 7.6 Hz, 1H), 6.40 (t, J = 7.2 Hz, 1H), 4.80-4.70 |
| | (m, 1H), 4.60-4.50 (m, 1H), 4.15-4.00 (m, 2H), 3.50-3.40 (m, 1H), 2.60- |
| | 2.50 (m, 1H), 2.45-2.40 (m, 2H), 2.25-2.20 (m, 1H), 2.10-2.00 (m, 2H), |
| | 1.50-1.40 (m, 1H), 1.30-1.20 (m, 3H), 1.00-0.90 (m, 2H), 0.85-0.70 (m, |
| | 4H). |
| 94 | 2-((1S,4S)-2-oxabicyclo[2.2.1]heptan-4-yl)-6-cyclopropoxy-N-(1- |
| | ((1S,2R)-2-methylcyclopropyl)-2-oxo-1,2-dihydropyridin-3-yl)-2H- |
| | pyrazolo[3,4-b]pyridine-5-carboxamide |
| | 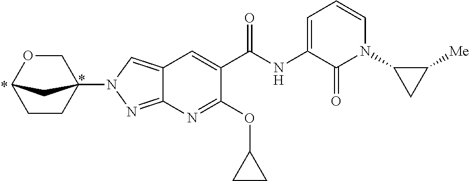 [SMILES: C[C@@H]1C[C@@H]1n1cccc(NC(=O)c2cc3cn([C@@]45CC[C@@H](C4)OC5)nc3nc2OC2CC2)c1=O] |
| | *absolute stereochemistry arbitrarily assigned |
| | RCO₂H: 2-((1S,4S)-2-oxabicyclo[2.2.1]heptan-4-yl)-6-cyclopropoxy- |
| | 2H-pyrazolo[3,4-b]pyridine-5-carboxylic acid (Preparation 112, |
| | stereochemistry arbitrarily assigned) |
| | RNH₂: 3-amino-1-((1S,2R)-2-methylcyclopropyl)pyridin-2(1H)-one |
| | (Preparation 73). |
| | Prep-HPLC-B; White solid (13.8 mg, 23%); LCMS m/z = 462.3 [M + H]⁺. |
| | ¹H NMR (400 MHz, MeOH-d₄) δ: 9.01 (s, 1H), 8.61 (m, J = 7.6 Hz, 1H), |
| | 8.55 (s, 1H), 7.33 (d, J = 7.6 Hz, 1H), 6.39 (t, J = 7.2 Hz, 1H), 4.80-4.70 |
| | (m, 1H), 4.60-4.50 (m, 1H), 4.15-4.00 (m, 2H), 3.50-3.40 (m, 1H), 2.60- |
| | 2.50 (m, 1H), 2.45-2.40 (m, 2H), 2.25-2.20 (m, 1H), 2.10-2.00 (m, 2H), |
| | 1.50-1.40 (m, 1H), 1.30-1.20 (m, 3H), 1.00-0.90 (m, 2H), 0.85-0.70 (m, |
| | 4H). |

### Example 95: 6-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide

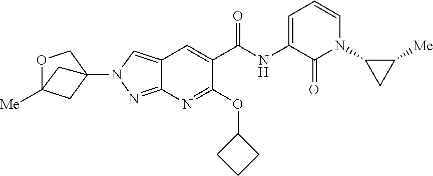 [SMILES: C[C@@H]1C[C@@H]1n1cccc(NC(=O)c2cc3cn(C45COC(C)(C4)C5)nc3nc2OC2CCC2)c1=O]

6-cyclobutoxy-2-(1-methyl-2-oxabicyclo[2.1.1]hexan-4-yl)-2H-pyrazolo[3,4-b]pyridine-5-carboxylic acid (Preparation 11, 51.9 mg, 0.158 mmol) was dissolved in DMF (1 mL) and HATU (59.9 mg, 0.158 mmol) and DIPEA (61.1 mg, 0.473 mmol) were added followed by 3-amino-1-((1S,2R)-2-methylcyclopropyl)pyridin-2(1H)-one (Preparation 73, 25.9 mg, 0.158 mmol) and the reaction stirred at rt overnight. The reaction was diluted with EtOAc and H₂O. The crude was purified by RPHPLC using a gradient of 5-70% ACN water in basic conditions to afford 6-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide. LCMS m/z=476.4 [M+H]⁺. ¹H NMR (600 MHz, DMSO-d₆) δ: 10.87 (s, 1H), 10.93-10.76 (m, 1H), 8.98-8.64 (m, 1H), 8.52-8.44 (m, 1H), 7.36-7.27 (m, 1H), 6.34-6.23 (m, 1H), 5.50-5.43 (m, 1H), 4.12-3.91 (m, 2H), 3.39-3.30 (m, 2H), 2.59-2.51 (m, 2H), 2.48-2.43 (m, 1H), 2.42-2.28 (m, 2H), 2.19-2.08 (m, 2H), 1.96-1.87 (m, 1H), 1.80-1.70 (m, 1H), 1.50-1.45 (m, 3H), 1.41-1.26 (m, 1H), 1.18-1.10 (m, 1H), 0.91-0.82 (m, 1H), 0.77-0.70 (m, 3H).

### Example 96: 6-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-indazole-5-carboxamide

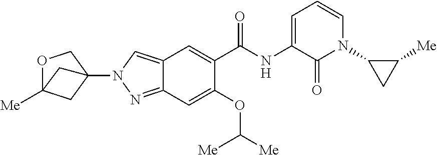 [SMILES: CC(C)Oc1cc2nn(C34COC(C)(C3)C4)cc2cc1C(=O)Nc1cccn([C@H]2C[C@H]2C)c1=O]

6-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-indazole-5-carboxamide (YIELD) was prepared from 6-isopropoxy-2-(1-methyl-2-oxabicyclo[2.1.1]hexan-4-yl)-2H-indazole-5-carboxylic acid (Preparation 49) and 3-amino-1-((1S,2R)-2-methylcyclopropyl)pyridin-2(1H)-one (Preparation 73) using an analogous method to that described for Example 95. Purification using Prep-HPLC-C. LCMS m/z=463.3 [M+H]⁺. ¹H NMR (600 MHz, DMSO-d₆) δ: 10.84 (s, 1H), 8.67-8.47 (m, 2H), 7.30 (d, J=7.27 Hz, 1H), 7.26 (s, 1H), 6.29 (t, J=7.27 Hz, 1H), 5.02-4.93 (m, 1H), 4.09 (s, 2H), 3.42-3.34 (m, 1H), 2.38 (d, J=4.36 Hz, 2H), 2.17 (d, J=4.36 Hz, 2H), 1.53-1.46 (m, 9H), 1.42-1.32 (m, 1H), 1.14 (q, J=8.48 Hz, 1H), 0.84 (q, J=5.57 Hz, 1H), 0.74 (d, J=6.54 Hz, 3H).

### Example 97: 6-isopropoxy-2-(1-methyl-2-oxabicyclo[2.1.1]hexan-4-yl)-N-(1-((1R,2S)-2-methylcyclopropyl)-2-oxo-1,2-dihydropyridin-yl)-2H-indazole-5-carboxamide

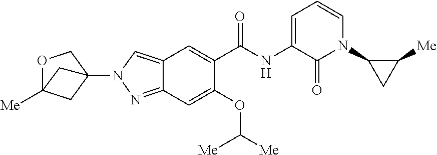 [SMILES: CC(C)Oc1cc2nn(C34COC(C)(C3)C4)cc2cc1C(=O)Nc1cccn([C@@H]2C[C@@H]2C)c1=O]

6-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)-2H-indazole-5-carboxamide (19 mg, 26%) was prepared from 6-isopropoxy-2-(1-methyl-2-oxabicyclo[2.1.1]hexan-4-yl)-2H-indazole-5-carboxylic acid (Preparation 49) and 3-amino-1-((1R,2S)-2-methylcyclopropyl)pyridin-2(1H)-one (Preparation 72) using an analogous method to that described for Example 96. Purification using Prep-HPLC-C. LCMS m/z=463.3 [M+H]⁺. ¹H NMR (600 MHz, DMSO-d₆) δ: 8.46-8.67 (m, 2H), 7.30 (br d, J=7.27 Hz, 1H), 7.26 (br s, 1H), 6.29 (t, J=7.27 Hz, 1H), 4.97 (td, J=5.90, 12.17 Hz, 1H), 4.09 (s, 2H), 3.44-3.36 (m, 2H), 2.38 (br d, J=3.63 Hz, 2H), 2.17 (d, J=4.36 Hz, 2H), 1.53-1.47 (m, 9H), 1.36 (td, J=6.99, 14.35 Hz, 1H), 1.14 (q, J=7.27 Hz, 1H), 0.83 (br d, J=5.09 Hz, 1H), 0.74 (d, J=6.54 Hz, 3H).

### Assays

Compounds of the present disclosure were assessed for their ability to inhibit IRAK4 activity. The inhibitory properties of the compounds of the disclosure described herein can be evidenced by testing in any one of the following assays.

### 1. Biochemical Assay

The 2-hour 1 mM ATP Biochemical Assay employed a MesoScale Detection (MSD) format. The kinase reaction was based on the IRAK4 phosphorylation of a biotin labeled peptide (IRAK1 activation loop sequence 360-389).

The kinase reaction in 30 μl was carried out in wells of a 384 well polypropylene assay plate, with 100 pM IRAK4, 1.6 μM of biotinylated peptide substrate and 1 mM ATP in 50 mM Hepes, pH 7.5, 60 mM NaCl, 5 mM MgCl₂, 0.25 mM MnCl₂, 2 mM DTT, 0.01% BSA, 0.01% BSA, and 1% DMSO (from compound DMSO stocks), for 2 hour at room temperature. The activity was quenched with 11 μl of 70 mM EDTA, pH 8.

To detect the phosphorylated biotinylated peptide substrate, 30 μl of the quenched reaction mixture was added to equivalent wells of a 384 well streptavidin coated MesoScale plate (Meso Scale Discovery #L21SA-1). After a 1 hour incubation of the plate for 1 hour at room temperature with gentle mixing, the plate wells were washed 3 times with 50 mM Tris, pH 7.5, 150 mM NaCl, 0.02% Tween-20.

A 25 μl volume of 1:500 anti-P-Threonine Rabbit polyclonal Antibody plus 1:500 Goat-anti-Rabbit Sulfo Tag Antibody (Meso Scale Discovery R³²AB-1) in 50 mM Tris, pH 7.5, 150 mM NaCl, 0.02% Tween-20 plus 2% BSA was then added to each well. After a 1-hour incubation of the plate for 1 hour at room temperature with gentle mixing, the plate wells were washed, 3 times with 50 mM Tris, pH 7.5, 150 mM NaCl, 0.02% Tween-20. A 40 μl volume of 2×MSD Read Buffer (Meso Scale Discovery R⁹²TC-1) was added to each well, and the plate was read immediately in an MSD Plate Reader (Meso Scale Discovery).

### 2. MDR1-MDCK Assay Procedure

Human MDR1 transfected MDCK cells (NIH cell line in-licensed from Absorption Systems) were used in the assay.
The compounds were tested at 1 μM concentration prepared in transport buffer (Hank's balanced salt solution with HEPES)
MDR1-MDCK cell were cultured for 7 days in 96 well transwell insert plates (Corning). Insert plates were washed before the assay and TEER (Trans epithelial electric resistance) was measured.
These plates were loaded with test compound solution 85 μL for A-B transport and 260 μL for B-A transport in the respective donor compartment. The volume of receiver buffer (Transport buffer supplemented with 1% BSA) in the respective receiver compartment was 250 and 75 μL.
10 μL samples was taken from donor compartment (T=0 timepoint)
Assay plates were incubated for 120 minutes.
At 120 minutes (T=120 timepoint) samples from respective donor (10 uL) and receiver (50 μL) compartments was taken.
After addition of 40 μL transport buffer with BSA to donor samples, crash solution (Acetonitrile with internal standard, 110 μL) was added to all samples.
After centrifugation 50 μL supernatant was transferred to separate plate and mixed with 50 μL water.
Samples were analyzed using LC-MS/MS coupled with high throughput injection system.
Analyte/internal standard area ratios were used for apparent permeability (Pₐₚₚ), efflux ratio and mass recovery estimation based on equations below.

$${P}_{app} = ( {dC}_{r} / dt ) \times {V}_{r} / ( A \times {C}_{E} )$$

$$Mass balance = 100 \times ( ( {V}_{r} \times {C}_{r}^{final} ) + ( {V}_{d} \times {C}_{d}^{final} ) ) / ( {V}_{d} \times {C}_{E} )$$

Where:
dCᵣ/dt is the cumulative concentration in the receiver compartment versus time in μM s⁻¹
Vᵣ is the volume of the receiver compartment in cm³
V_{d} is the volume of the donor compartment in cm³
A is the area of the insert (0.143 cm² for 96-well insert)
C_{E} is the estimated experimental concentration (Time=0) of the dosing solution Cᵣ^{final} is the concentration of the receiver at the end of the incubation period C_{d}^{final} is the concentration of the donor at the end of the incubation period.

### 3. Solubility Assay

### Sample Receipt and Preparation:

Samples received as 10 mM DMSO stock solutions for solubility analysis by Chemiluminescent Nitrogen Detection (CLND).
   Frozen on dry ice in 96 well plates
   Prior to setup: thaw, centrifuge, and sonicated in a water bath to facilitate dissolution.

### Buffer Preparation:

Potassium Phosphate Buffer, pH 6.8
   0.2M potassium phosphate, monobasic solution was prepared by dissolving 27.22 g/L of monobasic potassium phosphate in water
   62.5 mL of the 0.2M monobasic potassium phosphate solution was transferred to a 250 mL volumetric flask
   28 mL of 0.2N NaOH was added to the 250 mL volumetric flask
   Water was added to bring to volume
   Final pH was measured

### Kinetic Solubility Assay Setup:

Dilute the 10 mM DMSO stock solution 50-fold in buffer (2% DMSO) in 1 well of a Millipore solubility filter plate
   0.45 μm polycarbonate filter membrane
Seal the filter plate with heat sealing film
Incubate on a rotary shaker
   24 hours at ambient temperature
After incubation remove seal and vacuum filter, collecting filtrate
Seal collection plate containing filtrates for analysis.

### Kinetic Solubility Assay:

Inject filtrates into the nitrogen detector for quantification on Analiza's Automated Discovery Workstation (ADW).
Solubility results generated in μg/mL

### 4. Kpuu Assay

Generic Study Protocol for in vivo PK Studies (non-GLP)

### In Vivo

For the brain-to-plasma partition coefficient (Kp) evaluation, a dosing solution was intravenously infused into animals at a constant flow rate for 4 to 24 h. Blood samples were serially collected during infusion, and CSF and brain samples were harvested at the end of infusion.

For characterization of PK properties, a dosing solution was administered to animals via oral gavage or parenteral routes. Blood samples were collected after administration. Other biological samples, including tissue, bile, urine, and feces, can be collected during or at the end of the study if necessary.

All the animal experiments were conducted in accordance with the internally approved animal protocols.

### Bioanalysis

Tissue samples were typically homogenized in phosphate buffer saline (PBS) using a bead ruptor.

CSF samples were typically diluted with 8% BSA in PBS to prevent from non-specific binding. Artificial CSF (aCSF) is used as the surrogate matrix.

Dosing solutions were spiked into plasma for analysis when needed.

Calibration curves were prepared by spiking the analyte(s) into blank matrices, which were processed together with plasma, tissue homogenate and/or CSF samples by protein precipitation using a proper organic solvent (e.g. acetonitrile and methanol) containing generic analogue internal standards (e.g. verapamil, chrysin and glyburide). Matrix matching was used when analyzing multiple matrices in the same run. Samples above the upper limit of quantitation (ULOQ) needed to be diluted into the calibration range using either a pre-extraction or post-extraction dilution approach.

Processed samples were analyzed by LC-MS/MS using a proper method performing within the acceptable sensitivity, selectivity, precision and accuracy. For an analytical run to be accepted, over 75% of the calibration standards in the dual calibration curves needed to be within 20% of the nominal concentrations.

Compound- or study-specific bioanalytical methods that deviate from the typical procedure might be used when necessary, which will be documented in a study specific protocol included in the data upload.

### PK

Plasma concentrations were analyzed by non-compartmental analysis (NCA) using a “Linear up log down” fitting to generate basic PK parameters that include but are not limited to volume of distribution (Vd), maximal concentration (Cmax), time to reach maximal concentration (Tmax), area under the curve (AUC), half-life (t½), clearance (CL) and bioavailability (F). The PK parameters were normalized to the adjusted dose when dosing solution analysis was conducted.

Brain concentrations were compared against plasma concentrations at the corresponding timepoint for the calculation of partition coefficient (Kp).

Unbound drug partition coefficient (Kpuu), defined as the ratio of unbound drug partition across the blood-brain barrier, was calculated using the equation below:

$$Kpuu = \frac{{C}_{b} \times {F}_{ub}}{{C}_{p} \times {F}_{up}}$$

C_{b}: measured total drug concentration in brain
F_{ub}: unbound drug fraction in brain
Cₚ: measured total drug concentration in plasma
Fᵤₚ: unbound drug fraction in plasma
   Compound- or study-specific PK analysis that deviates from the typical procedure might be used when necessary, which will be documented in a study specific protocol included in the data upload.

### Determination of Fraction Unbound (Fu):

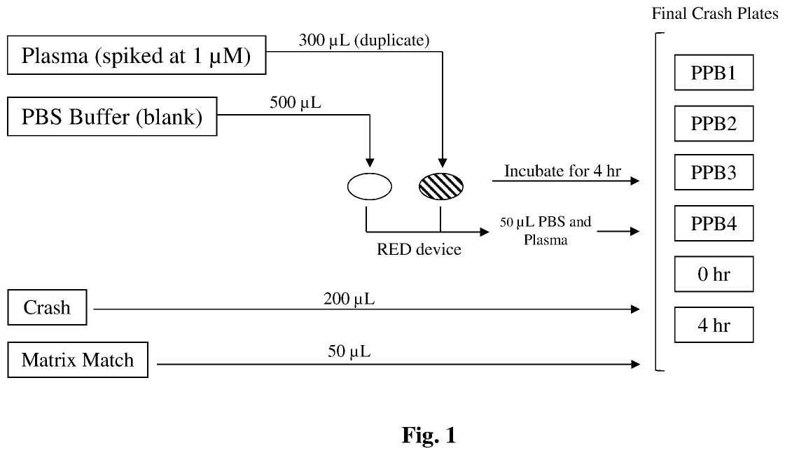

The unbound fraction of the test compound was determined based on the protocols described below.
1) Dilute initial 10 mM test article to 125 M by adding 5 μL to a total volume of 395 μL solvent solution (100% acetonitrile) in a 1 mL 96-well plate (Waters 186002481 Milford, MA). Ensure that the compounds are in solution.
2) Thaw frozen (rat, human, mouse, dog, and/or monkey) plasma (BIOIVT, Westbury, NY) in and warm the PBS buffer in a warm (37° C.) water bath.
   Dilute the 125 uM test article solutions by adding 8 μL to a final volume of 992 μL of plasma to make a final concentration of 1 μM in a 2 mL 96 well plate (Costar 3961). Mix thoroughly.
   This spiked plasma solution is shown in FIG. 1.
3) Prepare a chilled ‘crash’ solution of internal standard in a solvent solution.
   Pipette 200 μL of 25 ng/mL solution of internal standard, CPDPX (8-Cyclopentyl-1,3-dipropylxanthine, Sigma-Aldrich, C101) in 1:1 acetonitrile/methanol solvent solution into a 1 mL 96-well plate.
   Chill on ice or refrigerate at 4° C.
   This solution becomes the ‘Crash’ plate in FIG. 1.
4) From remaining spiked plasma, remove 50 μL (T=0 h) of each plasma sample and place into a crash plate which contains 200 μL. To matrix match, add 50 μL of blank buffer to the crashed sample (similar to PPB samples). Maintain remaining spiked plasma at 37° C. for 4 h time point
5) Transfer 500 μL of the warmed PBS buffer to the white side of the RED device (Thermo Scientific, Rockford IL, baseplate cat #89811, insert cat #89810) and 300 μL of the spiked plasma to the corresponding red ring side of the RED device.
6) Cover all the RED device plates with a lid and transfer them to a 37° C. incubator with 5% CO2 environment and shake at 200 rpm for 4 hours.
7) Reaction Termination after 4 hours:
   Add 50 μL of sample (plasma or buffer sample) and 50 μL of the opposite blank matrix (add blank buffer to the plasma samples and blank plasma to the buffer samples) to crash plates (same as above) to the crash plate containing 200 μL. Mix crash plate thoroughly.
   From remaining spiked plasma, remove 50 μL (T=4 h) of each plasma sample and place into a crash plate. To matrix match, add 50 μL of blank buffer to the crashed sample (similar to the protein binding samples).
   Centrifuge crash plate at 3900 rpm for 10 minutes at 4° C. (Eppendorf Centrifuge 5810R, Hamburg, Germany)
8) Sample preparation of LC/MS/MS:
   Transfer 30 μL of supernatant from the crash plates to 384-well plates containing 120 μL of 0.1 formic acid in 90:10 water:acetonitrile using the PPB 96 to 384 pretty method on the Tecan. Inject into the LC/MS.
   Volumes and diluent composition may be adjusted based on instrument (LC-MS/MS) sensitivity and test article sensitivity, solubility, and polarity to ensure adequate signal and retention of test articles within the linear limitation of the instrument.
9) Standard Curve
   Prepare standard curve of pooled test articles treated in a similar fashion as the reaction samples using plasma and buffer.
10) Data processing and analysis Multiquant will be chosen application used to process the data for PPB.

Equations:

### Equation 1. Calculation of % Free (% PPBunb)

% Free=(PAR of buffer side/PAR of plasma side)*100
PAR—Peak area ratio (PAR)

Fu=% Free/100
Fu=fraction unbound

### Equation 2. Final Calculation Using Dilution Factor (D)

This dilution factor formula is used only if tissue or plasma is diluted.

### 5. SPR Binding Assay

IRAK4 protein. N-terminal His-TEV-AVI tagged catalytical domain of human IRAK4 (a.a. 163-460) was co-expressed with Bir A in insect cells, and purified to >95% homogeneity by a combination of Ni-NTA affinity chromatography, ion-exchange and size-exclusion chromatography. Phosphorylation and mono-biotinylation of purified IRAK4 were confirmed by mass spectrometric analysis.

IRAK4 SPR. IRAK4 SPR was set up on Biacore T200 or S200 by using Biotin CAPture kit (Cytiva). In brief, purified IRAK4 in capture buffer (25 mM Hepes, 150 mM NaCl, 1 mM TCEP, pH7.4) was captured onto a CAP sensor surface via the interaction of biotin to streptavidin Typical capture level is between 1,000 RU to 2,000 RU, Compound binding kinetics to IRAK4 was examined with running buffer (25 mM Hepes, 150 mM NaCl, 1 mM TCEP, 2% DMSO, pH7.4). Serially diluted compounds were injected at 50 μl/min in single-cycle for 60-s association of each injection followed by 360-s dissociation at the end. The data was fitted to global 1:1 interaction model.

### Data for the Examples

| | | MDR1- | Solubility | | IRAK4 SPR |
|---|---|---|---|---|---|
| Example | IRAK4 | MDCK_Efflux | pH 6.8 | Brain | binding Half |
| number | IC₅₀ | Ratio | (ug/mL) | Kpu,u | Life (min) |
| 1 | 0.9 | | 70.95 | | |
| 2 | 0.4 | 1.58 | 51.7 | 0.9 | 52.85 |
| 3 | 0.5 | 1.59 | 7.9 | | |
| 4 | 0.4 | 6.36 | 42.97 | 0.4 | |
| 5 | 0.2 | 4.88 | 45.6 | 0.8 | |
| 6 | 0.4 | 1.59 | 5.57 | | |
| 7 | 0.7 | 1.43 | 10.28 | | |
| 8 | 0.3 | 2.57 | 15.87 | 0.7 | 54.4 |
| 9 | 0.3 | 3.26 | 18.7 | 0.8 | 80.4 |
| 10 | 0.2 | 3.4 | 22.68 | 0.6 | 73.4 |
| 11 | 0.2 | 4.8 | 36.01 | 0.9 | |
| 12 | 0.5 | 2.82 | 67.7 | 0.7 | |
| 13 | 0.7 | 3.41 | 19.8 | | |
| 14 | 1.4 | 2.27 | 43 | | |
| 15 | 0.5 | 1.4 | | 0.9 | |
| 16 | 0.2 | 2.09 | 44.6 | | |
| 17 | 0.2 | 2.6 | 50.4 | 0.5 | |
| 18 | 4.3 | 1.31 | | | |
| 19 | 6.6 | 1.91 | | | |
| 20 | 4.0 | 2.26 | | | |
| 21 | 1.1 | 3.33 | | | |
| 22 | 27.0 | 1.03 | | | |
| 23 | 1.3 | 1.15 | | | |
| 24 | 0.4 | 2.53 | 5.53 | | |
| 25 | 0.3 | 2.71 | 29.2 | | |
| 26 | 7.3 | 4.05 | 1.6 | | |
| 27 | 0.1 | 5.38 | 1.2 | 0.7 | |
| 28 | 0.4 | 4.72 | 2.7 | | |
| 29 | 0.2 | 2.79 | 74.2 | | |
| 30 | 0.2 | 2.58 | | 0.6 | |
| 31 | 0.2 | 2.5 | 61.8 | | |
| 32 | 0.2 | 4.18 | 16 | | 40.4 |
| 33 | 3.1 | 4.15 | 6.6 | | |
| 34 | 0.1 | 3.8 | 24.9 | 0.7 | 108 |
| 35 | 0.2 | | 65.7 | | 44.7 |
| 36 | 12.0 | 2.96 | 69.5 | | |
| 37 | 0.2 | 2.71 | 56 | | 186 |
| 38 | 0.3 | 1.29 | 1.7 | | |
| 39 | 0.5 | 1 | 2.3 | | |
| 40 | 0.4 | 1.32 | 19.89 | 0.8 | |
| 41 | 0.2 | 1.71 | 19.69 | 0.9 | 31.6 |
| 42 | 0.5 | 2.5 | 2.8 | | |
| 43 | 1.4 | 1.82 | 1.8 | | |
| 44 | 1.1 | 1.84 | 1.9 | | |
| 45 | 1.3 | 0.73 | 1.2 | | |
| 46 | 0.4 | 0.88 | 0.2 | | |
| 47 | 0.2 | 2.04 | 1.09 | 1.6 | 54 |
| 48 | 0.2 | 1.73 | 1.48 | | 58 |
| 49 | 0.1 | 9.67 | 62.3 | 0.4 | |
| 50 | 0.1 | 2.75 | 40.5 | 0.4 | 138 |
| 51 | 0.1 | 3.47 | 42.3 | 0.4 | 139 |
| 52 | 0.2 | 5.16 | 38.4 | 0.3 | 70.7 |
| 53 | 9.0 | 6.21 | | | |
| 54 | 0.6 | 1.77 | 66.5 | | |
| 55 | 0.8 | 3.71 | 60.5 | | |
| 56 | 0.8 | 3.31 | 63.9 | | |
| 57 | 1.7 | 2.39 | | | |
| 58 | 1.5 | 4.05 | 52.3 | | |
| 59 | 0.5 | 1.59 | 55.4 | 1.3 | |
| 60 | 0.5 | 1.75 | 52.9 | | |
| 61 | 0.5 | 1.43 | 51.5 | | |
| 62 | 0.6 | 1.6 | 56.1 | | |
| 63 | 0.5 | 5.06 | 58.8 | | |
| 64 | 0.4 | 4.35 | 58.9 | | 47.2 |
| 65 | 0.8 | 3.22 | 13 | | |
| 66 | 0.8 | 3.38 | 12.3 | | |
| 67 | 0.4 | 5.31 | 34 | | |
| 68 | 0.2 | 8.61 | 45.3 | 0.4 | |
| 69 | 0.5 | 1.74 | 8.2 | 0.7 | |
| 70 | 2.3 | 1.59 | | | |
| 71 | 22.2 | 1.8 | | | |
| 72 | 27.4 | 2.69 | | | |
| 73 | 43.2 | 1.51 | | | |
| 74 | 26.5 | 2.07 | | | |
| 75 | 36.9 | 3.63 | | | |
| 76 | 0.2 | 1.41 | 53 | 0.9 | 119 |
| 77 | 0.4 | 2.09 | 51 | 0.9 | |
| 78 | 0.7 | 2.05 | 36.1 | | |
| 79 | 0.8 | 2.56 | 55.8 | | |
| 80 | 0.5 | 4.42 | 32.4 | 1.0 | |
| 81 | 37.0 | 1.95 | | | |
| 82 | 0.7 | 2.17 | 33.8 | | |
| 83 | 2.6 | 4.48 | 16.7 | | |
| 84 | 0.4 | 3.06 | 15.8 | | |
| 85 | 0.2 | 7.21 | 20.1 | 0.3 | 48 |
| 86 | 0.2 | 18.74 | 13.9 | | 74 |
| 87 | 0.2 | 7.93 | 62.4 | | |
| 88 | 0.2 | 9.11 | 59.3 | 0.2 | |
| 89 | 0.9 | 2.28 | 38.1 | | |
| 90 | 1.1 | 5.39 | 65.2 | | |
| 91 | 1.1 | 5.09 | 46.7 | | |
| 92 | 1.7 | 5.77 | 12.8 | | |
| 93 | 0.5 | 2.5 | 47.6 | | |
| 94 | 0.6 | 2.73 | 14.7 | | |
| 95 | 0.8 | 1.09 | 48.3 | | |
| 96 | 0.2 | 1.4 | 13.6 | | |
| 97 | 83.4 | 2.25 | | | |

### COMPARATOR COMPOUNDS

### Comparator 1A: 6-isopropoxy-N-(1-methyl-2-oxo-1,2-dihydropyridin-3-yl)-2-(1-methyl-2-oxabicyclo[2.1.1]hexan-4-yl)-2H-indazole-5-carboxamide

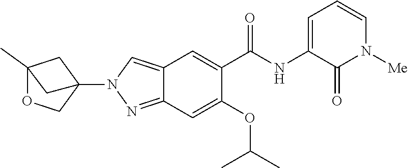 [SMILES: CC(C)Oc1cc2nn(C34COC(C)(C3)C4)cc2cc1C(=O)Nc1cccn(C)c1=O]

### Comparator 1B: 6-isopropoxy-N-(1-methyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide

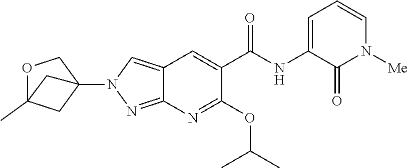 [SMILES: CC(C)Oc1nc2nn(C34COC(C)(C3)C4)cc2cc1C(=O)Nc1cccn(C)c1=O]

Title compound, 6-isopropoxy-N-(1-methyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide, was prepared from 6-isopropoxy-2-(1-methyl-2-oxabicyclo[2.1.1]hexan-4-yl)-2H-pyrazolo [3,4-b]pyridine-5-carboxylic acid [preparation 6] and 3-amino-1-methylpyridin-2(1H)-one in a manner similar to that described for Example 9. LCMS (ESI) m/z 424.2 [M+H]⁺. ¹H NMR (500 MHz, METHANOL-d₄) δ ppm=9.00 (s, 1H), 8.60 (d, J=7.5 Hz, 1H), 8.48 (s, 1H), 7.35 (d, J=6.5 Hz, 1H), 6.38 (t, J=7.0 Hz, 1H), 5.79 (t, J=6.0 Hz, 1H), 4.17 (s, 2H), 3.68-3.65 (m, 1H), 3.67-3.65 (m, 1H), 3.66 (s, 1H), 2.41 (d, J=4.5 Hz, 2H), 2.28 (d, J=4.5 Hz, 2H), 1.61 (d, J=6.5 Hz, 6H), 1.56 (s, 3H).

### Comparator 1C: 6-isopropoxy-N-(1-methyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide

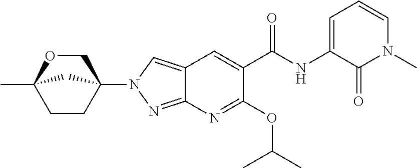

Title compound 6-isopropoxy-N-(1-methyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide was prepared from 6-isopropoxy-2-(1-methyl-2-oxabicyclo[2.2.1]heptan-4-yl)-2H-pyrazolo[3,4-b]pyridine-5-carboxylic acid [preparation 71] and 3-amino-1-methylpyridin-2(1H)-one in a manner similar to that described in Examples 40 and 41. The racemic product 6-isopropoxy-N-(1-methyl-2-oxo-1,2-dihydropyridin-3-yl)-2-(1-methyl-2-oxabicyclo[2.2.1]heptan-4-yl)-2H-pyrazolo[3,4-b]pyridine-5-carboxamide was purified by prep-SFC (Column: Phenomenex-Cellulose-2 (250 mm×30 mm, 10 um); Mobile Phase: from 45% to 45% of 0.1% NH₃H₂O ETOH; Flow Rate (ml/min): 80; Column temp: 35° C.) to give Peak 1, Comparator 1C, 6-isopropoxy-N-(1-methyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide, stereochemistry arbitrarily assigned (24 mg, 40% yield, >99% ee) as a white solid. LCMS (ESI) m/z 438.1 [M+H]⁺. ¹H NMR (500 MHz, CDCl₃) δ: 11.03 (brs, 1H), 9.00 (s, 1H), 8.64 (dd, J₁=7.5 Hz, J₂=1.5 Hz, 1H), 8.01 (s, 1H), 7.02 (dd, J₁=6.5 Hz, J₂=1.5 Hz, 1H), 6.25 (t, J=7.0 Hz, 1H), 5.97-5.91 (m, 1H), 4.24-4.18 (m, 2H), 3.65 (s, 3H), 2.50-2.46 (m, 1H), 2.44-2.40 (m, 1H), 2.36-2.31 (m, 1H), 2.29 (d, J=9.0 Hz, 1H), 2.07-1.95 (m, 2H), 1.63 (d, J=6.5 Hz, 6H), 1.51 (s, 3H).

Peak 2 obtained from the purification was 6-isopropoxy-N-(1-methyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide, stereochemistry arbitrarily assigned (25.4 mg, 42% yield, >99% ee) as a white solid. LCMS (ESI) m/z 438.0 [M+H]⁺. ¹H NMR (500 MHz, CDCl₃) δ:11.03 (brs, 1H), 9.00 (s, 1H), 8.64 (dd, J₁=7.5 Hz, J₂=1.5 Hz, 1H), 8.01 (s, 1H), 7.02 (dd, J₁=6.5 Hz, J₂=1.5 Hz, 1H), 6.25 (t, J=7.0 Hz, 1H), 5.97-5.91 (m, 1H), 4.24-4.18 (m, 2H), 3.65 (s, 3H), 2.50-2.46 (m, 1H), 2.44-2.40 (m, 1H), 2.37-2.30 (m, 1H), 2.29 (d, J=9.5 Hz, 1H), 2.06-1.95 (m, 2H), 1.63 (d, J=6.5 Hz, 6H), 1.51 (s, 3H).

| | IRAK4 | MDR1- | | | |
|---|---|---|---|---|---|
| | Bio- | MDCK | pH 6.8 | | IRAK4 SPR |
| Example | chemical | Efflux | Solubility | Brain | binding Half |
| number | IC₅₀ (nM) | Ratio | (μg/mL) | Kpu,u | Life (min) |
| Comparator 1A | 1.2 | 1.2 | 60.0 | 1.0 | 9.0 |
| Comparator 1B | 0.5 | 1.6 | 41.6 | 1.1 | 18.0 |
| Comparator 1C | 0.4 | 1.2 | 44.3 | 1.3 | 27.1 |

What is claimed is:

1. A compound represented by Formula (I): 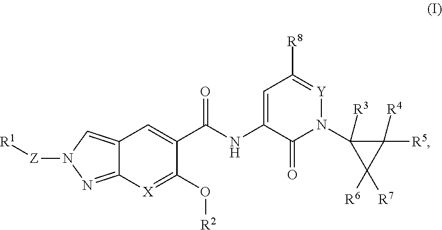
or a pharmaceutically acceptable salt thereof, wherein:
X is CH, CF or N;
Y is CH or N;
Z is ring A or —CH₂-ring A-*, wherein -* indicates the point of connection to R¹;
Ring A is 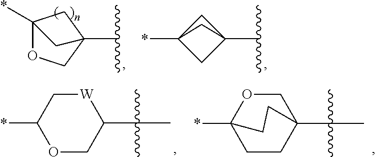
wherein n is 1 or 2; W is absent, CH₂ or O, and -* indicates the point of connection to R¹;
R¹ is H, —CN, C₁₋₃alkoxy or C₁₋₃alkyl optionally substituted with 1 to 3 substituents independently selected from halo and C₁-C₃alkoxy; or
R¹—Z is 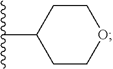 [SMILES: *C1CCOCC1]
R² is C₃₋₆cycloalkyl or C₁₋₄alkyl, wherein the C₃₋₆cycloalkyl or C₁₋₄alkyl is optionally substituted with 1 to 3 halo; and
R³, R⁴, R⁵, R⁶ and R⁷ are each independently selected from H, halo, CN, C₁₋₄alkyl, C₁₋₄haloalkyl, C₁₋₄alkoxy, and C₁₋₄alkoxyC₁₋₄alkyl, or any two of R³, R⁴, R⁵, R⁶ and R⁷ together with the carbon atoms from which they are attached form a C₃₋₆cycloalkyl or a 4 to 6 membered heterocyclyl containing one or two heteroatoms independently selected O, N, and S; and
R⁸ is H or halo.

2. The compound of claim 1, or a pharmaceutically acceptable salt thereof, wherein X is CH or N.

3. (canceled)

4. The compound of claim 1, or a pharmaceutically acceptable salt thereof, wherein Y is CH or N.

5. (canceled)

6. The compound of claim 1, or a pharmaceutically acceptable salt thereof, wherein Z is ring A and ring A is

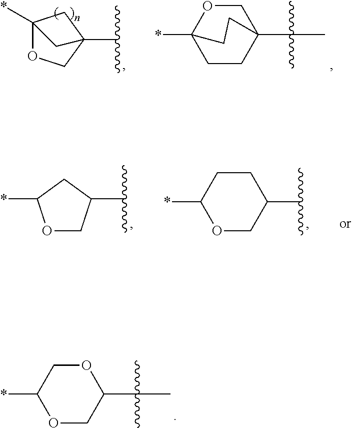

7.-8. (canceled)

9. The compound of claim 1, wherein the compound is represented by Formula (II), (III), (IV) (V), (IIA), (IIB), (IIIA), or (IIIB): 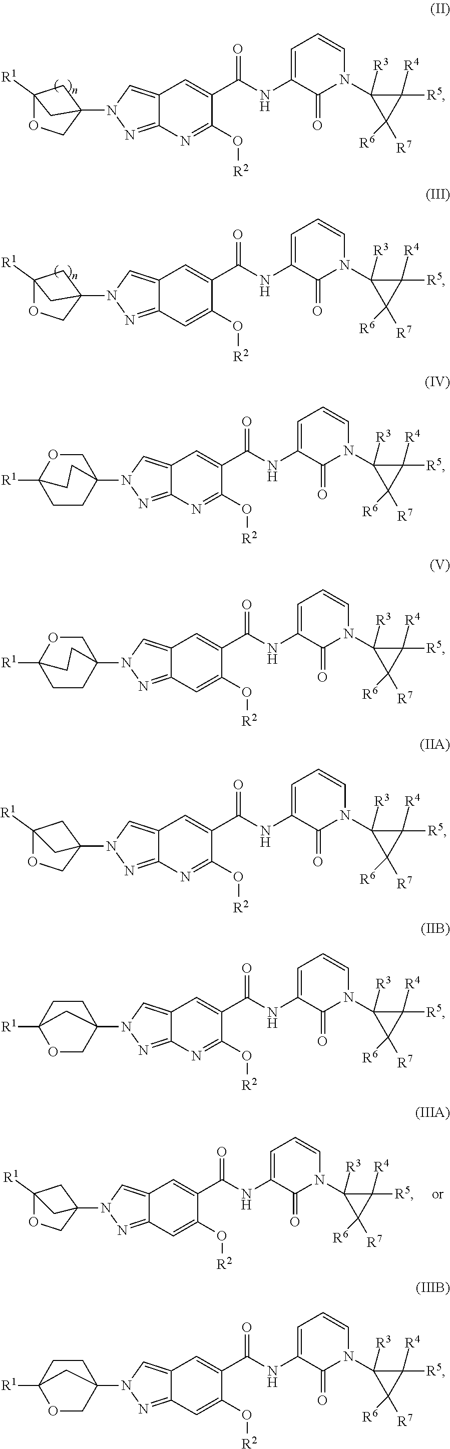
or a pharmaceutically acceptable salt thereof.

10. (canceled)

11. The compound of claim 9, or a pharmaceutically acceptable salt thereof, wherein R¹ is H or C₁₋₃alkyl optionally substituted with 1 to 3 substituents independently selected from halo and C₁-C₃alkoxy.

12.-13. (canceled)

14. The compound of claim 9, or a pharmaceutically acceptable salt thereof, wherein R¹ is H, —CH₃, —CH₂F, or —CH₂OCH₃.

15.-16. (canceled)

17. The compound of claim 9, or a pharmaceutically acceptable salt thereof, wherein R² is C₃₋₄alkyl or C₃₋₄cycloalkyl, wherein the C₃₋₄alkyl is optionally substituted with 1 to 3 fluoro.

18.-19. (canceled)

20. The compound of claim 9, or a pharmaceutically acceptable salt thereof, wherein R² is —CH(CH₃)₂, —CH(CH₃)CH₂CH₃, cycopropyl, or cyclobutyl.

21. (canceled)

22. The compound of claim 9, or a pharmaceutically acceptable salt thereof, wherein:
R¹ is H or C₁₋₃alkyl optionally substituted with 1 to 3 substituents independently selected from halo or C₁-C₃alkoxy; and
R² is C₃₋₄alkyl.

23. The compound of claim 9, or a pharmaceutically acceptable salt thereof, wherein R³, R⁴, R⁵, R⁶ and R⁷ are each independently selected from H, halo and C₁₋₃alkyl.

24. The compound of claim 9, or a pharmaceutically acceptable salt thereof, wherein R³, R⁴, R⁵, R⁶ and R⁷ are each independently selected from H, F, and —CH₃.

25.-26. (canceled)

27. The compound of claim 1, wherein the compound is represented by the following formula: 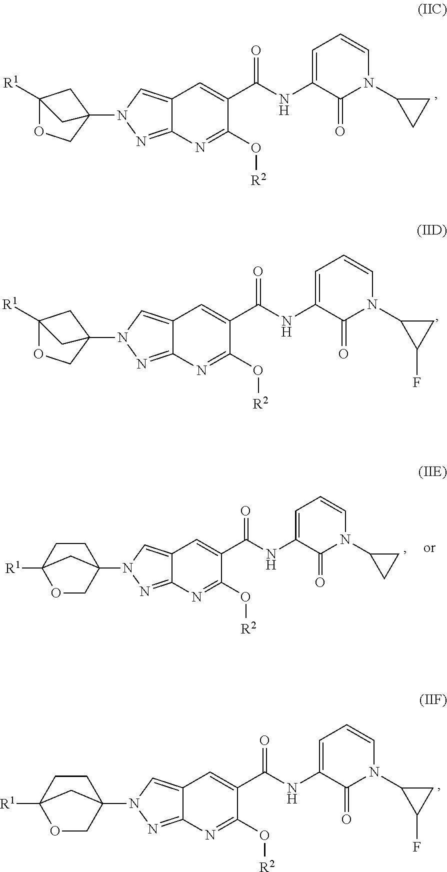
or a pharmaceutically acceptable salt thereof, wherein R¹ is C₁₋₃alkyl and R² is C₃₋₄alkyl.

28. The compound of claim 1, wherein the compound is;
(1) represented by the following formula: 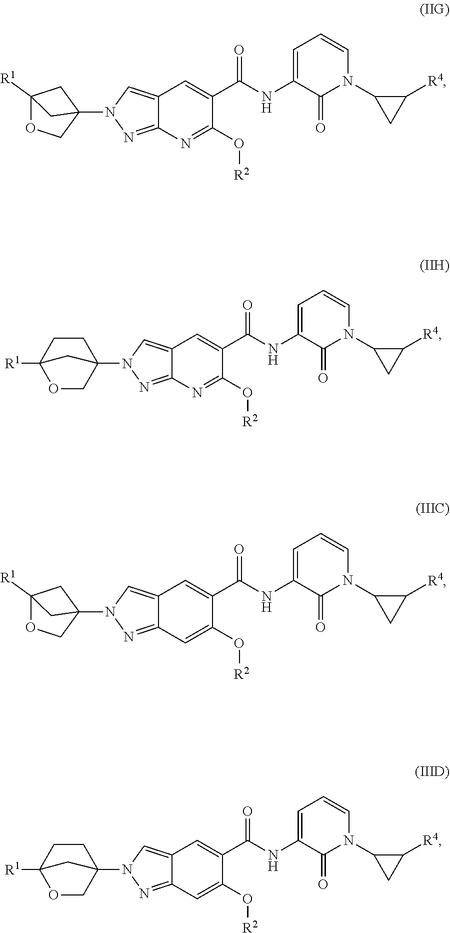
or a pharmaceutically acceptable salt thereof, wherein R¹ is C₁₋₃alkyl optionally substituted with 1 or 2 substituents independently selected from halo or C₁-C₃alkoxy; R² is C₃₋₄alkyl; and R⁴ is H, halo or C₁₋₃alkyl; or
(2) represented by the following formula: 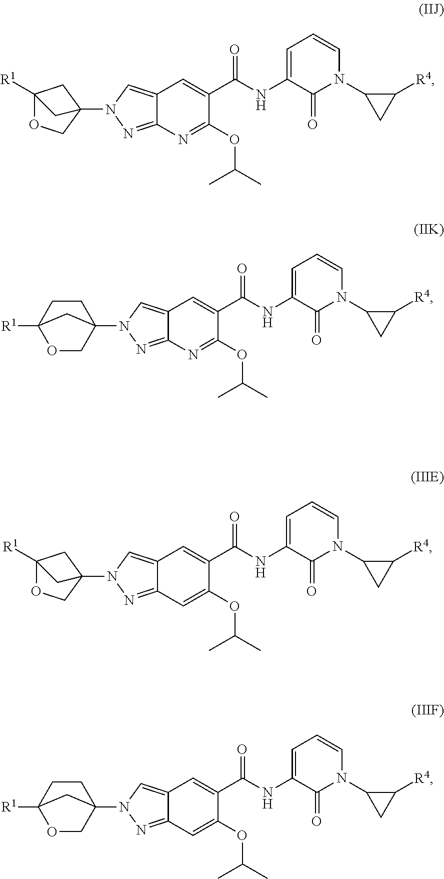
or a pharmaceutically acceptable salt thereof, wherein R¹ is C₁₋₃alkyl optionally substituted with 1 or 2 substituents independently selected from halo or C₁-C₃alkoxy, and R⁴ is H, halo or C₁₋₃alkyl.

29. (canceled)

30. The compound of 28 wherein R¹ is —CH₃, —CH₂F, or —CH₂OCH₃; and R⁴ is H, F, or —CH₃.

31. A compound according to claim 1, wherein said compound is selected from:
6-cyclobutoxy-N-(1-cyclopropyl-2-oxo-1,2-dihydropyridin-3-yl)-2-(1-methyl-2-oxabicyclo[2.1.1]hexan-4-yl)-2H-indazole-5-carboxamide;
N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide;
N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide;
(S)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide;
(R)—N-(1-cyclopropyl-2-oxo-1,2-dihydropyridin-3-yl)-6-isopropoxy-2-((tetrahydrofuran-3-yl)methyl)-2H-pyrazolo[3,4-b]pyridine-5-carboxamide;
6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-pyrazolo[3,4-b]pyridine-5-carboxamide;
6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-pyrazolo[3,4-b]pyridine-5-carboxamide;
N-(1-cyclopropyl-2-oxo-1,2-dihydropyridin-3-yl)-6-isopropoxy-2-(1-methyl-2-oxabicyclo[2.1.1]hexan-4-yl)-2H-indazole-5-carboxamide;
N-(1-cyclopropyl-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(1-cyclopropyl-2-oxo-1,2-dihydropyridin-3-yl)-2-(1-(fluoromethyl)-2-oxabicyclo[2.1.1]hexan-4-yl)-6-isopropoxy-2H-pyrazolo[3,4-b]pyridine-5-carboxamide;
N-(1-cyclopropyl-2-oxo-1,2-dihydropyridin-3-yl)-2-(1-(fluoromethyl)-2-oxabicyclo[2.1.1]hexan-4-yl)-6-isopropoxy-2H-pyrazolo[3,4-b]pyridine-5-carboxamide;
N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide;
N-(1-cyclopropyl-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
(R)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
(S)-6-(sec-butoxy)-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
(S)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide;
(R)—N-(1-cyclopropyl-2-oxo-1,2-dihydropyridin-3-yl)-6-isopropoxy-2-(tetrahydro-2H-pyran-3-yl)-2H-pyrazolo[3,4-b]pyridine-5-carboxamide;
6-Isopropoxy-2-(1-methyl-2-oxabicyclo[2.1.1]hexan-4-yl)-N-(1-(1-methylcyclopropyl)-2-oxo-1,2-dihydropyridin-3-yl)-2H-pyrazolo[3,4-b]pyridine-5-carboxamide;
2-(1-(Fluoromethyl)-2-oxabicyclo[2.1.1]hexan-4-yl)-6-isopropoxy-N-(1-(1-methylcyclopropyl)-2-oxo-1,2-dihydropyridin-3-yl)-2H-pyrazolo[3,4-b]pyridine-5-carboxamide;
(R)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide;
(S)-6-(sec-butoxy)-N-(1-cyclopropyl-2-oxo-1,2-dihydropyridin-3-yl)-2-(1-(fluoromethyl)-2-oxabicyclo[2.1.1]hexan-4-yl)-2H-pyrazolo[3,4-b]pyridine-5-carboxamide;
(R)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
(S)—N-(1-(2,2-dimethylcyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
6-cyclobutoxy-N-(1-cyclopropyl-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
6-cyclobutoxy-N-(1-(cis-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
6-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
6-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
2-(2-oxabicyclo[2.1.1]hexan-4-yl)-N-(1-cyclopropyl-2-oxo-1,2-dihydropyridin-3-yl)-6-isopropoxy-2H-pyrazolo[3,4-b]pyridine-5-carboxamide;
N-(1-cyclopropyl-2-oxo-1,2-dihydropyridin-3-yl)-6-isopropoxy-2-(1-(methoxymethyl)-2-oxabicyclo[2.2.1]heptan-4-yl)-2H-pyrazolo[3,4-b]pyridine-5-carboxamide;
N-(1-cyclopropyl-2-oxo-1,2-dihydropyridin-3-yl)-6-isopropoxy-2-((1S,4S)-1-(methoxymethyl)-2-oxabicyclo[2.2.1]heptan-4-yl)-2H-pyrazolo[3,4-b]pyridine-5-carboxamide;
N-(1-cyclopropyl-2-oxo-1,2-dihydropyridin-3-yl)-6-isopropoxy-2-((1R,4R)-1-(methoxymethyl)-2-oxabicyclo[2.2.1]heptan-4-yl)-2H-pyrazolo[3,4-b]pyridine-5-carboxamide;
N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-[1-[(1R,2S)-2-fluorocyclopropyl]-2-oxo-3-pyridyl]-6-isopropoxy-2-(1-methyl-2-oxabicyclo[2.1.1]hexan-4-yl)pyrazolo[3,4-b]pyridine-5-carboxamide;
N-[1-[(1S,2R)-2-fluorocyclopropyl]-2-oxo-3-pyridyl]-6-isopropoxy-2-(1-methyl-2-oxabicyclo[2.1.1]hexan-4-yl)pyrazolo[3,4-b]pyridine-5-carboxamide;
N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide;
N-(1-((1R,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide;
N-(1-((1S,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide;
6-isopropoxy-2-(1-methyl-2-oxabicyclo[2.1.1]hexan-4-yl)-N-(1-(2-methylcyclopropyl)-2-oxo-1,2-dihydropyridin-3-yl)-2H-pyrazolo[3,4-b]pyridine-5-carboxamide;
6-isopropoxy-2-(1-methyl-2-oxabicyclo[2.1.1]hexan-4-yl)-N-(1-(2-methylcyclopropyl)-2-oxo-1,2-dihydropyridin-3-yl)-2H-indazole-5-carboxamide;
6-cyclobutoxy-N-(1-cyclopropyl-2-oxo-1,2-dihydropyridin-3-yl)-2-((1S,4S)-1-methyl-2-oxabicyclo[2.2.1]heptan-4-yl)-2H-indazole-5-carboxamide;
6-cyclobutoxy-N-(1-cyclopropyl-2-oxo-1,2-dihydropyridin-3-yl)-2-((1R,4R)-1-methyl-2-oxabicyclo[2.2.1]heptan-4-yl)-2H-indazole-5-carboxamide;
N-(1-(2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(1-((1S,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(1-((1R,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(2-cyclopropyl-3-oxo-2,3-dihydropyridazin-4-yl)-6-isopropoxy-2-(1-methyl-2-oxabicyclo[2.1.1]hexan-4-yl)-2H-indazole-5-carboxamide;
N-(2-cyclopropyl-3-oxo-2,3-dihydropyridazin-4-yl)-6-isopropoxy-2-(1-methyl-2-oxabicyclo[2.1.1]hexan-4-yl)-2H-pyrazolo[3,4-b]pyridine-5-carboxamide;
N-(1-cyclopropyl-2-oxo-1,2-dihydropyridin-3-yl)-6-isopropoxy-2-((1S,4S)-1-methyl-2-oxabicyclo[2.2.1]heptan-4-yl)-2H-pyrazolo[3,4-b]pyridine-5-carboxamide;
N-(1-cyclopropyl-2-oxo-1,2-dihydropyridin-3-yl)-6-isopropoxy-2-((1R,4R)-1-methyl-2-oxabicyclo[2.2.1]heptan-4-yl)-2H-pyrazolo[3,4-b]pyridine-5-carboxamide;
N-(1-((1S,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide;
N-(1-((1S,2R)-2-fluorocyclopropyl)-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide;
N-(1-((1S,2R)-2-fluorocyclopropyl)-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide;
N-(1-((1S,2R)-2-fluorocyclopropyl)-2-oxo-1,2-dihydropyridin-3-yl)-2-(1-(fluoromethyl)-2-oxabicyclo[2.1.1]hexan-4-yl)-6-isopropoxy-2H-indazole-5-carboxamide;
N-(1-((1R,2S)-2-fluorocyclopropyl)-2-oxo-1,2-dihydropyridin-3-yl)-2-(1-(fluoromethyl)-2-oxabicyclo[2.1.1]hexan-4-yl)-6-isopropoxy-2H-indazole-5-carboxamide;
N-(1-((1R,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-indazole-5-carboxamide;
N-(1-((1R,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
N-(1-((1S,2S)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
6-cyclobutoxy-N-(1-((1R,2R)-2-fluorocyclopropyl)-2-oxo-1,2-dihydropyridin-3-yl)-2-(1-(methoxymethyl)-2-oxabicyclo[2.1.1]hexan-4-yl)-2H-indazole-5-carboxamide;
6-cyclobutoxy-N-(1-((1S,2S)-2-fluorocyclopropyl)-2-oxo-1,2-dihydropyridin-3-yl)-2-(1-(methoxymethyl)-2-oxabicyclo[2.1.1]hexan-4-yl)-2H-indazole-5-carboxamide;
N-(1-((1R,2R)-2-fluorocyclopropyl)-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide;
N-(1-((1S,2S)-2-fluorocyclopropyl)-2-oxo-1,2-dihydropyridin-3-yl)-6-isopropoxy-2-((1S,4S)-1-methyl-2-oxabicyclo[2.2.1]heptan-4-yl)-2H-indazole-5-carboxamide;
N-(1-((1R,2R)-2-fluorocyclopropyl)-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide;
N-(1-((1S,2S)-2-fluorocyclopropyl)-2-oxo-1,2-dihydropyridin-3-yl)-6-isopropoxy-2-((1R,4R)-1-methyl-2-oxabicyclo[2.2.1]heptan-4-yl)-2H-indazole-5-carboxamide;
N-(1-((1R,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide;
N-(1-((1S,2S)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-pyrazolo[3,4-b]pyridine-5-carboxamide;
6-cyclobutoxy-N-(1-((1R,2R)-2-fluorocyclopropyl)-2-oxo-1,2-dihydropyridin-3-yl)-2-(1-(methoxymethyl)-2-oxabicyclo[2.1.1]hexan-4-yl)-2H-pyrazolo[3,4-b]pyridine-5-carboxamide;
6-cyclobutoxy-N-(1-((1S,2S)-2-fluorocyclopropyl)-2-oxo-1,2-dihydropyridin-3-yl)-2-(1-(methoxymethyl)-2-oxabicyclo[2.1.1]hexan-4-yl)-2H-pyrazolo[3,4-b]pyridine-5-carboxamide;
N-(1-((1S,2R)-2-fluorocyclopropyl)-2-oxo-1,2-dihydropyridin-3-yl)-6-isopropoxy-2-(1-(methoxymethyl)-2-oxabicyclo[2.1.1]hexan-4-yl)-2H-indazole-5-carboxamide;
2-(2-oxabicyclo[2.1.1]hexan-4-yl)-N-(1-((1S,2R)-2-fluorocyclopropyl)-2-oxo-1,2-dihydropyridin-3-yl)-6-isopropoxy-2H-indazole-5-carboxamide;
2-(1-(fluoromethyl)-2-oxabicyclo[2.1.1]hexan-4-yl)-6-isopropoxy-N-(1-((1R,2R)-2-methylcyclopropyl)-2-oxo-1,2-dihydropyridin-3-yl)-2H-indazole-5-carboxamide;
2-(1-(fluoromethyl)-2-oxabicyclo[2.1.1]hexan-4-yl)-6-isopropoxy-N-(1-((1S,2S)-2-methylcyclopropyl)-2-oxo-1,2-dihydropyridin-3-yl)-2H-indazole-5-carboxamide;
6-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)-2H-pyrazolo[3,4-b]pyridine-5-carboxamide;
6-isopropoxy-2-(1-(methoxymethyl)-2-oxabicyclo[2.1.1]hexan-4-yl)-N-(1-((1R,2S)-2-methylcyclopropyl)-2-oxo-1,2-dihydropyridin-3-yl)-2H-pyrazolo[3,4-b]pyridine-5-carboxamide;
6-isopropoxy-2-(1-(methoxymethyl)-2-oxabicyclo[2.1.1]hexan-4-yl)-N-(1-((1R,2S)-2-methylcyclopropyl)-2-oxo-1,2-dihydropyridin-3-yl)-2H-indazole-5-carboxamide;
2-(2-oxabicyclo[2.1.1]hexan-4-yl)-6-isopropoxy-N-(1-((1R,2S)-2-methylcyclopropyl)-2-oxo-1,2-dihydropyridin-3-yl)-2H-indazole-5-carboxamide;
2-(2-oxabicyclo[2.1.1]hexan-4-yl)-6-isopropoxy-N-(1-((1R,2S)-2-methylcyclopropyl)-2-oxo-1,2-dihydropyridin-3-yl)-2H-pyrazolo[3,4-b]pyridine-5-carboxamide;
6-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-isopropoxy-2-(1-(methoxymethyl)-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-isopropoxy-2-(1-(methoxymethyl)-2-oxabicyclo[2.1.1]hexan-4-yl)-N-(1-((1S,2R)-2-methylcyclopropyl)-2-oxo-1,2-dihydropyridin-3-yl)-2H-indazole-5-carboxamide;
2-(2-oxabicyclo[2.1.1]hexan-4-yl)-6-isopropoxy-N-(1-((1S,2R)-2-methylcyclopropyl)-2-oxo-1,2-dihydropyridin-3-yl)-2H-indazole-5-carboxamide;
2-(2-oxabicyclo[2.1.1]hexan-4-yl)-6-isopropoxy-N-(1-((1S,2R)-2-methylcyclopropyl)-2-oxo-1,2-dihydropyridin-3-yl)-2H-pyrazolo[3,4-b]pyridine-5-carboxamide;
2-(1-(fluoromethyl)-2-oxabicyclo[2.1.1]hexan-4-yl)-6-isopropoxy-N-(1-((1R,2S)-2-methylcyclopropyl)-2-oxo-1,2-dihydropyridin-3-yl)-2H-indazole-5-carboxamide;
2-(1-(fluoromethyl)-2-oxabicyclo[2.1.1]hexan-4-yl)-6-isopropoxy-N-(1-((1S,2R)-2-methylcyclopropyl)-2-oxo-1,2-dihydropyridin-3-yl)-2H-indazole-5-carboxamide;
N-(1-((1R,2S)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
N-(1-((1S,2R)-2-fluorocyclopropyl)-2-oxo-1,2-dihydropyridin-3-yl)-6-isopropoxy-2-(1-methyl-2-oxabicyclo[2.1.1]hexan-4-yl)-2H-pyrazolo[3,4-b]pyridine-5-carboxamide;
6-cyclopropoxy-N-(1-((1S,2R)-2-fluorocyclopropyl)-2-oxo-1,2-dihydropyridin-3-yl)-2-(1-methyl-2-oxabicyclo[2.1.1]hexan-4-yl)-2H-pyrazolo[3,4-b]pyridine-5-carboxamide;
6-cyclopropoxy-N-(1-((1S,2R)-2-fluorocyclopropyl)-2-oxo-1,2-dihydropyridin-3-yl)-2-(1-(methoxymethyl)-2-oxabicyclo[2.1.1]hexan-4-yl)-2H-pyrazolo[3,4-b]pyridine-5-carboxamide;
2-((1S,4S)-2-oxabicyclo[2.2.1]heptan-4-yl)-6-cyclopropoxy-N-(1-((1S,2R)-2-fluorocyclopropyl)-2-oxo-1,2-dihydropyridin-3-yl)-2H-pyrazolo[3,4-b]pyridine-5-carboxamide;
2-((1R,4R)-2-oxabicyclo[2.2.1]heptan-4-yl)-6-cyclopropoxy-N-(1-((1S,2R)-2-fluorocyclopropyl)-2-oxo-1,2-dihydropyridin-3-yl)-2H-pyrazolo[3,4-b]pyridine-5-carboxamide;
6-cyclopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-cyclopropoxy-2-(1-(methoxymethyl)-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
2-(2-oxabicyclo[2.1.1]hexan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-(2-oxabicyclo[2.1.1]hexan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-((1S,4S)-2-oxabicyclo[2.2.1]heptan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
2-((1S,4S)-2-oxabicyclo[2.2.1]heptan-4-yl)-6-cyclopropoxy-N-(1-((1S,2R)-2-methylcyclopropyl)-2-oxo-1,2-dihydropyridin-3-yl)-2H-pyrazolo[3,4-b]pyridine-5-carboxamide;
6-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-pyrazolo[3,4-b]pyridine-5-carboxamide;
6-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)-2H-indazole-5-carboxamide; and
6-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)-2H-indazole-5-carboxamide;
or a pharmaceutically acceptable salt thereof.

32. A pharmaceutical composition comprising a compound of claim 1, or a pharmaceutically acceptable salt thereof, and a pharmaceutically acceptable carrier.

33. (canceled)

34. A method of treating an IRAK4 mediated disease in a subject comprising administering to the subject a compound or a pharmaceutically acceptable salt thereof of claim 1.

35. The method of claim 34, wherein the IRAK4 mediated disease is selected from the group consisting from ophthalmology, uveitis, rheumatoid arthritis, psoriatic arthritis, osteoarthritis, lupus, systemic lupus erythematosus, lupus nephritis, neuropsychiatric lupus, ankylosing spondylitis, osteoporosis, systemic sclerosis, multiple sclerosis, neuromyelitis optica, psoriasis, type I diabetes, type II diabetes, inflammatory bowel disease, Cronh's disease, ulcerative colitis, hyperimmunoglobulinemia D, periodic fever syndrome, Cryopyrin-associated periodic syndromes, Schnitzler's syndrome, systemic juvenile idiopathic arthritis, adult's onset Still's disease, gout, pseudogout, SAPHO syndrome, Castleman's disease, sepsis, stroke, atherosclerosis, celiac disease, deficiency of IL-1 receptor antagonist, Alzheimer's disease, Parkinson's disease, and cancer.

36.-37. (canceled)

38. The method of claim 37, wherein the IRAK4 mediated disease is selected from the group consisting from is selected from an autoimmune disease, an inflammatory disease, bone diseases, metabolic diseases, neurological and neurodegenerative diseases and/or disorders, cardiovascular diseases, allergies, asthma, hormone-related diseases, ischemic stroke, cerebral ischemia, hypoxia, traumatic brain injury, chronic traumatic encephalopathy, epilepsy, Parkinson's disease, and amyotrophic lateral sclerosis.
